(19) United States
(12) Patent Application Publication
(10) Pub. No.: US 2022/0290226 A1
(43) Pub. Date: Sep. 15, 2022

(54) MICROBIOME FINGERPRINTS, DIETARY FINGERPRINTS, AND MICROBIOME ANCESTRY, AND METHODS OF THEIR USE

(71) Applicant: Zoe Global Limited, London (GB)

(72) Inventors: Jonathan Thomas Wolf, London (GB); Nicola Segata, Trento (IT)

(73) Assignee: Zoe Global Limited, London (GB)

(21) Appl. No.: 17/203,678

(22) Filed: Mar. 16, 2021

Related U.S. Application Data
(60) Provisional application No. 62/992,740, filed on Mar. 20, 2020.
(60) Provisional application No. 63/048,959, filed on Jul. 7, 2020.

(51) Int. Cl. C12Q 1/6874; G16B 50/10; G16H 50/20; G16B 50/30; G16B 40/20; G16H 10/40; G16H 20/60; G16H 50/50

(52) U.S. Cl. CPC C12Q 1/6874; G16B 50/10; G16H 50/20; G16H 50/50; G16B 40/20; G16H 10/40; G16H 20/60; G16B 50/30

(57) ABSTRACT

A deep metagenomic sequencing of more than 1000 individual gut microbiomes, coupled with detailed long-term diet, fasting, and same-meal postprandial cardiometabolic blood markers analyses, is described. Strong associations between a set of microbes and specific nutrients, foods, food groups, and general dietary indices are demonstrated. Microbial biomarkers of obesity were reproducible across cohorts, but blood markers of cardiovascular disease and impaired glucose tolerance were more strongly associated with microbiome structures. Panels of intestinal microbial species associated with different conditions and/or habits are identified, enabling stratification of the gut microbiome into generalizable health levels among individuals even without clinically manifest disease.

### CROSS-REFERENCE TO RELATED APPLICATION

This application claims priority to the U.S. Provisional Application No. 62/992,740, filed on Mar. 20, 2020 and U.S. Provisional Application No. 63/048,959 filed Jul. 7, 2020. The disclosure of each of these earlier filed applications is hereby incorporated by reference in its entirety.

### FIELD OF THE DISCLOSURE

The present disclosure relates generally to microbiome analyses, as well as methods of modifying the microbiome of an individual, methods of diagnosis, and compositions based on such analyses.

### BACKGROUND OF THE DISCLOSURE

Dietary contributions to health, and particularly to long-term chronic conditions such as obesity, metabolic syndrome, and cardiac events, are of universal importance. This is especially true as obesity and associated mortality and morbidity have risen dramatically over the past decades and continue to do so worldwide. The reasons for this relatively rapid change have remained unclear, with the gut microbiome implicated as one of several potentially causal human-environmental interactions (Brown & Hazen, Nat. Rev. Microbiol. 16:171-181, 2018; Mozaffarian, Circulation 133:187-225, 2016; Musso et al., Annu. Rev. Med. 62, 361-380, 2011; Le Chatelier et al., Nature 500:541-546, 2013). Surprisingly, the details of the microbiome's role in obesity and cardiometabolic health have proven difficult to define reproducibly in large, diverse human populations—contrary to their behavior in mice—likely due to the complexity of habitual diets, the difficulty of measuring them at scale, and the highly personalized nature of the microbiome (Gilbert et al., Nat. Med. 24:392-400, 2018).

Today, individuals can measure a large number of health characteristics without having to go to a lab or clinic. For example, individuals may obtain an analysis of their microbiome by mailing a sample, collected at home, to a company for analysis. Generally, a microbiome analysis includes determining the composition and function of a community of microbes in a particular location, such as within the gut of an individual. A microbiome of the gut is made up of trillions of microorganisms, such as bacteria, and their genetic material that live in the intestinal tract, including bacteria, archaea or archaebacteria, viruses, and microeukaryotes.

These microorganisms appear to be an important part of digesting food, assisting with absorbing and synthesizing nutrients, regulating metabolism, body weight, and immune regulation, as well as contributing to regulating brain functions and mood. Microbiomes of different individuals, however, vary greatly. For instance, it is estimated that only ten to thirty percent of the bacterial species in a microbiome is common across different individuals. Much of this diversity of microbiomes remains unexplained, yet diet, environment, and host genetics appear to play a part. Determining how to utilize the results of the microbiome analysis, however, can be challenging.

Growing evidence also implicates the gut microbiome as a factor in the development of a number of disease processes, including inflammatory bowel diseases, atherosclerosis, obesity, diabetes, and colon cancer. The association of these disease processes with an altered microbial community structure suggests that interventions that restore the normal resilient gut microbial community might be an innovative intervention, as well as a way to influence overall health and wellness.

### SUMMARY OF THE DISCLOSURE

Described herein is the Personalized Responses to Dietary Composition Trial (PREDICT 1) observational and interventional study of diet-microbiome interactions in metabolic health. PREDICT 1 included over 1,000 participants in the United Kingdom (UK) and the United States (US) who were profiled pre- and post-standardized dietary challenges using a combination of intensive in-clinic biometric and blood measures, nutritionist-administered free-living dietary recall and logging, habitual dietary data collection, continuous glucose monitoring, and stool shotgun metagenomic sequencing. The study was inspired by and generally concordant with previous large-scale diet-microbiome interaction profiles, identifying both overall gut microbiome configurations and specific microbial taxa and functions associated with postprandial glucose responses (Zeevi et al., Cell 163:1079-1094, 2015; Mendes-Soares et al., Am. J. Clin. Nutr. 110, 63-75, 2019), obesity-associated biometrics such as body mass index (BMI) and adiposity (Falony et al., Science 352, 560-564, 2016; Zhernakova et al., Science 352, 565-569, 2016; Thingholm et al., Cell Host Microbe 26, 252-264.e10, 2019), and blood lipids and inflammatory markers (Schirmer et al., Cell 167:1897, 2016; Fu et al., Circ. Res. 117:817-824, 2015; Org et al., Genome Biol. 18:70, 2017). By combining PREDICT's extensive dietary and blood biomarker measures with high-precision microbiome analysis, these findings were able to extend to specific beneficial (e.g. Faecalibacterium prausnitzii) and detrimental (e.g. Ruminococcus gnavus) organisms, as well as to a highly-reproducible gut microbial signature of overall health that reproduced across multiple blood and dietary measures within PREDICT and in several previously published cohorts (Pasolli et al., Nat. Methods 14:1023-1024, 2017).

The current disclosure provides methods of using a group of microbes to determine a health condition in a human subject, wherein the group of microbes includes: at least two pro-health indicator microbes; or at least two poor health indicator microbes; or at least two pro-health indicator microbes and at least two poor health indicator microbes; wherein at least one of the pro-health indicator microbes is selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, and Paraprevotella xylaniphila; and wherein at least one of the poor health indicator microbes is selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, and Flavonifractor plautii. In another embodiment, at least one of the pro-health indicator microbes is selected from the group including Firmicutes bacterium CAG 95, Haemophilus parainfluenzae, Oscillibacter sp 57 20, Firmicutes bacterium CAG 170, Roseburia sp CAG 182, Clostridium sp CAG 167, Oscillibacter sp PC13, Eubacterium eligens, Prevotella copri, Veillonella dispar, Veillonella infantium, Faecalibacterium prausnitzii, Bifidobacterium animalis, Romboutsia ilealis, and Veillonella atypica; and at least one of the poor health indicator microbes is selected from the group including Clostridium leptum, Ruthenibacterium lactatiformans, Collinsella intestinalis, Escherichia coli, Blautia hydrogenotrophica, Clostridium sp CAG 58, Eggerthella lenta, Ruminococcus gnavus, Clostridium spiroforme, Clostridium bolteae CAG 59, Clostridium innocuum, Anaerotruncus colihominis, Clostridium symbiosum, Clostridium bolteae, and Flavonifractor plautii.

Another embodiment provides methods of predicting a health condition in a subject, the method including: determining presence, absence, or relative abundance of at least three pro-health indicator microbes in a microbiome of the subject; determining presence, absence, or relative abundance of at least three poor health indicator microbes in a microbiome of the subject; and predicting the health condition of the subject, based on the presence, absence, or relative abundance of the pro-health and/or poor health indicator microbes in the microbiome of the subject; wherein at least one of the pro-health indicator microbes is selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, and Paraprevotella xylaniphila; and wherein at least one of the poor health indicator microbes is selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, and Flavonifractor plautii. In another embodiment, at least one of the pro-health indicator microbes is selected from the group including Firmicutes bacterium CAG 95, Haemophilus parainfluenzae, Oscillibacter sp 57 20, Firmicutes bacterium CAG 170, Roseburia sp CAG 182, Clostridium sp CAG 167, Oscillibacter sp PC13, Eubacterium eligens, Prevotella copri, Veillonella dispar, Veillonella infantium, Faecalibacterium prausnitzii, Bifidobacterium animalis, Romboutsia ilealis, and Veillonella atypica; and at least one of the poor health indicator microbes is selected from the group including Clostridium leptum, Ruthenibacterium lactatiformans, Collinsella intestinalis, Escherichia coli, Blautia hydrogenotrophica, Clostridium sp CAG 58, Eggerthella lenta, Ruminococcus gnavus, Clostridium spiroforme, Clostridium bolteae CAG 59, Clostridium innocuum, Anaerotruncus colihominis, Clostridium symbiosum, Clostridium bolteae, and Flavonifractor plautii.

Also provided are methods to predict overall good or poor general health in a non-diseased human subject, which methods include: obtaining a microbiome sample from the human subject; isolating a nucleic acid fraction from the microbiome sample; detecting, within the nucleic acid fraction, presence, absence, or relative abundance of at least one unique marker sequence indicative of: a pro-health indicator microbe selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, and Paraprevotella xylaniphila; or a poor health indicator microbes selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, and Flavonifractor plautii; and at least one of predicting the human subject has overall good general health if the pro-health indicator microbes outnumber or are relatively more abundant than the poor-health indicator microbes; or predicting the human subject has overall poor general health if the poor health indicator microbes outnumber or are relatively more abundant than the pro-health indicator microbes. In another example of this embodiment, at least one of the pro-health indicator microbes is selected from the group including Firmicutes bacterium CAG 95, Haemophilus parainfluenzae, Oscillibacter sp 57 20, Firmicutes bacterium CAG 170, Roseburia sp CAG 182, Clostridium sp CAG 167, Oscillibacter sp PC13, Eubacterium eligens, Prevotella copri, Veillonella dispar, Veillonella infantium, Faecalibacterium prausnitzii, Bifidobacterium animalis, Romboutsia ilealis, and Veillonella atypica; and at least one of the poor health indicator microbes is selected from the group including Clostridium leptum, Ruthenibacterium lactatiformans, Collinsella intestinalis, Escherichia coli, Blautia hydrogenotrophica, Clostridium sp CAG 58, Eggerthella lenta, Ruminococcus gnavus, Clostridium spiroforme, Clostridium bolteae CAG 59, Clostridium innocuum, Anaerotruncus colihominis, Clostridium symbiosum, Clostridium bolteae, and Flavonifractor plautii.

This disclosure further provides an assay, which includes: subjecting nucleic acid extracted from a test sample of a human subject to a genotyping assay that detects at least one of Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica, the test sample including microbiota from a gut of the subject; determining a relative abundance of the at least one of Prevotella copri, Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica that is below a predetermined abundance; and selecting, when the relative abundance is below the predetermined abundance, a treatment regimen that includes at least one of: (i) modifying microbiota of the gut of the subject using at least one of a prebiotic, probiotic, or pharmaceutical, or (ii) altering the diet of the human subject.

Another embodiment is an assay, which includes: subjecting nucleic acid extracted from a test sample of a human subject to a genotyping assay that detects at least one of Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli, the test sample including microbiota from a gut of the subject; determining a relative abundance of the at least one Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli that is above a predetermined abundance; and selecting, when the relative abundance is above the predetermined abundance, a treatment regimen that includes at least one of: (i) modifying microbiota of the gut of the subject using at least one of a prebiotic, probiotic, or pharmaceutical, or (ii) altering the diet of the human subject.

Yet another embodiment is a method of diagnosing a human subject as having a healthy diet, including detecting in a microbiome sample from the subject the presence of Firmicutes CAG95 and/or the absence of Firmicutes CAG94.

Another embodiment is a method of diagnosing a human subject as having an unhealthy diet, including detecting in a microbiome sample from the subject the presence of Firmicutes CAG94 and/or the absence of Firmicutes CAG95.

Also described herein are microbial signatures (fingerprints) for good health, which include presence or relatively high abundance of at least three microbes selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica, and/or absence or relatively low abundance of at least three microbes selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli.

This disclosure also describes microbial signatures (fingerprints) for poor health, including absence or relatively low abundance of at least three microbes selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica, and/or presence or relatively high abundance of at least three microbes selected from the group including R. gnavus, F. plautii, C. innocuum, C. symbiosum, C. bolteae, A. colihominis, C. intestinalis, B. obeum, R. inulinivorans, E. ventriosum, B. hydrogenotrophica, Clostridium CAG 58, E. lenta, C. bolteae CAG 59, C. spiroforme, C. leptum, R. lactatiformans, and E. coli.

Another embodiment provides methods for targeting a microbiome of a human subject to promote health, which methods include: (A) detecting in a microbiome sample from the human subject one or more pro-health indicator microbes selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; and administering to the human a composition that increases growth or survival of the pro-health indicator microbe(s); and/or (B) detecting in a microbiome sample from the human subject one or more poor-health indicator microbe selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli; and administering to the human a composition that decreases growth or survival of the poor health indicator microbe(s).

Also described are probiotic compositions for ingestion by a human subject, which include at least one of Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica. Also provided are methods of altering abundance of one or more microbes in gut microflora of a subject, which including administering such a probiotic composition to the subject.

Yet another embodiment is a system to assay a biological condition in a subject, which system includes: a nucleic acid sample isolation device, which is adapted to isolate a nucleic acid sample from the subject; a sequencing device, which is connected to the nucleic acid sample isolation device and adapted to sequence the nucleic acid sample, thereby obtaining a sequencing result; and an alignment device, which is connected to the sequencing device and adapted to align the sequencing result against sequence from one or more of microbes in order to determine presence or absence of the microbe(s) based on the alignment result, wherein the microbes include one or more of: pro-health indicator microbes selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Osciffibacter sp PC13, Clostridium sp CAG 167, Ruminococcaceae bacterium D5, Paraprevotella xylaniphila, Faecalibacterium prausnitzii, Romboutsia ilealis, and Veillonella atypica; and/or poor health indicator microbes selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli.

### BRIEF DESCRIPTION OF THE DRAWINGS

FIG. 1 is a block diagram depicting an illustrative operating environment in which microbiome data is analyzed to generate microbiome fingerprints, dietary fingerprints, and microbiome ancestry for users.

FIG. 2 is a block diagram depicting an illustrative operating environment in which a data ingestion service receives, and processes test data associated with at home tests and sample collections.

FIG. 3 is a flow diagram showing a process illustrating aspects of a mechanism disclosed herein for obtaining and utilizing microbiome data for a user to generate microbiome fingerprints, dietary fingerprints, and microbiome ancestry for users.

FIG. 4 is a flow diagram showing a process illustrating aspects of a mechanism disclosed herein for generating a microbiome fingerprint for a user.

FIG. 5 is a flow diagram showing a process illustrating aspects of a mechanism disclosed herein for generating a dietary fingerprint for a user.

FIG. 6 is a flow diagram showing a process illustrating aspects of a mechanism disclosed herein for generating a microbiome ancestry for a user.

FIG. 7 is a flow diagram showing a process illustrating aspects of a mechanism disclosed herein for obtaining test data, including microbiome data, that may be utilized for generating microbiome fingerprints, dietary fingerprints, and microbiome ancestry for users.

FIG. 8 is a computer architecture diagram showing one illustrative computer hardware architecture for implementing a computing device that might be utilized to implement aspects of the various examples presented herein.

FIGS. 9A, 9B. The PREDICT 1 study associates gut microbiome structure with habitual diet and blood cardiometabolic markers. (FIG. 9A) The PREDICT 1 study assessed the gut microbiome of 1,098 volunteers from the UK and US via metagenomic sequencing of stool samples. Phenotypic data obtained through in-person assessment, blood/biospecimen collection, and the return of validated study questionnaires queried a range of relevant host/environmental factors including (1) personal characteristics, such as age, BMI, and estimated visceral fat; (2) habitual dietary intake using semi-quantitative food frequency questionnaires (FFQs); (3) fasting; and (4) postprandial cardiometabolic blood and inflammatory markers, total lipid and lipoprotein concentrations, lipoprotein particle sizes, apolipoproteins, derived metabolic risk scores, glycemic-mediated metabolites, and metabolites related to fatty acid metabolism. (FIG. 9B) Overall microbiome alpha diversity, estimated as the total number of confidently identified microbial species in a given sample (richness), was correlated with HDL-D (high-density lipoprotein density; positive) and estimated hepatic steatosis (negative). Up to ten strongest absolute Spearman correlations are reported for each category with q<0.05. Top species based on Shannon diversity are reported in FIG. 11A.

FIG. 10 Distributions of BMI in each curatedMetagenomicData dataset. The figure shows the distributions of BMI values for the datasets available in curatedMetagenomicData. This was used to further select those datasets with a comparable range of values (interquartile range between 3.5 and 7.5) as the one in the PREDICT 1 UK dataset (IQR of 5.5), to be used as validation datasets for the associations found. Along the X-axis (labeled “Dataset_name”), the dataset names are: A—“CosteaPI_2017” (Costea et al., Mol. Syst. Biol. 13:960, 2017), B—“DhakanDB_2019” (Dhakan et al., Gigascience 8, 2019), C—“FerrettiP_2018” (Ferretti et al., Cell Host & Microbe, 24(1), 133-145, 2018), D—“HansenLBS_2018” (Hansen et al., Nat. Commun. 9, 4630, 2018), E—“JieZ_2017” (Jie et al., Nat. Commun. 8, 845, 2017), F—“NielsenHB_2014” Nielsen et al., Nat. Biotechnol. 32, 822-828, 2014), G—“Obregon-TitoAJ_2015” (Obregon-Tito et al., Nature communications, 6 (1), 1-9, 2015), H—“RaymondF_2016” (Raymond et al., ISME J. 10(3):707-720, 2016), I—“SchirmerM_2016” (Schirmer et al., Cell 167, 1897, 2016), J—“YeZ_2018” (Ye et al., Microbiome 6(1):135, 2018), K—“ZellerG_2014” (Zeller et al., Mol. Syst. Biol. 10, 2014), and L—Zoe (described herein).

FIGS. 11A-11D Alpha diversity linked with personal factors, habitual diet, fasting, and postprandial markers. (FIG. 11A) Microbiome alpha diversity computed using the Shannon index correlated markers from the four categories: personal, habitual diet, fasting, and post-prandial. Reported are the top-ten strongest absolute Spearman correlations for each category with p<0.05. The y-axis reads (from top to bottom): ASCVD_10 yr_risk, person_md_age, person_clinic_bnni, ROE, PEACHES, BACON, WHOLEMEAL_BREAD, SPREAD_OLIVE_OIL, CEREAL_SUGAR_TOPPED, BROWN_RICE, KETCHUP, HFD, XL_HDL_L_0, LDL_size_0, IDL_L_0, L_HDL_L_0, HDL_size_0, IL-6_0, XXL_VLDL_L_0, VLDL_size_0, GlycA_0, MUFA_pct_0, IDL_L_360, XL_HDL_L_360, XS_VLDL_L_360, Total_C_360, HDL_size_360, and VLDL_size_360. (FIG. 11B) Inter-sample microbiome distances (beta-diversity) were substantially lower, i.e. closer, among samples from the same individuals (two weeks apart) compared to those amongst different individuals. Gut microbial communities in monozygotic twins were slightly more similar than in dizygotic twins (Mann-Whitney U test p=0.06), which, in turn, were more similar than unrelated individuals (p<1e-12), even after adjusting for age (p<1e-12). (FIG. 11C) After excluding twin status (i.e. non-twin, vs. mono vs. dizygotic twins) from the model, personal factors still accounted for the greatest proportion of variance explained in overall microbial diversity, followed by dietary habits, fasting and postprandial cardiometabolic blood markers (by cumulative stepwise dbRDA). (FIG. 11D) Cumulative distributions for each metadata variable based on Aitchison distance and Bray-Curtis dissimilarity are reported in FIGS. 13A-13C, 14A and 14B. The labels along the x-axis from left to right are: bristo_stool_score_average_last_3_months, FAw6.FA_0, person_clinic_weigth, XS.VLDL.C_360, abx_courses_last_12_months, bowel_movements_last_7_days, AcAce_0, person_md_age, visceral_fat, Healthy_PDI_Score_sum, maltose_g_kcal, starch_g_kcal, LDL.D_360, M.VLDL.C_360_rise, pulse, Meal_JJ_Hospital_meal_insulin_120_iacu, quicki_score, and cigarettes_a_day.

FIGS. 12A-1, 12A-2, 12B-12E, 12F-1, 12F-2. Food quality, regardless of source, is linked to overall and feature-level composition of the gut microbiome. (FIGS. 12A-1 & 12A-2) Specific components of habitual diet including foods, nutrients, and dietary indices are linked to the composition of the gut microbiome with variable strengths as estimated by machine learning regression and classification models. Boxplots report the correlation between the real value of each component and the value predicted by regression models across 100 training/testing folds (Methods). Circles denote median area-under-the-curve (AUC) values across 100 folds for a corresponding binary classifier between the highest and lowest quartiles (Methods). (FIG. 12B) The association between the gut microbiome and coffee consumption in UK participants is dose-dependent, i.e. stronger when assessing heavy (e.g. >4 cups/d) vs. never drinkers and was validated in the US cohort when applying the UK model. (FIG. 12C) Among general dietary patterns and indices, the Healthy Food Diversity index (HFD) and the (FIG. 12D) Alternate Mediterranean Diet score (aMED) were validated in the US cohort, thus showing consistency between the two populations on these two important dietary indices. Other validations of the UK model applied to the US cohort are reported in FIGS. 13A-13C. (FIG. 12E) Number of significant positive and negative associations (Spearman's correlation p<0.2) between foods and taxa categorized by more and less healthy plant-based foods and more and less healthy animal-based foods according to the PDI. Taxa shown are the 20 species with the highest total number of significant associations regardless of category. (FIGS. 12F-1 & 12F-2) Single Spearman correlations adjusted for BMI and age between microbial species and components of habitual diet with asterisks denoting significant associations (FDR q<0.2). The 30 microbial species with the highest number of significant associations across habitual diet categories are reported. All indices of dietary patterns are reported, whereas only food groups and nutrients (energy-adjusted) with at least 7 associations among the top 30 microbial species are reported. Full heatmaps of foods and unadjusted nutrients are reported in FIGS. 14A, 14B, and the full set of correlations is provided in Table 3. The species listed on the y-axis from top to bottom include: R. hominis, Roseburia CAG 182, A. butyriciproducens, A. hadrus, Clostridium CAG167, R. lactaris, Firmicutes CAG 95, E. eligens, Oscillibacter sp 57 20, H parainfluenzae, B. animalis, S. thermophilus, B. adolescentis, B. longum, C. leptum, B. bifidum, B. catenulatum, L. asaccharolyticus, Clostridium CAG 58, R. lactatiformans, C. innocuum, C. symbiosum, A. colihominis, F. plautii, P. merdae, Pseudofiavonifractor An184, Anaeromassilibacillus An250, Firmicutes CAG 94, C. saccharolyticum, and C. spiroforme. The x-axis from left to right reads: Meat, Desserts, Sugary drinks, Potatoes, Animal-based, Tea & coffee, Alcohol, Whole grain, Fruits, Legumes, Eggs, Vegetables, Nuts, Lactose, Maltose, Carbohydrates, Sucrose, Starch, Galactose, Vit. B2, Calcium, Vit. B12, Potassium, Phosphorus, Zinc, Selenium, Fructose, Vitamin B1, Folate, Vit. C, Carotene equiv., Beta-carotene, NSP, Manganese, Magnesium, Iron, Vit. E equiv., PUFAs, Copper, U-plant (n), U-plant (%), uPDI, Tot. plants (n), Tot. PDI, Tot. plant (%), H-plant (%), H-plant (n), aMED, hPDI, HEI, Animal soccer, and HFD. Positive Spearman correlation values are enclosed in dashed outline; asterisks indicate statistical significance.

FIGS. 13A-13C Top foods, food groups, nutrients, and dietary patterns validated in the PREDICT 1 US cohort. The application of the RF regression model trained on the PREDICT 1 UK cohort on the PREDICT 1 US participants, validating the associations with food-related variables found in the PREDICT 1 UK.

FIGS. 14A, 14B Species-level correlation with single foods. The figure shows the species-level correlations (Spearman) with single food quantities as estimated from the food frequency questionnaires. Only foods with at least 5 significant associations (q-value≤0.2) are displayed. Species are sorted by the number of significant associations, and the top 30 are reported in the figure. The species listed along the y-axis from top to bottom are: Bifidobacterium animalis, Haemophilus parainfluenzae, Firmicutes bacterium CAG 95, Oscillibacter sp 57 20, Ruminococcus lactaris, Oscillibacter sp PC13, Eubacterium eligens, Faecalibacterium prausnitzii, Agathobaculum butyriciproducens, Anaerostipes hadrus, Roseburia hominis, Roseburia sp CAG 182, Harryflintia acetispora, Clostridium saccharolyticum, Clostridium sp CAG 58, Clostridium spiroforme, Pseudofiavonifractor sp An184, Anaeromassilibacillus sp An250, Firmicutes bacterium CAG 94, Clostridium leptum, Bifidobacterium bifidum, Bifidobacterium catenulatum, Alistipes finegoldii, Ruthenibacterium lactatiformans, Clostridium bolteae, Anaerotruncus colihominis, Flavonifractor plautii, Eggerthella lenta, Clostridium innocuum, and Clostridium symbiosum.

FIGS. 15A-15D. Random forest machine learning models based on microbial or functional profiles are capable of predicting obesity phenotypic markers, even when tested against separate, independent cohorts. (FIG. 15A) Whole-microbiome machine learning models can assess personal factors with RF regression (boxplots and left-side vertical axis) using only taxonomic or functional (i.e. pathway) microbiome features. Classification models (circles and right-side vertical axis) exceed AUC 0.65 except for waist-to-hip ratio (WHR) and smoking. (FIG. 15B) The highest correlations were observed between the relative abundance of microbial species and age, BMI, and visceral fat. The link between microbial features and visceral fat was of greater effect and more often significant than with traditional BMI. (FIG. 15C) Using several independent datasets (Pasolli et al., Nat Methods, 14, 1023-1024, 2017) correlations were confirmed between single microbial species and BMI with blue points denoting significant associations at p<0.05. (FIG. 15D) The machine learning model for BMI trained on PREDICT 1 data is reproducible in several external datasets (FIG. 10), achieving correlations with true values exceeding those obtained in cross-validation of a single given dataset in five of seven cases. When the PREDICT 1 microbiome model is expanded to include other datasets (excluding those ones used for testing, i.e. leave-one-dataset-out/LODO approach) the performance remains comparable, affirming the generalizability of the PREDICT 1 model on obesity-related indicators.

FIGS. 16A-16H. Fasting and postprandial cardiometabolic responses to standardized test meals associated with the microbiome. (FIG. 16A) The strongest observed links according to correlation of the predicted versus collected measures between the gut microbiome and fasting metabolic blood markers. For measures of lipid concentration in lipoproteins, only the five strongest correlations were reported. Indices are grouped in nine distinct categories, and boxplots report the correlation between the prediction of RF regression models trained on microbial taxa or pathway abundances across 100 training/testing folds. Circles denote AUC values for RF classification, while stars report regressor performance when trained on the UK cohort and evaluated on the independent US validation cohort. (FIG. 16B) RF regression and classification performance in predicting postprandial metabolic responses for clinic Meal 1 (breakfast) measured as iAUC at 6 h for triglycerides (TG) and iAUC at 2 h for glucose, C-peptide, and insulin. (FIG. 16C) Glycemic-mediated postprandial iAUCs at 2 h for the other meals, and (FIG. 16D) glycemic-mediated markers absolute levels vs. rise. (FIG. 16E) Postprandial inflammatory measures (concentration and rise). (FIG. 16F) RF microbiome-based model performance with postprandial changes (concentrations and rise) in lipoprotein concentration, composition, and size. (FIG. 16G) Spearman's correlation for regression and classification of US validation studies. (FIG. 16H) Fasting and postprandial performance indices (correlation of the regressors' outputs) were more tightly linked to gut community structure than were their corresponding postprandial rises. (FIGS. 16B-16F) Performance of the microbiome-based ML-model in estimating postprandial absolute levels and postprandial increases in cardiometabolic markers. Stars denote regression model results in the US validation cohort for postprandial measurements (not rises; FIGS. 18 and 19).

FIG. 17 Performance for random Forest regression and classification on microbiome functional potential in predicting fasting measurements. FIG. 17 shows the performance of both RF regression and classification tasks trained on microbiome gene families' profiles in predicting the fasting measurements presented in FIG. 16A. Boxplots show the distribution of the Spearman correlations (left axis) between real and predicted values using RF regression. Circles show the median AUC (right axis) of RF classification in predicting the bottom quartile of the distribution vs. the top quartile. Fasting measurements are sorted as in FIG. 16A.

FIG. 18 Random Forest regression and classification performances for total cholesterol in different lipoproteins. The figure shows the performances of both RF regression and classification tasks in predicting the total cholesterol in different size lipoproteins. For each lipoprotein, its concentration values were considered at both fasting and postprandial (6 h), and the difference (rise) between the post-prandial concentration and the fasting one. Boxplots show the distribution of the Spearman correlations (left axis) between real and predicted values using RF regression. Circles show the median AUC (right axis) of RF classification in predicting the bottom quartile of the distribution vs. the top quartile. Lipoproteins are sorted descending according to the median of the RF regression for the fasting measure.

FIG. 19 Random Forest regression and classification performances for triglycerides in different lipoproteins. The figure shows the performances of both RF regression and classification tasks in predicting triglycerides in different size lipoproteins. For each lipoprotein, its concentration values were considered at both fasting and postprandial (6 h), and also the difference (rise) between the post-prandial concentration and the fasting one. Boxplots show the distribution of the Spearman correlations (left axis) between real and predicted values using RF regression. Circles show the median AUC (right axis) of RF classification in predicting the bottom quartile of the distribution vs. the top quartile. Lipoproteins are sorted descending according to the median of the RF regression for the fasting measure.

FIGS. 20A, 20B-1, 20B-2, 20C-20D. Species-level segregation into healthy and unhealthy microbial signatures of fasting and postprandial cardiometabolic markers. (FIG. 20A) Associations (Spearman correlation, q<0.2 marked with stars) between single microbial species and fasting clinical risk measures and (FIGS. 20B-1 & 20B-2) glycemic, inflammatory, and lipemic indices. (FIG. 20C) Correlation between microbial species and the iAUC for glucose and C-peptide estimations based on clinical measurements before and after standardized meals. The 30 species with the highest number of significant correlations with distinct fasting and postprandial indices are shown. In each of FIGS. 20A-20C, positive Spearman correlation values are enclosed in dashed outline; asterisks indicate statistical significance. (FIG. 20D) Microbe-metabolite correlations are very consistent when evaluated for fasting versus postprandial (6 h) conditions (left panel). Associations with postprandial variations (rise) conversely often show opposing relationships, with several species positively correlated with fasting measures being negatively correlated with postprandial variation of the same metabolite (or vice versa, central panel). This was mitigated somewhat when comparing absolute postprandial responses with rise (right panel).

FIG. 21 (in two parts, FIG. 21-1 & FIG. 21-2) Species-level correlations with total lipids in lipoproteins. The heatmap shows the species-level correlations with total lipids in lipoprotein variables at fasting, post-prandial (6 h) and the difference (rise) between the postprandial and fasting concentrations. The 30 species with the highest number of significant associations (FDR0.2) are shown. The asterisk indicates a significant correlation between species and metadata variable using t-test, corrected with FDR with q<0.2. The species listed along the y-axis from top to bottom are: Ruminococcus gnavus, Anaerotruncus colihominis Clostridium symbiosum, Clostridium bolteae sp CAG 58, Clostridium innocuum, Prevotella copri, Firmicutes bacterium_CAG_170, Roseburia sp_CAG_182, Firmicutes bacterium_CAG_95, Haemophilus parainfluenzae, Coprobacter secundus, Oscillibacter sp_PC13, Faecalibacterium prausnitzii, Veillonella parvula, Turicibacter sanguinis, Oscillibacter sp 57 20, Clostridium disporicum, and Firmicutes bacterium CAG 110. Positive Spearman correlation values are enclosed in dashed outline; asterisks indicate statistical significance.

FIG. 22 (in two parts, FIG. 22-1 & FIG. 22-2) Species-level correlations with total cholesterol in lipoproteins. The heatmap shows the species-level correlations with total cholesterol in lipoprotein variables at fasting, post-prandial (6 h) and the difference (rise) between the postprandial and fasting concentrations. The 30 species with the highest number of significant associations (FDR0.2) are shown. The asterisk indicates a significant correlation between species and metadata variable using t-test, corrected with FDR with q<0.2. The species listed along the y-axis from top to bottom are: Clostridium citroniae, Hungatella hathewayi, Clostridium sp_CAG_58, Gemella sanguinis, Blautia hydrogenotrophica, Eggerthella lenta, Bacteroides uniformis, Eisenbergiella tayi, Ruthenibacterium lactatiformans, Clostridium spiroforme, Flavonifractor plautii, Clostridium bolteae, Ruminococcus gnavus, Anaerotruncus colihominis, Clostridium symbiosum, Clostridium bolteae_CAG_59, Clostridium innocuum, Prevotella copri, Firmicutes bacterium CAG 170, Roseburia sp CAG182, Firmicutes bacterium CAG 95, Haemophilus parainfluenzae, Coprobacter secundus, Oscillibacter sp_PC13, Faecalibacterium prausnitzii, Veillonella parvula, Turicibacter sanguinis, Oscillibacter sp_57_20, Clostridium disporicum, and Firmicutes bacterium_CAG_110. Positive Spearman correlation values are enclosed in dashed outline; asterisks indicate statistical significance.

FIG. 23 (in two parts, FIG. 23-1 & FIG. 23-2) Species-level correlations with triglycerides in lipoproteins. The heatmap shows the species-level correlations with triglycerides in lipoprotein variables at fasting, post-prandial (6 h) and the difference (rise) between the postprandial and fasting concentrations. The 30 species with the highest number of significant associations (FDR≤0.2) are shown. The asterisk indicates a significant correlation between species and metadata variable using t-test, corrected with FDR with q<0.2. The species listed along the y-axis from top to bottom are the same as those listed in FIGS. 22-1 & 22-2. Positive Spearman correlation values are enclosed in dashed outline; asterisks indicate statistical significance.

FIG. 24 (in two parts, FIG. 24-1 & FIG. 24-2) Gene families' correlations with clinical and metabolic risk scores, glycemic and inflammatory measures, and lipoproteins. The heatmap shows gene families correlations with the set of metadata presented in FIGS. 20A-20C reporting the top 2,000 genes selected those with at least 20% prevalence on their number of significant correlations (q<0.2). Gene families' correlations are showing the same clusters as the species-level correlations in FIGS. 20A-20C. A color version of this Figure can be found in Asnicar et al. (Nat Med. 27:321-323, 2021).

FIG. 25 (in two parts, FIG. 25-1 & FIG. 25-2) Pathway abundances correlations with clinical and metabolic risk scores, glycemic and inflammatory measures, and lipoproteins. The heatmap shows pathway abundances correlations with the set of metadata presented in FIGS. 20A-20C reporting all the pathways at 20% prevalence (349 in total). Pathway abundances correlations are showing the same cluster structure as the species-level correlations in FIGS. 20A-20C. A color version of this Figure can be found in Asnicar et al. (Nat Med. 27:321-323, 2021).

FIGS. 26A-26F Concordance of Random Forest scores with species-level partial correlations. Volcano plots of the scores assigned to each species by Random Forest and their partial correlation, showing an overall concordance between the two independent approaches. The top 5 metadata variables were considered for the six metadata categories: (FIG. 26A) Foods, bacon (g) (corr. 0.496), unsalted nuts (g) (0.466), pork (g) (0.424), dark chocolate (g) (0.41), and garlic (g) (0.401) (FIG. 26B) Food groups, nuts (0.436), legumes (0.403), meat (0.393), sweets and desserts (0.369), and potatoes (0.323). (FIG. 26C) Nutrients, polyunsaturated fatty acids (FAs) (g) (0.524), vitamin B12 μg (0.406), niacin equivalent (mg) (0.406), cis-polyunsaturated FAs (g) (0.358), and starch (g) (0.351). (FIG. 26D) Nutrients normalized by energy intake, polyunsaturated FAs (g % E) (0.528), fat (g % E) (0.512), vitamin B12 (μg % E) (0.48), niacin equivalent (mg % E) (0.462), and cis-polyunsaturated FAs (g % E) (0.436). (FIG. 26E) Dietary patterns, healthy PDI (0.528), unhealthy PDI (0.381), healthy plant percentage (0.373), unhealthy plants number (0.363), and total PDI (0.361). (FIG. 26F) Lipoproteins, ApoA1 6 h rise (0.493), XL-VLDL-TG 6 h (0.413), VLDL-D 6 h (0.396), M-HDL-TG 6 h (0.393), and M-VLDL-TG 6 h (0.387). VLDL=very low density lipoprotein. Key-filled dots are those for which the correlation coefficient is statistically significant

FIGS. 27A-27E Prevotella copri and/or Blastocystis spp. presence are indicators of a more favorable postprandial glucose response to meals. (FIGS. 27A-27C) Differential analysis of visceral fat, HFD and glucose iAUC 2 h after standardized breakfast according to presence-absence of one and both of P. copri and Blastocystis spp. The analysis reveals that both these species are indicators of reduced visceral fat, good cholesterol and meal-driven increase of glucose. (FIGS. 27D-27E) Differential analysis of C-peptide and triglycerides at different time points according to presence-absence of one and both of P. copri and Blastocystis spp. The distributions of the concentrations for C-peptide and triglycerides were typically lower when one or both are absent. An asterisk between two boxplots represents a significant p-value (p<0.05) according to the Mann-Whitney U test (Table 4). In FIGS. 27A-27E, the left bar of each pair is “Absent”; the right bar of each pair is “Present”.

FIG. 28 (in two parts, FIG. 28-1 & FIG. 28-2) The panel of 30 species showing the strongest overall correlations with a selection of markers of nutritional and cardiometabolic health. The 30 species with the highest and lowest average ranks with diverse positive and negative health indicators, respectively, are shown here. The rank of each microbe's correlation with individual health indicators is written within cells when significant (p<0.05). For each of the main categories of indices, up to five representative quantitative markers were selected (for “Personal” only four were considered as the remaining were highly correlated with visceral fat or not relevant in this context). Indices can be considered “positive” and “negative” depending on whether higher or lower values are a proxy for more or less healthy conditions. A color version of this Figure can be found in Asnicar et al. (Nat Med. 27:321-323, 2021).

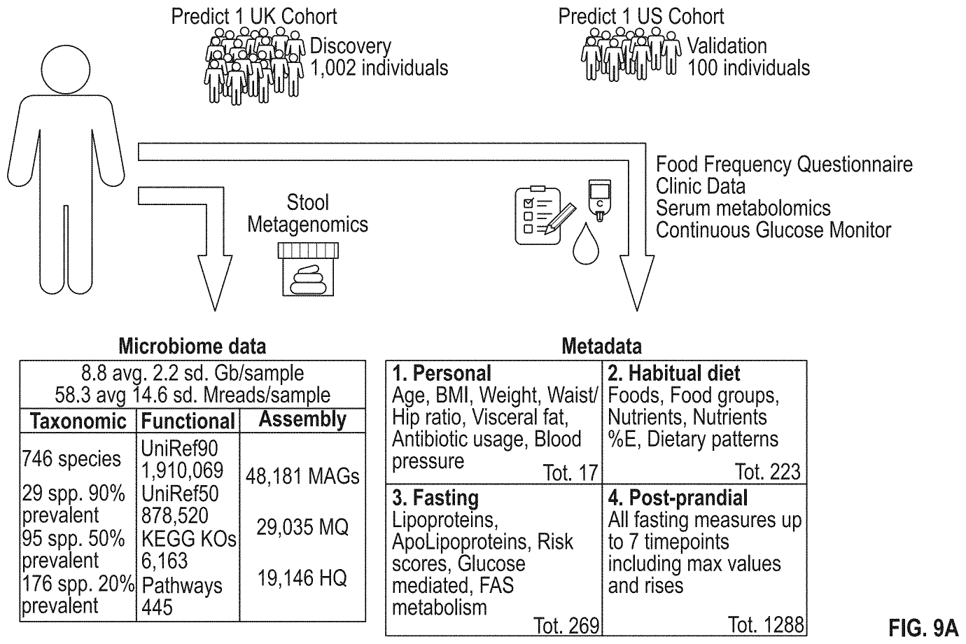

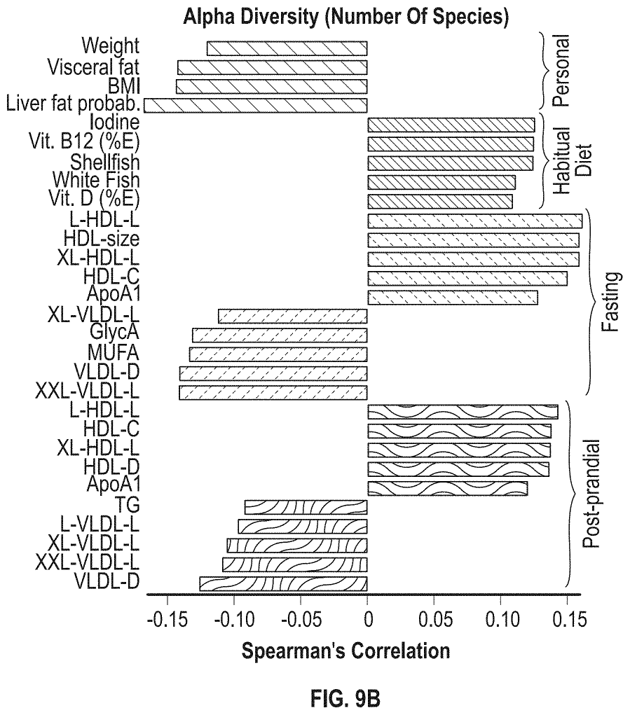

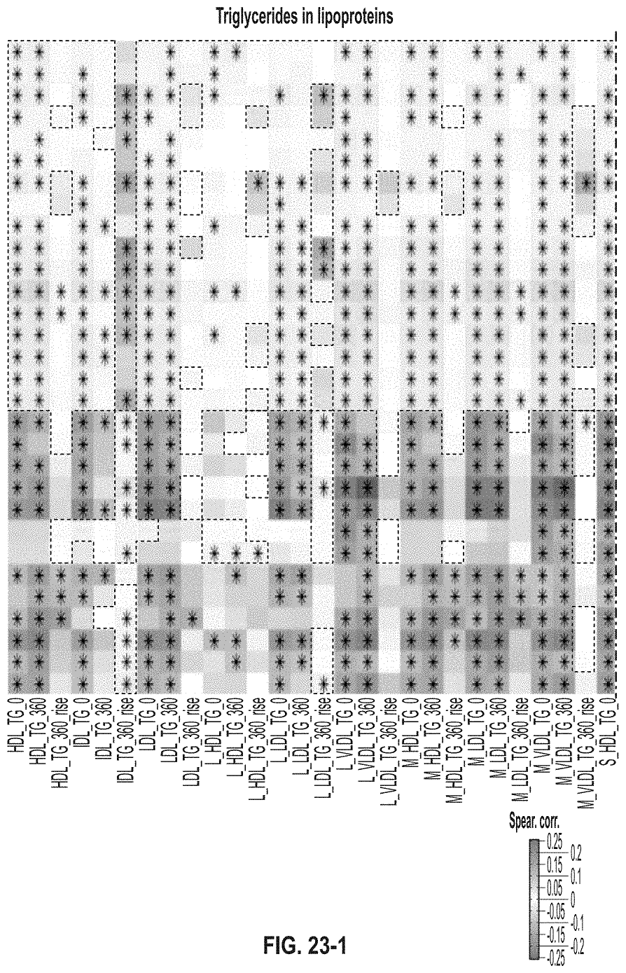

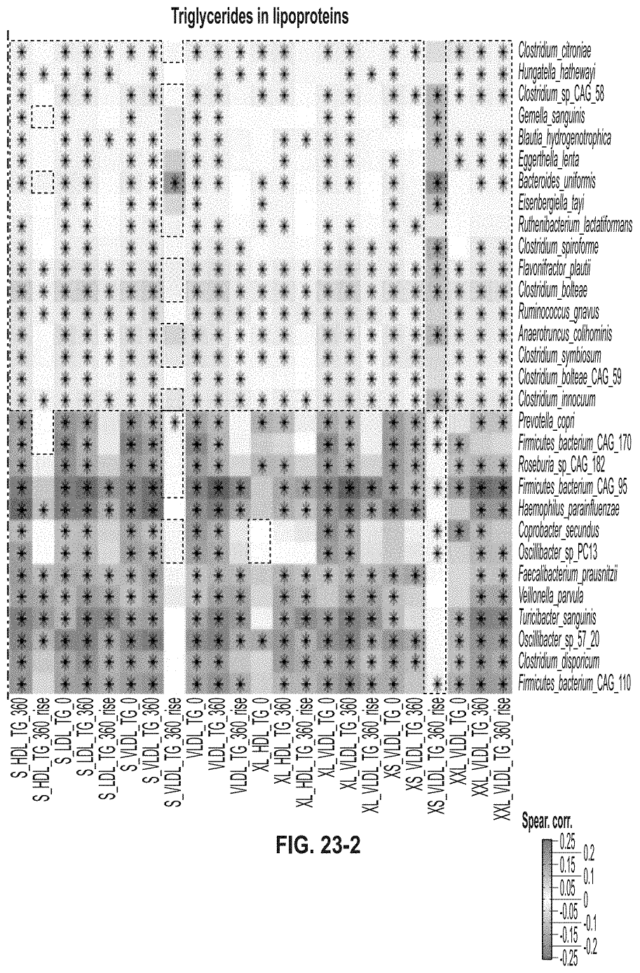

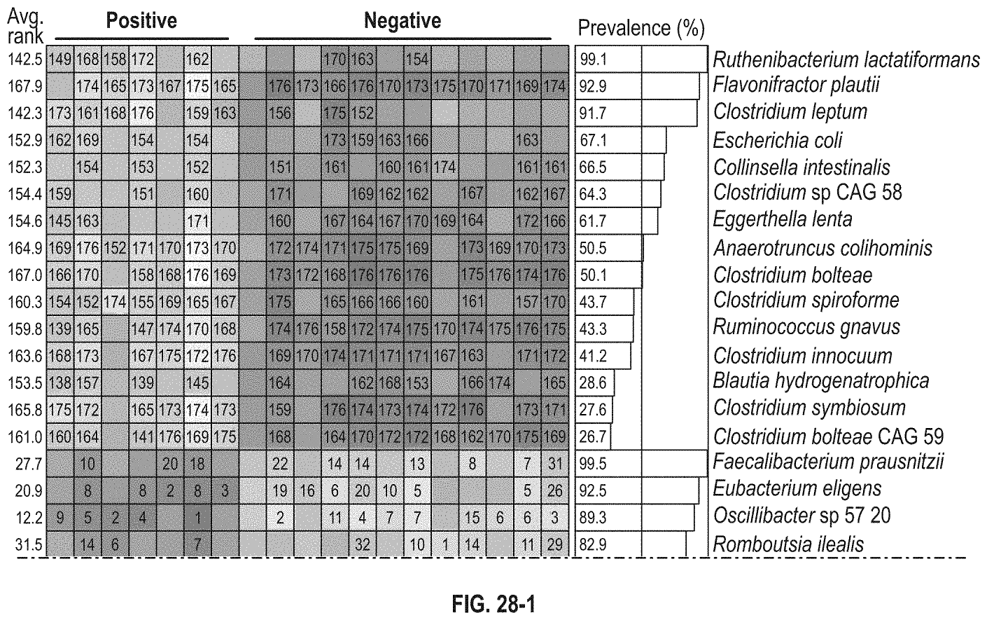

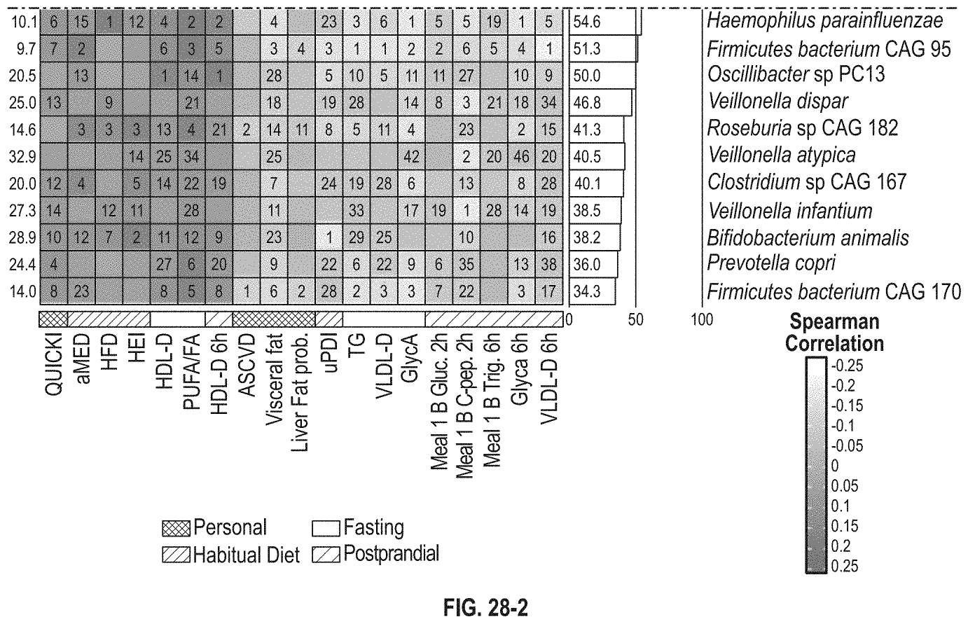

Several of FIGS. 9-28, or versions thereof, were published in Asnicar et al. (Nat Med. 27:321-323, 2021, Epub 11 Jan. 2021; which is incorporated herein by reference for all it teaches); at least some of these Figures may be clearer in color, as they are depicted in Ansicar et al., and Applicant considers that color information to be included in this filing.

### DETAILED DESCRIPTION

Using the technologies described herein, microbiome data associated with an individual and other data are analyzed to generate a microbiome fingerprint, a dietary fingerprint, and microbiome ancestry data for a user. As used herein, a “microbiome fingerprint” is data that uniquely identifies the microbiome of a user at a particular point in time, and a “dietary fingerprint” is data that identifies how the microbiome of a user at a particular point in time is associated with one or more different indexes associated with a diet and/or health characteristics. The indexes may include, but are not limited to a Mediterranean diet index, a vegetarian diet index, a fast food index, an internal fat index, a fat-digesting index, a carbohydrate-digesting index, a health index, a fasting index, a ketogenic index, and the like. According to some configurations, one or more computers of a microbiome service generate a score, such as from 0-100, (or some other indicator) that indicates how closely the microbiome of the user is associated with a particular index.

As an example, the Mediterranean diet index score for a user indicates how closely the microbiome of the user resembles the typical microbiome of someone on a Mediterranean diet. The vegetarian diet index score indicates how closely the microbiome of the user resembles someone on a vegetarian diet. The fast food index score indicates how closely the microbiome of the user resembles someone on a fast food diet. The internal fat index score indicates how closely the microbiome of the user resembles someone with high or low visceral fat. The fat-digesting index score indicates how closely the microbiome of the user resembles someone with low postprandial triacylglycerol (TAG) rises. The carbohydrate-digesting index score indicates how closely the microbiome of the user resembles someone with low postprandial glucose rises. The health index score indicates how closely the microbiome of the user resembles someone that is healthy. The fasting index score indicates how closely the microbiome of the user resembles someone that fasts regularly. The ketogenic index score indicates how closely the microbiome of the user resembles someone who is ketogenic.

The microbiome service may utilize microbiome data generated from a microbiome sample and/or other data to generate a microbiome fingerprint, dietary fingerprint, and/or microbiome ancestry data for a user, or for a delegate of a user. For example, the microbiome service may perform an analysis of the microbiome data associated with a microbiome sample to identify the microbial composition (e.g., the species, genes, taxa, and the like); such identification may include the unique, detailed characterization of each and every microbial strain in the sample, but it is not necessary to identify every strain present in the sample. For instance, the analysis of the microbiome data may identify as few as 2% of the strains in the sample; as few as 5%, as few as 8%, as few as 10%, as few as 15%, as few as 20%, or more than 30% of the strains in the sample. In certain embodiments, the characterization will identify more than 25% of the strains; for instance, more than 30%, more than 35%, more than 40%, more than 45%, more than 50%, more than 55%, more than 60%, more than 65%, more than 70%, more than 75%, more than 80%, more than 85%, more than 90%, or even more than 95% of the strains in the sample.

In some examples, some/all of the analysis of the microbiome service may be performed by a service provider that is external from the microbiome service. The microbiome service may obtain this portion of the microbiome data from the external service provider(s). The microbiome service may also generate reconstructed microbial genomes, determine a diversity of the microbiome, identify functions of the microbiome, identify a uniqueness of the microbiome, identify interesting species, and the like.

In some examples, the microbiome data of the user is utilized with other data that is gathered about the user, as well as other users. For instance, users may provide responses to questionnaires, data about food that is eaten, data about supplements or medicines that are eaten, sleep habits, and the like.

Among other uses, data in addition to the microbiome data may be utilized to assist in determining a “microbiome ancestry” of a user. A “microbiome ancestry” for a user indicates that the user has relationships with other users and/or locations based on a similarity of the microbiome data (e.g., the microbiome fingerprint) fora particular user with other users.

In some examples, the microbiome service generates a microbiome ancestry by analyzing the microbiome data of the user and determining how closely the microbiome of the user is related to one or more other users, and/or locations. For instance, the microbiome service may determine a number of other users to which the microbiome of the user is most closely related to. In some configurations, the microbiome service compares the microbiome data, such as the microbiome fingerprint, of the user to microbiome data, such as the microbiome fingerprints, of other users to determine whether the user is related to any of the other users.

As briefly discussed, the microbiome service may also identify one or more locations to which the microbiome of the user is associated with. For example, the microbiome service may identify the countries the microbiome of the user is associated with (e.g. 75% North America, 25% Mexico). This identification may be based on microbiome data of users at different locations and/or different populations (e.g., English, American, French, Mexican, Italian, . . . ). For instance, the microbiome service may determine that the microbiome fingerprint of the user is more similar to a microbiome of a user in France even though the user is from England.

According to some configurations, a user may “opt-in” to allow use of the microbiome data and/or other data associated with a user. In some examples, the user “opts-in” to participate in a social network and/or some other communication mechanism to discuss issues related to the microbiome data such as a microbiome ancestry (e.g., compare diets and background with other users). The microbiome service may also compare the microbiome of the user with other family members, and/or other users when the users have “opted-in” to allow this. For instance, the microbiome service may identify how many strains they share (with respect to sharing with unrelated persons) and overall how similar they are compared to the average.

In some examples, the microbiome service may provide a user interface (UI), such as a graphical user interface (GUI) for a user to view and interact with microbiome data and/or other data associated with the microbiome fingerprints, dietary fingerprints, and microbiome ancestry. For instance, the GUI may display microbiome fingerprint data that shows various characteristics of the microbiome fingerprint, dietary fingerprint data that shows various characteristics of the dietary fingerprint, microbiome ancestry data that shows various characteristics of the microbiome ancestry, recommendation data that identifies one or more recommendations relating to changing the microbiome of the user, and the like.

As an example, the microbiome service may provide recommendations to increase the diversity of foods eaten, as there is no one good food for a healthy microbiome. The recommendations may include to eat different gut-healthy foods, eat fermented foods, minimize highly processed foods (things like emulsifiers and artificial sweeteners may affect the microbiome), consume prebiotic substances, administer a probiotic preparation, or any combination thereof. The microbiome service may base the recommendations on data obtained from the user, from other users, and/or from both.

The microbiome service may also track the state of the microbiome of the user over time. For example, the microbiome service may provide data related to different microbiome analysis. In this way, the user may see how changes made by the user (e.g., eating different foods, changing exercise patterns, consuming prebiotic substance(s), taking a probiotic preparation, and so forth) have affected the microbiome.

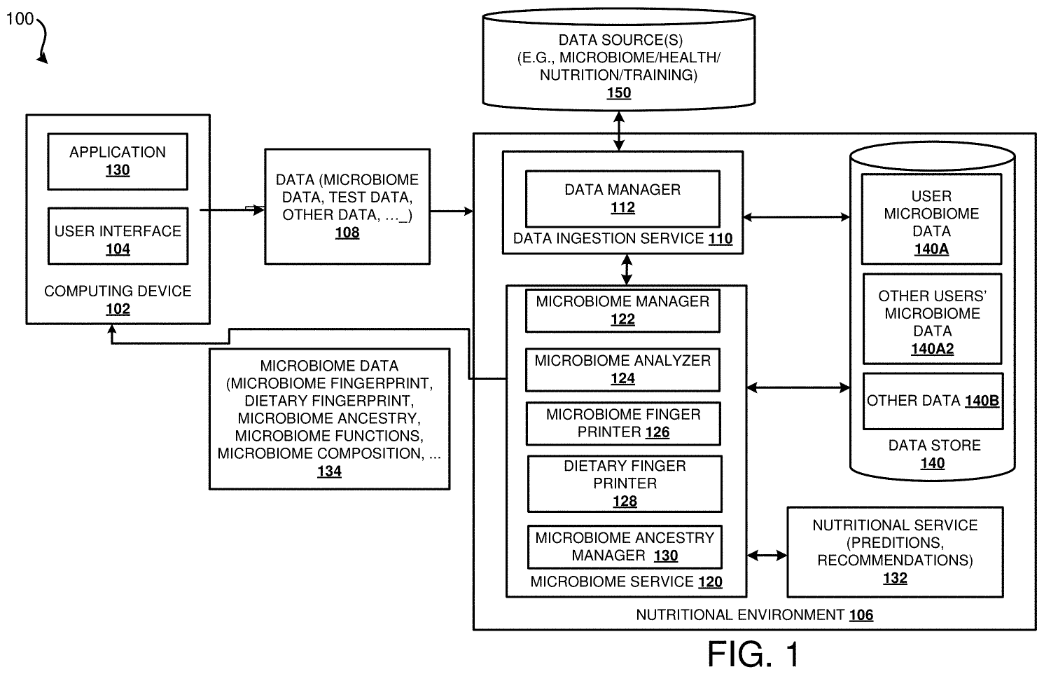

Additional details regarding the various components and processes described above relating to generating microbiome fingerprints, dietary fingerprints, and microbiome ancestry are presented below with regard to FIGS. 1-8.

It will be appreciated that the subject matter presented herein may be implemented as a computer process, a computer-controlled apparatus, a computing system, or an article of manufacture, such as a computer-readable storage medium. While the subject matter described herein is presented in the general context of program modules that execute on one or more computing devices, those skilled in the art will recognize that other implementations may be performed in combination with other types of program modules. Generally, program modules include routines, programs, components, data structures and other types of structures that perform particular tasks or implement particular abstract data types.

Those skilled in the art will also appreciate that aspects of the subject matter described herein may be practiced on or in conjunction with other computer system configurations beyond those described herein, including multiprocessor systems, microprocessor-based or programmable consumer electronics, minicomputers, mainframe computers, handheld computers, personal digital assistants, e-readers, mobile telephone devices, tablet computing devices, special-purposed hardware devices, network appliances and the like.

In the following detailed description, references are made to the accompanying drawings that form a part hereof, and that show, by way of illustration, specific examples or examples. The drawings herein are not drawn to scale. Like numerals represent like elements throughout the several figures (which may be referred to herein as a “FIG.” or “FIGs.”).

Provided below is additional description in support of this technology, which is organized in the following sections: (I) Generation, Collection, and Analysis of Microbiome Data; (II) Representative Computer Architecture; (III) Detection and Identification of Individual Microbes; (IV) Methods of Use; (V) Kits and Arrays; (VI) Systems; (VII) Exemplary Embodiments; (VIII) Example(s); (IX) Incorporation of Appendix I; and (X) Closing Paragraphs.

### (I) GENERATION, COLLECTION, AND ANALYSIS OF MICROBIOME DATA

FIG. 1 is a block diagram depicting an illustrative operating environment 100 in which microbiome data is analyzed to generate microbiome fingerprints, dietary fingerprints, and microbiome ancestry for users. An individual, such as an individual interested in obtaining microbiome fingerprints, dietary fingerprints, and microbiome ancestry information, may communicate with the nutritional environment 106 using a computing device 102 and possibly other computing devices, such as mobile electronic devices.

In some configurations, an individual may generate and provide data 108, such as microbiome data, test data, and/or other data. According to some examples, the user may utilize a variety of at home biological collection devices, which collect a biological sample. These devices may include but are not limited to “At Home Blood Tests” which use blood extraction devices such as finger pricks which in some examples are used with dried blood spot cards, button operated blood collection devices using small needles and vacuum to collect liquid capillary blood and the like. In some examples there may be home biological collection devices such as a stool test which is then assayed to produce biomarker test data such as gut microbiome data. As exemplified herein, the subject from which the biological sample is obtained may be a human subject. Other animal subjects are also contemplated, including non-human primates, companion animals, domestic animals, livestock, endangered and threatened animals, laboratory animals, and so forth.

A computing device, such as a mobile phone or a tablet computing device can also be used to improve the accuracy of the measurements. For instance, instead of relying on an individual to accurately record the time a test was taken or a sample was obtained, the computing device 102 can record information that is associated with the event. The computing device 102 may also be utilized to capture the timing data associated with the test (e.g., the time the test was performed, . . . ), or the sample was collected, and provide that data to a data ingestion service 110. As an example, a clock (or some other tinning device) of the computing device 102 may be used to record the time the measurement(s) were collected and/or samples were obtained.

As illustrated in FIG. 1, the operating environment 100 includes one or more computing devices 102, in communication with a nutritional environment 106. In some examples, the nutritional environment 106 may be associated with and/or implemented by resources provided by a service provider network such as provided by a cloud computing company. The nutritional environment 106 includes a data ingestion service 110, a microbiome service 120, a nutritional service 132, and a data store 140. The nutritional service 132 can be utilized to generate personalized nutritional recommendations. For example, the personalized nutritional recommendations can be generated using techniques described in U.S. Patent Publication No. US 2019-0252058 A1, published Aug. 15, 2019. According to some examples, the nutritional service 132 may provide recommendations based on the microbiome fingerprint, dietary fingerprint, microbiome ancestry data and/or other data.

The nutritional environment 106 may include a collection of computing resources (e.g., computing devices such as servers). The computing resources may include a number of computing, networking and storage devices in communication with one another. In some examples, the computing resources may correspond to physical computing devices and/or virtual computing devices implemented by one or more physical computing devices.

It should be appreciated that the nutritional environment 106 may be implemented using fewer or more components than are illustrated in FIG. 1. For example, all or a portion of the components illustrated in the nutritional environment 106 may be provided by a service provider network (not shown). In addition, the nutritional environment 106 could include various Web services and/or peer to peer network configurations. Thus, the depiction of the nutritional environment 106 in FIG. 1 should be taken as illustrative and not limiting to the present disclosure.

The data ingestion service 110 facilitates submission of data utilized by the microbiome service 120 and, in some configurations, the nutritional service 132. Accordingly, utilizing a computing device 102, an electronic collection device, an at home biological collection device or via in clinic biological collection, an individual may submit data 108 to the nutritional environment 106 via the data ingestion service 110. Some of the data 108 may be sample data, biomarker test data, and some of the data 108 may be non-biomarker test data such as photos, barcode scans, timing data, and the like.

A “biomarker” or biological marker generally refers to one or more measurable indicators (that may be combined using various techniques) of some biological state or condition associated with an individual. Stated another way, a biomarker may be anything that can be used as an indicator of particular disease, condition, health, state, or some other physiological state of an organism. A biomarker typically can be measured accurately (either objectively and/or subjectively) and the measurement is reproducible. By way of example, the following are considered biomarkers: blood glucose, triglycerides (TG), insulin, c-peptides, ketone body ratios, IL-6 inflammation markers, the expression of any specified gene or protein, hunger, fullness, body mass index (BMI), composition of a microbiome (including not only what strains are present, but the relative abundance of two or more strains in a microbiome), and the like. In practice, a good biomarker is often a combination of two or more measurable indicators combined in a simple or complex way; in some cases, the combination of more than one measurable indicator makes the biomarker more closely linked to the disease, condition, health, state, or some other physiological state of an organism.

The measured biomarkers can include many different types of health data such as microbiome data which may be referred to herein as “microbiome data”, blood data, glucose data, lipid data, nutrition data, wearable data, genetic data, biometric data, questionnaire data, psychological data (e.g., hunger, sleep quality, mood, . . . ), objective health data (e.g., age, sex, height, weight, medical history, . . . ), as well as other types of data. Generally, “health data” refers to any psychological, subjective, and/or objective data that relates to and is associated with one or more individuals. The health data might be obtained through testing, self-reporting, and the like. Some biomarkers change in response to eating food, such as blood glucose, insulin, c-peptides, and triglycerides and their lipoprotein components.

To understand the differences in nutritional responses for different users, dynamic changes in biomarkers caused by eating food such as a standardized meal (“postprandial responses”) can be measured. By understanding an individual's nutritional responses, in terms of blood biomarkers such as glucose, insulin, and triglyceride levels, or non-blood biomarkers such as the microbiome, a nutritional service may be able to choose or recommend food(s) that is/are more suited for that particular person.

Data may also be obtained by the data ingestion service 110 from other data sources, such as data source(s) 150. For example, the data source(s) 150 can include, but are not limited to microbiome data associated with one or more users, nutritional data (e.g., nutrition of particular foods, nutrition associated with the individual, and the like), health data records associated with the individual and/or other individuals, and the like.

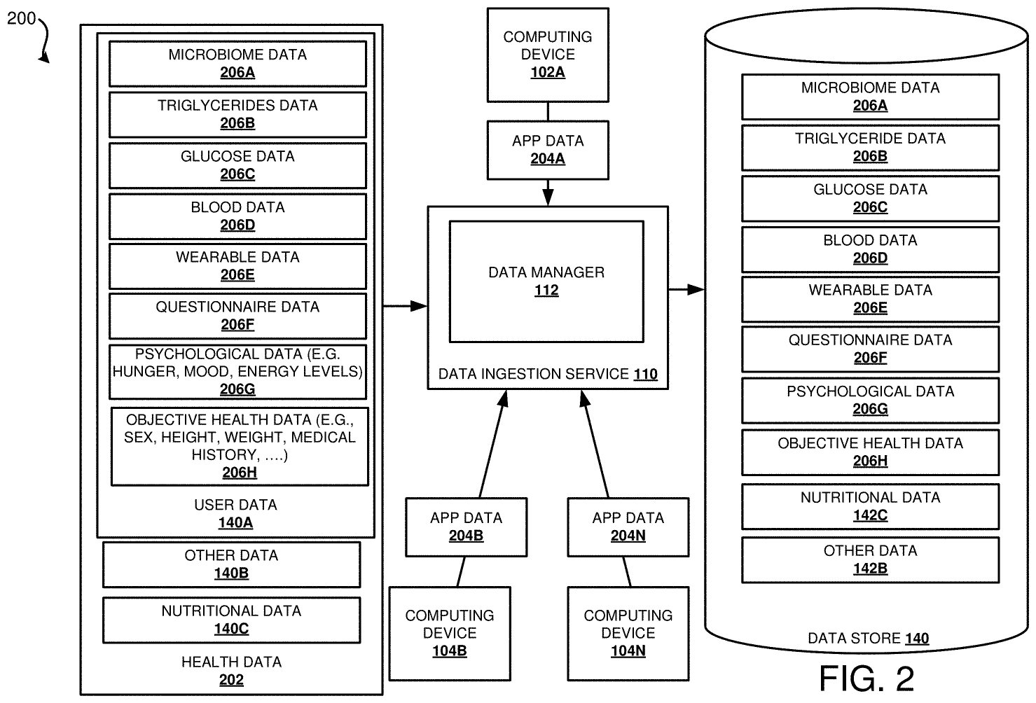

The data, such as data 108, or data obtained from one or more data sources 150, may then be processed by the data manager 112 and/or the microbiome manager 122 and included in a memory, such as the data store 140. As illustrated, the data store 140 can be configured to store user microbiome data 140A, other users' microbiome data 140A2, and other data 140B (see FIG. 2 for more details on the data ingestion service 110). In some examples, the user microbiome data 140A and other users' microbiome data 140A2 includes microbiome data.

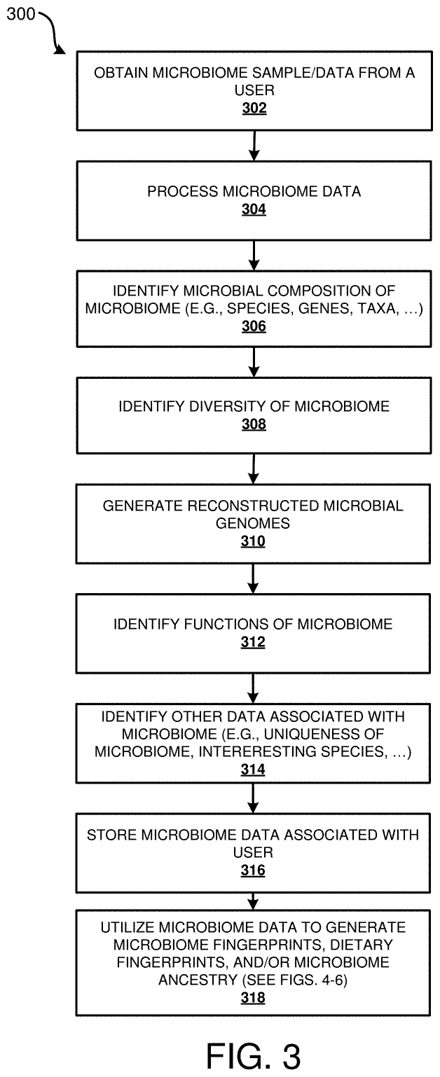

As discussed in more detail below (see FIGS. 3-7 for more details), the microbiome service 120 utilizing the microbiome manager 122, the microbiome analyzer 124, the microbiome finger printer 126, the microbiome dietary finger printer 128, and the microbiome ancestry manager 130, analyzes the data 108 associated with a user and generate a microbiome fingerprint, a dietary fingerprint, and microbiome ancestry data for the user. According to some configurations, the microbiome service 120 utilizes both data 108 associated with the user and data from other users.

In some examples, the microbiome manager 122 may utilize one or more machine learning mechanisms. For example, the microbiome manager 122 can use a classifier to classify the microbiome within a classification category (e.g., associate with a particular dietary index, a geographic location, . . . ). In other examples, the microbiome manager 122 may use a scorer to generate scores that may provide an indication of the dietary index associated with a user, how closely related the user is to other users based on the microbiome data, and the like.

The data ingestion service 110 and/or the microbiome service 120 can generate one or more user interfaces, such as a user interface 104 and/or user interface 104B, through which an individual, utilizing the computing device 102, or some other computing device, may provide/receive data from the nutritional environment 106. For example, the data ingestion service 110 may provide a user interface 104 that allows an individual of the computing device 102A to submit data 108 to the nutritional environment 106.

In some cases, the individual can also provide biological samples to a lab for testing, for instance using a biological collection device. According to some configurations, this will include At Home Blood Tests. According to some configurations, individuals can provide a sample (such as a stool sample) for microbiome analysis. As an example, metagenomic testing can be performed using the sample to allow the DNA of the microbes in the microbiome of an individual to be digitalized. Generally, a microbiome analysis includes determining the composition and functional potential (here called just “function”) of a community of microbes in a particular location, such as within the gut of an individual. An individual's microbiome appears to have a strong relationship to metabolism, weight, and health, yet only ten to thirty percent of the bacterial species in a microbiome is estimated to be common across different individuals. Embodiments described herein combine different techniques to assist in improving the accuracy of the data captured outside of a clinical setting, such as calculating accurate glucose responses to individual meals, which can then be linked to measures like the microbiome.

According to some configurations, individuals can provide a sample or samples of their stool for microbiome analysis as part of the at home biological collection. In some cases, this sample may be collected without using a chemical buffer. The sample can then be used to culture live microbes, or for chemical analysis such as for metabolites or for genetic related analysis such as metagenomic or metatranscriptomic sequencing. In such cases, the sample may suffer from changes in microbial composition due to causes including microbial blooming from oxygen in the period between being collected and when it is received in the lab, where it usually will be immediately assayed or frozen. In some cases, to avoid this change in bacterial composition after collection, the sample obtained a home may be frozen at low temperatures very rapidly after collection. The sample can then be used to culture live bacteria, or for chemical analysis or for metagenomic sequencing. This collection can be done as part of an in clinic biological collection or at home where the collection kit is configured to deliver such low temperatures and maintain them until a courier has taken the sample to a lab.

A stool sample may be combined with a chemical preservation buffer, such as ethanol, as part of the at home collection process to stop further microbial activity, which allows a sample to be kept at room temperature before being received at the lab where the assay is done. In some examples, the buffer may be a proprietary chemical product sold and validated by another company for the task of freezing microbial activity while still allowing the sample to be processed for metagenomics sequencing. A buffer allows for such a sample to be posted in the mail without (or minimizing) issues of microbial blooming or other continuing changes in microbial composition. The buffer may however prevent some biochemical analyses from being done, and because preservation buffers are likely to kill a large fraction of the microbial population, it is unlikely that samples conserved in preservation buffers can be used for cultivation assays.

In some cases, a user may do multiple stool tests over time, so that changes in the microbiome over time can be measured, or changes in the microbiome in response to meals, or changes in the microbiome in response to other clinical or lifestyle variations.

In some examples, the stool sample is collected using a scoop or swab from a stool that is collected by the user using a stool collection kit that prevents the stool from contamination, such as for instance the contamination that would occur from stool falling into a toilet. Because there is a very high microbial load in the gut microbiome compared, for example, to the skin microbiome, it is also possible that in some cases the stool sample is taken from paper that is used to clean the user's behind after they have passed a stool. This is only possible if the quantity of stool is large enough that the microbes from the stool greatly exceed the microbes that will be picked up from the user's skin or environmental contaminants. In any of these cases the scoop, swab, or tissue may be placed inside a collection device, such as a vial that contains a buffer solution. If the user ensures the stool comes into contact with the buffer, for example by shaking, then further microbial activity is stopped and the solution can be kept at room temperature without a significant change in microbial composition.

In some cases, a sterile synthetic tissue is used that does not have biological origins such as paper, so that when the DNA of the sample is extracted there is no contamination from DNA originating in the tissue.

According to some examples, the tissue is impregnated with a liquid to help capture more stool from the user's skin, where the liquid does not interfere with the results of the stool test and is not potentially dangerous for the human body.

In some cases, the timing and quality of the stool sample can be recorded using the computing device 102, for example using a camera. Where there are multiple stool tests the computing device 102 can use a barcode (or some other identifier) to confirm the timing and identity of that particular sample. Other data can also be collected. For example, data about how the sample was stored, how long the sample was stored before being supplied to the lab for analysis, and the like.

While the data ingestion service 110, the microbiome service 120, the nutritional service 132 are illustrated separately, all or a portion of these services may be located in other locations or together with other components. For example, the data ingestion service 110 may be located within the microbiome service 120. Similarly, the microbiome manager 122 may be part of a different service, and the like.

According to some examples, some individuals may be asked to visit a clinic to combine at home data with data collected at a clinic. The purpose of the clinic visit is to allow much higher accuracy of measurement for a subset of the individual's data, which can then be combined with the lower quality at home data. This may be used by the microbiome service 120 to improve the quality of the at home data.

According to some examples, the day before the visit to the clinic, the individuals are asked to avoid taking part in any strenuous exercise and to limit the intake of alcohol. In some configurations, the microbiome service 120 can analyze the data 108, such as data obtained from an activity tracker, to determine whether the individual followed the instructions of avoiding strenuous exercise. Similarly, the nutritional service 132, or some other device or component, may analyze the foods eaten by the individual by analyzing food data that indicates the foods eaten by the user. Individuals may be provided with instructions for the tests (e.g., avoid eating high fat or high fiber meals that may interfere with test results, fasting, drinking water, . . . ).

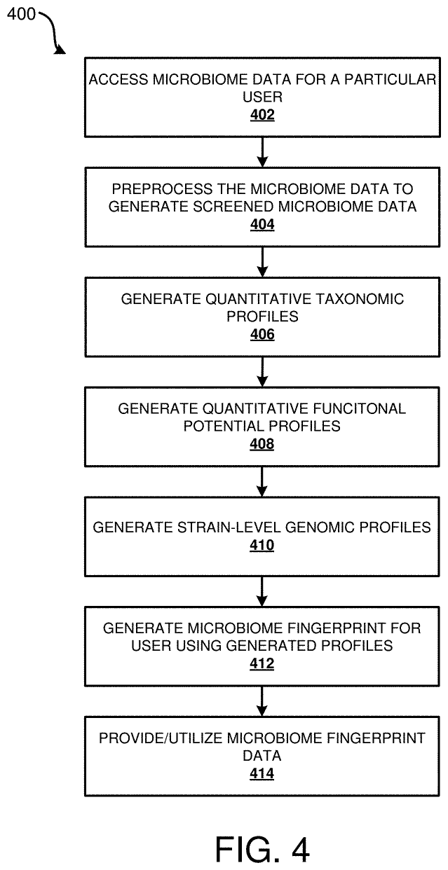

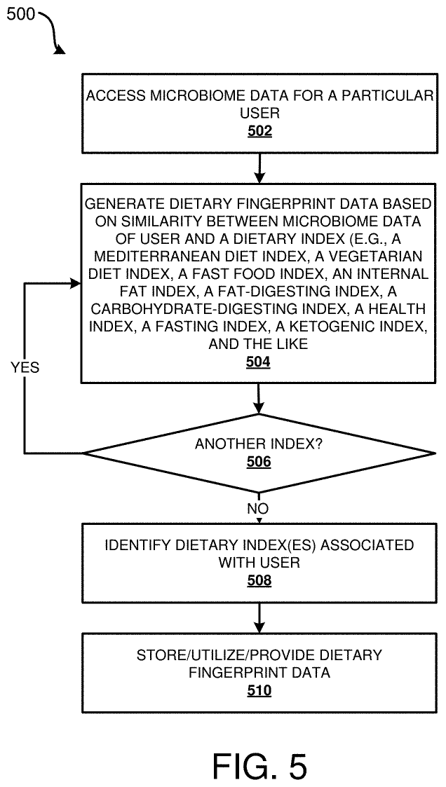

As described in more detail below with regard to FIGS. 4 and 5, the microbiome service 120 may use the microbiome manager 122 to generate a microbiome fingerprint, and a dietary fingerprint for a user. As discussed above, a “microbiome fingerprint” is data that uniquely identifies the microbiome of a user at a particular point in time. According to some configurations, the microbiome finger printer 126 generates a microbiome fingerprint from a user based on different profiles generated from the microbiome data, such as but not limited to quantitative taxonomic profiles, quantitative functional potential profiles, and strain-level genomic profiles. In some examples, the profiles are generated by the microbiome finger printer 126 and/or the microbiome analyzer 124.

According to some configurations, the microbiome fingerprint is a combination of descriptors, including, but not limited to (1) the quantitative (i.e. relative abundance) taxonomic profiles (i.e., the names or more generally identifiers (IDs) in case of unknown entities of microbial species or other taxonomic units), (2) the quantitative (i.e. relative abundance) functional potential profiles, (i.e., the names or generally identifiers (IDs) in case of unknown entities of microbial gene families, microbial pathways, and microbial functional modules), and (3) the strain-level genomic profiles (i.e., the reconstruction of the genomes or part of the genomes of as many microbes present in the microbiome as possible).

The microbiome fingerprint may be generated by the microbiome finger printer 126 using various techniques and methods. In some configurations, generation of the microbiome fingerprint includes obtaining the microbiome sample, generating DNA from the sample, preprocessing the raw sequencing data to the generate quality-screened sequencing data, and transforming the sequencing data is transformed into the numerical and genomics sets for the descriptors utilized to generate the microbiome fingerprint (e.g., quantitative taxonomic profiles, quantitative functional potential profiles, and strain-level genomic profiles).

The microbiome analyzer 124 may also be configured to perform processing associated with the microbiome data. For example, the microbiome analyzer 124 may be configured to generate and/or process sequencing data associated with the microbiome of the user. See FIG. 4 for more details on generating the profiles. After generating the profiles, the microbiome finger printer 126 may generate the microbiome fingerprint for the user. In some examples, the dietary finger printer 128 combines the data associated with the different profiles generated.

The dietary finger printer 128 is configured to generate a dietary fingerprint for the user. As discussed above, the “dietary fingerprint” of a user indicates how the microbiome of a user is associated with one or more different indexes that may be associated with a particular diet and/or a health characteristic. The indexes may include, but are not limited to a Mediterranean diet index, a vegetarian diet index, a fast food index, an internal fat index, a fat-digesting index, a carbohydrate-digesting index, a health index, a fasting index, a ketogenic index, and the like.

According to some configurations, the dietary finger printer 128 generates a score for each of the different indexes, such as from 0-100 (or some other indicator), to indicate how closely the microbiome of the user is associated with a particular index. For example, the dietary finger printer 128 may generate a score for each of the indexes based on how closely the microbiome of the user resembles a typical microbiome of someone that is known to follow a specific diet. For example, a score of 100 may indicate that the diet is strongly correlated to a particular diet, a score of 0 would indicate no correlation, and a score between 0 and 100 would indicate a different correlation. According to some configurations, the dietary finger printer 128 generates a Mediterranean diet index score, a vegetarian diet index score, a fast food index score, an internal fat index score, a fat-digesting index score, a carbohydrate-digesting index score, a health index score, fasting index score, ketogenic index score, and the like.

The Mediterranean diet index score for a user indicates how closely the microbiome of the user resembles the typical microbiome of someone on a Mediterranean diet. The vegetarian diet index score indicates how closely the microbiome of the user resembles someone on a vegetarian diet. The fast food index score indicates how closely the microbiome of the user resembles someone on a fast food diet. The internal fat index score indicates how closely the microbiome of the user resembles someone with high or low visceral fat. The fat-digesting index score indicates how closely the microbiome of the user resembles someone with low postprandial triacylglycerol (TAG) rises. The carbohydrate-digesting index score indicates how closely the microbiome of the user resembles someone with low postprandial glucose rises. The health index score indicates how closely the microbiome of the user resembles someone that is healthy. The fasting index score indicates how closely the microbiome of the user resembles someone that fasts regularly. The ketogenic index score indicates how closely the microbiome of the user resembles someone who is ketogenic.

In other configurations, the dietary finger printer 128, or some other service or component may utilize different mechanisms to determine whether the microbiome of the user resembles a particular diet and/or group. For instance, the dietary finger printer 128 may utilize a machine learning mechanism to classify the microbiome of the user within a classification and/or generate a score, or some other indicator that indicates how closely the microbiome data of the user matches the microbiome data of a representative user associated with the particular index.

The microbiome ancestry manager 130 is configured to generate microbiome ancestry data for a user. A “microbiome ancestry” refers to microbiome data that indicates that the user has relationships with other users and/or locations. In some examples, the microbiome service analyzes the microbiome data of the user and determines how closely the microbiome of the user is related to other users, and/or locations. For instance, the microbiome service may determine a number of other users to which the microbiome of the user is most closely related to. In some configurations, the microbiome ancestry manager 130 compares the microbiome data of the user to microbiome data of other users to identify a relationship. Similar to generating the scores for the different indexes performed by the dietary finger printer 128, the microbiome ancestry manager 130 may generate a score for each comparison between the user and the other users. The scores that indicate a close relationship (e.g., above a specified value) with the user may be identified as related.

The microbiome service may also identify one or more locations to which the microbiome of the user is associated with. For example, the microbiome service may identify the countries the microbiome of the user is associated with (e.g. 75% North America, 25% Mexico). This identification may be based on microbiome data of users at different locations and/or different populations (e.g., English, American, French, Mexican, Italian, . . . ). See FIG. 7 for additional details for generating the microbiome ancestry data.

The microbiome analyzer 124, or some other device or component, may analyze the microbiome data of a user before/after generating the microbiome fingerprint, dietary fingerprint, and/or microbiome ancestry for a user. For example, the microbiome analyzer 124 may perform an analysis of the microbiome data to identify the microbial composition of the microbiome (e.g., the species, genes, taxa, and the like). The microbiome service may also generate reconstructed microbial genomes, determine a diversity of the microbiome, identify functions of the microbiome, identify a uniqueness of the microbiome, identify interesting species, and the like.

In some examples, the microbiome data of the user is compared (e.g., by the microbiome service 120) with other data that is gathered about the user, as well as other users. For instance, users may provide responses to questionnaires, data about food that is eaten, sleep habits, and the like. Among other uses, this data may be utilized to determine a “microbiome ancestry” of a user.

In some examples, the microbiome service may provide a user interface (UI), such as a graphical user interface (GUI) 104 for a user to view and interact with data associated with the microbiome fingerprints, dietary fingerprints, and microbiome ancestry. For instance, the GUI may display microbiome fingerprint data that shows various characteristics of the microbiome fingerprint, dietary fingerprint data that shows various characteristics of the dietary fingerprint, microbiome ancestry data that shows various characteristics of the microbiome ancestry, recommendation data that identifies one or more recommendations relating to changing the microbiome of the user, and the like. In some configurations, the user may utilize an application 130 on the computing device 102 to interact with the nutritional environment. In some configurations, the application 130 may include functionality relating to processing at least a portion of the data 108.

As an example, the microbiome service 120 may provide recommendations generated by the nutritional service 132 to increase the diversity of foods eaten as there is no one good food for a microbiome. The recommendations may include to eat different gut-healthy foods, eat fermented foods, minimize highly processed foods (things like emulsifiers and artificial sweeteners may affect the microbiome). The microbiome service may base the recommendations on data obtained from the user, and other users.

The microbiome service 120 may also track the state of the microbiome of the user over time. For example, the microbiome service may provide data related to different microbiome analysis. In this way, the user may see how changes made by the user (e.g., eating different foods, changing exercise patterns, . . . ) have affected the microbiome.

FIG. 2 is a block diagram depicting an illustrative operating environment 200 in which a data ingestion service 110 receives and processes data associated with data associated with at home tests and sample collections. As illustrated in FIG. 2, the operating environment 200 includes the data ingestion service 110 that may be utilized in ingesting data utilized by the microbiome service 120.

In some configurations, the data manager 112 is configured to receive data such as, health data 202 that can include, but is not limited to microbiome data 206A, triglycerides data 206B, glucose data 206C, blood data 206D, wearable data 206E, questionnaire data 206F, psychological data (e.g., hunger, sleep quality, mood, . . . ) 206G, objective health data (e.g., height, weight, medical history, . . . ) 206H, nutritional data 140B, and other data 140C.

According to some examples, the microbiome data 206A includes data about the gut microbiome of an individual. The gut microbiome can host a large number of microbial species (e.g., >1000) that together have millions of genes. Microbial species include bacteria, fungi, parasites, viruses, and archaea. Imbalance of the normal gut microbiome has been linked with gastrointestinal conditions such as inflammatory bowel disease (IBD) and irritable bowel syndrome (IBS), and wider systemic manifestations of disease such as obesity and type 2 diabetes (T2D). The microbes of the gut undertake a variety of metabolic functions and are able to produce a variety of vitamins, synthesize essential and nonessential amino acids, and provide other functions. Amongst other functions, the microbiome of an individual provides biochemical pathways for the metabolism of non-digestible carbohydrates; some oligosaccharides that escape digestion; unabsorbed sugars and alcohols from the diet; and host-derived mucins.

The triglycerides data 206B may include data about triglycerides for an individual. In some examples, the triglycerides data 206B can be determined from an At Home Blood Test which in some cases is a finger prick on to a dried blood spot card.

The glucose data 206C includes data about blood glucose. The glucose data 206C may be determined from various testing mechanisms, including at home measurements, such as a continuous glucose meter.

The blood data 206D may include blood tests relating to a variety of different biomarkers. As discussed above, at least some blood tests can be performed at home. In some configurations, the blood data 206D is associated with measuring blood sugar, insulin, c-peptides, triglycerides, IL-6 inflammation, ketone bodies, nutrient levels, allergy sensitivities, iron levels, blood count levels, HbA1c, and the like.

The wearable data 206E can include any data received from a computing device associated with an individual. For instance, an individual may wear an electronic data collection device 103, such as an activity-monitoring device, that monitors motion, heart rate, determines how much an individual has slept, the number of calories burned, activities performed, blood pressure, body temperature, and the like. The individual may also wear a continuous glucose meter that monitors blood glucose levels.

The questionnaire data 206F can include data received from one or more questionnaires, and/or surveys received from one or more individuals. The psychological data 206G, that may be subjectively obtained, may include data received from the individual and/or a computing device that generates data or input based on a subjective determination (e.g., the individual states that they are still hungry after a meal, or a device estimates sleep quality based on the movement of the user at night perhaps combined with heart rate data). The objective health data 206H includes data that can be objectively measured, such as but not limited to height, weight, medical history, and the like.

The nutritional data 140B can include data about food, which is referred to herein as “food data”. For example, the nutritional data can include nutritional information about different food(s) such as their macronutrients and micronutrients or the bioavailability of its nutrients under different conditions (raw vs cooked, or whole vs ground up). In some examples, the nutritional data 140C can include data about a particular food. For instance, before an individual consumes a particular meal, information about that food can be determined. As briefly discussed, the user might scan a barcode on the food item(s) being consumed and/or take one or more pictures of the food to determine the food, as well as the amount of food, being consumed.

The nutritional data can include food data that identifies foods consumed, a quantity of the foods consumed, food nutrition (e.g., obtained from a nutritional database), food state (e.g., cooked, reheated, frozen, etc.), food timing data (e.g., what time was the food consumed, how long did it take to consume, . . . ), and the like. The food state can be relevant for foods such as carbohydrates (e.g., pasta, bread, potatoes or rice), since carbohydrates may be altered by processes such as starch retrogradation. The food state can also be relevant for quantity estimation of the foods, since foods can change weight dramatically during cooking. In some instances, the user may also take a picture before and/or after consuming a meal to determine what food was consumed as well as how much of the food was consumed. The picture can also provide an indication as to the food state.

The other data 142B can include other data associated with the individual. For example, the other data 142B can include data that can be received directly from a computer application that logs information for an individual (e.g., food eaten, sleep, . . . ) and/or from the user via a user interface.

In some examples, different computing devices 102 associated with different users provide application data 204 to the data manager 112 for ingestion by the data ingestion service 110. As illustrated, computing device 102A provides app data 204A to the data manager 112, computing device 104B provides app data 204B to the data manager 112, and computing device 104N provides app data 204N to the data manager 112. There may be any number of computing devices utilized.

As discussed briefly above, the data manager 112 receives data from different data sources, processes the data when needed (e.g., cleans up the data for storage in a uniform manner), and stores the data within one or more data stores, such as the data store 140.

The data manager 112 can be configured to perform processing on the data before storing the data in the data store 140. For example, the data manager 112 may receive data for ketone bodies and then use that data to generate ketone body ratios. Similarly, the data manager 112 may process food eaten and generate meal calories, number of carbohydrates, fat to carbohydrate rations, how much fiber consumed during a time period, and the like. The data stored in the data store 140, or some other location, can be utilized by the microbiome service 120 to determine an accuracy of at home measurements of nutritional responses performed by users. The data outputted by the microbiome service 120 to the nutritional service may therefore contain different values than are stored in the data store 140, for example if a food quantity is adjusted.

FIGS. 3-7 are flow diagrams showing processes 300, 400, 500, 600, and 700, respectively that illustrate aspects of generating microbiome fingerprints, dietary fingerprints, and microbiome ancestry data in accordance with examples described herein. It should be appreciated that at least some of the logical operations described herein with respect to FIGS. 3-7, and the other FIGs., may be implemented (1) as a sequence of computer implemented acts or program modules running on a computing system and/or (2) as interconnected machine logic circuits or circuit modules within the computing system.

The implementation of the various components described herein is a matter of choice dependent on the performance and other requirements of the computing system. Accordingly, the logical operations described herein are referred to variously as operations, structural devices, acts, or modules. These operations, structural devices, acts, and modules may be implemented in software, in firmware, in special purpose digital logic and any combination thereof. It should also be appreciated that more or fewer operations may be performed than shown in the FIGs. and described herein. These operations may also be performed in parallel, or in a different order than those described herein.

FIG. 3 is a flow diagram showing a process 300 illustrating aspects of a mechanism disclosed herein for obtaining and utilizing microbiome data for a user to generate microbiome fingerprints, dietary fingerprints, and microbiome ancestry for users.

The process 300 may begin at 302, where microbiome sample/data is obtained from a user. As discussed above, a user may provide one or more microbiome samples that may be obtained at home or in a clinical setting. For example, the user may provide a sample or samples of their stool for microbiome analysis as part of the at home biological collection, and/or the sample(s) may be collected in a lab, or other clinical setting. In some configurations, the user may also provide other data that may be utilized when processing the sample. For instance, the user may provide timing data indicating when the sample was taken, conditions under which the sample was obtained, and/or other health data.

At 304, the microbiome data is processed. As discussed above, microbiome service 120 may generate DNA data from the sample. In some examples, the DNA is extracted from the cells of the microbiome sample and purified. Different techniques that are commercially available can be utilized for DNA extraction from the microbiome sample. Generally, the use of different extraction techniques may result in different biases that may affect an accurate microbial representation.

At 306, the microbial composition of the microbiome sample may be identified. According to some configurations, the microbiome service 120, or some other device or component, identifies the microbial composition of the microbiome (e.g., the species, genes, taxa, and the like). The microbiome service 120 may also generate reconstructed microbial genomes, determine a diversity of the microbiome, identify functions of the microbiome, identify a uniqueness of the microbiome, identify interesting species, and the like.

At 308, the diversity of the microbiome may be determined. As discussed above, the microbiome service 120 may determine the diversity of the microbiome associated with a user. In some examples, the diversity determined by the microbiome service 120 is the number of individual bacteria from each of the bacterial species present in the microbiome. Having a more diverse microbiome may have health benefits. According to some configurations, the microbiome service 120 may provide this data, possibly along with recommendations, to the user via a UI, or some other interface.

At 310, reconstructed microbial genomes are generated. The microbiome service 120, or some other component or device may generate the reconstructed microbial genomes. Reconstruction of DNA fragments into genomes may utilize different techniques and methods and generally incorporates sequence assembly and sorting/clustering of assembled sequences into different bins associated with characteristic of a genome.

At 312, the functions of a microbiome may be determined. As discussed above, the microbiome service 120, or some other device or component, may determine the functions of a microbiome. Different techniques and methods may be utilized to determine the functions. Generally, the microbiome service 120 may map the sequencing reads against sequences of DNA (or amino acids) representing known genes (or proteins) and gene families (or protein families) to determine the functional potential of the microbiome.

At 314, other data associated with the microbiome of the user may be determined. As discussed above, the microbiome service 120, or some other device or component, may determine data such as the uniqueness of the microbiome (e.g., compared to the microbiome of other users), species identified as interesting, and the like.

At 316, the microbiome data associated with the user is stored. As discussed above, the microbiome service 120, or some other device or component, may store the microbiome data in a data store, such as user microbiome data 140A within data store 140.

At 318, the microbiome data associated with the user is utilized to generate microbiome fingerprints, dietary fingerprints, and microbiome ancestry for the user. As discussed above, the microbiome service 120, or some other device or component, may perform these tasks. See FIGS. 4-6 and related discussion for more details.

FIG. 4 is a flow diagram showing a process 400 illustrating aspects of a mechanism disclosed herein for generating a microbiome fingerprint for a user. As discussed above, the microbiome fingerprint may be generated using various techniques and methods. The following process is an example of generating a microbiome fingerprint.

At 402, microbiome data for a particular user is accessed. As discussed above, the microbiome service 120, or some other device or component, may access the microbiome data 140A within data store 140 to obtain the microbiome data for a user. In other examples, the microbiome data may be obtained/accessed using some other technique (e.g., accessing a different memory, receiving the data from some other source, such as data source(s) 150, and the like).

At 404, the microbiome data may be preprocessed to generate screened microbiome data. As discussed above, the microbiome service 120, or some other device or component, may process the sequencing data to generate screened sequencing data. The screened sequence data may make the generation of the different profiles described below be more accurate.

At 406, the quantitative taxonomic profiles are generated. As discussed above, the microbiome service 120, or some other device or component, may generate the quantitative taxonomic profiles. The quantitative taxonomic profiles can be obtained by mapping (i.e. matching the sequences) the sequencing reads against sequences representing the known microbial organisms. The mapping is then processed to produce relative abundances of the reference microbes. Many open source algorithms and corresponding implementations are available for this step, including for example, the techniques as described by Truong et al. (Nature Methods 12 (10): 902-3, 2015) and the newer versions of the associated software.

At 408, the quantitative functional potential profiles are generated. As discussed above, the microbiome service 120, or some other device or component, may generate the quantitative functional potential profiles. The quantitative functional potential profiles can be obtained by mapping the sequencing reads against sequences of DNA (or amino acids) representing known genes (or proteins) and gene families (or protein families). Based on the number of reads matching each gene or gene family the presence and abundance of the gene families and pathways are inferred. Several open source algorithms and corresponding implementations are available for this step, including for example the technique HUMAnN2 as described by Abubucker et al. (PLoS Computational Biology 8 (6), 2012) and Franzosa et al. (Nature Methods, 15(11), 962, 2018) and any newer versions of the associated software.

At 410, the strain-level genomic profiles are generated. As discussed above, the microbiome service 120, or some other device or component, may generate the strain-level genomic profiles. The strain-level genomic profiles, or the third descriptor, can be obtained with reference-based and assembly-based approaches. For reference-based approaches the methods use specific genetic markers against which the reads are mapped, and single-nucleotide polymorphisms are inferred. The combinations of single-nucleotide polymorphisms provide strain-specific profiles. Some open source algorithms and implementations for this step are available, including for example the techniques described by Truong et al. (Genome Research 27 (4): 626-38, 2017). In assembly-based approaches, reads may be first concatenated to form longer contiguous sequences such as described by Li et al. (Bioinformatics 31 (10): 1674-76, 2015).

Contigs may then be clustered in bins representing the sequences of whole genomes, such as described by Kang et al. (PeerJ 7: e7359, 2019). The resulting draft genomes may be quality controlled using for example the techniques described by Parks et al. (Genome Research 25 (7): 1043-55, 2015). The quality-controlled genomes represent single strains in the microbiome.

At 412, the microbiome fingerprint for the user is generated. As discussed above, the microbiome service 120, or some other device or component, may combine the data associated with the different indexes generated at 406, 408, and 410 to generate the microbiome fingerprint for the user.

FIG. 5 is a flow diagram showing a process 500 illustrating aspects of a mechanism disclosed herein for generating a dietary fingerprint for a user.

The process 500 may begin at 502, where microbiome data for a particular user are accessed. As discussed above, the microbiome service 120, or some other device or component, may access the microbiome data 140A within data store 140 to obtain the microbiome data for a user. In other examples, the microbiome data may be obtained/accessed using some other technique (e.g., accessing a different memory, receiving the data from some other source, such as data source(s) 150, and the like).

At 504, dietary fingerprint data is generated. As discussed above, the microbiome service 120, or some other device or component, may generate dietary fingerprint data that identifies a similarity between the microbiome of a particular user and a “dietary fingerprint” is data that identifies how the microbiome of a user is associated with one or more different indexes. The indexes may include, but are not limited to a Mediterranean diet index, a vegetarian diet index, a fast food index, an internal fat index, a fat-digesting index, a carbohydrate-digesting index, a health index, a fasting index, a ketogenic index, and the like. According to some configurations, one or more computers of a microbiome service generate a score, such as from 0-100, (or some other indicator) that indicates how closely the microbiome of the user is associated with a particular index.

As an example, the Mediterranean diet index score for a user indicates how closely the microbiome of the user resembles the typical microbiome of someone on a Mediterranean diet. The vegetarian diet index score indicates how closely the microbiome of the user resembles someone on a vegetarian diet. The fast food index score indicates how closely the microbiome of the user resembles someone on a fast food diet. The internal fat index score indicates how closely the microbiome of the user resembles someone with high or low visceral fat. The fat-digesting index score indicates how closely the microbiome of the user resembles someone with low postprandial triacylglycerol (TAG) rises. The carbohydrate-digesting index score indicates how closely the microbiome of the user resembles someone with low postprandial glucose rises. The health index score indicates how closely the microbiome of the user resembles someone that is healthy. The fasting index score indicates how closely the microbiome of the user resembles someone that fasts regularly. The ketogenic index score indicates how closely the microbiome of the user resembles someone who is ketogenic.

At 506, a determination is made as to whether another dietary index is to be compared. As discussed above, there may be a variety of dietary indexes, including but not limited to a Mediterranean diet index, a vegetarian diet index, a fast food index, an internal fat index, a fat-digesting index, a carbohydrate-digesting index, a health index, a fasting index, a ketogenic index, and the like. When there is another index, the process 500 returns to 504. When there is not another index, the process 500 moves to 508.

At 508, the dietary index(es) associated with the user are identified. As discussed above, the microbiome service 120, or some other device or component, may identify one or more diets that resemble the microbiome of the user. In some examples, the microbiome service 120 identifies the closest dietary index (e.g., based on a score). In other examples, the microbiome service 120 may rank the dietary index.

At 510, the dietary fingerprint data may be utilized. As discussed above, the microbiome service 120, or some other device or component, may utilize the dietary fingerprint data when providing data to the user, when generating the microbiome ancestry data, generating recommendations for the user (e.g., nutritional), and/or performing some other task.

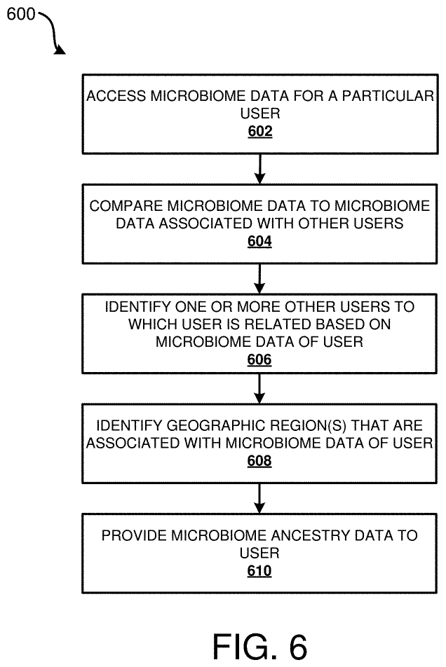

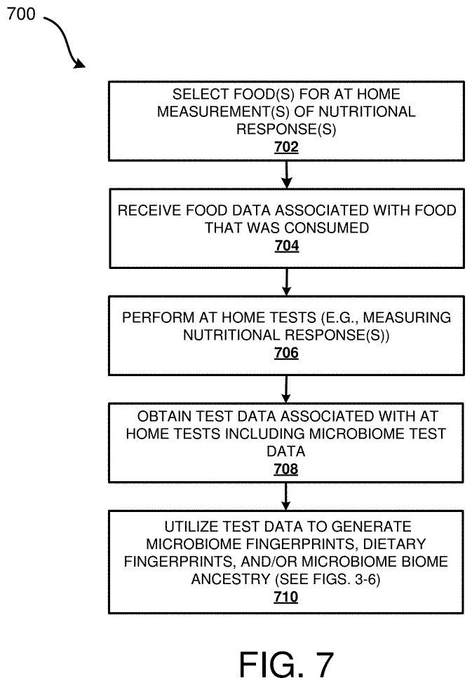

FIG. 6 is a flow diagram showing a process 600 illustrating aspects of a mechanism disclosed herein for generating a microbiome ancestry for a user.

The process 600 may begin at 602, where microbiome data for a particular user is accessed. As discussed above, the microbiome service 120, or some other device or component, may access the microbiome data 140A within data store 140 to obtain the microbiome data for a user. In other examples, the microbiome data may be obtained/accessed using some other technique (e.g., accessing a different memory, receiving the data from some other source, such as data source(s) 150, and the like).

At 604, the microbiome data is compared to microbiome data from other users. As discussed above, the microbiome service 120, or some other device or component, may utilize the microbiome data, such as the microbiome fingerprint data of a particular user, and compare microbiome fingerprint data of other users. According to some configurations, the microbiome service 120 may generate one or more indicators that identify how close another user is to the user based on a similarity of the microbiome data.

At 606, one or more other users are identified based on a similarity of the microbiome data between the users. As discussed above, the microbiome service 120, or some other device or component, may identify the related users based on a score generate the microbiome service 120, or some other indicators.

At 608, the geographic region(s) that are commonly associated with the microbiome data of a user are identified. As discussed above, the microbiome service 120, or some other device or component, may identify that different geographic regions are more closely linked to certain microbiomes.

At 610, the microbiome ancestry data may be utilized. As discussed above, the microbiome service 120, or some other device or component, may utilize the microbiome ancestry data when providing data to the user, when generating the microbiome ancestry data, generating recommendations for the user (e.g., nutritional), and/or performing some other task.

FIG. 7 is a flow diagram showing a process 700 illustrating aspects of a mechanism disclosed herein for obtaining test data, including microbiome data, to be utilized for generating microbiome fingerprints, dietary fingerprints, and microbiome ancestry for users.

At 702, food(s) for at home measurements of nutritional responses may be selected. As briefly discussed above, different foods may be selected for a user to eat before a test is performed in order to evoke a desired response. The foods can include foods for a series of standardized meals, a single food, or some other combination of foods.

At 704, food data is received. As discussed above, the food data is associated with foods that are utilized to evoke a nutritional response. The food data can include foods for a series of standardized meals, a single food, or some other combination of foods. The food data can include data such as foods consumed, a quantity of the foods consumed, food nutrition (e.g., obtained from a nutritional database), food state (e.g., cooked, reheated, frozen, etc.), food timing data (e.g., what time was the food consumed, how long did it take to consume, . . . ), and the like. The food state can be relevant for foods such as carbohydrates (e.g., pasta, bread, potatoes or rice), since carbohydrates may be altered by processes such as starch retrogradation. The food state can also be relevant for quantity estimation of the foods, since foods can change weight dramatically during cooking.

At 706, at home test(s) are performed. The tests may include at home tests as described above and/or the collection of one or more samples (e.g., stool for microbiome analysis).

At 708, test data associated with the at home tests including microbiome data is received. As discussed above, microbiome data may be associated with one or more tests. In some configurations, the microbiome data includes a stool sample, timing data for the sample (e.g., when collected, how long stored before providing to a lab), data associated with collection of the sample (e.g., how was sample stored, was the sample contaminated), as well as other data. For example, a user may be instructed to take a picture of the sample and provide the image to the service.

At 710, the test data is utilized to generate microbiome fingerprints, dietary fingerprints, and microbiome ancestry. In some examples, the test data is used by the microbiome service 120 to generate the microbiome fingerprints, dietary fingerprints, and microbiome ancestry. The nutritional service 132 may also use the test data to generate nutritional recommendations that are personalized for a particular user.

### (II) REPRESENTATIVE COMPUTER ARCHITECTURE

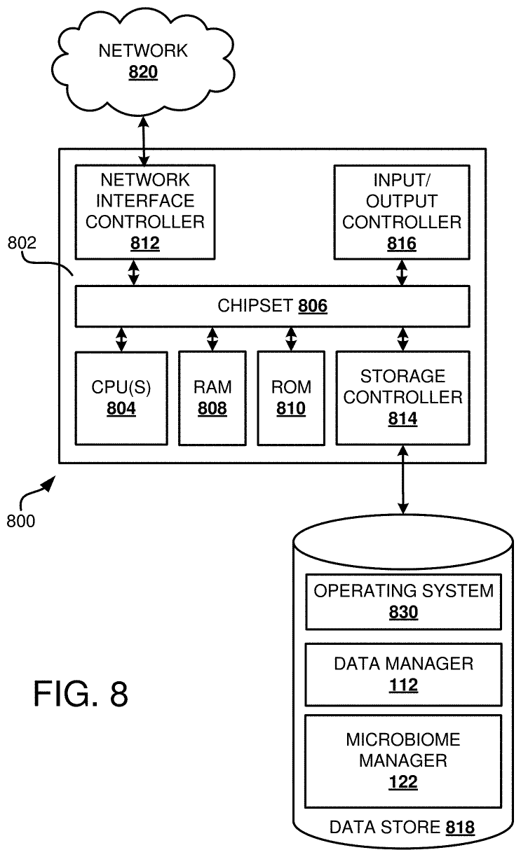

FIG. 8 shows an example computer architecture for a computer 800 capable of executing program components for generating microbiome fingerprints, dietary fingerprints, and microbiome ancestry for users in the manner described above. The computer architecture shown in FIG. 8 illustrates a conventional server computer, workstation, desktop computer, laptop, tablet, network appliance, digital cellular phone, smart watch, or other computing device, and may be utilized to execute any of the software components presented herein. For example, the computer architecture shown in FIG. 8 may be utilized to execute software components for performing operations as described above. The computer architecture shown in FIG. 8 might also be utilized to implement a computing device 102, or any other of the computing systems described herein.

The computer 800 includes a baseboard 802, or “motherboard,” which is a printed circuit board to which a multitude of components or devices may be connected by way of a system bus or other electrical communication paths. In one illustrative example, one or more central processing units (CPUs) 804 operate in conjunction with a chipset 806. The CPUs 804 may be standard programmable processors that perform arithmetic and logical operations necessary for the operation of the computer 800.

The CPUs 804 perform operations by transitioning from one discrete, physical state to the next through the manipulation of switching elements that differentiate between and change these states. Switching elements may generally include electronic circuits that maintain one of two binary states, such as flip-flops and electronic circuits that provide an output state based on the logical combination of the states of one or more other switching elements, such as logic gates. These basic switching elements may be combined to create more complex logic circuits, including registers, adders-subtractors, arithmetic logic units, floating-point units and the like.

The chipset 806 provides an interface between the CPUs 804 and the remainder of the components and devices on the baseboard 802. The chipset 806 may provide an interface to a random-access memory (RAM) 808, used as the main memory in the computer 800. The chipset 806 may further provide an interface to a computer-readable storage medium such as a read-only memory (ROM) 810 or non-volatile RAM (NVRAM) for storing basic routines that help to startup the computer 800 and to transfer information between the various components and devices. The ROM 810 or NVRAM may also store other software components necessary for the operation of the computer 800 in accordance with the examples described herein.

The computer 800 may operate in a networked environment using logical connections to remote computing devices and computer systems through a network, such as the network 820. The chipset 806 may include functionality for providing network connectivity through a network interface controller (NIC) 812, such as a mobile cellular network adapter, WiFi network adapter or gigabit Ethernet adapter. The NIC 812 is capable of connecting the computer 800 to other computing devices over the network 820. It should be appreciated that multiple NICs 812 may be present in the computer 800, connecting the computer to other types of networks and remote computer systems.

The computer 800 may be connected to a mass storage device 818 that provides non-volatile storage for the computer. The mass storage device 818 may store system programs, application programs, other program modules and data, which have been described in greater detail herein. The mass storage device 818 may be connected to the computer 800 through a storage controller 814 connected to the chipset 806. The mass storage device 818 may include one or more physical storage units. The storage controller 814 may interface with the physical storage units through a serial attached SCSI (SAS) interface, a serial advanced technology attachment (SATA) interface, a fiber channel (FC) interface, or other type of interface for physically connecting and transferring data between computers and physical storage units.

The computer 800 may store data on the mass storage device 818 by transforming the physical state of the physical storage units to reflect the information being stored. The specific transformation of physical state may depend on various factors, in different implementations of this description. Examples of such factors may include, but are not limited to, the technology used to implement the physical storage units, whether the mass storage device 818 is characterized as primary or secondary storage and the like.

For example, the computer 800 may store information to the mass storage device 818 by issuing instructions through the storage controller 814 to alter the magnetic characteristics of a particular location within a magnetic disk drive unit, the reflective or refractive characteristics of a particular location in an optical storage unit, or the electrical characteristics of a particular capacitor, transistor, or other discrete component in a solid-state storage unit. Other transformations of physical media are possible without departing from the scope and spirit of the present description, with the foregoing examples provided only to facilitate this description. The computer 800 may further read information from the mass storage device 818 by detecting the physical states or characteristics of one or more particular locations within the physical storage units.

In addition to the mass storage device 818 described above, the computer 800 may have access to other computer-readable storage media to store and retrieve information, such as program modules, data structures, or other data. It should be appreciated by those skilled in the art that computer-readable storage media is any available media that provides for the non-transitory storage of data and that may be accessed by the computer 800.

By way of example, and not limitation, computer-readable storage media may include volatile and non-volatile, removable and non-removable media implemented in any method or technology. Computer-readable storage media includes, but is not limited to, RAM, ROM, erasable programmable ROM (EPROM), electrically-erasable programmable ROM (EEPROM), flash memory or other solid-state memory technology, compact disc ROM (CD-ROM), digital versatile disk (DVD), high definition DVD (HD-DVD), BLU-RAY, or other optical storage, magnetic cassettes, magnetic tape, magnetic disk storage or other magnetic storage devices, or any other medium that can be used to store the desired information in a non-transitory fashion.

The mass storage device 818 may store an operating system 830 utilized to control the operation of the computer 800. According to one example, the operating system includes the LINUX® (Linus Torvalds, Boston, Mass.) operating system. According to another example, the operating system includes the WINDOWS® SERVER® (Microsoft Corporation, Redmond, Wash.) operating system from MICROSOFT® (Microsoft Corporation, Seattle, Wash.). According to another example, the operating system includes the iOS® (Cisco Technology Inc., San Jose, Calif.) operating system from Apple® (Apple Inc., Cupertino, Calif.). According to another example, the operating system includes the Android® (Google LLC, Mountain View, Calif.) operating system from Google® (Google LLC) or its ecosystem partners. According to further examples, the operating system may include the UNIX® (The Open Group Limited, Reading, Berkshire, England) operating system. It should be appreciated that other operating systems may also be utilized. The mass storage device 818 may store other system or application programs and data utilized by the computer 800, such as components that include the data manager 122, the microbiome manager 122 and/or any of the other software components and data described above. The mass storage device 818 might also store other programs and data not specifically identified herein.

In one example, the mass storage device 818 or other computer-readable storage media is encoded with computer-executable instructions that, when loaded into the computer 800, create a special-purpose computer capable of implementing the examples described herein. These computer-executable instructions transform the computer 800 by specifying how the CPUs 804 transition between states, as described above. According to one example, the computer 800 has access to computer-readable storage media storing computer-executable instructions which, when executed by the computer 800, perform the various processes described above with regard to FIGS. 4-8. The computer 800 might also include computer-readable storage media for performing any of the other computer-implemented operations described herein.

The computer 800 may also include one or more input/output controllers 816 for receiving and processing input from a number of input devices, such as a keyboard, a mouse, a touchpad, a touch screen, an electronic stylus, or other type of input device. Similarly, the input/output controller 816 may provide output to a display, such as a computer monitor, a flat-panel display, a digital projector, a printer, a plotter, or other type of output device. It will be appreciated that the computer 800 may not include all of the components shown in FIG. 8, may include other components that are not explicitly shown in FIG. 8, or may utilize an architecture completely different than that shown in FIG. 8.

### (III) DETECTION AND IDENTIFICATION OF INDIVIDUAL MICROBES

Described herein are specific methods for detecting and identifying individual member microbes in the microbiome of a subject, as well as methods for identifying and quantifying (in relative or absolute terms) the members of a microbiome. It will be understood, however, that other methods known to those of skill in the art can also be used with the methods described herein. See, for instance: Davidson & Epperson (Methods Mol. Biol., 1706:77-90, 2018), Nagpal et al. (Front Microbiol., 8:2897, doi:10.3389/fmicb.2018.02897, 2018), Nagpal et al. (Sci Rep. 8(1):12649, 2018), The Integrative HMP (iHMP) Research Network Consortium (Nature 569:641-648, 2019; and publications cited therein), Wu et al. (Gut. 65(1):63-72, 2016). Additional resources are available online, for instance, through the NIH Human Microbiome Project (at hmpdacc.org), including tools and protocols related to Microbial Reference Genomes, Sampling, Sequence & Analysis of 16S RNA, and Sampling, Sequencing & Analysis of Whole Metagenomic Sequence.

Having provided in this disclosure specific individual microbes and sets of microbes associated with and/or linked to poor health and others associated with and/or linked to pro-health conditions, profiles can now be detected without needing to sequence or otherwise assay the entire microbiome of the subject. For instance, the following are pro-health linked/indicator microbes: Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Osciffibacter sp PC13, Clostridium sp CAG 167, Ruminococcaceae bacterium D5, Paraprevotella xylaniphila, Faecalibacterium prausnitzii, Romboutsia ilealis, and Veillonella atypica; and the following are poor health linked/indicator microbes: Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli. These strains can be further identified by their respective NCBI Taxonomy ID Number (see ncbi.nlm.nih.gov/taxonomy), as shown in Table 6. Additional specific taxonomic information can be found, for instance, using MetaPhIAn2 (Metagenomic Phylogenetic Analysis; version 2.9.21 and marker database release 2.9.4; Truong et al., Nat. Methods 12, 902-903, 2015).

TABLE 6
NCBI Taxonomy Identification Numbers for Select Indicator Microbes

| NCBI: txid | Species label | Indicator |
|---|---|---|
| 537011 | Prevotella copri | Pro-Health |
| 12967 | Blastocystis spp. | |
| 28025 | Bifidobacterium animalis | |
| 1262777 | Clostridium sp CAG 167 | |
| 39485 | Eubacterium eligens | |
| 1263006 | Firmicutes bacterium CAG 170 | |
| 1262988 | Firmicutes bacterium CAG 95 | |
| 729 | Haemophilus parainfluenzae | |
| 1897011 | Oscillibacter sp 57 20 | |
| 1855299 | Oscillibacter sp PC13 | |
| 454155 | Paraprevotella xylaniphila | |
| 301301 | Roseburia hominis | |
| 1262942 | Roseburia sp CAG 182 | |
| 43675 | Rothia mucilaginosa | |
| 1520815 | Ruminococcaceae bacterium D5 | |
| 39778 | Veillonella dispar | |
| 1911679 | Veillonella infantium | |
| 853 | Faecalibacterium prausnitzii | |
| 1115758 | Romboutsia ilealis | |
| 39777 | Veillonella atypica | |
| 169435 | Anaerotruncus colihominis | Poor Health |
| 53443 | Blautia hydrogenotrophica | |
| 40520 | Blautia obeum | |
| 208479 | Clostridium bolteae | |
| 1263064 | Clostridium bolteae CAG 59 | |
| 1522 | Clostridium innocuum | |
| 1262824 | Clostridium sp CAG 58 | |
| 29348 | Clostridium spiroforme | |
| 1512 | Clostridium symbiosum | |
| 147207 | Collinsella intestinalis | |
| 84112 | Eggerthella lenta | |
| 39496 | Eubacterium ventriosum | |
| 292800 | Flavonifractor plautii | |
| 360807 | Roseburia inulinivorans | |
| 33038 | Ruminococcus gnavus | |
| 1535 | Clostridium leptum | |
| 1550024 | Ruthenibacterium lactatiformans | |
| 562 | Escherichia coli | |

A collection of two or more microbes described or illustrated herein as associated with a biological status or condition can be referred to as a microbial signature, or a microbiome fingerprint. For instance, any two, any three, any four, any five, any six, any seven, any eight, any nine, any 10, any 11, any 12, any 13, any 14, any 15, or more microbes listed in Table 6 may be included in a microbial signature for a biological status or condition. Such microbes may be selected from the Pro-Health or the Poor Health indicators, or some from both. All seventeen of the listed pro-health indicator microbes for instance may be included in a single microbial signature. Similarly, all fifteen poor health indicator microbes may be included in a single microbial signature. Additional microbes useful in the assembling of a microbial signature, or microbiome fingerprint, are provided for instance in Table 5, and are discussed more fully in Example 1.

### (IV) METHODS OF USE

Based on the research reported herein, including specifically in Example 1, there are now enabled a number of methods of using the results of the microbiome metagenomic analyses.

For instance, one embodiment is a method of using a group of microbes to determine a health condition in a human subject. By way of example, the group of microbes includes: at least two pro-health indicator microbes; or at least two poor health indicator microbes; or at least two pro-health indicator microbes and at least two poor health indicator microbes. Lists of pro-health and poor health indicator microbes are described herein, for instance in Example 1 and Table 6. By way of example, in some embodiments the pro-health indicator microbes are selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, and Paraprevotella xylaniphila. By way of further example, in some embodiments the poor health indicator microbes are selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, and Flavonifractor plautii. In another example embodiment, at least one of the pro-health indicator microbes is selected from the group including Firmicutes bacterium CAG 95, Haemophilus parainfluenzae, Oscillibacter sp 57 20, Firmicutes bacterium CAG 170, Roseburia sp CAG 182, Clostridium sp CAG 167, Oscillibacter sp PC13, Eubacterium eligens, Prevotella copri, Veillonella dispar, Veillonella infantium, Faecalibacterium prausnitzii, Bifidobacterium animalis, Romboutsia ilealis, and Veillonella atypica; and at least one of the poor health indicator microbes is selected from the group including Clostridium leptum, Ruthenibacterium lactatiformans, Collinsella intestinalis, Escherichia coli, Blautia hydrogenotrophica, Clostridium sp CAG 58, Eggerthella lenta, Ruminococcus gnavus, Clostridium spiroforme, Clostridium bolteae CAG 59, Clostridium innocuum, Anaerotruncus colihominis, Clostridium symbiosum, Clostridium bolteae, and Flavonifractor plautii.

In further examples of such methods, the method of using a group of microbes to determine a health condition in a human subject includes obtaining a biological sample from the human subject (for instance, a microbiome sample, such as a stool sample); and analyzing the biological sample to determine presence, absence, or abundance of the at least two pro-health indicator microbes and/or the at least two poor health indicator microbes.

In additional examples of such methods, the method of using a group of microbes to determine a health condition in a human subject includes obtaining a biological sample from the human subject; identifying in the biological sample at least 10, at least 20, at least 30, at least 40, at least 50, at least 60, at least 70, at least 80, at least 90, at least 100, at least 125, at least 150, at least 175, at least 200, or more than 200 different microbes in the biological sample; and determining the health condition of the human subject based on presence, absence, and/or absolute or relative abundance of the identified microbes in the biological sample.

In any of these methods using a group of microbes to determine a health condition in a human subject, the group of microbes may include at least three pro-health indicator microbes; at least five pro-health indicator microbes; at least ten pro-health indicator microbes; or more than 10 pro-health indicator microbes. Optionally, the group of microbes includes all of the following pro-health indicator microbes: Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, and Paraprevotella xylaniphila. In another example, the group of microbes includes all of the following pro-health indicator microbes: Firmicutes bacterium CAG 95, Haemophilus parainfluenzae, Oscillibacter sp 57 20, Firmicutes bacterium CAG 170, Roseburia sp CAG 182, Clostridium sp CAG 167, Oscillibacter sp PC13, Eubacterium eligens, Prevotella copri, Veillonella dispar, Veillonella infantium, Faecalibacterium prausnitzii, Bifidobacterium animalis, Romboutsia ilealis, and Veillonella atypica.

In any of these methods using a group of microbes to determine a health condition in a human subject, the group of microbes may include: at least three poor health indicator microbes; at least five poor health indicator microbes; at least ten poor health indicator microbes; or more than 10 poor health indicator microbes. Optionally, the group of microbes includes all of the following poor health indicator microbes: Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, and Flavonifractor plautii. In another example, the group of microbes includes all of the following poor health indicator microbes: Clostridium leptum, Ruthenibacterium lactatiformans, Collinsella intestinalis, Escherichia coli, Blautia hydrogenotrophica, Clostridium sp CAG 58, Eggerthella lenta, Ruminococcus gnavus, Clostridium spiroforme, Clostridium bolteae CAG 59, Clostridium innocuum, Anaerotruncus colihominis, Clostridium symbiosum, Clostridium bolteae, and Flavonifractor plautii.

In exemplary method embodiments, the group of microbes includes Clostridium innocuum, C. symbiosum, C. spiroforme, C. leptum, C. saccharolyticum. In exemplary method embodiments, the group of microbes includes P. copri and Blastocystis spp.

In any of these methods of using a group of microbes to determine a health condition in a human subject, the health condition may include at least one of: overall good health, overall poor health, obesity, BMI, diabetes risk, cardiometabolic risk, cardiovascular disease risk, or postprandial response to food intake.

Optionally, any of the provided methods of using a group of microbes to determine a health condition in a human subject may include detecting the presence, absence, or relative abundance of at least one of the microbes in a microbiome sample from the human subject. For instance, in this context the detecting may include one or more of: sequencing one or more nucleic acids of a pro-health or poor health microbe, hybridizing a nucleic acid probe to a nucleic acid of a pro-health or poor health microbe, detecting one or more proteins from a pro-health or poor health microbe, or measuring activity of one or more proteins a pro-health or poor health microbe. For instance, the detecting may include shotgun metagenomics.

Also provided herein are methods of predicting a health condition in a subject. Such methods involve determining presence, absence, or relative abundance of at least three pro-health indicator microbes in a microbiome of the subject; determining presence, absence, or relative abundance of at least three poor health indicator microbes in a microbiome of the subject; and predicting the health condition of the subject, based on the presence, absence, or relative abundance of the pro-health and/or poor health indicator microbes in the microbiome of the subject. By way of example, in some such methods the pro-health indicator microbes are selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis and, Veillonella atypica. By way of further example, in some such methods the poor health indicator microbes are selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli.

It is contemplated that in some methods of predicting a health condition in a subject, the health condition includes at least one of obesity, increased cardiometabolic risk, diabetes risk, or overall poor health; and the health condition is predicted by the presence and/or abundance of more poor health indicator microbes than pro-health indicator microbes; and/or the health condition includes at least one of overall good health or absence of obesity, reduced cardiometabolic risk, or reduced diabetes risk; and the health condition is predicted by the presence and/or abundance of more pro-health indicator microbes than poor health indicator microbes.

Another embodiment is a method to predict overall good or poor general health in a non-diseased human subject. In examples of such methods, the methods involve obtaining a microbiome sample (for instance, a stool sample) from the human subject; isolating a nucleic acid fraction from the microbiome sample; detecting, within the nucleic acid fraction, presence, absence, or relative abundance of at least one unique marker sequence indicative of: a pro-health indicator microbe selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; or a poor health indicator microbes selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli; and at least one of predicting the human subject has overall good general health if the pro-health indicator microbes outnumber or are relatively more abundant than the poor-health indicator microbes; or predicting the human subject has overall poor general health if the poor health indicator microbes outnumber or are relatively more abundant than the pro-health indicator microbes.

Examples of the methods to predict overall good or poor general health in a non-diseased human subject further include providing to the human subject a dietary recommendation based on the presence, absence, or relative abundance of one or more poor health indicator microbes and/or one or more pro-health indicator microbes. Such dietary recommendation may be provided as a prescription. Optionally, the method may further include administering to the subject one or more compounds or substances intended to alter the presence or quantity or relative proportion of at least one pro-health indicator microbe or at least one poor health indicator microbe in the subject.

Also enabled by this disclosure are methods for targeting a microbiome of a human subject to promote health, which methods include (A) detecting in a microbiome sample from the human subject one or more pro-health indicator microbes selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; and administering to the human a composition that increases growth or survival of the pro-health indicator microbe(s); and/or (B) detecting in a microbiome sample from the human subject one or more poor health indicator microbe selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli; and administering to the human a composition that decreases growth or survival of the poor health indicator microbe(s).

Examples of such methods for targeting a microbiome of a human subject to promote health involve detecting: at least three pro-health indicator microbes; at least five pro-health indicator microbes; at least ten pro-health indicator microbes; or more than ten pro-health indicator microbes. All of the following pro-health indicator microbes are detected in some embodiments: Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, and Paraprevotella xylaniphila. Alternatively, the indicator microbes include at least P. copri and Blastocystis spp. Alternatively, the indicator microbes include all of: Prevotella copri, Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Veillonella infantium, Oscillibacter sp PC13, Clostridium sp CAG 167, Faecalibacterium prausnitzii, and Romboutsia ilealis, Veillonella atypica.

Further examples of such methods for targeting a microbiome of a human subject to promote health involve detecting: at least three poor health indicator microbes; at least five poor health indicator microbes; at least ten poor health indicator microbes; or more than ten poor health indicator microbes. All of the following poor health indicator microbes are detected in some embodiments: Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, and Flavonifractor plautii. Alternatively, the indicator microbes include Clostridium innocuum, C. symbiosum, C. spiroforme, C. leptum, and C. saccharolyticum. Alternatively, the indicator microbes include all of: Clostridium leptum, Ruthenibacterium lactatiformans, Collinsella intestinalis, Escherichia coli, Blautia hydrogenotrophica, Clostridium sp CAG 58, Eggerthella lenta, Ruminococcus gnavus, Clostridium spiroforme, Clostridium bolteae CAG 59, Clostridium innocuum, Anaerotruncus colihominis, Clostridium symbiosum, Clostridium bolteae, and Flavonifractor plautii.

Also provided are methods of altering abundance of one or more microbes in gut microflora of a subject, including administering to the subject a probiotic composition, or administering to the subject a prebiotic composition, or administering to the subject an antibiotic composition.

(V) Kits and Arrays.

Also provided herein are various different types of kits. Examples of such kits include kits useful to gather data or information from a subject, for instance. Examples of the information/data-gathering kits include one or more device(s) to in/with which to collect a microbiome sample (for instance, a stool sample collection device, surface swab, etc.), and optionally one or more devices in/with which to collect biological samples (such as blood samples; for instance, a device for the collection of blood spots). Optionally, the kits will also include instructions for how the subject, or a health care provider, is to collect the samples; how those samples are to be treated and/or stored before they are forwarded for analysis; and additional instructions regarding recording information other than biological samples that can inform or influence the interpretation of results from analyses of the biological sample(s). For instance, kits may include instructions on how to install or access computer software useful to collect information from the subject, such as food intake, exercise, and other objective or subject information.

In some kit embodiments, the kit will further include a device or system for monitoring blood glucose of the subject. By way of example, such device may be a continues blood glucose monitor. Alternatively, the kit may provide a system for intermittently monitoring blood glucose, for instance through periodic blood sampling and analysis such as is routine for monitoring the blood glucose of Type 1 diabetics.

It is also contemplated that some kit embodiments will include instructions to enable the subject being tested to undergo one or more additional sampling or testing procedures, for instance at a laboratory or other device outside of their home. For instance, some kits may include instructions for how to provide a fasting blood sample, or more generally a blood sample useful to detect or measure metabolic action.

Additional kit embodiments are provided for the analysis of samples collect from a subject. By way of example, such testing kits include one or more marker molecules capable of detecting the presence (and/or quantity) of at least one indicator microbe in a sample (e.g., a stool or other microbiome sample) from a subject. For instance, marker molecules are nucleic acids (e.g., oligonucleotides) or amino acids (e.g., peptides) specific for a single indicator microbe. Such marker molecules may optionally be attached to a solid surface, such as an array. Marker molecules may optionally be labeled for ease of detection.

A kit can include a device as described herein, and optionally additional components such as buffers, reagents, and instructions for carrying out the methods described herein. The choice of buffers and reagents will depend on the particular application, e.g., setting of the assay (point-of-care, research, clinical), analyte(s) to be assayed, the detection moiety used, the detection system used, etc.

The kit can also include informational material, which can be descriptive, instructional, marketing, or other material that relates to the methods described herein and/or the use of the devices for the methods described herein. In embodiments, the informational material can include information about production of the device, physical properties of the device, date of expiration, batch or production site information, and so forth.

Also contemplated are arrays of biological macromolecules (markers), such as nucleic acids (e.g., oligonucleotides) or amino acids (e.g., peptides or proteins), that enable the detection and/or quantification of microbes from a microbiome of a subject, such as a human subject. With the provision herein of lists of specific pro-health and specific poor health indicator microbes, arrays can be prepared that specifically can detect and/or quantify such indicator microbes. By way of example, an array may include markers specific for individual pro-health or poor health microbes. Such examples may be genomic sequence determined to be or recognized as being specific for an individual microbe listed, for instance, in Table 5.

Specific arrays are pro-health indicator detection arrays, which contain two or more markers each of which is specific for a pro-health indicator microbe as describe herein, including for instance microbes indicated to be associated with generally good health of the subject from which the microbe is isolated. By way of example, such pro-health indicator microbes may include: Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica. Thus, contemplated herein are pro-health indicator arrays that include at least one marker for each of at least two of these listed pro-health indictor microbes; each of at least three; each of at least four; each of at least five; each of at least six; each of at least seven; each of at least eight; each of at least nine; each of at least ten; or more than ten of these listed pro-health indictor microbes. Some arrays will include all seventeen of the listed pro-health indictor microbes. Optionally, any of these pro-health indicator arrays may also include markers for additional microbes; these may be other pro-health indicator microarrays or poor health indictor microbes, for instance.

Additional specific arrays are poor health indicator detection arrays, which contain two or more markers each of which is specific for a poor health indicator microbe as describe herein, including for instance microbes indicated to be associated with generally poor health of the subject from which the microbe is isolated. By way of example, such poor health indicator microbes include: Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli. Thus, contemplated herein are poor health indicator arrays that include at least one marker for each of at least two of these listed poor health indictor microbes; each of at least three; each of at least four; each of at least five; each of at least six; each of at least seven; each of at least eight; each of at least nine; each of at least ten; or more than ten of these listed poor health indictor microbes. Some arrays will include all fifteen of the listed poor health indictor microbes. Optionally, any of these poor health indicator arrays may also include markers for additional microbes; these may be other poor health indicator microarrays or pro-health indictor microbes, for instance.

The arrays may be utilized in myriad applications. For example, the arrays in some embodiments are used in methods for detecting association between a behavior (such as a food choice, or more generally, a diet) and a health condition. For instance, such a health condition may include balance (or imbalance) of the normal gut microbiome; gastrointestinal conditions such as inflammatory bowel disease (IBD) and irritable bowel syndrome (IBS); wider systemic manifestations of disease or disorder, such as obesity, type 2 diabetes (T2D), diabetes risk, metabolic syndrome, prediabetes, and obesity; as well as overall good health, overall poor health, BMI, cardiometabolic risk, cardiovascular disease risk, and postprandial response to food intake. This method typically includes incubating a sample from a subject (e.g., from the microbiome of the subject) with the array under conditions such that biomolecules in the sample may associate with marker biomolecules attached to the array. The association is then detected, using means commonly known in the art. In this context, the term association may include hybridization, covalent binding, or ionic binding, for instance. A skilled artisan will appreciate that conditions under which association occurs will vary depending on the biomolecules, the markers, the substrate, and the detection method utilized. As such, suitable conditions can be optimized for each individual array created or assay carried out with an array.

In yet another embodiment, the array is used as a tool in a method to determine whether a compound or composition is effective to modify a biological condition, such as the balance or imbalance of the microbiome in a subject, or for a treatment of a disease or disorder in a subject.

In another embodiment, the array is used as a tool in a method to determine whether a compound increases or decreases the relative abundance in a subject of any of the pro-health or poor health indicator microbes describe herein. Typically, such methods include comparing the presence, absence, and/or quantity of one or more indicator microbes in a subject's microbiome before and after administration of a compound or composition. If the abundance of biomolecule(s) associated with at least one pro-health microbe increases after treatment, or the abundance of biomolecule(s) associated with at least one poor health microbe decreases, or if the relative abundance of biomolecule(s) shifts to be more similar to a “healthy” profile or fingerprint discussed herein, the compound or composition may be effective in improving the health of the subject.

### (VI) SYSTEMS

Also provided are systems to assay a biological condition in a subject, such as a human or other mammalian subject. By way of example, such a system includes: a nucleic acid sample isolation device, which is adapted to isolate a nucleic acid sample from the subject; a sequencing device, which is connected to the nucleic acid sample isolation device and adapted to sequence the nucleic acid sample, thereby obtaining a sequencing result; and an alignment device, which is connected to the sequencing device and adapted to align the sequencing result against sequence from one or more of microbes in order to determine presence or absence of the microbe(s) based on the alignment result. In examples of such systems, the microbes include one or more of: pro-health indicator microbes selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; and/or poor health indicator microbes selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli.

Optionally, the systems may further include an information delivery device capable of delivering to the subject information about the results of the alignment. Such information may include one or more of: the identity and/or relative or absolute quantity of one or more microbes, such as microbes found or not found in the microbiome of the subject; information on the subject's gut microbiome health; information on the health of the subject, for instance based the presence, absence, or relative abundance of one or microbes in the subject's microbiome; one or more recommendations for how to modify the subject's diet; a specific recommendation for a food to eat, or a food to avoid; information on general diet plan(s); options for lifestyle choices; and so forth.

The Exemplary Embodiments and Example(s) below are included to demonstrate particular embodiments of the disclosure. Those of ordinary skill in the art will recognize in light of the present disclosure that many changes can be made to the specific embodiments disclosed herein and still obtain a like or similar result without departing from the spirit and scope of the disclosure.

### (VII) EXEMPLARY EMBODIMENTS

1. A method of using a group of microbes to determine a health condition in a human subject, wherein the group of microbes includes: at least two pro-health indicator microbes; or at least two poor health indicator microbes; or at least two pro-health indicator microbes and at least two poor health indicator microbes; wherein the pro-health indicator microbes are selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; and wherein the poor health indicator microbes are selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli.
2. The method of embodiment 1, including: obtaining a biological sample from the human subject; and analyzing the biological sample to determine presence, absence, or abundance of the at least two pro-health indicator microbes and/or the at least two poor health indicator microbes.
3. The method of embodiment 1, including: obtaining a biological sample from the human subject; identifying in the biological sample at least 10, at least 20, at least 30, at least 40, at least 50, at least 60, at least 70, at least 80, at least 90, at least 100, at least 125, at least 150, at least 175, at least 200, or more than 200 different microbes in the biological sample; and determining the health condition of the human subject based on presence, absence, and/or absolute or relative abundance of the identified microbes in the biological sample.
4. The method of embodiment 1, wherein the group of microbes includes: at least three pro-health indicator microbes; at least five pro-health indicator microbes; at least ten pro-health indicator microbes; or more than 10 listed pro-health indicator microbes.
5. The method of embodiment 1, wherein the group of microbes includes: at least three poor health indicator microbes; at least five poor health indicator microbes; at least ten poor health indicator microbes; or more than 10 listed poor health indicator microbes.
6. The method of embodiment 1, wherein the group of microbes includes Clostridium innocuum, C. symbiosum, C. spiroforme, C. leptum, C. saccharolyticum.
7. The method of embodiment 1, wherein the group of microbes includes P. copri and Blastocystis spp.
8. The method of any one of embodiments 1-3, wherein the health condition includes at least one of: overall good health, overall poor health, obesity, BMI, diabetes risk, cardiometabolic risk, cardiovascular disease risk, or postprandial response to food intake.
9. The method of any one of embodiments 1-8, including detecting the presence, absence, or relative abundance of at least one of the microbes in a microbiome sample from the human subject.
10. The method of embodiment 9, wherein the detecting includes one or more of: sequencing one or more nucleic acids of a pro-health or poor health microbe, hybridizing a nucleic acid probe to a nucleic acid of a pro-health or poor health microbe, detecting one or more proteins from a pro-health or poor health microbe, or measuring activity of one or more proteins a pro-health or poor health microbe.
11. The method of embodiment 9, wherein the detecting includes shotgun metagenomics.
12. The method of any one of embodiments 1-10, wherein the biological sample includes a stool sample.
13. A method of predicting a health condition in a subject, including: determining presence, absence, or relative abundance of at least three pro-health indicator microbes in a microbiome of the subject; determining presence, absence, or relative abundance of at least three poor health indicator microbes in a microbiome of the subject; and predicting the health condition of the subject, based on the presence, absence, or relative abundance of the pro-health and/or poor health indicator microbes in the microbiome of the subject;
wherein the pro-health indicator microbes are selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; and
wherein the poor health indicator microbes are selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli.
   14. The method of embodiment 13, wherein: the health condition includes at least one of obesity, increased cardiometabolic risk, diabetes risk, or overall poor health; and the health condition is predicted by the presence and/or abundance of more poor health indicator microbes than pro-health indicator microbes; and/or the health condition includes at least one of overall good health or absence of obesity, reduced cardiometabolic risk, or reduced diabetes risk; and the health condition is predicted by the presence and/or abundance of more pro-health indicator microbes than poor health indicator microbes.
   15. A method to predict overall good or poor general health in a non-diseased human subject, including: obtaining a microbiome sample from the human subject; isolating a nucleic acid fraction from the microbiome sample; detecting, within the nucleic acid fraction, presence, absence, or relative abundance of at least one unique marker sequence indicative of: a pro-health indicator microbe selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, and Paraprevotella xylaniphila; or a poor health indicator microbes selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli; and at least one of predicting the human subject has overall good general health if the pro-health indicator microbes outnumber or are relatively more abundant than the poor-health indicator microbes; or predicting the human subject has overall poor general health if the poor health indicator microbes outnumber or are relatively more abundant than the pro-health indicator microbes.
   16. The method of embodiment 15, further including providing to the human subject a dietary recommendation based on the presence, absence, or relative abundance of one or more poor health indicator microbes and/or one or more pro-health indicator microbes.
   17. An assay, including: subjecting nucleic acid extracted from a test sample of a human subject to a genotyping assay that detects at least one of Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica, the test sample including microbiota from a gut of the subject; determining a relative abundance of the at least one of Prevotella copri, Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica that is below a predetermined abundance; and selecting, when the relative abundance is below the predetermined abundance, a treatment regimen that includes at least one of: (i) modifying microbiota of the gut of the subject using at least one of a prebiotic, probiotic, or pharmaceutical, or (ii) altering the diet of the human subject.
   18. An assay, including: subjecting nucleic acid extracted from a test sample of a human subject to a genotyping assay that detects at least one of Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli, the test sample including microbiota from a gut of the subject; determining a relative abundance of the at least one Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli, that is above a predetermined abundance; and selecting, when the relative abundance is above the predetermined abundance, a treatment regimen that includes at least one of: (i) modifying microbiota of the gut of the subject using at least one of a prebiotic, probiotic, or pharmaceutical, or (ii) altering the diet of the human subject.
   19. A method of diagnosing a human subject as having a healthy diet, including detecting in a microbiome sample from the subject the presence of Firmicutes CAG95 and/or the absence of Firmicutes CAG94.
   20. A method of diagnosing a human subject as having an unhealthy diet, including detecting in a microbiome sample from the subject the presence of Firmicutes CAG94 and/or the absence of Firmicutes CAG95.
   21. A microbial signature (fingerprint) for good health, including presence or relatively high abundance of at least three microbes selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica, and/or absence or relatively low abundance of at least three microbes selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli.
   22. A microbial signature for poor health, including absence or relatively low abundance of at least three microbes selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica, and/or presence or relatively high abundance of at least three microbes selected from the group including R. gnavus, F. plautii, C. innocuum, C. symbiosum, C. bolteae, A. colihominis, C. intestinalis, B. obeum, R. inulinivorans, E. ventriosum, B. hydrogenotrophica, Clostridium CAG 58, E. lenta, C. bolteae CAG 59, C. spiroforme, Clostridium leptum, Ruthenibacterium lactatiformans, and Escherichia coli.
   23. The microbial signature of embodiment 21, wherein the signature includes: at least three pro-health indicator microbes; at least five pro-health indicator microbes; at least ten pro-health indicator microbes; or more than 10 listed pro-health indicator microbes.
   24. The microbial signature of embodiment 21, wherein the group of microbes includes P. copri and Blastocystis spp.
   25. The microbial signature of embodiment 22, wherein the group of microbes includes: at least three poor health indicator microbes; at least five poor health indicator microbes; at least ten poor health indicator microbes; or more than 10 listed poor health indicator microbes.
   26. The microbial signature of embodiment 22, wherein the group of microbes includes Clostridium innocuum, C. symbiosum, C. spiroforme, C. leptum, C. saccharolyticum.
   27. Use of the microbial signature of any one of embodiments 2-26, to guide treatment decisions for a human subject.
   28. The use of embodiment 27, wherein the treatment decision includes selecting one or more of: modifying overall diet, increasing intake of at least one specified food or supplement, decreasing intake of at least one specified food or supplement, administration of a probiotic composition, administration of a prebiotic composition, or administration of an antibiotic compound.
   29. A method for targeting a microbiome of a human subject to promote health, including: (A) detecting in a microbiome sample from the human subject one or more pro-health indicator microbes selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, and Paraprevotella xylaniphila; and administering to the human a composition that increases growth or survival of the pro-health indicator microbe(s); and/or (B) detecting in a microbiome sample from the human subject one or more poor health indicator microbe selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, and Flavonifractor plautii; and administering to the human a composition that decreases growth or survival of the poorhealth indicator microbe(s).
   30. The method of embodiment 29, including detecting: at least three pro-health indicator microbes; at least five pro-health indicator microbes; at least ten pro-health indicator microbes; or more than 10 listed pro-health indicator microbes. 31. The method of embodiment 29 or embodiment 30, wherein the indicator microbes include P. copri and Blastocystis spp.
   32. The microbial signature of embodiment 29, including detecting: at least three poor health indicator microbes; at least five poor health indicator microbes; at least ten poor health indicator microbes; or more than 10 listed poor health indicator microbes.
   33. The microbial signature of embodiment 29, wherein the indicator microbes include Clostridium innocuum, C. symbiosum, C. spiroforme, C. leptum, C. saccharolyticum.
   34. A probiotic composition for ingestion by a human subject, including at least one of Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica.
   35. The probiotic composition of embodiment 34, including at least three, at least five, at least seven, at least 9, at least 10, at least 12, at least 14, or all of the listed microbes.
   36. The probiotic composition of embodiment 34 or embodiment 35, including Prevotella copri or Blastocystis spp. or both.
   37. A method of altering abundance of one or more microbes in gut microflora of a subject, including administering the probiotic composition of embodiment 34 to the subject. 38. A system to assay a biological condition in a subject, including: a nucleic acid sample isolation device, which is adapted to isolate a nucleic acid sample from the subject; a sequencing device, which is connected to the nucleic acid sample isolation device and adapted to sequence the nucleic acid sample, thereby obtaining a sequencing result; and an alignment device, which is connected to the sequencing device and adapted to align the sequencing result against sequence from one or more of microbes in order to determine presence or absence of the microbe(s) based on the alignment result, wherein the microbes include one or more of: pro-health indicator microbes selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; and/or poor health indicator microbes selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli.

### (VIII) EXAMPLE(S)

### Example 1: Microbiome Connections with Host Metabolism and Habitual Diet from the PREDICT 1 Metagenomic Study

The gut microbiome is shaped by diet and influences host metabolism, but these links remain poorly characterized, are complex and can be unique to each individual. This example describes the deep metagenomic sequencing of more than 1,100 gut microbiomes from individuals with detailed long-term diet information, as well as hundreds of fasting and same-meal postprandial cardiometabolic blood markers. Strong associations were found between microbes and specific nutrients, foods, food groups, and general dietary indices, driven especially by the presence and diversity of healthy and plant-based foods. Microbial biomarkers of obesity were reproducible across cohorts, and blood markers of cardiovascular disease and impaired glucose tolerance were more strongly associated with microbiome structures. Although some microbes, such as Provotella copri and Blastocystis spp., were indicators of reduced postprandial glucose metabolism, several species were more directly predictive for postprandial triglycerides and C-peptide. The panel of intestinal species associated with healthy dietary habits overlapped with those associated with favorable cardiometabolic and postprandial markers, indicating this large-scale resource can potentially stratify the gut microbiome into generalizable health levels among individuals without clinically manifest disease. At least some of the material described in this Example was published as Asnicar et al. (“Microbiome connections with host metabolism and habitual diet from 1,098 deeply phenotyped individuals”, Nat Med. 27:321-323, 2021; associated metagenomes deposited in European Bioinformatics Institute European Nucleotide Archive under accession no. PRJEB39223; all of which is incorporated herein by reference for all it teaches).

Introduction

Dietary contributions to health, and particularly to long-term chronic conditions such as obesity, metabolic syndrome, and cardiac events, are of universal importance. This is especially true as obesity and associated mortality and morbidity have risen dramatically over the past decades, and continue to do so worldwide. The reasons for this relatively rapid change have remained unclear, with the gut microbiome implicated as one of several potentially causal human-environmental interactions (Brown & Hazen, Nat. Rev. Microbiol. 16:171-181, 2018; Mozaffarian, Circulation 133:187-225, 2016; Musso et al., Annu. Rev. Med. 62, 361-380, 2011; Le Chatelier et al., Nature 500:541-546, 2013). Surprisingly, the details of the microbiome's role in obesity and cardiometabolic health have proven difficult to define reproducibly in large, diverse human populations, contrary to their behavior in mice. This is likely due to the complexity of habitual diets, the difficulty of measuring them at scale, and the highly personalized nature of the microbiome (Gilbert et al., Nat. Med. 24:392-400, 2018).

This example describes the Personalized Responses to Dietary Composition Trial (PREDICT 1) observational and interventional study of diet-microbiome interactions in metabolic health. PREDICT 1 included over 1,000 participants in the United Kingdom (UK) and the United States (US) who were profiled pre- and post-standardized dietary challenges using a combination of intensive in-clinic biometric and blood measures, nutritionist-administered free-living dietary recall and logging, habitual dietary data collection, continuous glucose monitoring, and stool shotgun metagenomic sequencing. This study was inspired by and generally concordant with previous large-scale diet-microbiome interaction profiles, identifying both overall gut microbiome configurations and specific microbial taxa and functions associated with postprandial glucose responses (Zeevi et al., Cell 163:1079-1094, 2015; Mendes-Soares et al., Am. J. Clin. Nutr. 110, 63-75, 2019), obesity-associated biometrics such as body mass index (BMI) and adiposity (Falony et al., Science 352, 560-564, 2016; Zhernakova et al., Science 352, 565-569, 2016; Thingholm et al. Cell Host Microbe 26, 252-264.e10, 2019), and blood lipids and inflammatory markers (Schirmer et al., Cell 167:1897, 2016; Fu et al., Circ. Res. 117:817-824, 2015; Org et al., Genome Biol. 18:70, 2017). By combining PREDICT's extensive dietary and blood biomarker measures with high-precision microbiome analysis, these findings were able to extend to specific beneficial (e.g. Faecalibacterium prausnitzii) and detrimental (e.g. Ruminococcus gnavus) organisms, as well as to a highly-reproducible gut microbial signature of overall health that is validated across multiple blood and dietary measures within PREDICT and in several previously published cohorts (Pasolli et al., Nat. Methods 14:1023-1024, 2017).

Materials and Methods

The PREDICT 1 Study

The PREDICT 1 clinical trial (NCT03479866) aimed to quantify and predict individual variations in metabolic responses to standardized meals. Data was integrated from a cohort of twins and unrelated adults from the UK to explore genetic, metabolic, microbiome composition, meal composition and meal context data to distinguish predictors of individual responses to meals. These predictions were then validated in an independent cohort of adults from the US. The trial was a single-arm, single-blinded intervention study that commenced in June 2018 and completed in May 2019.

For full protocol, see Berry et al. (Protocol Exchange, 2020). In brief; 1,002 generally healthy adults from the United Kingdom (UK; non-twins, and identical [monozygotic; MZ] and non-identical [dizygotic; DZ] twins) and 100 healthy adults from the United States (US) (non-twins; validation cohort) were enrolled in the study and completed baseline clinic measurements. The study included a 1-day clinical visit at baseline followed by a 13-day at-home period. At baseline (Day 1), participants arrived fasted and were given a standardized metabolic challenge meal for breakfast (0 h; 86 g carbohydrate, 53 g fat) and lunch (4 h; 71 g carbohydrate, 22 g fat). Fasting and postprandial (9 timepoints; 0-6 h) venous blood was collected to determine serum concentrations of glucose, triglycerides (TG), insulin, C-peptide (as a surrogate for insulin) and metabolomics (by NMR). Stool samples, anthropometry, and a questionnaire querying habitual diet, lifestyle and medical health were obtained at baseline. During the home-phase (Days 2-14), participants consumed standardized test meals in duplicate varying in sequence and macronutrient composition, while wearing digital devices to continuously monitor their blood glucose (continuous glucose monitor; CGM), physical activity and sleep. Capillary blood was collected using dried blood spot cards, during the clinic visit and at home, to analyze fasting and postprandial concentrations of TG and C-peptide. Participants were supported throughout the study with reminders and communication from study staff delivered through the ZOE® (Zoe Global Limited, London, England) study app. A second stool sample was collected at home by participants following completion of the study and all devices and samples were mailed back to study staff. To monitor compliance, all test meals consumed by participants were logged in the ZOE® (Zoe Global Limited) app (with an accompanying picture) and reviewed in real-time by the study nutritionists. Only test meals that were consumed according to the standardized meal protocol were included in the analysis.

The recruitment criteria, meal intervention challenges, outcome variables, and sample collection and analysis procedures relevant to this paper are described elsewhere (Berry et al., Protocol Exchange, 2020). The trial was approved in the UK by the Research Ethics Committee and Integrated Research Application System (IRAS 236407) and in the US by the Partners Healthcare Institutional Review Board (IRB 2018P002078). The core characteristics of study participants at baseline were not significantly different between UK and US cohorts.

Overview of Microbiome Sequencing and Profiling

Deep shotgun metagenomic sequencing was performed (mean 8.8±2.2 gigabases/sample) in stool samples from a total of 1,098 PREDICT 1 participants (UK n=1,001; US n=97). From a random subset of these participants (n=70), fecal metagenomes were sequenced from a second stool sample collected 14 days after the first collection (FIG. 9A) fora total of 1,168 metagenomes. Computational analysis was performed using the bioBakery suite of tools (McIver et al., Bioinformatics 34, 1235-1237, 2018) to obtain species-level microbial abundances for the 769 taxa identified using the newly updated MetaPhIAn 2.96 tool (version 2.14; Kang et al., PeerJ 7, e7359, 2019), functional potential profiling of >1.91 M microbial gene families, 445 KEGG pathways with HUMAnN 2.0 (version 0.11.2 and UniRef database release 2014-07; Franzosa et al., Nat. Methods 15, 962-968, 2018), and reconstruction of 48,181 metagenome-assembled genomes (MAGs) of medium or high-quality using the validated pipeline (Pasolli et al., Cell 176, 649-662.e20, 2019), which includes assembly with MegaHIT (Li et al., Bioinformatics 31, 1674-1676, 2015), binning with MetaBAT2 (Kang et al., PeerJ 7, e7359, 2019), and quality-control with CheckM (version 1.0.18; Parks et al., Genome Res. 25:1043-1055, 2015).

Microbiome Sample Collection

Participants were mailed a pre-visit study pack with a stool collection kit and relevant questionnaires and asked to collect an at-home stool sample at two timepoints (one day prior to their in-person clinical visit on day 0 and the next at the conclusion of their home-phase, day 14). Those who did not collect a sample prior to their in-person, baseline visit completed the collection as soon as possible during the home-phase. Baseline samples in the UK were collected using the EasySampler collection kit (ALPCO, NH), whereas post-study samples, as well as the entirety of the US collection was conducted using the Fecotainer collection kit (Excretas Medical BV, Enschede, the Netherlands). For baseline samples, one fresh unfixed sample was deposited into a sterile universal collection container (Sarstedt, Australia, Cat #L0263-10) and one into a tube containing DNA/RNA Shield buffer (Zymo Research, CA, US, Cat #R1101). Samples were stored at ambient temperature until return to the study staff. Follow-up samples were collected similarly, but only sampled into a DNA/RNA Shield buffer tube and sent by standard mail to study staff. Upon receipt in the laboratory, samples were homogenized, aliquoted, and stored at −80° C. in Qiagen PowerBeads 1.5 mL tubes (Qiagen, Germany). This sample collection procedure was tested and validated internally comparing different storage conditions (fresh, frozen, buffer), different DNA extraction kits (PowerSoilPro, FastDNA, ProtocolQ, Zymo), and different sequencing technologies (16S rRNA, shotgun metagenomics, and arrays).

DNA Extraction and Sequencing

DNA was isolated by QIAGEN Genomic Services using DNeasy® (Qiagen) 96 PowerSoil® (Qiagen) Pro from all Day 0 (baseline) DNA/RNA shield fixed microbiome samples. A random subset of Day 14 (end of at-home phase) samples (n=70) were also extracted. Optical density measurement was done using Spectrophotometer Quantification (Tecan Infinite 200). Before library preparation and sequencing, the quality and quantity of the samples were assessed using the Fragment Analyzer (Agilent Technologies, Inc., Santa Clara, Calif.) according to manufacturer's guidelines. Samples with a high-quality DNA profile were further processed. The NEBNext® (New England Biolabs, Ipswich, Mass.) Ultra II FS DNA module (Cat #NEB #E7810S/L) was used for DNA fragmentation, end-repair, and A-tailing. For adapter ligation, the NEBNext® (New England Biolabs) Ultra II Ligation module (Cat #NEB #E7595S/L) was used. The quality and yield after sample preparation were measured with the Fragment Analyzer. The size of the resulting product was consistent with the expected size of 500-700 bp. Libraries were sequenced for 300 bp paired-end reads using the Illumina NovaSeq® (Illumina, San Diego, Calif.) 6000 platform according to manufacturer's protocols. 1.1 nM library was used for flow cell loading. NovaSeq® (Illumina) control software NCS v1.5 was used. Image analysis, base calling, and the quality check were performed with the Illumina data analysis pipeline RTA3.3.5 and Bcl2fastq v2.20.

Metagenome Quality Control and Pre-Processing

All sequenced metagenomes were QCed using the pre-processing pipeline as implemented in the BiotBucket Computational Metagenomics Lab, available online at github.com/SegataLab/preprocessing. Pre-processing includes three main steps: (1) read-level quality control; (2) screening of contaminant i.e. host sequences; and (3) split and sorting of cleaned reads. Initial quality control involves the removal of low-quality reads (quality score <Q20), fragmented short reads (<75 bp), and reads with >2 ambiguous nucleotides. Contaminant DNA was identified using Bowtie 2 (Langmead & Salzberg, Nat Methods 9(4):357-359, 2012) using the --sensitive-local parameter, allowing confident removal of the phiX174 Illumina spike-in and human-associated reads (hg19). Sorting and splitting allowed for the creation of standard forward, reverse, and unpaired reads output files for each metagenome.

Microbiome Taxonomic and Functional Potential Profiling

The metagenomic analysis was performed following the general guidelines described by Quince et al. (Nat. Biotechnol. 35, 833-844, 2017) and relying on the bioBakery computational environment (McIver et al., Bioinformatics 34, 1235-1237, 2018). The taxonomic profiling and quantification of organisms' relative abundances of all metagenomic samples were quantified using MetaPhIAn2 (Metagenomic Phylogenetic Analysis; version 2.9.21 and marker database release 2.9.4; Truong et al., Nat. Methods 12, 902-903, 2015). The updated species-specific database of markers was built using 99,237 reference genomes representing 16,797 species retrieved from GenBank (January 2019). From this set of reference genomes, a total of 1,077,785 markers were extracted and 10,586 species were profiled. Compared to the previous version of the MetaPhIAn2 database (mpa_v20_m200), the updated database is able to profile 8,102 more species. Metagenomes were mapped internally in MetaPhIAn2 against the marker genes database with Bowtie2 version 2.3.4.3 with the parameter “very-sensitive”. The resulting alignments were filtered to remove reads aligned with a MAPQ value <5, representing an estimated probability of the likelihood of the alignments.

For estimating the microbiome species richness of an individual, from the taxonomic profiles of the PREDICT 1 participants, two alpha diversity measures were computed: the number of species found in the microbiome (“observed richness”), and the Shannon entropy estimation. Microbiome dissimilarity between participants (beta diversity) was computed using the Bray-Curtis dissimilarity and the Aitchison distance on microbiome taxonomic profiles.

Functional potential analysis of the metagenomic samples was performed using HUMAnN2 (version 0.11.2 and UniRef database release 2014-07; Franzosa et al., Nat. Methods 15, 962-968, 2018) that computed pathway profiles and gene-family abundances.

Metagenomic Assembly

Metagenomic samples were processed to obtain metagenome-assembled genomes (MAGs) following the procedure used elsewhere (Pasolli et al., Cell 176, 649-662.e20, 2019). In brief, MEGAHIT (version 1.2.9; Li et al., Bioinformatics 31, 1674-1676, 2015) was used with parameters “--k-max 127” for assembly and assembled contigs 1.5 kb were considered for the binning step performed using MetaBAT2 (version 2.14; Kang et al., PeerJ 7, e7359, 2019) with parameters: “-m 1500 --unbinned”. Quality control of the obtained MAGs was performed using CheckM (version 1.0.18; Parks et al., Genome Res. 25:1043-1055, 2015) using default parameters. High-quality and medium-quality microbial genomes were integrated into the existing database of >150,000 human MAGs.

Collection and Processing of Habitual Diet Information

Habitual diet information was collected using food frequency questionnaires (FFQ). For the UK, the European Prospective Investigation into Cancer and Nutrition (EPIC) FFQ was used and in the US, the Harvard semi-quantitative FFQ was used.

For the UK, the 131-item EPIC FFQ that was developed and validated against pre-established nutrient biomarkers was used for the EPIC Norfolk (Bingham et al., Public Health Nutr. 4, 847-858, 2001). The questionnaire captured average intakes in the past year. Nutrient intakes were determined via consultation with McCance and Widdowson's 6th edition, an established nutrient database (Holland et al., McCance and Widdowson's The Composition of Foods. (Royal Society of Chemistry, 1991)). US participants completed the Harvard 2007 Grid 131-item FFQ previously validated against two week dietary records (Rimm et al., Am J Epidemiol 135(10:1114-1126, 1992).

Nutrient Intakes were Estimated Using the Harvard Nutrient Database.

Submitted FFQs were excluded if greater than 10 food items were left unanswered, or if the total energy intake estimate derived from FFQ as a ratio of the subject's estimated basal metabolic rate (determined by the Harris-Benedict equation; Frankenfield et al., J. Am. Diet. Assoc. 98, 439-445, 1998) was more than two standard deviations outside the mean of this ratio (<0.52 or >2.58).

The following dietary indices were calculated as described below and according to categorization listed in Tables 1 and 3:

Healthy Food Diversity Index: The Healthy Food Diversity (HFD) index considers the number, distribution, and health value of consumed foods. To obtain this index, food frequency questionnaire foods were first aggregated into 15 food groups according to the HFD (Vadiveloo et al., Br. J. Nutr. 112, 1562-1574, 2014). Health values were then derived from the German Nutrition Society (DGE) dietary guidelines (available online at dge.de/en/); and the weight of each food group was multiplied by its corresponding health value (hv). Scores were divided by the maximum (hv=0.26) to bind values between 0-1 before multiplication with the Berry-Index. The original HFD was used instead of the US-HFD for the following reasons: the original HFD gives greater emphasis to plant-based foods and less to meat than the US-HFD which would more closely align with hypothesized microbiome-plant food/fibre interactions, and converting UK g/serving to US volume measures (as required for the US-HFD) would introduce additional error to the FFQ estimates.

The plant-based diet index: Three versions of the plant-based diet index (Satija et al., J. Am. Coll. Cardiol. 70, 411-422, 2017) were considered: the original plant-based diet index (PDI), the healthy plant-based index (h-PDI) and the unhealthy plant-based index (u-PDI). Eighteen food groups (amalgamated from the FFQ food groups; Table 1) were assigned either positive or reverse scores after segregation into quintiles, as outlined in Table 3 (Part 1) and Satija et al. (J. Am. Coll. Cardiol. 70, 411-422, 2017). Participants with an intake above the highest quintile for the positive score received a score of 5. Those below the lowest quintile intake received a score of 1. A reverse value was applied for the reverse scores. The scores for each participant were summed to create the final score. For the PDI, a positive score was applied to the “healthy” and “less-healthy”/“unhealthy” plant foods, and a reverse score applied to the animal-based foods. For the h-PDI, positive scores were applied to the “healthy” plant foods, and a reverse score to the “less-healthy”/“unhealthy” plant foods and the animal-based foods. For the u-PDI, a positive score was applied to the “less-healthy”/“unhealthy” plant foods and a reverse score applied to the “healthy” plant foods and the animal-based foods.

Animal score: The animal-based score categorized animal foods into “healthy” and “less-healthy”/“unhealthy” categories according to previous epidemiological studies. A similar approach to the PDI scoring was applied to the animal-based food groups, with either a positive (“healthy”) or reverse (“less-healthy”/“unhealthy”) quintile scoring; Tables 1 and 3.

The aMED score (Mediterranean Diet): Adherence to the aMED diet was calculated by following the method outlined by Fung et al. (Am. J. Clin. Nutr. 82, 163-173, 2005). Nine food/nutrient categories were included (Table 3, Part 5) and the score ranged from 0 to 9 (“least” to “most” Mediterranean). To form groups, weekly intake frequencies were first multiplied for assigned foods by the amount in grams per serving and then divided by 7 to determine grams per day. Next, food gram amounts were summed to make the final category total. For all food categories as well as the fatty acid intake ratio, the median intake of each category was calculated. A score of 0 (no aMED) or 1 (aMED) was given for each category depending on whether the twin was above or below the median intake. For alcohol intake, a range was used for score assignment: females: 5-25 g/d; males: 10-50 g/d were assigned a score of 1, while those above or below this range were assigned a score of 0. Finally, the aMED was then generated by summation of each category score.

Food groups: For individual analyses of food groups-microbe interaction, food groups were formed by aggregation of FFQ foods into the 18 PDI food groups plus margarine and alcohol (Table 3, Part 1).

Percentage of plants within diet: The percentage of plants within diet was calculated as weight in grams of plant foods within total weight (g) of diet after adjustment of FFQ foods into quantities (g) per week.

Number of plant foods. For the number of plant foods, each plant food item within the FFQ above the value of 0 g was allocated a score of 1 and summed for each participant. For the total number of plants and the number of “healthy” and “unhealthy” plants, FFQ food items were allocated into groups according to the PDI food groupings.

Collection and Processing of Fasting and Postprandial Markers

Venous blood samples were collected as described in Berry et al. (Protocol Exchange, 2020). In brief, participants were cannulated and venous blood was collected at fasting (prior to a test breakfast) and at 9 timepoints postprandially (15, 30, 60, 120, 180, 240, 270, 300, and 360 minutes). Plasma glucose and serum C-peptide and insulin were measured at all timepoints. Serum TG was measured at hourly intervals and serum metabolomics (NMR by Nightingale Health, Helsinki, Finland) at 0, 4 and 6 h). Fasting samples were analyzed for lipid profile, thyroid-stimulating hormone, alanine aminotransferase, liver function panel, and complete blood count (CBC) analysis.

Continuous glucose monitoring (CGM) on days 2-14 were measured every 15 minutes using Freestyle Libre Pro continuous glucose monitors (Abbott, Abbott Park, Ill., US), fitted on the upper, non-dominant arm at participants' baseline clinical visit. Given the CGM device requires time to calibrate once fitted to a participant, CGM data collected 12 hours and onwards after activating the device was used for analysis.

Dry blood spot (DBS) analysis of TG and C-peptide was completed by participants on the first four days of the home-phase while consuming test meals. The timepoints were dependent on the test meal as described elsewhere (Berry et al., Protocol Exchange, 2020). Test cards were stored in aluminum sachets with desiccant once completed and placed in the refrigerator at the end of the study day or until participants mailed them back to the study site. DBS cards were frozen at −80° C. upon receipt in the laboratory until being shipped to Vitas for analysis (Vitas Analytical Services, Oslo, Norway).

Specific timepoints and increments for TG, glucose, insulin, and C-peptide were selected for the current analysis to reflect the different pathophysiological processes for each measure as described in the protocol (Berry et al., Protocol Exchange, 2020). The incremental area under the postprandial TG (0-6 h), glucose (0-2 h), and insulin (0-2 h) curves (iAUC) were computed using the trapezium rule (Matthews et al., BMJ 300, 230-235, 1990).

For a detailed description of sample collection, processing and analysis see Berry et al., Protocol Exchange, 2020.

Machine Learning

The machine learning (ML) framework employed is based on the scikit-learn Python package (Pedregosa et al., J. Mach. Learn. Res. 12, 2825-2830, 2011). The ML algorithms used for the prediction and classification of personal, habitual diet, fasting, and postprandial metadata are based on Random Forest (RF) regressor and classification. RF-based methods were selected a priori as it has been repeatedly shown to be particularly suitable and robust to the statistical challenges inherent to microbiome abundance data (Thomas et al., Nat. Med. 25, 667-678, 2019; Pasolli et al., PLoS Comput. Biol. 12, e1004977, 2016). For both the regression and classification tasks, a cross-validation approach was implemented, based on 100 bootstrap iterations and an 80/20 random split of training and testing folds. To specifically avoid overfitting as a result of the twin population and their shared factors, any twin was removed from the training fold if their twin was present in the test fold.

For the regression task, an RF regressor was trained to learn the feature to predict, and simple linear regression to calibrate the output for the test folds on the range of values in the training folds. From the scikit-learn package, the RandomForestRegressor was used with “n_estimators=1000, criterion=‘mse” parameters and LinearRegression with default parameters. For the classification task, the continuous features were divided into two classes: the top and bottom quartiles. From the scikit-learn package, the RandomForestClassifier function was used with “n_estimators=1000” parameter.

RF classification and regression on both species-level taxonomic relative abundance and functional potential profiles were used. For taxonomic abundances, the relative abundances of MetaPhIAn2 (see above) were used with all the abundances of all microbial clades from phylum to species normalized using the arcsin-sqrt transformation for compositional data. For functional profiles, both raw relative abundance estimates of single microbial gene families as well as pathway-level relative abundance as provided by HUMAnN2 were considered.

As an additional control, it was verified that when random swapping the target labels or values (classification and regression, respectively), the performances were reflecting a random prediction, hence an AUC very close to 0.5 and a non-significant correlation between the predicted with values approaching 0.

Statistical Analysis

Spearman's correlations (reported with “ρ” in the text) have been computed using the cor.test from the stats R package and a modified version of the pcor.test from the ppcor package (available online at yilab.gatech.edu/pcor.R) that permits to control for a set of covariates rather than single ones, respectively. Correlations and the p-values were computed for each couple of metadata and species and p-values were corrected using FDR through the Benjamini-Hochberg procedure, which are reported in the text as q-values. Significant correlations with q<0.2 were considered. Significant species have been selected by ranking them according to their number of significant associations for the panel of metadata considered, and then the top thirty unique species are considered for each panel of metadata. In the heatmaps for partial correlations, the asterisk indicates that the correlation index for the corresponding species-metadata pair is significant at FDR≤0.2.

The contribution of metadata variables to microbiota community variation was determined by distance-based redundancy analysis (dbRDA) on species-level Bray-Curtis dissimilarity and Aitchison distance with the capscale function in the vegan R package 93. Correction for multiple testing (Benjamini-Hochberg, FDR) was applied and significance was defined at FDR <0.1. The cumulative contribution of metadata variables or metadata categories was determined by forward model selection on dbRDA (stepwise dbRDA) with the ordiR2step function in vegan, with variables that showed a significant contribution to microbiota community variation in the previous step. Only metadata variables with <15% missing data and without high collinearity with other variables (Spearman's rho <0.8) were used as input in the stepwise model.

Data Validation on the US Cohort and on the cMD Datasets

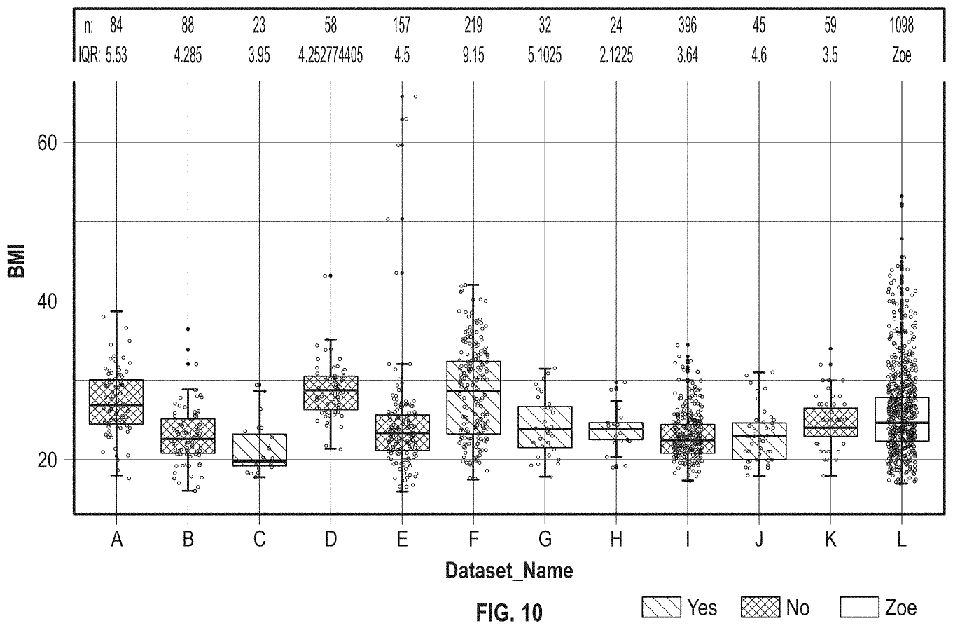

As independent validation, the publicly available datasets collected in the curatedMetagenomicData version 1.16.0 R package (cMD; Pasolli et al., Nat. Methods 14, 1023-1024, 2017) were considered. Of the 57 datasets available, those that have samples with the following characteristics were selected: (1) gut samples collected from healthy adult individuals at first collection (“days_from_first_collection”=0 or NA), (2) samples with age and BMI data available and BMI interquartile range (IQR) of these samples between 3.5 and 7.5 (±2 with respect to the PREDICT 1 UK IQR of 5.5, FIG. 10). For each dataset with samples meeting the above criteria, only datasets with at least 50 samples were considered: CosteaPI_2017 (84 samples out of 279), DhakanDB_2019 (88 samples out of 110), HanenLBS_2018 (58 samples out of 208), JieZ_2017 (157 samples 385), SchirmerM_2016 (396 samples out of 471), and ZellerG_2014 (59 samples out of 199).

The previously selected validation datasets were used from cMD in two analyses: one based on machine learning to verify the reproducibility of the ML model trained using the PREDICT 1 UK samples, and the second to verify the species-level correlations found in the PREDICT 1 UK cohort. For the first task, a regression algorithm was applied to predict BMI and age. Three different cross-validation approaches were used. First, using each dataset independently in 100 bootstrap iterations and an 80/20 random split of training and testing folds. Second, one more iteration was performed using the PREDICT 1 UK dataset as training fold and each dataset as testing fold. Third, a final prediction was made using Leave-One-Dataset-Out cross-validation (LODO), meaning that all datasets (PREDICT 1 UK, PREDICT 1 UK, and the cMD datasets) were considered together and each validation dataset was successively used as the test fold while all others were used for training. An additional validation performed using the cMD datasets was done by applying a pairwise Spearman correlation for each species in each cMD dataset against BMI and age. For each correlation, the top associated species were selected in PREDICT 1 UK (FDR q<=0.05) and their correlation was reported in cMD. For those species also found in the PREDICT 1 US, their correlation was reported as well.

Results and Discussion

Large Metagenomically-Profiled Cohorts with Rich Clinical, Cardiometabolic, and Dietary Information

A multi-national, single-arm (pre-post) intervention study of diet-microbiome-cardiometabolic interactions was performed, including a discovery cohort based in the United Kingdom (UK) and a validation population in the United States (US). The UK cohort recruited 1,002 generally healthy adults (non-twins, identical [monozygotic; MZ] and non-identical [dizygotic; DZ] twins), with detailed demographic information, quantitative habitual diet data, cardiometabolic blood biomarkers, and assessed postprandial responses to both standardized test meals in the clinic and in free-living setting (Berry et al., Protocol Exchange, 2020; FIG. 9A). At-home collection of stool by the validated protocol (Methods) yielded 1,001 baseline samples for gut microbiome analysis. The US population employed the same enrollment and biospecimen collection protocols for 100 healthy, unrelated individuals (97 stool samples from 1,098 PREDICT 1 participants (UK n=1,001; US n=97). From a random subset of these received). The data from the US cohort was analyzed separately to the UK data to test the machine learning models trained in the UK cohort and independently validate microbiome-feature correlations. From a randomly selected subset of UK participants (n=70), fecal metagenomes were additionally sequenced from a second stool sample collected 14 days after the first collection (FIG. 9A) for a total of 1,168 metagenomes. All metagenomes were shotgun sequenced, taxonomically and functionally profiled, and assembled to provide metagenome-assembled genomes (MAGs). Computational analysis was performed using the bioBakery suite of tools (McIver et al., Bioinformatics 34, 1235-1237, 2018) to obtain species-level microbial abundances for the 769 taxa identified using an updated version of MetaPhIAn2 (Truong et al., Nat. Methods 12, 902-903, 2015), functional potential profiling of >1.91 M microbial gene families and 445 KEGG pathways with HUMAnN2 (Franzosa et al., Nat. Methods 15, 962-968, 2018), and reconstruction of 48,181 MAGs of medium or high-quality using the validated pipeline (Pasolli et al., Cell 176, 649-662.e20, 2019) which includes assembly with MegaHIT (Li et al., Bioinformatics 31, 1674-1676, 2015), binning with MetaBAT2 (Kang et al., PeerJ 7, e7359, 2019), and quality-control with Check-M (Parks et al., Genome Res. 25, 1043-1055, 2015). Collectively, these UK and US-based results include the PREDICT 1 study.

Microbial Diversity and Composition are Linked with Diet and Fasting and Postprandial Biomarkers

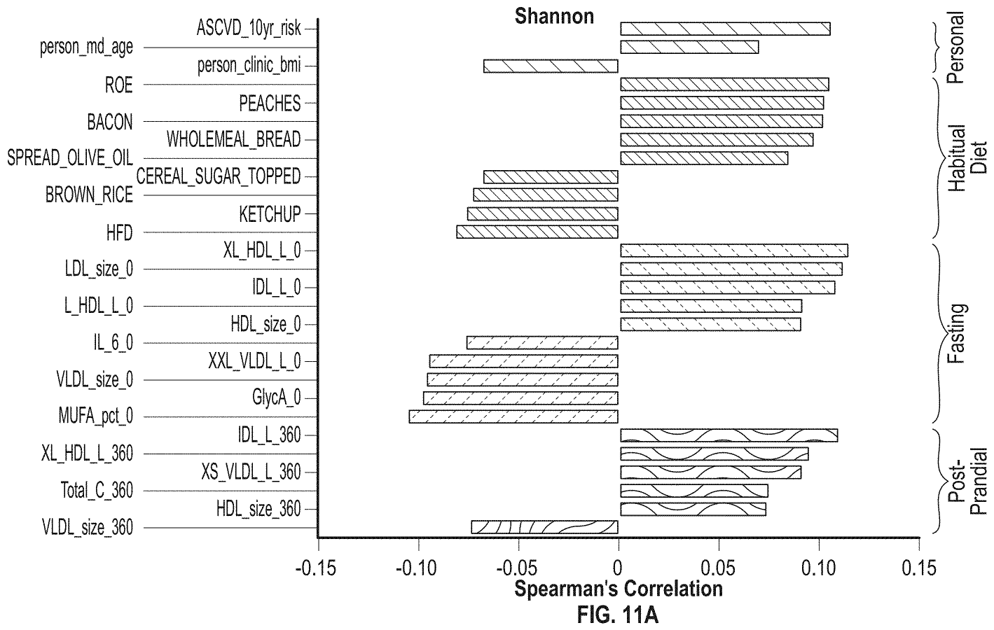

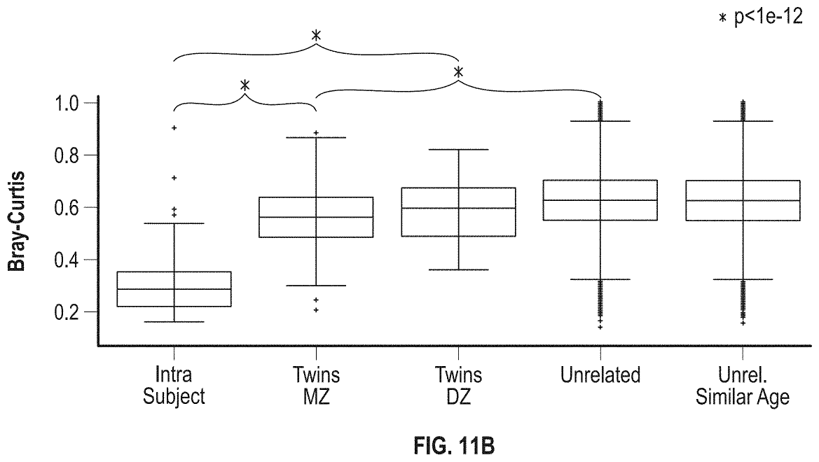

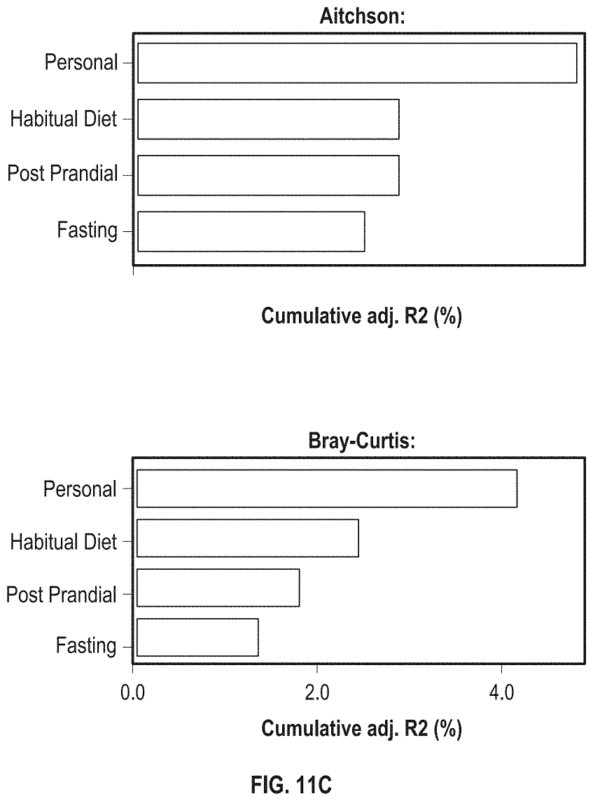

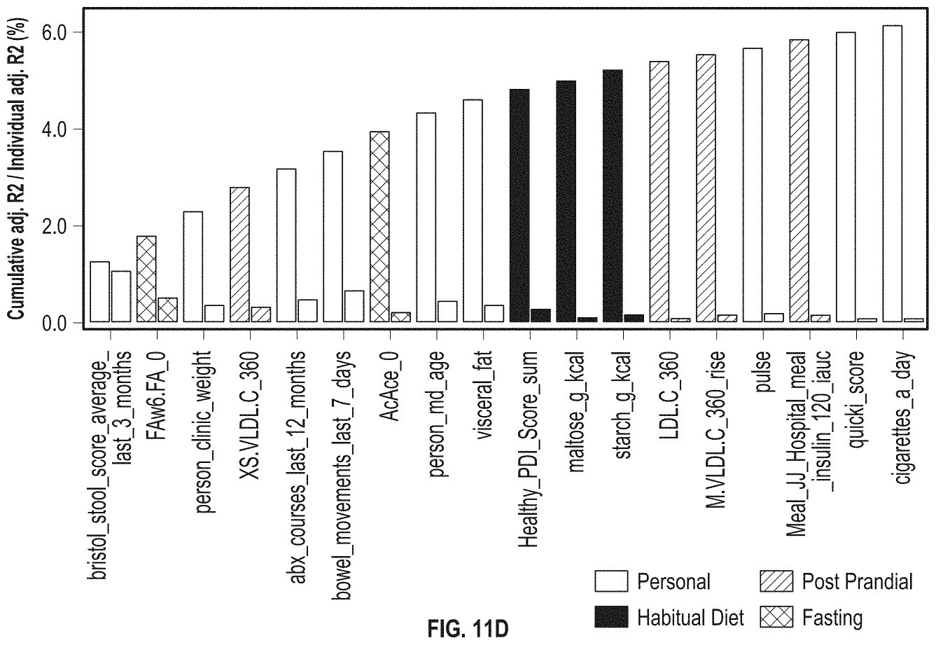

A unique subpopulation of the study was first leveraged including 480 twins to disentangle the confounding effects of shared genetics from other factors on microbiome composition. The data confirmed that host genetics influences microbiome composition only to a small extent (Xie et al., Cell Syst. 3, 572-584.e3, 2016), as intra-twin pair microbiome similarities were significantly greater than those among unrelated individuals (p<1e-12, FIG. 11B), and monozygotic twins showed slightly more similar microbiomes than dizygotic twins (p=0.06). Intra twin-pair microbiome similarity, regardless of zygosity, remained substantially lower than intra-subject longitudinal sampling (day 0 vs. day 14, p<1e-12, FIG. 11B), a testament to the highly personalized nature of the gut microbiome attributable to a variable extent to non-genetic factors (FIGS. 11C, 11D).

The overall intra-sample (alpha) diversity of the gut microbiome as a broad summary statistic of microbiome structure (Ravel et al., Proc. Natl. Acad. Sci. U.S.A. 108 Suppl 1, 4680-4687, 2011) was investigated. In the cohort of healthy individuals, links were found between alpha diversity (specifically species richness) and personal characteristics (e.g. age and anthropometry), habitual diet, and metabolic indices (FIG. 9B) with 109 significant associations (p<0.05) among the total 295 Spearman's correlation tests, and 56 after FDR-correction (q<0.05). Participant BMI, absorptiometry-based visceral fat measurements, and probability of fatty liver (using a validated prediction model; Atabaki-Pasdar et al., Genetic and Genomic Medicine, doi:10.1101/2020.02.10.20021147, 2020) were inversely associated with species richness. Consistent with previous findings for BMI (Le Chatelier et al., Nature 500, 541-546, 2013; Turnbaugh et al., Nature 457, 480-484, 2009), the findings suggest that the link between the microbiome and body habitus may be mediated in part by hepatic insulin resistance, particularly given the gut microbiome's strong association with liver disease and activity observed in this cohort and previously (Qin et al., Nature 513, 59-64, 2014). With respect to habitual dietary factors, 18 of 126 total nominally significant (p<0.05) correlations (5 at q<0.05, FIG. 9B) were found.

Among clinical circulating measures, HDL cholesterol (HDL-C) was positively correlated with species richness. However, emerging cardiometabolic biomarkers with strong associations with cardiometabolic diseases Wirtz et al., Circulation 131, 774-785, 2015; Ahola-Olli et al., Diabetologia 62, 2298-2309, 2019; Vojinovic et al., Nat. Commun. 10, 5813, 2019; Duprez et al., Clin. Chem. 62, 1020-1031, 2016) that are not routinely used clinically, including lipoprotein particle size (diameter, “-D”), lipoprotein composition (cholesterol “-C” and TG “-TG”), apo-lipoproteins and GlycA (inflammatory biomarker; glycoprotein acetyls), were even more strongly associated with richness than the remaining traditional clinical measures (TG, Total-C, LDL-C and fasting glucose). LDL stands for low density lipoprotein and VLDL stands for very low density lipoprotein. These emerging biomarkers of reduced risk of chronic disease were positively associated with microbial diversity (e.g., extra-large and large HDL-C, HDL-D, Apolipoprotein-A1) both at fasting and postprandially, whilst those associated with increased risk of chronic disease were inversely correlated with microbial diversity (e.g. GlycA, VLDL-D small-HDL-TG). These results for species richness provide initial evidence that the microbiome is modestly, but significantly, associated with some key classical and emerging cardiometabolic health indicators and diet, motivating more detailed investigations of the links between cardiometabolic health, diet, and specific gut microbiome components.

Diversity of Healthy Plant-Based Foods in Habitual Diet Shapes Gut Microbiome Composition

Links between habitual diet (over the past year) and the microbiome in PREDICT 1 using detailed, validated semi-quantitative food frequency questionnaires (FFQs) were assessed. These links were quantified using random forest (RF) regression and classification models, each trained on the whole set of quantitative microbiome features to predict one habitual diet feature (with training/testing via repeated bootstrapping, Methods). The performance of the models was evaluated with receiver operating characteristic (ROC) AUCs for classification and with correlation between predicted and collected values for regression, thus quantifying the degree to which each dietary feature could be estimated based on microbiome composition.

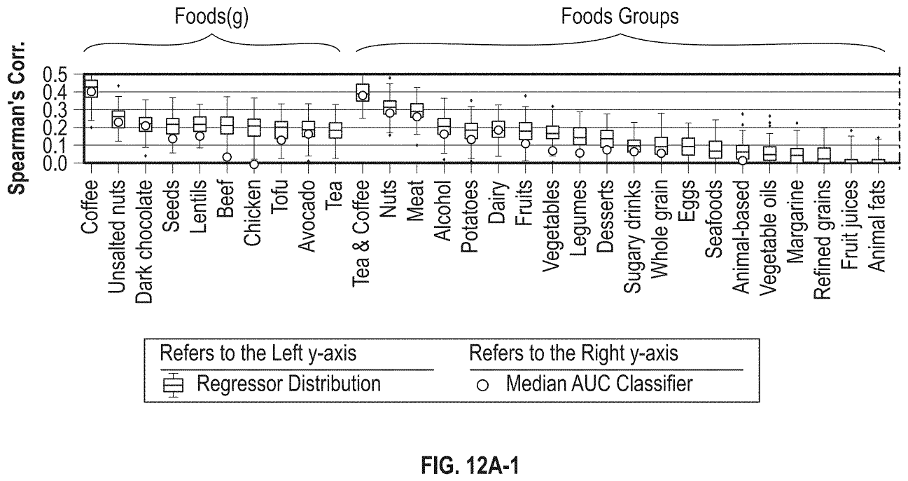

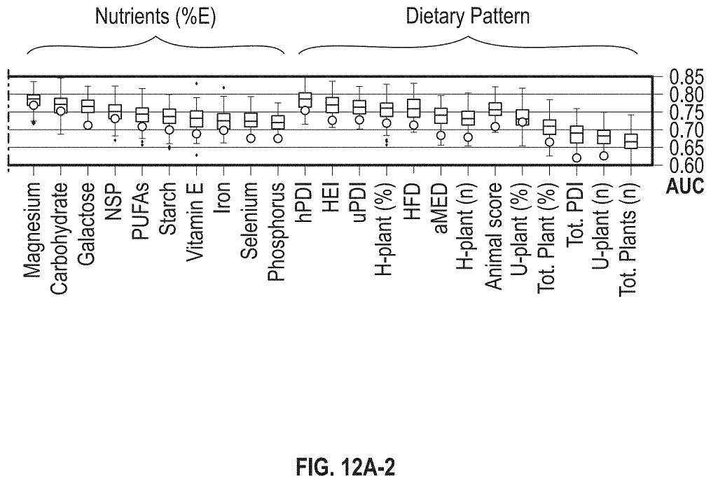

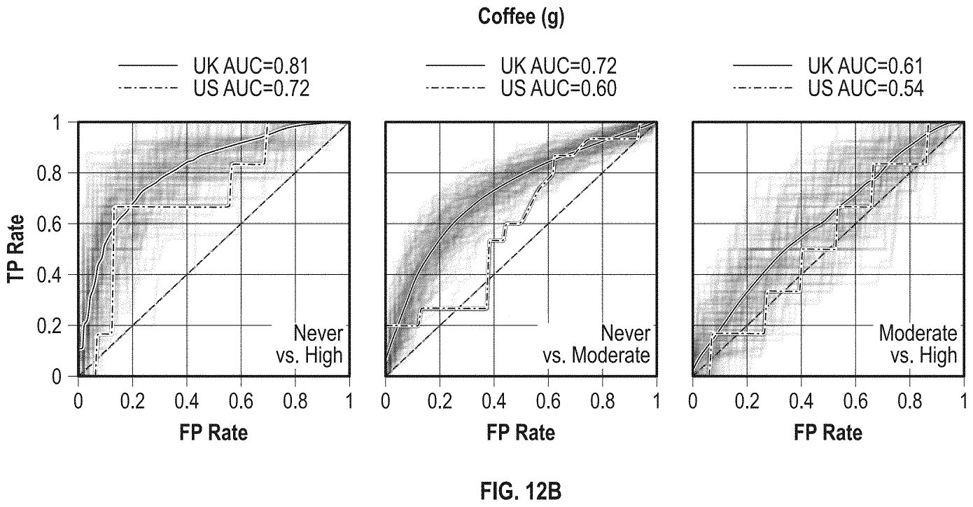

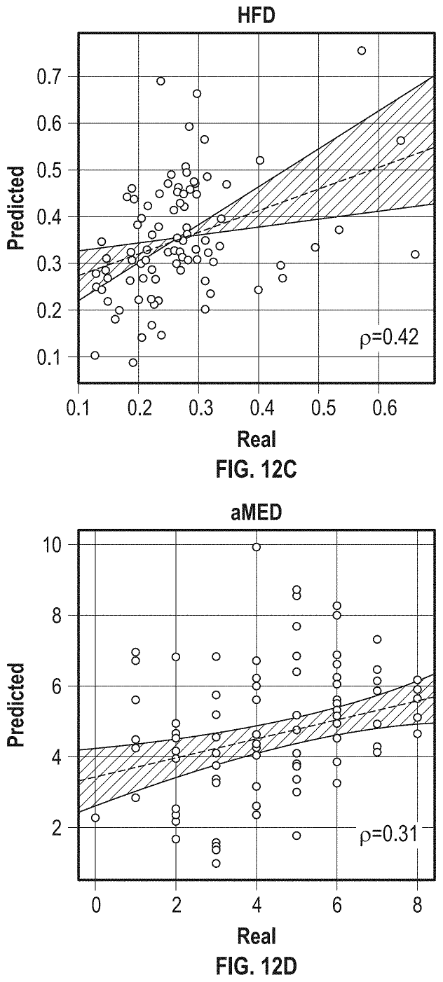

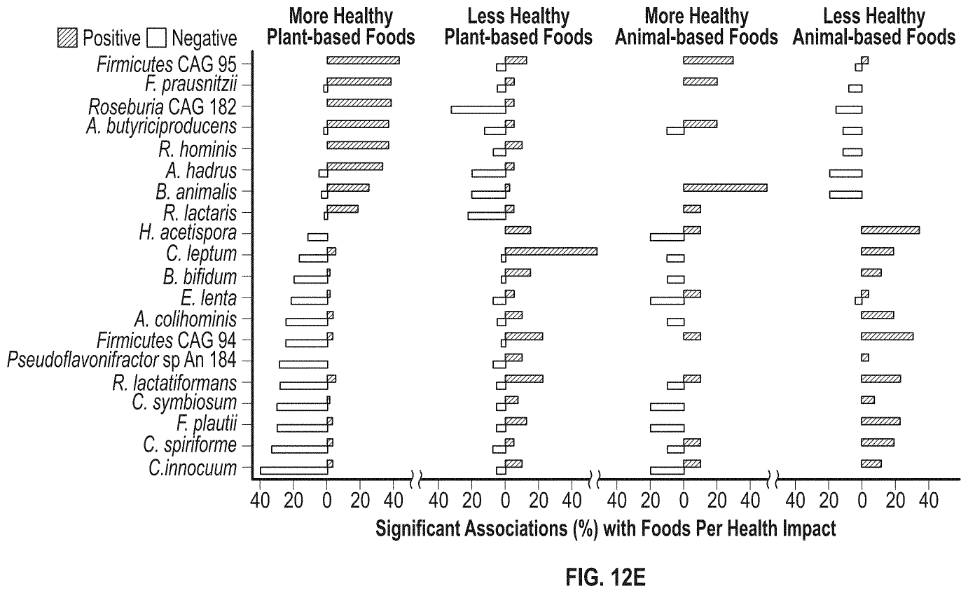

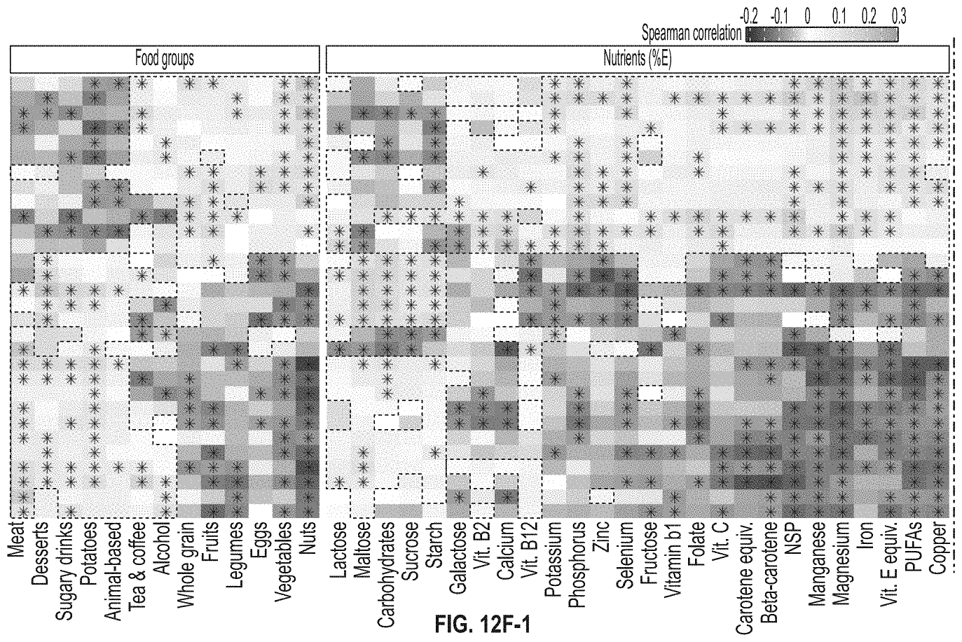

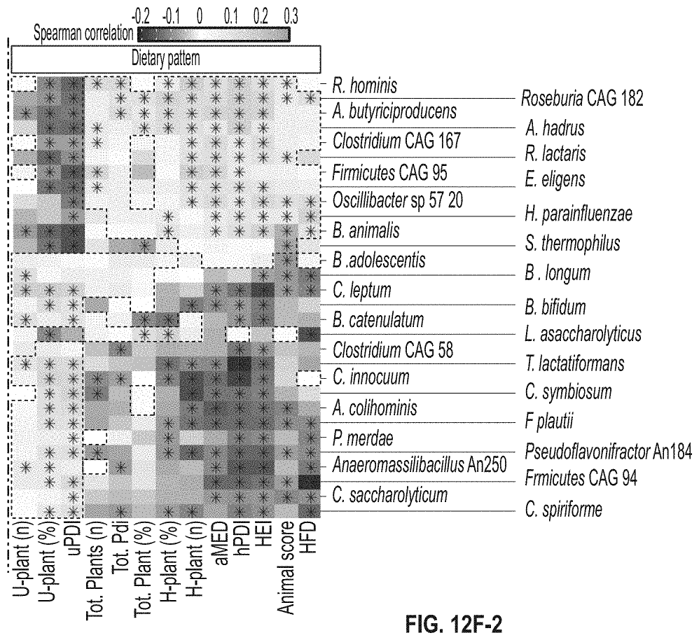

Dietary features assessed in this manner included individual food items, food groups, nutrients (energy adjusted and non-adjusted), and dietary patterns (FIGS. 12A-12F). Individual foods and food groups were assessed, the latter after collapsing items into bins according to Plant-based Diet Index (PDI; Satija et al., PLoS Med. 13, e1002039, 2016) groupings (Table 1). Several foods and food groups exceeded 0.15 median Spearman's correlation over bootstrap folds (denoted as “p”) between predicted and FFQ-estimated values (20/165 or 12.1%) and AUC>0.65 (14/165, 8.5%; FIGS. 12A-1 & 12A-2). The strongest association among food items was coffee (ρ=0.45), which appeared to be dose-dependent (FIG. 12B) and validated in the US cohort when the model trained in the UK cohort was applied in the US. Particularly tight coupling was found between energy-adjusted derived nutrients and the taxonomic composition of the microbiome, especially compared to foods and food groups (FIGS. 12A-1 & 12A-2). Almost one-third of the energy-normalized nutrients (Table 1) had correlations above 0.3 (14/47) with the highest correlations achieved for saturated fatty acids (SFAs, ρ=0.46, AUC 0.82), zinc (ρ=0.39, AUC 0.76), and starch (ρ=0.39, AUC 0.75).

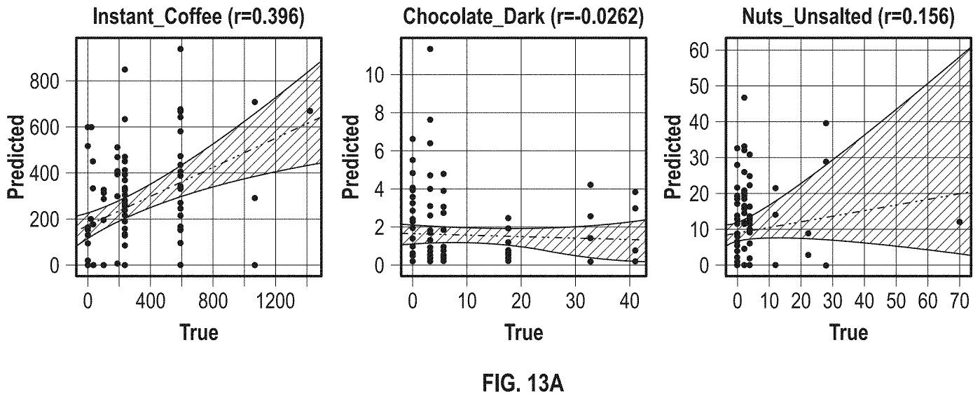

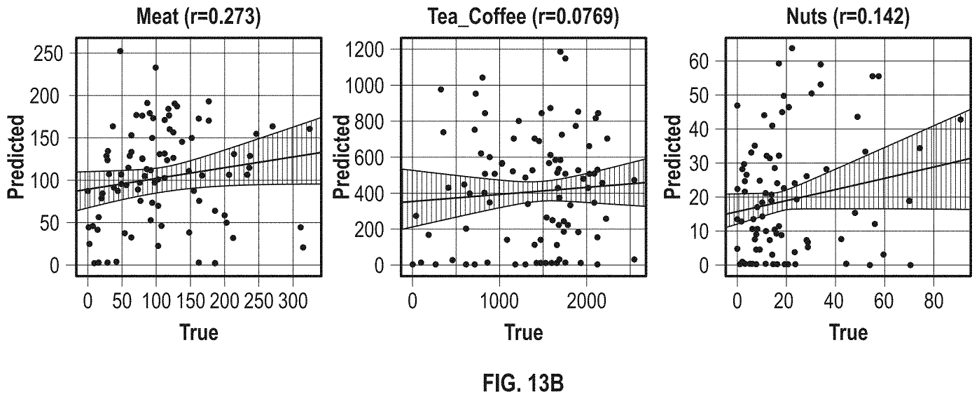

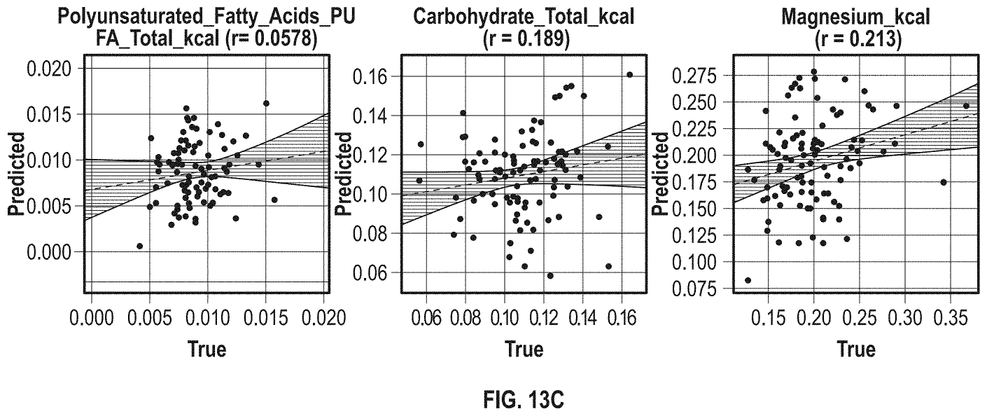

Because of the complex and interacting nature of dietary intake, as well as to offer practical recommendations, constituent foods and food groups were summarized into several established dietary indices (Table 1), including the Healthy Food Diversity index (HFD), Vadiveloo et al., Br. J. Nutr. 112, 1562-1574, 2014 the Healthy and Unhealthy Plant-based Dietary Indices (H-PDI and U-PDI), and the Alternate Mediterranean Diet score (aMED; Fung et al., Am. J. Clin. Nutr. 82, 163-173, 2005). The HFD, unlike the other food scores, incorporates a measure of dietary diversity (greater is considered better) and food quality according to dietary guidelines, whereas the PDI characterizes a given diet on the basis of type and quantity of the plant-based foods categorized as ‘more-healthy/healthy’ or ‘less-healthy’/‘unhealthy’ based on epidemiological evidence (Satija et al., PLoS Med. 13, e1002039, 2016). These scores have been associated with lower cardiovascular disease risk 29, type 2 diabetes (T2D) risk (Satija et al., PLoS Med. 13, e1002039, 2016), metabolic syndrome (Vadiveloo et al., J. Nutr. 145, 564-571, 2015), and all-cause mortality (Kim Hyunju et al., J. Am. Heart Assoc. 8, e012865, 2019). The aMED dietary score is based on dietary patterns in Mediterranean countries and has been associated with reduced risk of chronic disease and mortality (Reedy et al., J. Nutr. 144, 881-889, 2014; Mitrou et al., Arch. Intern. Med. 167, 2461-2468, 2007). Tight correlations were demonstrated between values predicted from gut microbial composition and all the indices (HFD, H-PDI, U-PDI, and aMED) in the UK (ρ=0.36, 0.34, 0.33, and 0.23, respectively) and in the US validation cohort (ρ=0.39, 0.23, 0.31, and 0.38, respectively; FIG. 12A and FIGS. 13A-13C), highlighting the relationship between the microbiome and healthy dietary patterns. Additionally, these results indicate that diet-microbiome associations are consistent and generalizable from UK to US populations, adding confidence to the suggested biological targets explored below and alleviating concerns of overfitting.

Microbial Species Segregate into Groups Associated with More Healthy and Less Healthy Plant- and Animal-Based Foods

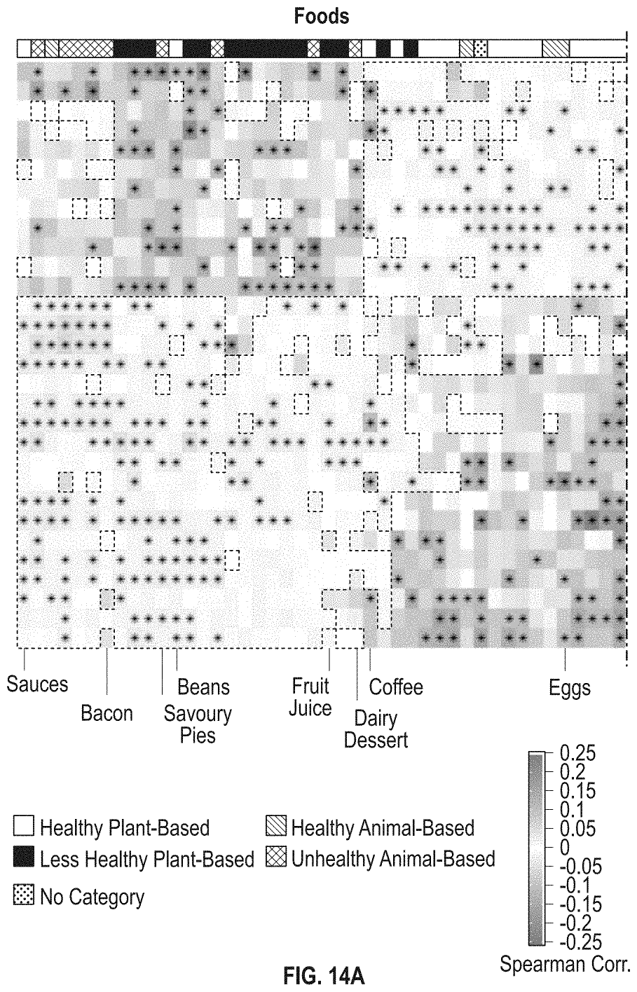

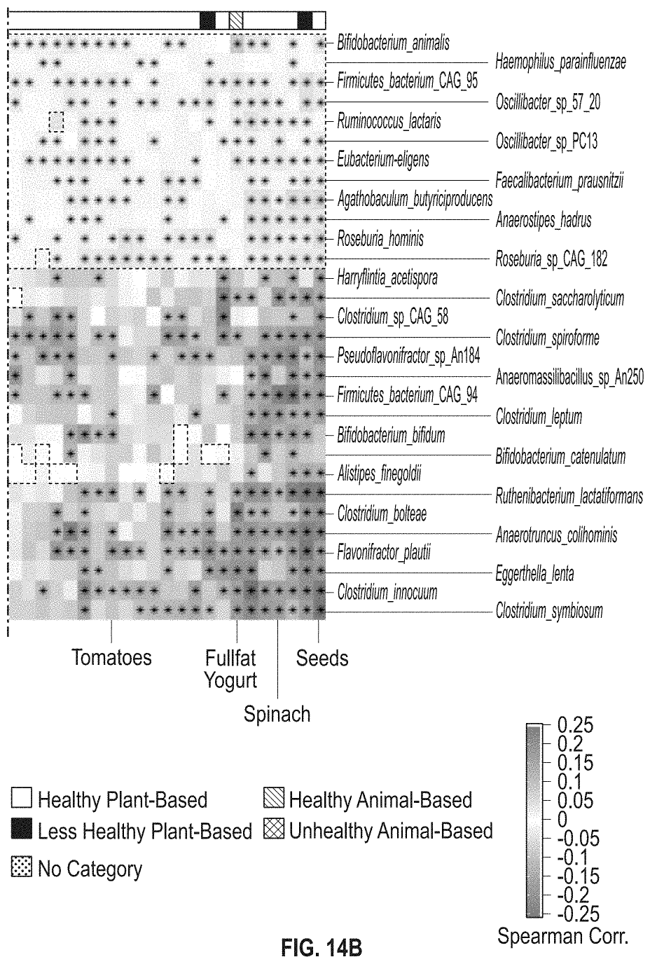

Feature-level testing to identify the specific microbial taxa most responsible for these diet-based community associations (FIGS. 12F-1 & 12F-2) was undertaken. By focusing on prevalent species (i.e., those detected in >20% of samples) and adjusting for age and BMI, 30 species (17%) were found to be significantly correlated with at least five defined dietary exposures at False Discovery Rate (FDR) q<0.2 (Table 3). This included a confirmation of expected associations (FIGS. 14A, 14B), such as the relative enrichment of the probiotic taxa Bifidobacterium animalis (Redondo-Useros et al., Nutrients 11, 2019) and Streptococcus thermophilus with greater full-fat yogurt consumption (ρ=0.22 and 0.20 respectively). The strongest food/microbe association was between the recently characterized butyrate-producing Lawsonibacter asaccharolyticus (Sakamoto et al., Int. J. Syst. Evol. Microbiol. 68, 2074-2081, 2018) and coffee consumption (FIGS. 12F-1 & 12F-2).

However, due to the low precision of dietary data collected by FFQ, the complexity of dietary patterns, nutrient-nutrient interactions, and clustering of ‘healthy’/‘less-healthy’ food items within diets, it is challenging to disentangle the independent associations of single nutrients and single foods with microbial species. Indeed, considering the top 30 species most strongly associated with various dietary determinants (based on number of significant correlations; FIGS. 12F-1 & 12F-2), a clear segregation of species into two distinct clusters was found with either more healthy plant-based foods (e.g. spinach, seeds, tomatoes, broccoli) or with less healthy plant-based (e.g. juices, sweetened beverages, and refined grains) and animal-based foods, as defined by the PDI (Satija et al., J. Am. Coll. Cardiol. 70, 411-422, 2017; Table 3).

Taxa linked to diets rich in more healthy plant-based foods (FIGS. 12F-1 & 12F-2, 12E and FIGS. 14A, 14B) mostly included butyrate producers, such as Roseburia hominis, Agathobaculum butyriciproducens, Faecalibacterium prausnitzii, and Anaerostipes hadrus, as well as other uncultivated species from clades typically capable of butyrate production (Roseburia CAG 182) or predicted to have this metabolic capability (Firmicutes CAG 95, with 92% of its 166 MAGs encoding for butyrate kinases). Clades correlating with several ‘less-healthy’ plant-based and animal-based foods included several Clostridium species (Clostridium innocuum, C. symbiosum, C. spiroforme, C. leptum, C. saccharolyticum). The relationship between C. leptum and the intake of unhealthy foods is particularly worth noting, as prior experimental evidence has demonstrated their counts can be modulated by diet in mice (Eslinger et al., Nutr. Res. 34, 714-722, 2014). The segregation of species according to animal-based ‘healthy’ foods (e.g. eggs, white and oily fish) or animal-based ‘less-healthy’ foods (e.g. meat pies, bacon and dairy desserts) using a novel categorization developed for this analysis based on epidemiological evidence outlined in Methods, was also distinct and was similar to taxa linked to patterns for ‘healthy’ and ‘less-healthy’ plant foods (FIG. 12E and FIGS. 14A, 14B). The few food items that did not fit into the ‘healthy’ cluster despite being categorized as ‘healthy plant’ foods, were (ultra) processed foods according to the NOVA classification (Monteiro et al., Public Health Nutr. 21:5-17, 2018; e.g. sauces, tomato ketchup, and baked beans; Group 4 and 3, respectively; FIGS. 14A, 14B). This emphasizes the importance of food quality (e.g. highly processed vs. unprocessed), food source (e.g. plant vs. animal), and food heterogeneity (i.e. not all plant foods are healthy and animal foods unhealthy, nor vice versa) both in overall health and in microbiome ecology.

Poorly Characterized Microbes Drive the Strongest Microbiome-Habitual Diet Associations

Many of the strongest microbial associations with food items, food groups, and dietary indices occurred with only recently isolated organisms or still uncultured taxa including, for example, five species defined using co-abundance gene groups (CAGs) from metagenomics (Nielsen et al., Nat. Biotechnol. 32, 822-828, 2014). Among indices, the HFD, which prioritizes diversity of all food items while considering dietary guidelines, was most tightly coupled to feature-level abundances (FIG. 12A), significantly correlated with 41 of the 174 prevalent species (i.e. those found in >20% samples), highlighting the synergistic impact of dietary diversity, dietary quality, and gut microbial responsiveness. Among species whose abundance was highly correlated to the HFD (FIGS. 12F-1 & 12F-2) were taxa also associated with ‘healthy’ or ‘less-healthy’ foods, such as Firmicutes CAG 94 (ρ=−0.25) and Roseburia CAG 182 (ρ=0.13). The highest correlation was observed for Lawsonibacter asaccharolyticus (ρ=−0.29), the aforementioned and recently characterized (Sakamoto et al., Int. J. Syst. Evol. Microbiol. 68, 2074-2081, 2018) and sequenced species (Sakamoto et al., Genome Announc. 6, 2018). This microbe has two additional known genomes with the conflicting species name of Clostridium phoceensis (Hosny, et al., New Microbes New Infect 14, 85-92, 2016), and it is predicted that it encodes butyrate-producing enzymes from metagenome-assembled genomes enzymes (Pasolli et al., Cell 176, 649-662.e20, 2019; 49 of the 53 MAGs in the L. asaccharolyticus SGB15154 encode for butyrate kinase EC 2.7.2.7). The link between the HFD and L. asaccharolyticus is particularly noteworthy and not likely a consequence of the previously observed association with coffee, as the HFD index does not include non-caloric beverages, including coffee, mineral water, and tea, as well as alcoholic beverages. This may suggest alternative and complementary strategies to modulate this microbe through both coffee intake and adherence to a diverse diet.

Among other dietary indices and nutrients, general concordance with the two sets of microbes associated with healthy and less-healthy foods was observed. A greater animal-based food score, which is derived based on the relative amount of ‘healthy’ (positive score) and ‘less-healthy’ (inverse score) animal foods consumed (Table 3), was associated with the ‘healthy’ cluster, suggesting that a diet rich in healthier animal-based foods is associated with the more favorable diet-microbiome signature, although this likely also reflects an overall healthier dietary pattern by healthy animal-based food consumers. The healthy and unhealthy PDI, which have been shown to differentially affect disease risk (Satija et al., PLoS Med. 13, e1002039, 2016; Satija et al., J. Am. Coll. Cardiol. 70, 411-422, 2017) also had distinct clusters, again emphasizing the oversimplification of conventional plant and animal-based food groupings. The strongest representatives for the two clusters (i.e. taxa with the highest correlations) are Firmicutes CAG 95 and Firmicutes CAG 94 for healthy and unhealthy diet, respectively, and the lack of cultivated representatives for these two candidate species may explain why these links were previously overlooked even in large analyses (Zeevi et al., Cell 163, 1079-1094, 2015; Zhernakova et al., Science 352, 565-569, 2016). The PREDICT 1 validation cohort in the US generally confirmed these associations despite its comparatively smaller sample size: among the subset of derived pattern/index scores shared between the UK and US cohorts, of the 52 associations that were significant both in the UK cohort (FDR q<0.2) and in the US cohort (p<0.05), 78.8% were concordant for the direction of the correlation.

Microbial Indicators of Obesity are Reproducible Across Varied Populations

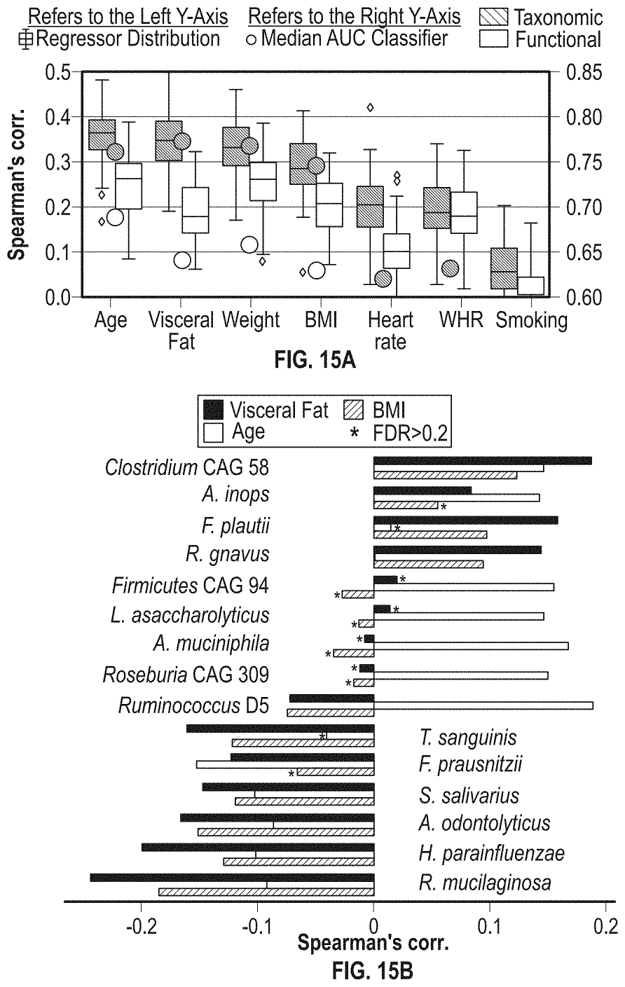

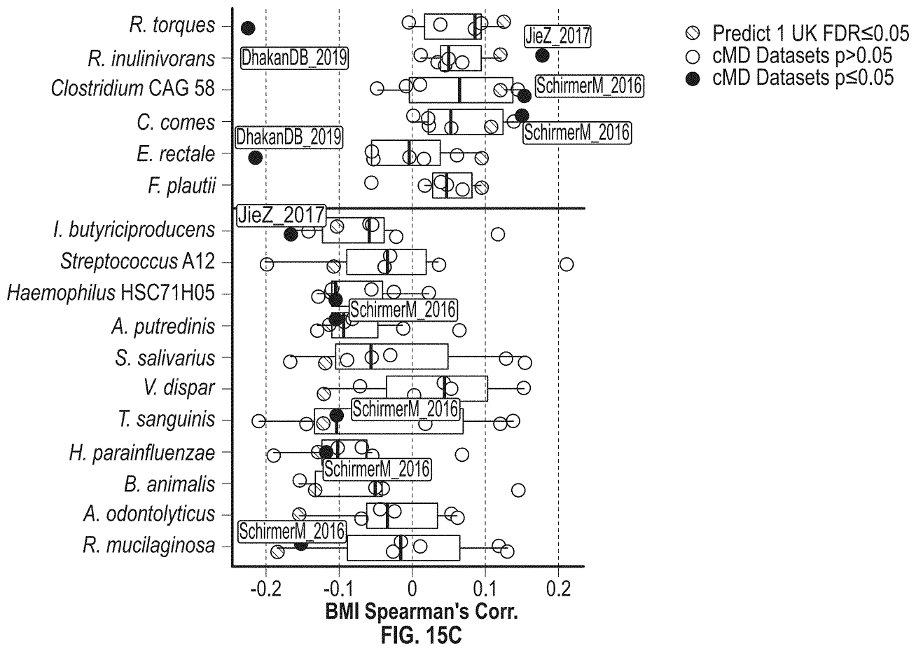

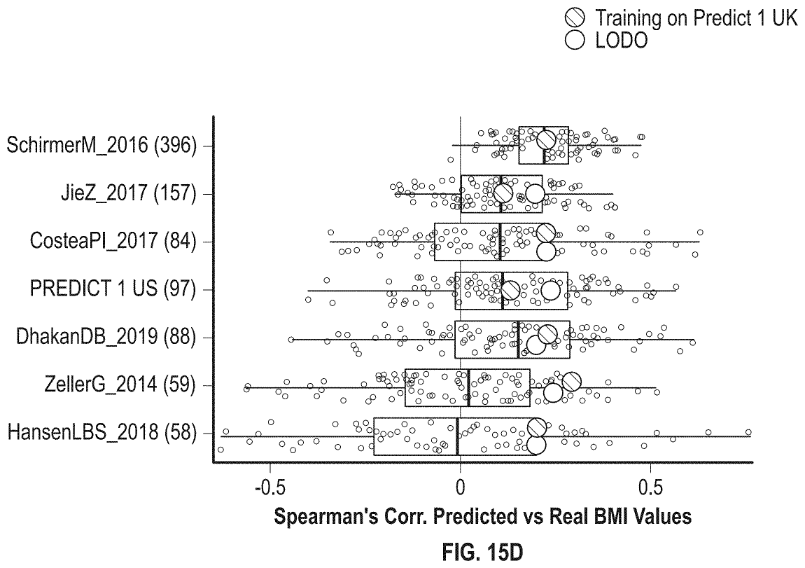

Microbiome links to obesity have attracted much interest although results have varied in human populations (Le Chatelier et al., Nature 500, 541-546, 2013; Sze & Schloss, MBio 7, 2016). They were explored in the PREDICT 1 populations with RF regression and classification (as above, Methods) using either taxonomic or functional features. Visceral fat measured by DEXA scan was found to be more strongly linked to gut microbial composition than BMI (Beaumont et al., Genome Biol. 17, 189, 2016), a finding validated in the US participants when applying UK-trained models (FIG. 15A). Some obesity-associated taxa—assessed either by BMI or visceral fat—were also associated with poor dietary patterns after controlling for BMI (e.g. Clostridium CAG 58, Flavonifractor plautii), whereas markers of healthier low visceral fat mass (e.g. Faecalibacterium prausnitzii) were more strongly linked to healthier foods and patterns of intake, illustrating that diet and obesity signatures overlap but are not identical (FIG. 15B).

Microbiome models to predict BMI developed and trained on the UK-based cohort were validated not only in the PREDICT US cohort, but also in six additional independent datasets (Schirmer et al., Cell 167, 1897, 2016; Zeller et al., Mol. Syst. Biol. 10, 2014; Hansen et al., Nat. Commun. 9, 4630, 2018; Costea et al., Mol. Syst. Biol. 13, 960, 2017; Jie et al., Nat. Commun. 8, 845, 2017; Dhakan et al., Gigascience 8, 2019) that have been uniformly pre-processed and harmonized using curatedMetagenomicData (Pasolli et al., Nat. Methods 14, 1023-1024, 2017; cMD), lending credence and generalizability to the findings. Despite substantial differences (Falony et al., Science, 352(6285): 560-4, 2016; Truong et al., Genome Res. 27, 626-638, 2017) in the microbiomes among people from different populations, the PREDICT 1 UK model improved cohort-specific cross-validation accuracy in the majority of cases, on par with the leave-one-out approach that notably also includes the UK cohort (FIG. 15D). Interestingly, BMI was not predictable at all for two included datasets when using just their own samples. However, predictions and classification improved when using the PREDICT 1 UK model. Of the 17 species surpassing the FDR threshold of q<0.05, three had an (absolute) p>0.1 in the smaller US cohort and two of these three were concordant with those in the UK cohort (I. butyriciproducens negatively and R. torques positively correlated with BMI; FIG. 15C). Across the harmonized independent cMD datasets, all but two median association estimates were consistent with the PREDICT 1 UK signatures, and 12 of the 14 were concordant despite different sample collection and DNA extraction methods.

Fasting Cardiometabolic Markers Associated with Specific Microbiome Structures

To explore the connections between the gut microbiome and markers of cardiometabolic health, fine-scale evaluations of microbial community membership and their biochemical functions against established clinical and emerging cardiometabolic biomarkers were performed. ML prediction models were developed for each of these outcomes built using both species-level taxonomic abundances and functional potential profiles and tested how accurately they were able to estimate host biomarkers.

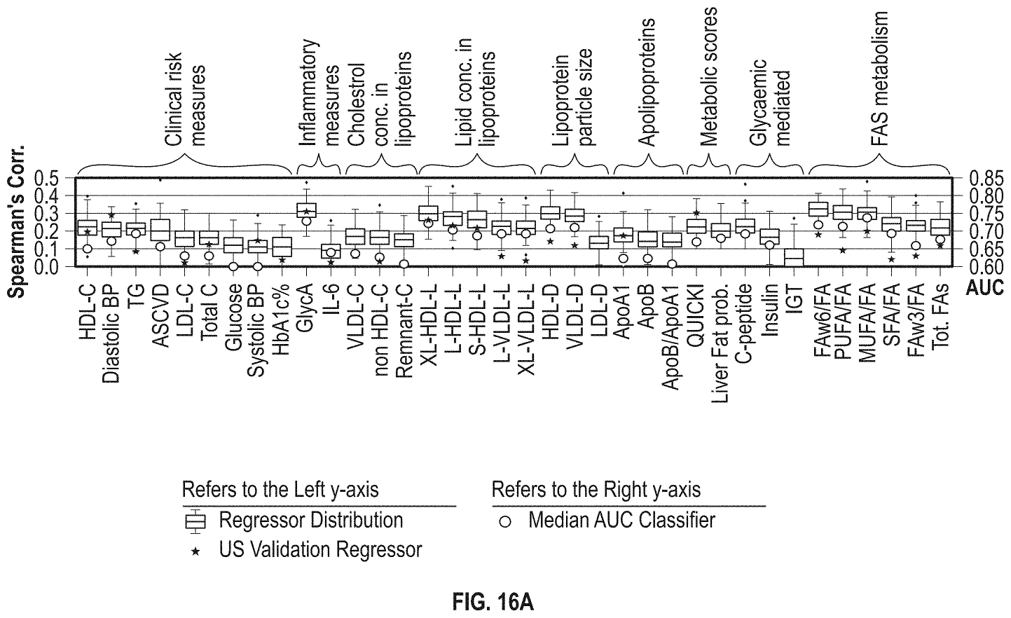

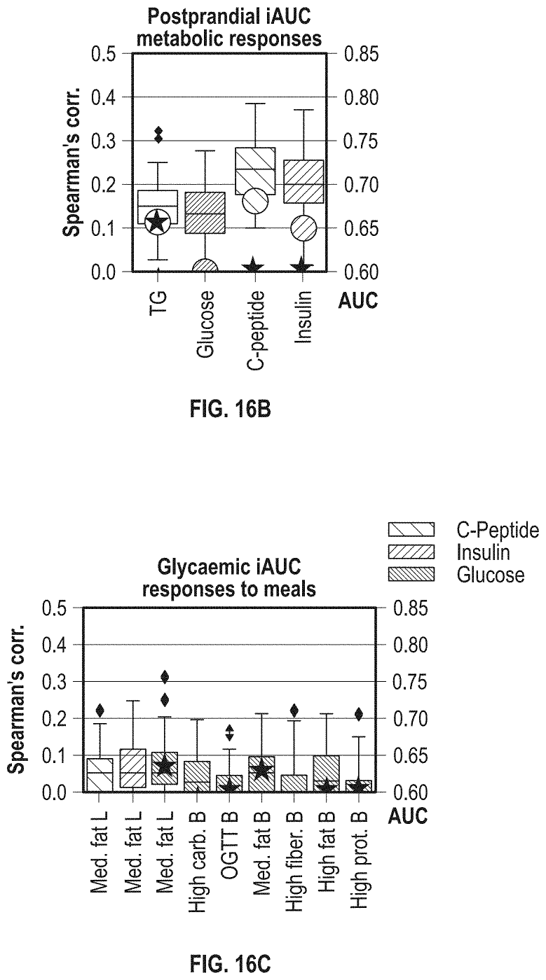

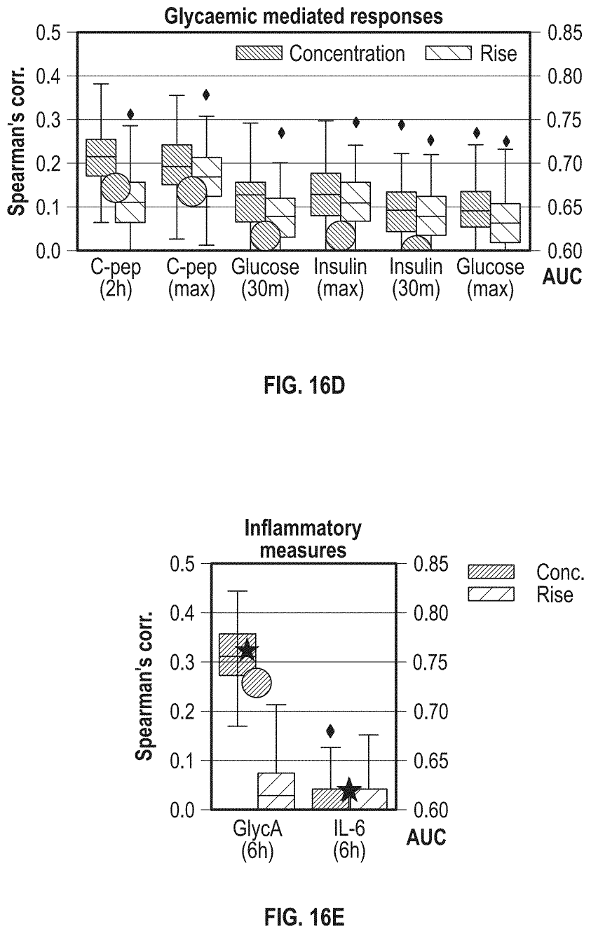

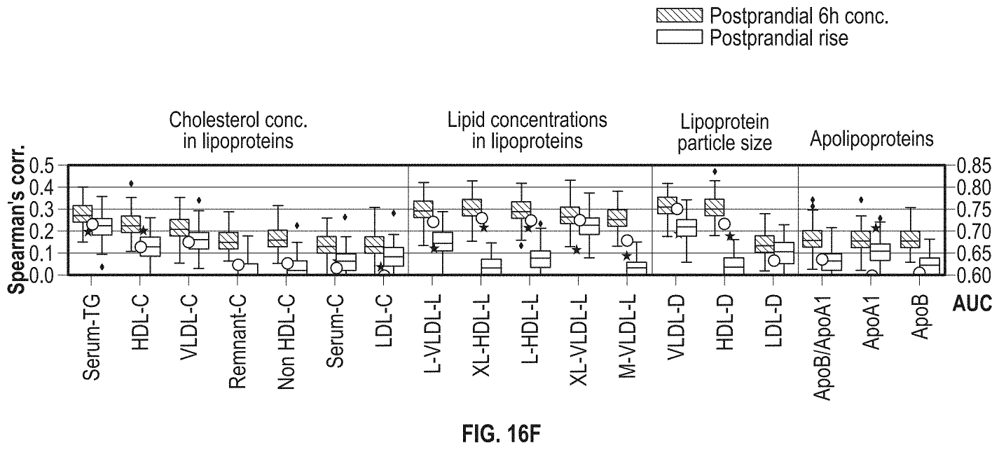

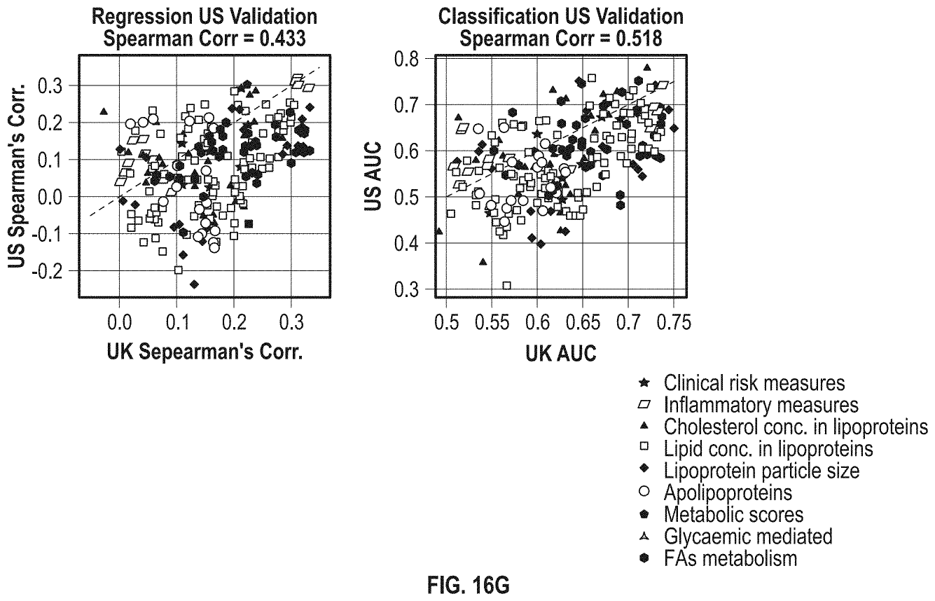

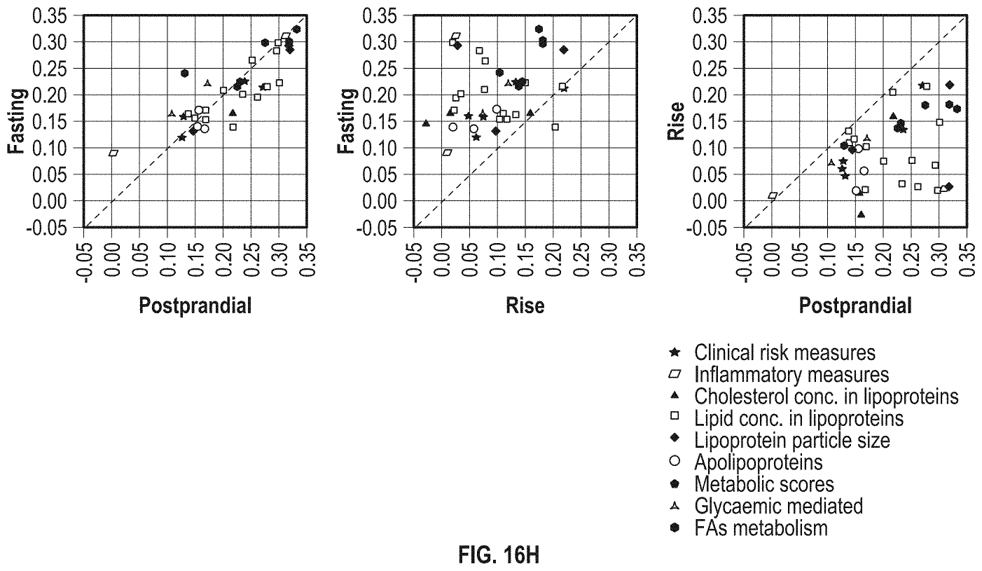

Modest concordance between microbiome classifiers and several traditional clinical fasting cardiometabolic biomarkers (FIG. 16A). These include near-term metrics, such as systolic and diastolic blood pressure, heart rate, lipids (TG, TC, HDL-C, LDL-C) and fasting glucose, as well as glycosylated hemoglobin (HbA1c), a widely-used clinical test reflecting mean glucose levels over weeks-to-months. Notably, the difference between total and high-density lipoprotein (HDL) cholesterol (e.g. non-HDL), recently considered a clinically useful aggregate count of atherogenic cholesterol fractions (Cui et al., Arch. Intern. Med. 161, 1413-1419, 2001), was also linked to gut microbial features (ρ=0.17; AUC 0.61). These associations were largely recapitulated in a clinical prediction model incorporating most of these factors to estimate latent 10-year risk of heart disease or stroke using the AtheroSclerotic CardioVascular Disease (ASCVD) algorithm (D'Agostino et al., Circulation 117, 743-753, 2008).

From the remaining compendium of blood biomarkers (FIG. 9A), stronger correlations were found between the microbiome and an inflammatory surrogate (glycoprotein acetyls, GlycA, FIG. 16A), as well as various emerging lipid measures linked to host health, such as HDL and VLDL particle size (HDL-D and VLDL-D, ρ=0.3 and 0.28 respectively), the lipid content of lipoprotein subfractions (including XL-HDL-L and L-HDL-L, ρ=0.39 and 0.37 respectively), and circulating polyunsaturated fatty acids (PUFA) fatty acid (omega-6 [FAcω6/FA] and PUFA [PUFA/FA] to total fatty acid ratios, ρ=0.31 for both). GlycA (Duprez et al., Clin. Chem. 62, 1020-1031, 2016) and VLDL-D have been strongly associated with increased risk for the metabolic syndrome, CVD, and T2D, whereas HDL-D and its lipid constituents, omega-6, and PUFA have strong inverse associations (Würtz et al., Circulation 131, 774-785, 2015; Ahola-Olli et al., Diabetologia 62, 2298-2309, 2019; Kettunen et al., Circ Genom Precis Med 11, e002234, 2018). The strongest association for all circulating markers was observed for large HDL particle lipid concentrations (XL-HDL-L and L-HDL-L, with ρ=0.41 and 0.38, and AUC=0.70 and 0.69, respectively), which also have the strongest inverse association with CVD and T2D of all the lipid measures (Würtz et al., Circulation 131, 774-785, 2015; Ahola-Olli et al., Diabetologia 62, 2298-2309, 2019; Kettunen et al., Circ Genom Precis Med 11, e002234, 2018). Similarly, the majority of glycemic indicators such as insulin, C-peptide (a surrogate of insulin secretion), and to a much lesser extent, impaired glucose tolerance (IGT) were also coupled to human gut microbiome composition (FIG. 16A). Derived predictors of insulin sensitivity (Quantitative Insulin sensitivity Check Index or QUICKI; Hrebicek et al., J. Clin. Endocrinol. Metab. 87, 144-147, 2002) and hepatic steatosis (Liver Fat Probability) were also reasonably captured using microbiome-based ML classifiers (ρ=0.22 and 0.18; AUC 0.66 and 0.64 respectively).

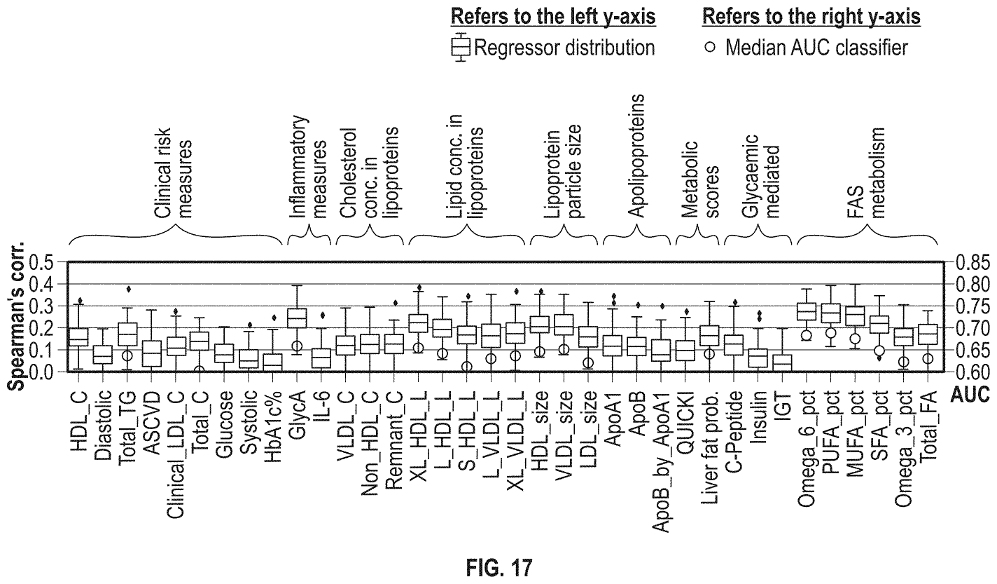

Species-based predictors proved more accurate for RF-based learning tasks than pathway abundance profiles (FIG. 17), consistent with other microbiome-wide training exercises (Thomas et al., Nat Med 25,667-678, 2019). Despite a smaller study population and a more restricted panel of fasting circulating metabolites, the primary findings were generally replicated in the US validation cohort (FIG. 16A), corroborating the existence of a strong, previously overlooked link between the gut microbiome and surrogate markers of cardiometabolic health.

The Gut Microbiome is a Better Predictor of Postprandial Triglycerides and Insulin Concentrations than of Glucose Levels

Fasting blood assays are the standard for most research and clinical investigations; however, in free-living conditions, individuals consume multiple meals throughout the day and therefore spend most of their waking hours in the postprandial state. Mixed nutrient meals (carbohydrate, fat and protein) result in person-specific food-induced elevations in triglycerides (TG), glucose, insulin, and other related metabolites, impacting personalized cardiometabolic responses and downstream health outcomes. Whilst prior efforts have demonstrated that postprandial glucose responses may, in part, be predicted by the gut microbiome (Zeevi et al., Cell 163, 1079-1094, 2015), the relationship between the microbiome and ‘real-life’ variations in both postprandial lipid and glucose-mediated metabolites has not been explored. Postprandial metabolic responses to foods of varying nutrient composition were therefore assessed in the clinic and free-living settings by considering the overall magnitude of the response by iAUC, as well as its peak concentrations, and its change from fasting (i.e. rise).

Firstly, postprandial TGs, glucose, C-peptide, insulin, and circulating metabolite concentrations were measured at regular intervals (0-6 h) in the clinic after the administration of two formulated, sequential test meals (890 kcal, 50 g fat and 85 g carb at 0 h [breakfast] and 500 kcal, 22 g fat and 71 g carb at 4 h [lunch]; FIGS. 16B, 16C). Notably, it was found that the magnitude of postprandial TG (0-6 h iAUC), insulin, and C-peptide (both 0-2 h iAUC) responses were more strongly associated with the gut microbiome (ρ=0.15, 0.19, and 0.21, respectively; AUC >0.63 for each) compared with postprandial glucose (0-2 h iAUC) responses (ρ=0.12 and AUC 0.59, FIG. 16B), findings replicated in the US validation cohort (FIG. 16B).

Following the in-person clinic day, glucose concentrations were also measured via continuous glucose monitoring over the subsequent 13-day at-home period (Berry et al., Protocol Exchange, 2020) that included responses to isocaloric standardized meals, in duplicate, with different macronutrient compositions (fat, carbohydrate, protein and fiber; Table 2). However, contrary to the clinic meal responses (FIG. 16B) and previous work (Zeevi et al., Cell 163, 1079-1094, 2015), the glucose 0-2 h iAUCs following these meals did not achieve high correlations with the microbiome regardless of their macronutrient composition (all p<0.11 and AUC<0.58, FIG. 16C). Whilst this may be due to the lower energy, fat, and carbohydrate dose in at-home isocaloric meals (500 kcal) compared to the successive clinic meals (total 1,390 kcal for breakfast and lunch), reducing discrimination between interindividual responses, Zeevi et al. (Cell 163, 1079-1094, 2015) found associations using meals of <500 kcal. However, the stool sample in this study was collected within 24 h of the metabolic clinic meal(s), whereas the standardized at-home meals were consumed (in random order) between days 2-13 post-home stool collection, introducing additional variability due to short-term fluctuations in microbiome composition (David et al., Nature 505, 559-563, 2014). Taken together, these results suggest that the microbiome is a stronger predictor of postprandial lipemia (TG) than glycaemia, with the strength of association for glycemic responses influenced by overall metabolic load and short-term variations in microbial composition rather than differences in macronutrient composition.

Postprandial Rises in Lipid- and Glucose-Mediated Measures are Differentially Predicted by the Microbiome Compared with Fasting Levels

Postprandial measures (iAUC and peak) depend both on the corresponding fasting measure and the meal-induced rise. Therefore, the differential prediction accuracy of the gut microbiome for fasting levels, postprandial (peak) total levels, and postprandial rises (FIG. 16H) were compared. When looking at lipid and glucose-mediated metabolites from the clinic day measures, despite a similar strength of association between peak (6 h), magnitude (iAUC) and fasting TG concentrations, the rise (6-0 h) was not similarly correlated (FIGS. 16A, 16E, 16F). In contrast, the microbiome associations with glycemic measures were comparable between fasting, peak, and rise (FIGS. 16A, 16D).

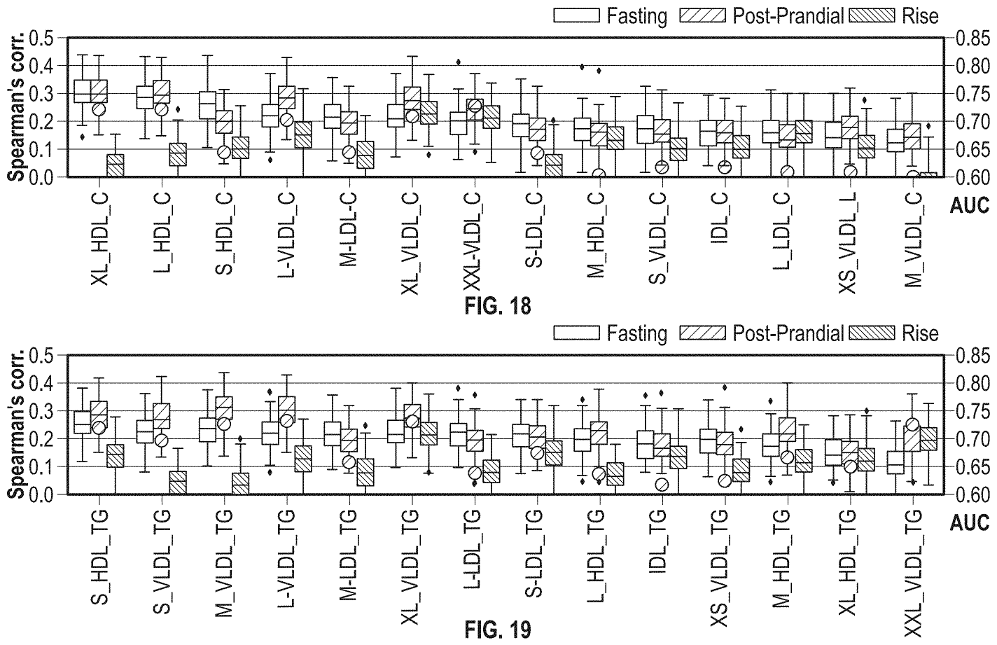

Of particular interest were the lipoprotein subfraction concentrations, composition, and size (FIGS. 18 and 19), which are remodeled postprandially, resulting in the generation of atherogenic lipoproteins (e.g. Large VLDL particles and TG-enriched LDL, and HDL particles). These atherogenic particles were predicted at comparable accuracy for both fasting and postprandial peak 6 h concentrations (FIGS. 16A, 16F, 16H), and notably, HDL and VLDL size (“-D”, key lipoproteins associated with cardiometabolic risk) achieve modestly stronger correlations (ρ=0.32 and 0.31, respectively) postprandially (FIG. 16F). However, as with TG, the microbiome was substantially less predictive for the postprandial rise (6 h—fasting) in all lipid metabolite measures compared with fasting and postprandial 6 h peak concentration (FIGS. 16A, 16F, 16H). For example, HDL-D is closely associated with gut microbial composition at fasting and 6 h postprandially (ρ=0.30 and 0.32; AUC 0.71 and 0.72 respectively; FIGS. 16A, 16F, 16H), but not with the rise (FIG. 16F).

These differential associations suggest that the microbiome may influence postprandial lipid-mediated measures via effects on fasting measures but may impact the postprandial glucose rise more independently of fasting levels.

Distinct Microbial Signatures Discriminate Between Positive and Negative Metabolic Health Indices Under Fasting Conditions

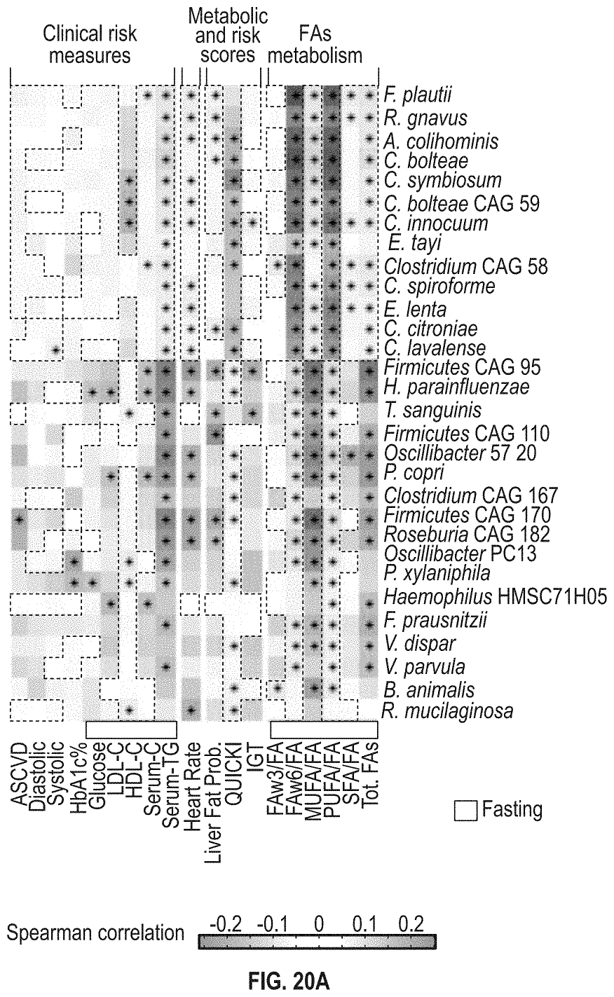

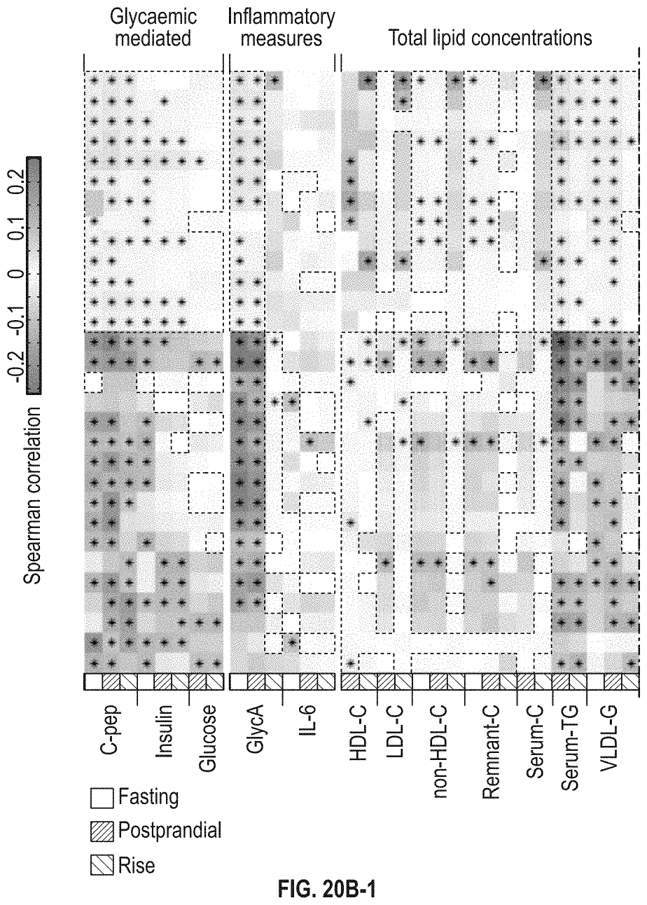

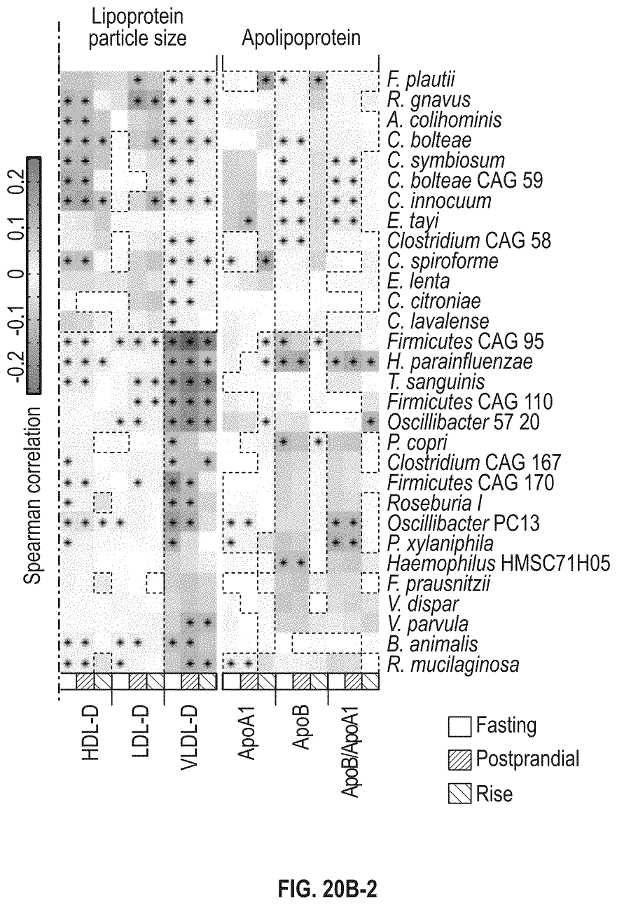

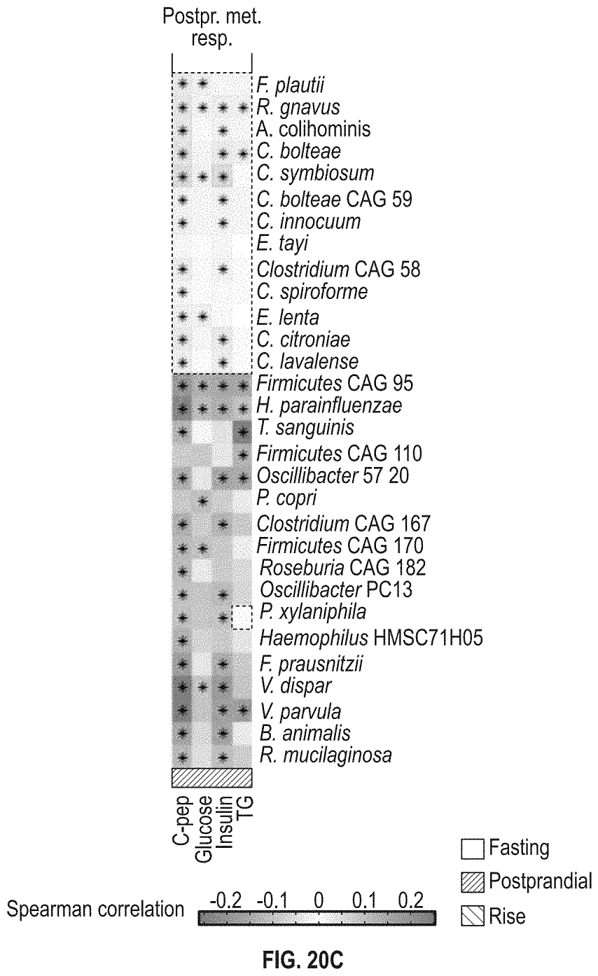

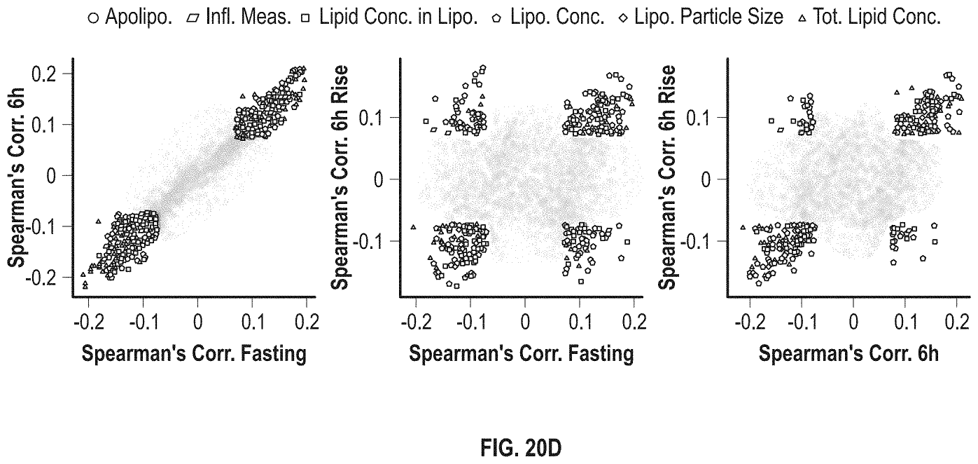

Motivated by the observed potential of the gut microbiome to predict the fasting and postprandial levels of circulating metabolic markers, identifying the specific taxa and functions driving these associations was next sought. Among three general risk indices of cardiovascular health (ASCVD, liver fat probability, and insulin sensitivity or quantitative insuli-sensitivity check index (QUICKI)) which demonstrated significant although rather modest correlation of predictions (0.2) using the microbiome-wide RF model (FIG. 16A), eight species were found that were significantly correlated with all three (negatively or positively, p<0.05). Seven of these eight were concordantly correlated in the direction of a more healthful metabolic profile (i.e. correlated for greater QUICKI values and lower ASCVD and fatty liver risk), hinting at a global underlying microbial signature of improved metabolic health. These taxa included Flavonifractor plautii and Clostridium innocuum (higher cardiometabolic risk, FIGS. 20A-20C) and Oscillibacter sp 57 20, Haemophilus parainfluenzae, and Eubacterium eligens (lower risk, FIGS. 20A-20C) that had previously been linked with healthy and less-healthy dietary habits.

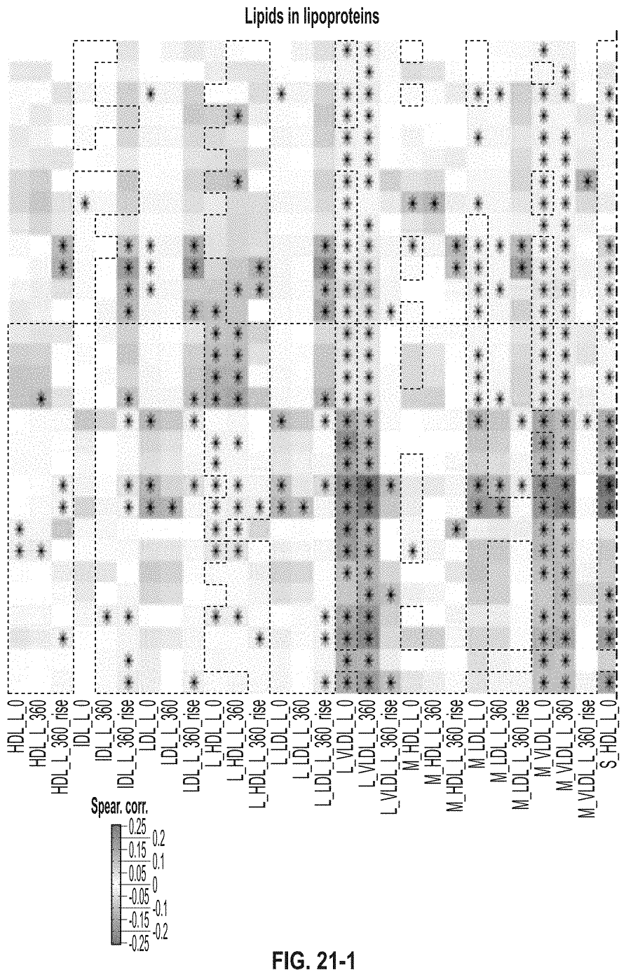

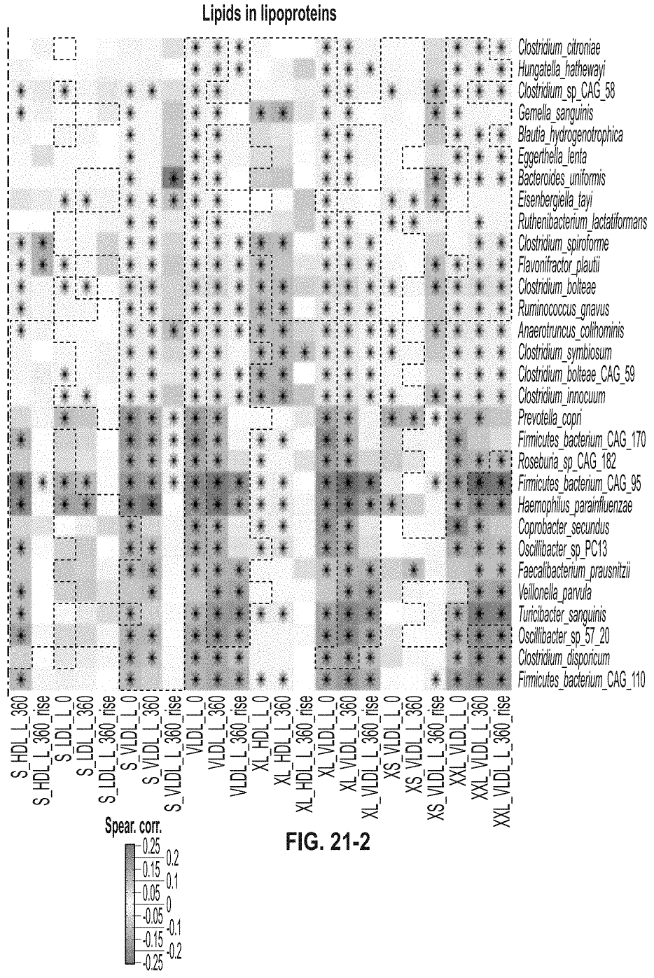

Similarly, distinct separations were found between two opposing and clearly defined clusters of species either positively or negatively correlated with fasting cardiometabolic measures (FIG. 20A), including blood pressure, inflammatory markers, lipid concentrations, lipoprotein sizes and fractions, and apolipoproteins (FIGS. 20A, 20B-1, 20B-2). As per the association with diet, species correlated with positive markers included some taxa generally regarded as healthy (e.g. F. prausnitzii) but also many uncultivated and under-characterized bacteria (7 from the cluster of 18). With the notable exception of three species of Provotella (P. copri, P. clara, and P. xylaniphila) the positive cluster included many distinct genera, pointing at a large functional richness and diversity. In contrast, the cluster of species negatively correlated with positive markers again included many Clostridium species (5 of the 12 in the cluster) and the recurrent negatively connotated R. gnavus and F. plautii. Large HDL particles (and their lipid compositions, FIGS. 21-23), which have strong inverse associations with cardiometabolic outcomes (Würtz et al., Circulation 131, 774-785, 2015; Ahola-Olli et al., Diabetologia 62, 2298-2309, 2019) as well as with the microbiome (FIG. 16A), were associated with the healthy cluster. Conversely, lipoproteins associated with increased risk of CVD and T2D (VLDL of all sizes; XXL, XL, L, M, S and lipid composition) and atherogenicity (Skeggs et al., J. Lipid Res. 43, 1264-1274, 2002; S-LDL, M-HDL and S-HDL TG), were associated with the less-healthy cluster (FIGS. 21-23).

Circulating omega-6 and total polyunsaturated fatty acids (PUFA), which reflect dietary intake due to the lack of endogenous production of these fatty acids (Hodson et al., Prog. Lipid Res. 47, 348-380, 2008), were associated with the healthy cluster for which Firmicutes bacterium CAG95 was the most correlated representative, and F. plautii the strongest negative correlation (FIG. 20A). Both omega-6 and PUFA have been linked to reduced risk of chronic disease, whether measured from dietary inventories (Li et al., Am. J. Clin. Nutr., doi:10.1093/ajcn/nqz349, 2020) or directly assayed from the circulation (Würtz et al., Circulation 131, 774-785, 2015; Ahola-Olli et al., Diabetologia 62, 2298-2309, 2019; Marklund et al., Circulation 139, 2422-2436, 2019). In contrast, circulating monounsaturated fatty acids (MUFA) in blood were associated with the unhealthy cluster, with an under-characterized Osciffibacter species (sp. 57_20) and Clostridium bolteae responsible for the strongest negative and positive associations respectively. Measures of circulating MUFA but not dietary intake of MUFA (Chowdhury et al., Ann. Intern. Med. 160, 398-406, 2014; Zong et al., BMJ 355, i5796, 2016) have been associated with increased risk of CVD and T2D. Differences in circulating vs. estimated dietary intakes of MUFA may be a function of endogenous MUFA production, as well as the divergent animal and plant dietary sources of MUFA (Wu et al., Nat. Rev. Cardiol. 16, 581-601, 2019; Zong et al., Am. J. Clin. Nutr. 107, 445-453, 2018), complicating their relationship with chronic health outcomes (Hodson et al., Prog. Lipid Res. 47, 348-380, 2008). Taken together with the findings, these results suggest that food sources of MUFA play an important role in the relationship between MUFA and health.

Both Favorable and Unfavorable Microbial Signatures of Metabolic Health were Maintained Under Postprandial Conditions

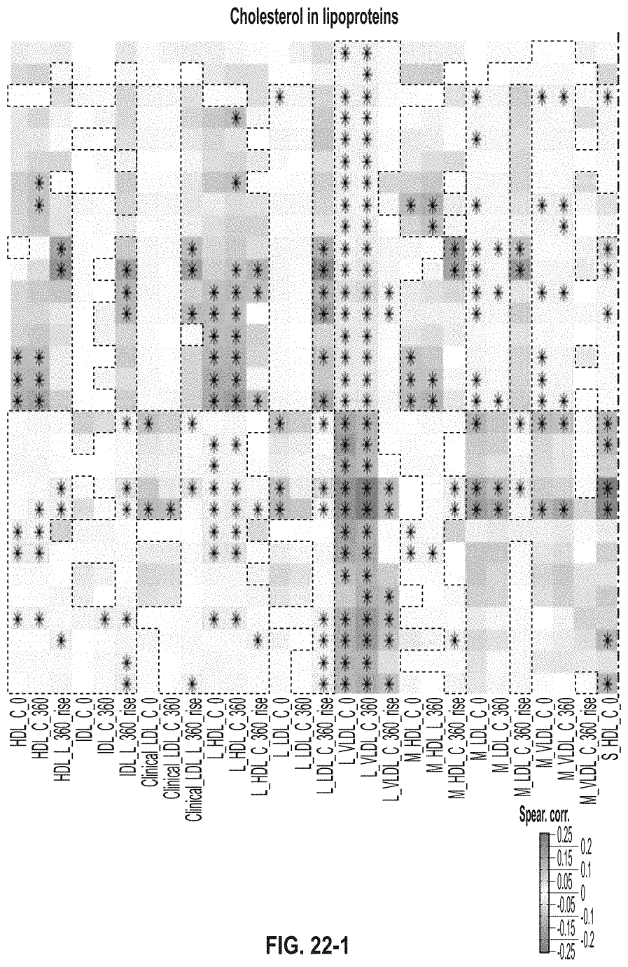

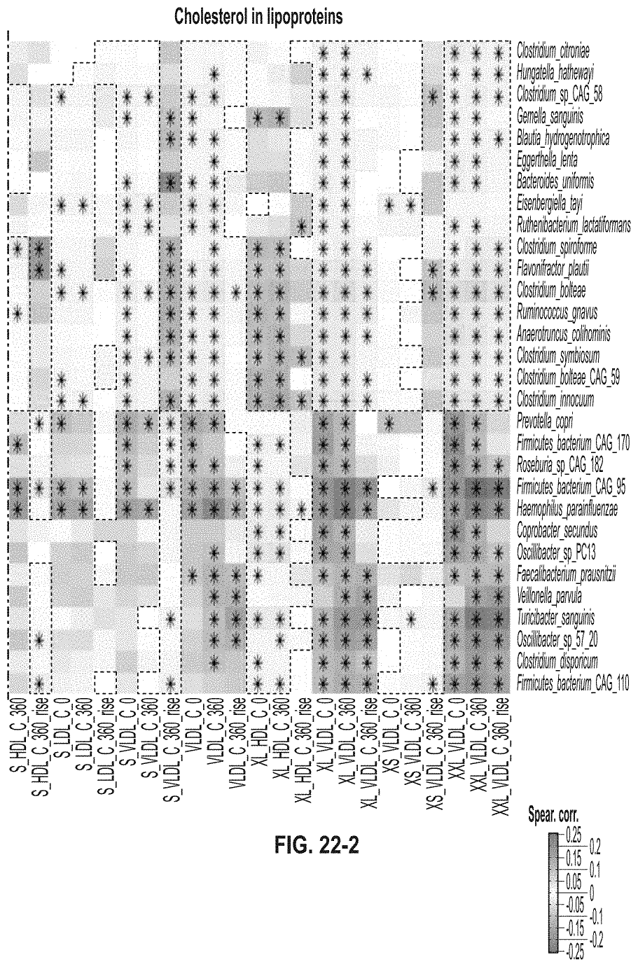

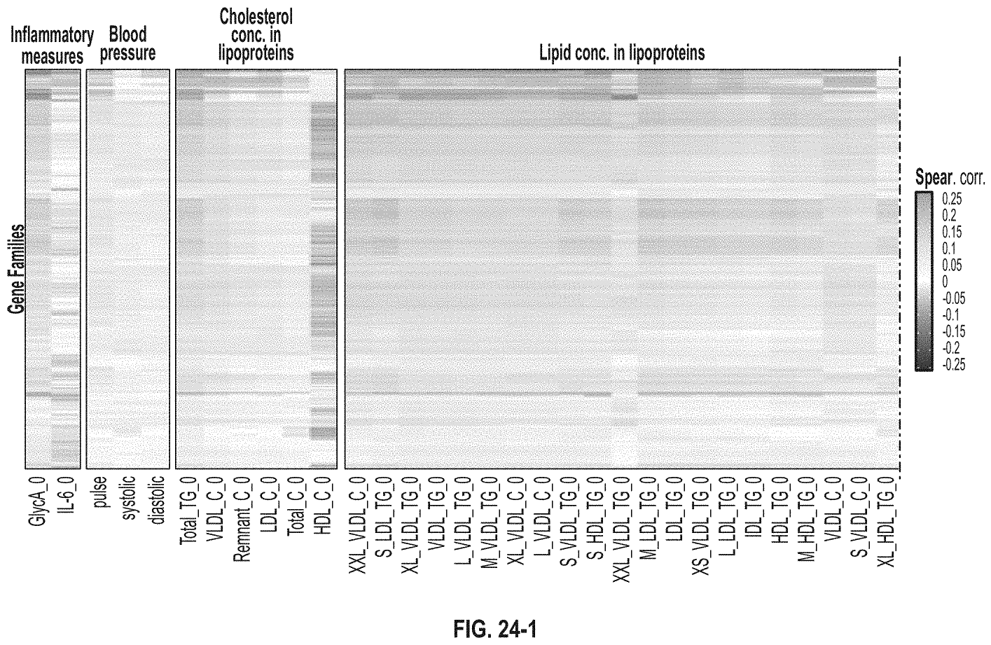

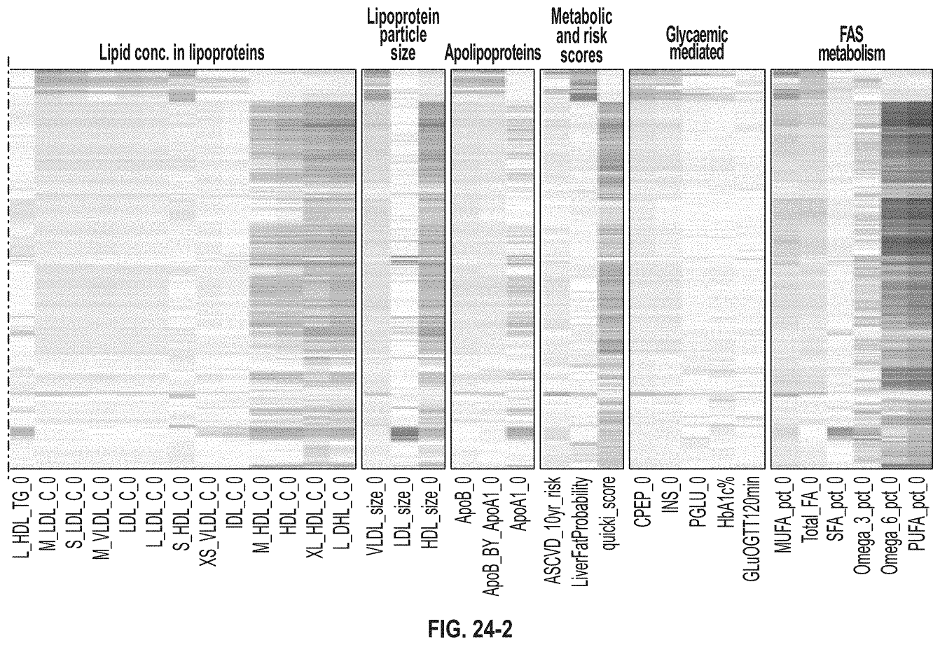

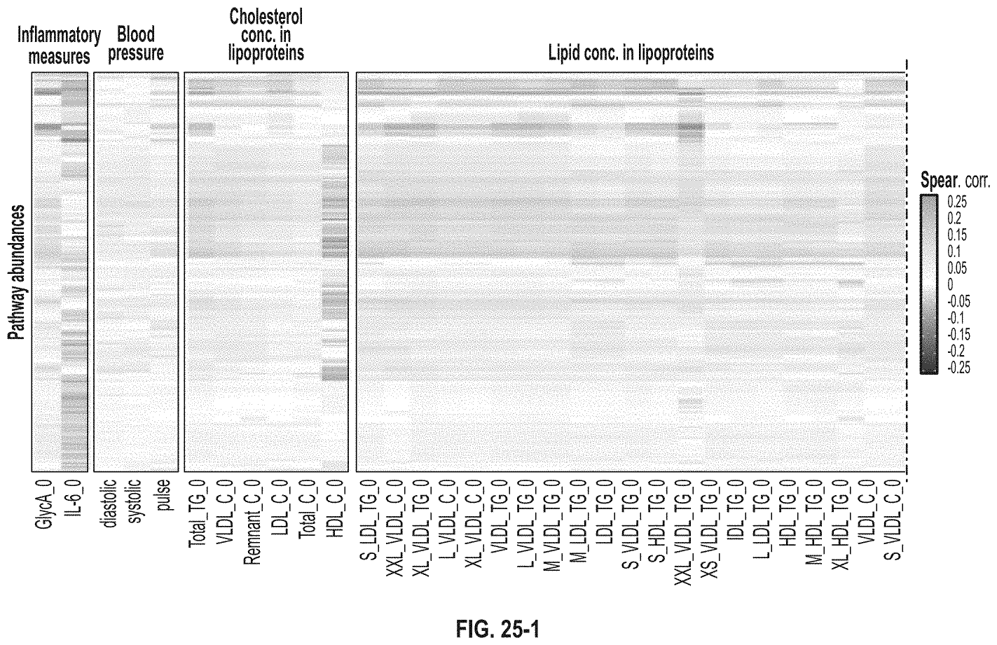

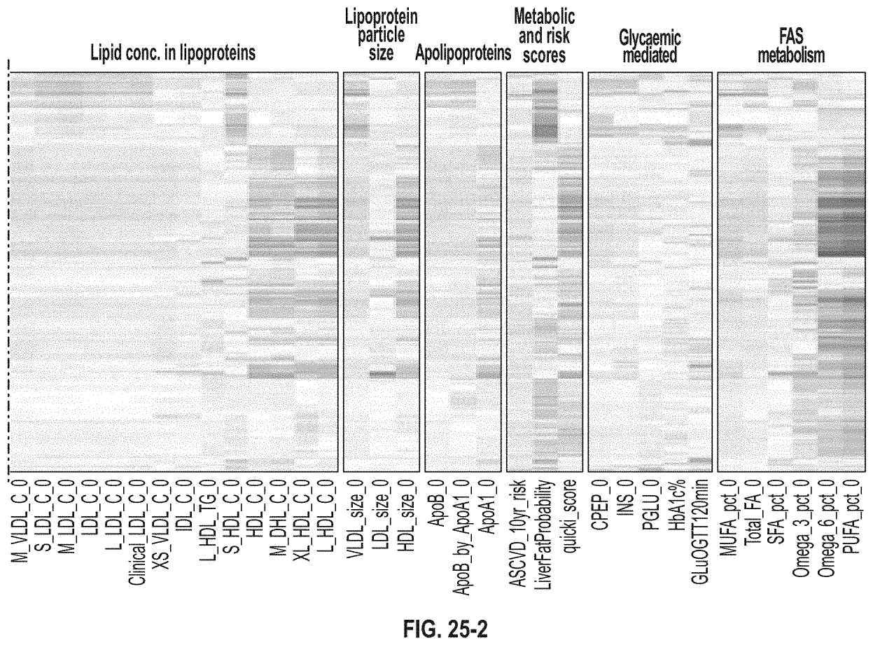

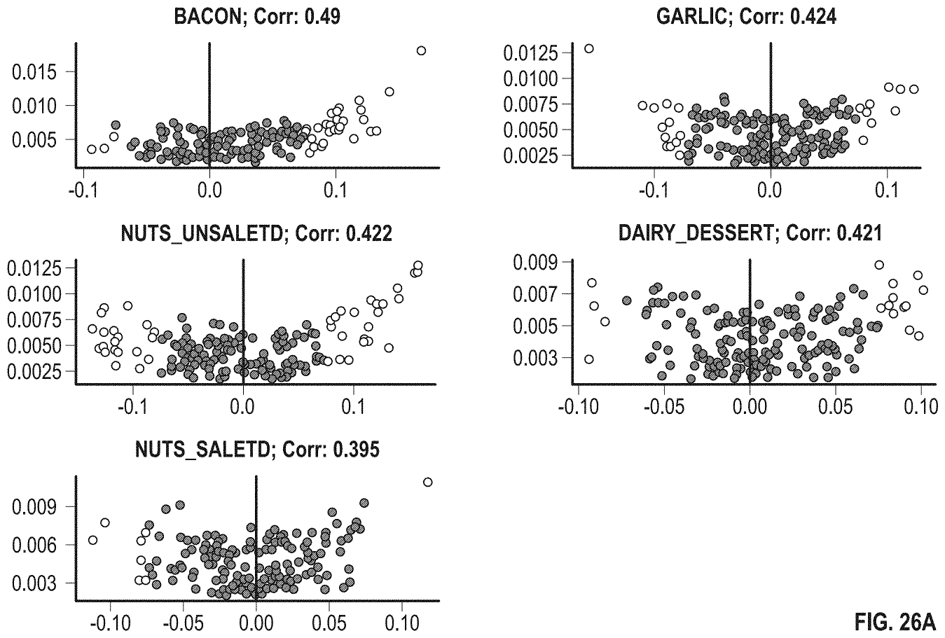

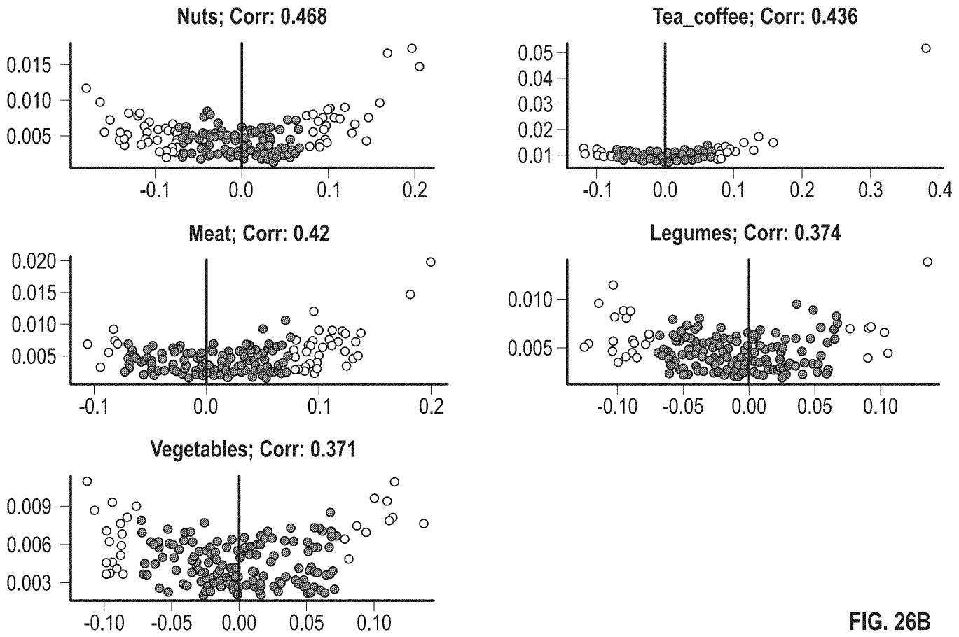

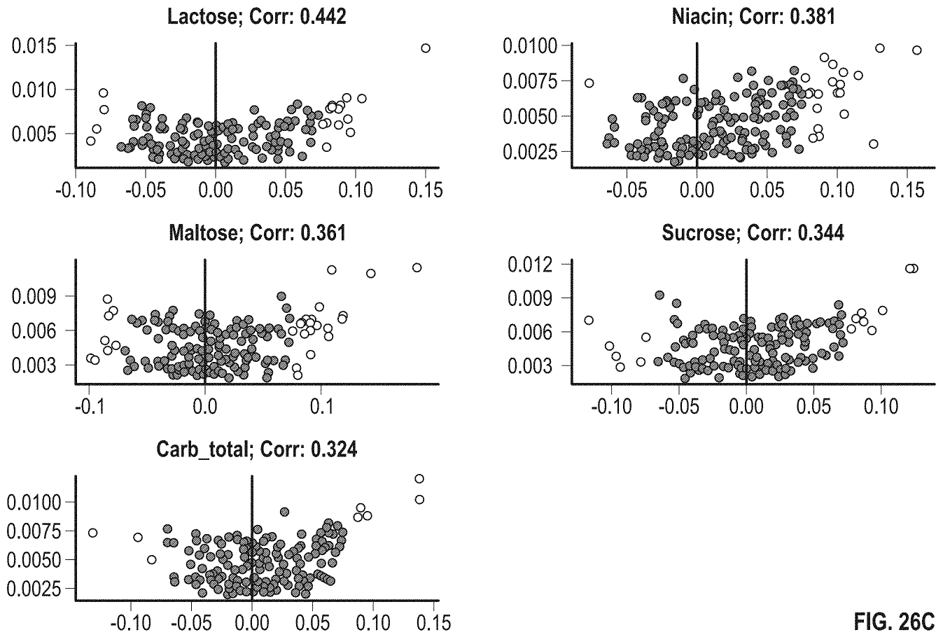

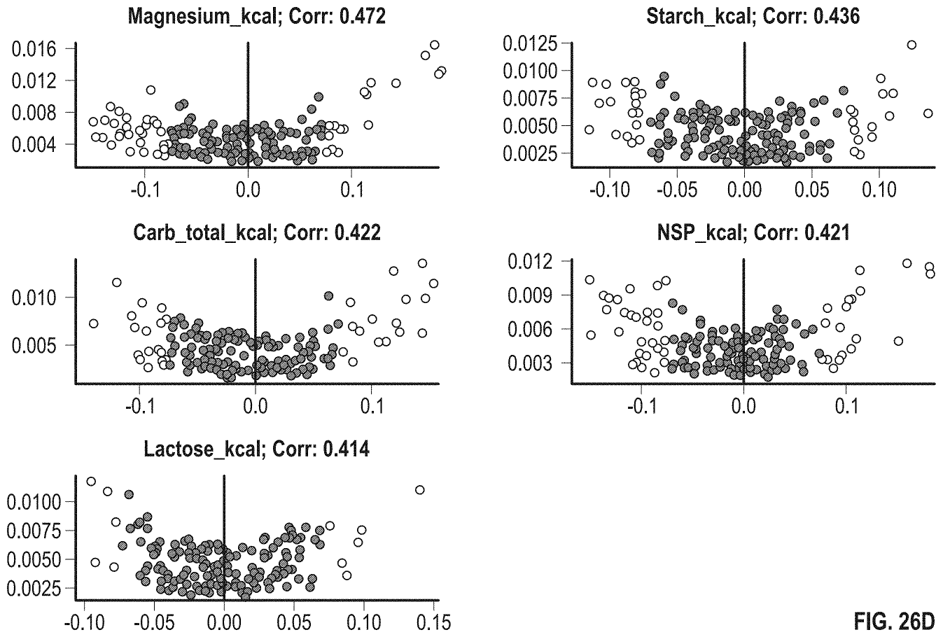

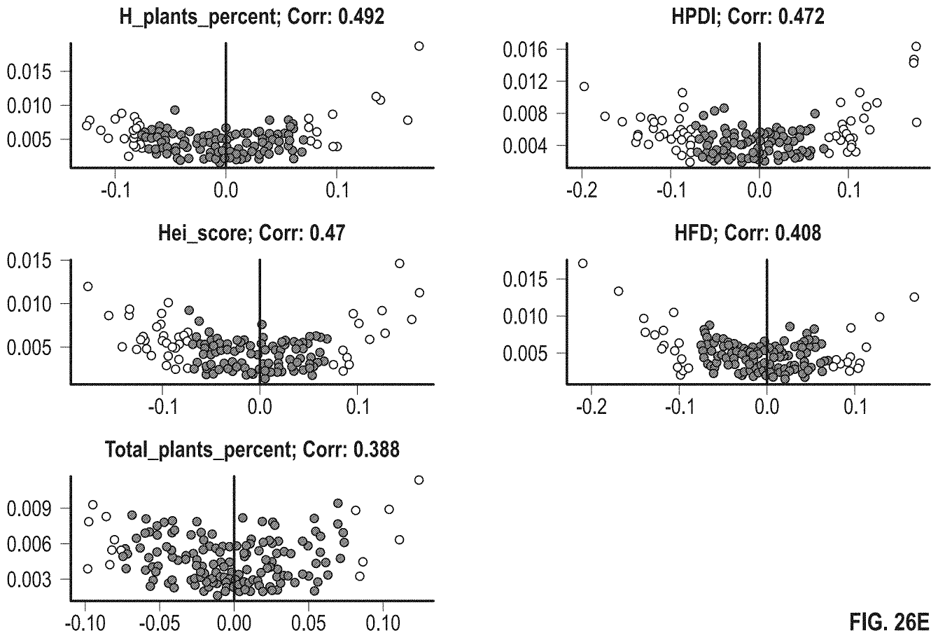

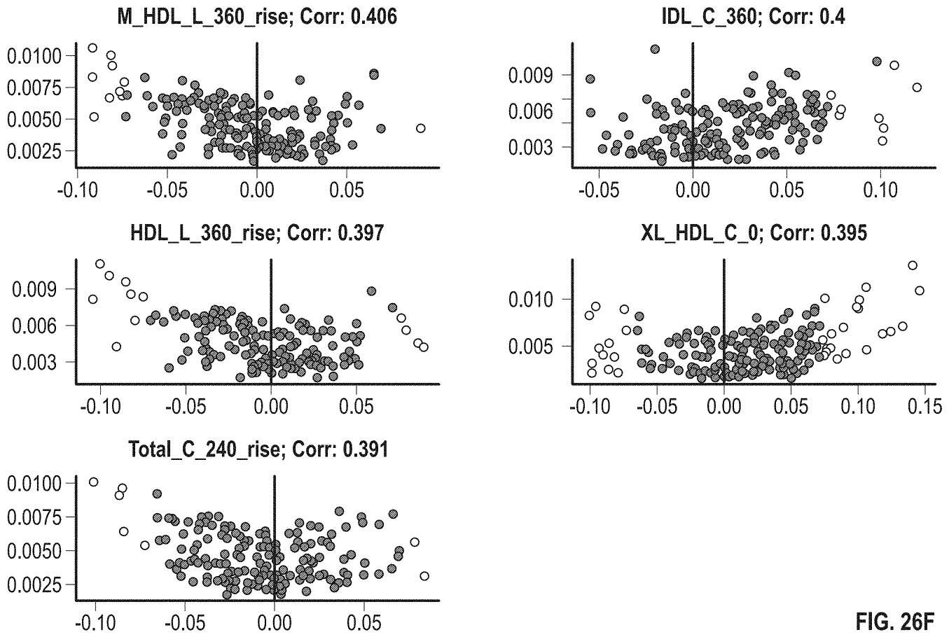

Links between postprandial levels of cardiometabolic and inflammatory measures corresponded with the segregation of healthful vs. detrimental taxa observed under fasting conditions (FIGS. 20B-1 & 20B-2 and FIGS. 21-23). Notably, fasting and postprandial GlycA, which were found to be highly correlated with postprandial TG concentrations, were strongly linked with the microbiome (62 species significantly correlated at 6 hours and 67 at fasting), substantially exceeding IL-6 (5 and 26 significant postprandial and fasting associations, FIGS. 20B-1 & 20B-2). F. plautii and R. gnavus were the two species most correlated with increased inflammation both in fasting and postprandial conditions, whereas H. parainfluenzae and Firmicutes bacterium CAG95 were the strongest associations with reduced GlycA levels. VLDL lipoprotein subfractions (markers of adverse cardiometabolic effects) were also consistently associated with the less-healthy cluster both at fasting and postprandially. Postprandial rises, rather than absolute postprandial levels, were frequently uncoupled from the microbial associations with fasting markers; several positive correlations between microbial species and fasting and peak metabolites measures became negative when correlating the same species with the rise from fasting (and vice versa, FIG. 20D). For example, the rise in total LDL cholesterol and size (-D, FIGS. 20B-1 & 20B-2) was differentially associated with clusters compared to fasting levels (especially for T. sanguinis, B. animalis, and R. mucilaginosa). S- and XL- HDL total lipid (-L) and cholesterol (-C) levels also paralleled this behavior (FIGS. 21, 22), possibly reflecting postprandial lipoprotein remodeling and reciprocal exchange of TG and cholesterol, between these particles and TG-rich lipoproteins (chylomicrons and VLDL; Cohn, J Can. J. Cardiol. 14 Suppl B, 18B-27B, 1998). In contrast, the associations of the microbial species with absolute fasting and postprandial peak levels were fully consistent (FIG. 20D), again reflecting the close relationship between fasting levels and postprandial responses. The same “favorable” vs. “unfavorable” clustering of microbiome features was observed when analyzing microbial pathways and gene families (FIGS. 24 and 25). This supports the segregation of many taxa, even at the species level (and likely more so among strains), by their underlying biochemical activities in the microbiome. The strengths of microbe-blood marker associations measured using Spearman's correlation were consistent with the estimated microbe relevance by the random forest model (FIG. 26F). Importantly, these associations were confirmed in the PREDICT 1 US validation cohort; there was a total of 62,366 microbe-index correlations for indices present in both cohorts, and for the 292 that were significant both in the UK cohort (q<0.2) and in the US cohort (p<0.05) the concordance in the sign of the correlation reached 90.8% for the associations in fasting conditions and 91.2% postprandially.

Prevotella copri Diversity and Blastocystis Spp. Presence are Markers of Improved Postprandial Glucose Responses

Some ecologically unusual microbes hypothesized to have population-scale health effects solely based on their presence or absence appeared among the microbial signatures. Among them, Prevotella copri is a frequent and highly abundant inhabitant of the gut (Human Microbiome Project Consortium. Nature 486, 207-214, 2012; Arumugam et al., Nature 473, 174-180, 2011), but its beneficial or detrimental role in human health remains controversial (Cani, Gut 67, 1716-1725, 2018; Ley, Nat. Rev. Gastroenterol. Hepatol. 13, 69-70, 2016). Previous reports have yielded conflicting accounts of P. copri in glucose homeostasis, with some studies suggesting health benefits (Kovatcheva-Datchary et al., Cell Metab. 22, 971-982, 2015; De Vadder et al., Cell Metab. 24, 151-157, 2016) and others suggesting deleterious effects (Pedersen et al., Nature 535, 376-381, 2016) possibly due to subspecies diversity (Tett et al., Cell Host Microbe, doi:10.1016/j.chom.2019.08.018, 2019; De Filippis et al., Cell Host Microbe 25, 444-453.e3, 2019). These data largely find P. copri to be associated with beneficial cardiometabolic markers, being weakly negatively correlated with estimated visceral fat (ρ=−0.09, p=0.009, q=0.098), fasting VLDL-D (ρ=−0.07, p=0.06, q=0.21), and fasting GlycA (ρ=−0.12, p=0.0001, q=0.005) among others (Table 3). While almost no habitual diet foods, nutrients, or scores were associated with P. copri, this bacterium showed a very strong correlation with postprandial increases of several circulating metabolic markers when compared with corresponding absolute fasting or postprandial levels. Postprandial rises in glucose (ρ=−0.12, p<0.0002) and polyunsaturated and omega-6 fatty acids (ρ=0.11 and 0.10, respectively, and p<0.001) were among the top-scoring correlations and were more strongly connected with the microbiome than were corresponding fasting and postprandial levels, in sharp contrast with what was observed for the overall microbiome (FIGS. 16A, 16B), suggesting a potentially unique role for P. copri in host metabolism.

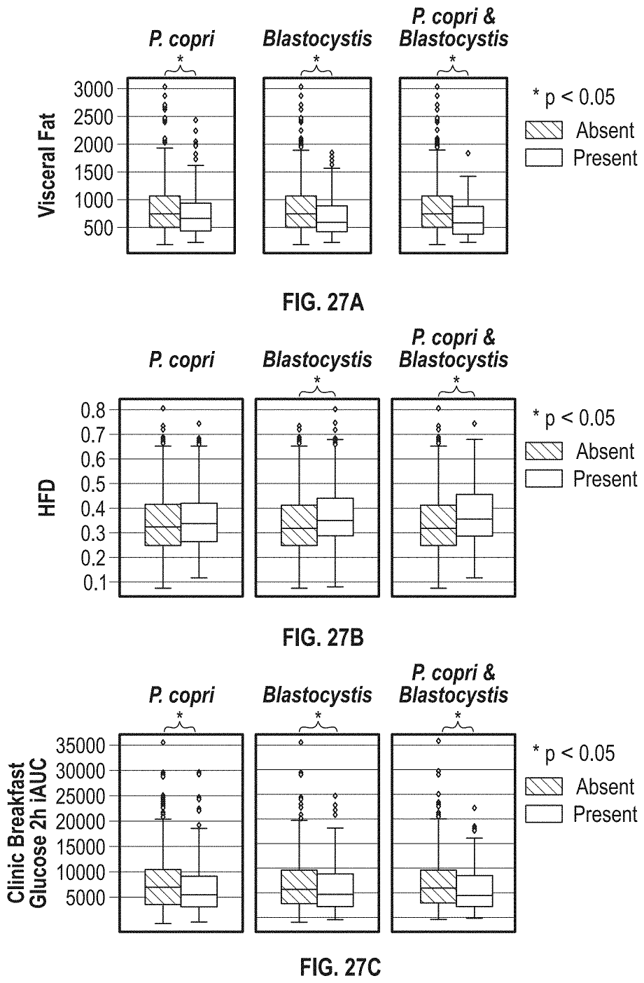

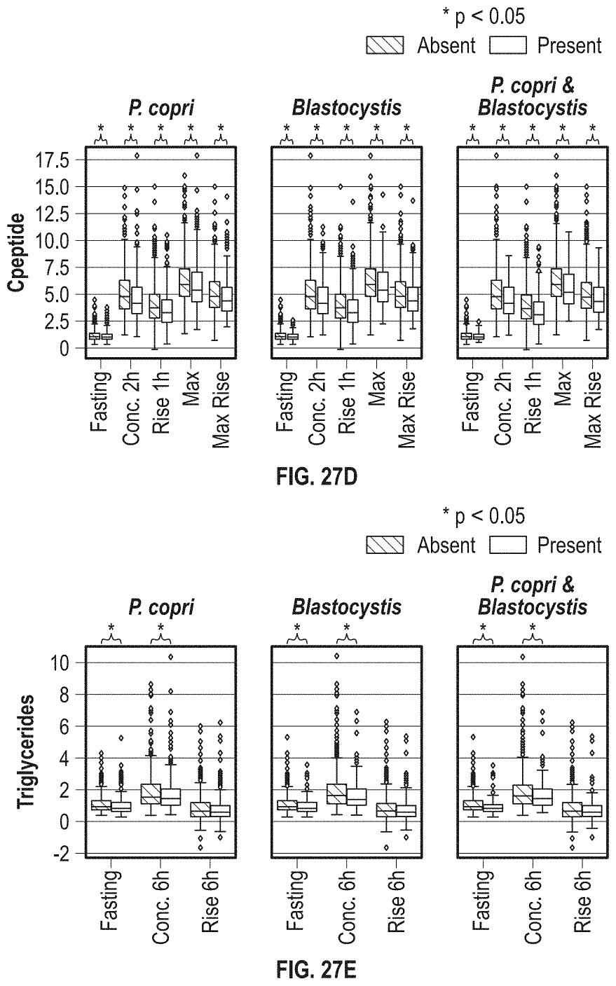

As P. copri has a relatively low prevalence in Western-lifestyle populations but is highly abundant when present (Tett et al., Cell Host Microbe, doi:10.1016/j.chom.2019.08.018, 2019), the presence of one or more of the subtypes of this species was tested (Tett et al., 2019) to determine whether it is associated with markers of improved glucose metabolism. P. copri is present in the form of at least one of its subtypes in 29.8% of the PREDICT 1 individuals, and significant differences were identified in P. copri carriers including lower C-peptide (−9.2%, p=0.002) (FIG. 27D), insulin (−14%, p=0.006), and lower TG levels (−3.2%, p=0.003) (FIG. 27E) compared to individuals without this species. Similarly, postprandial blood glucose spikes after breakfast were significantly less pronounced in individuals with P. copri (−20.4% glucose iAUC at 2 h, p=0.002, FIG. 27C), and visceral fat was significantly lower (−12.5%, p=3E-7, FIG. 27A). Although these observations are only associative, and the direct effect of P. copri on these markers of glucose metabolism is unknown, this positive association further supports that the presence of P. copri in the gut microbiome could be beneficial in glucose homeostasis.

Blastocystis spp. is a unicellular eukaryotic parasite increasingly regarded as a commensal member of the gut microbiome rather than a potential pathogen (Clark et al., Adv. Parasitol. 82, 1-32, 2013; Alfellani et al., Acta Trop. 126, 11-18, 2013; Lukeš et al., PLoS Pathog. 11, e1005039, 2015). It shares with P. copri a limited prevalence in Western-lifestyle populations (Beghini et al., ISME J. 11, 2848-2863, 2017) coupled with high relative abundance when present, unique among eukaryotic organisms in the gut to date. By assessing microbiome characteristics in presence or absence of Blastocystis spp., evidence was found that Blastocystis-positive individuals (28.1% in the cohort) also have a favorable glucose homeostasis and lower estimated visceral fat (−14.9% glucose iAUC, −21.7% visceral fat, p<0.01, FIGS. 27A and 27C). The latter confirms that Blastocystis spp. is less prevalent in overweight and obese individuals compared to individuals with BMI in the normal range, as previously shown (Beghini et al., ISME J. 11, 2848-2863, 2017) in multiple cohorts (Le Chatelier et al., Nature 500, 541-546, 2013; Nielsen et al., Nat. Biotechnol. 32, 822-828, 2014; Andersen et al., FEMS Microbiol. Ecol. 91, 2015; Qin et al., Nature 464, 59-65, 2010). Interestingly, the effect of the simultaneous presence of P. copri and Blastocystis spp. (12.8% of the individuals) appears to further promote healthier metabolic function. Visceral fat is 9.4% lower on average (p=0.028, Table 4) for individuals positive for both P. copri and Blastocystis spp. compared to individuals with only one or the other and 22.6% lower (p=3.3E-7) compared with individuals lacking both. Triglycerides and C-peptide were also consistently lower (although not individually significant, Table 4) when both microbes were present.

A Clear Microbial Signature of Health Levels Consistent Across Diet, Obesity Indicators, and Cardiometabolic Risks

In the preceding analyses, a consistent set of microbial species was observed that were strongly linked to (1) foods and food indices reflecting different levels of a “healthy” diet, (2) indicators of obesity and of general health, (3) fasting circulating metabolites connected with cardiometabolic risks, and (4) postprandial responses to food. To test the consistency of such a signature, a representative set of “health” indicators were selected from each of the four categories (diet, personal characteristics, fasting and postprandial biomarkers) and ranked each microbial species based on their correlation coefficient. By averaging the ranks of the association (or inverted ranks for “unhealthy” indicators), remarkable agreement among microbes associated with different positive or negative indicators of health was found (FIGS. 28-1 and 28-2, Table 5).

In particular, Firmicutes CAG 95 is the uncultivated species with the most beneficial score (average rank 7.14) and ranked within the top 5 correlated species for 13 of the 20 indicators. Of the “health”-associated microbial species only R. hominis (23.76) was already convincingly linked with health in case/control disease investigations (Machiels et al., Gut 63, 1275-1283, 2014), even though others such as F. prausnitzii (Sokol et al., Proc. Natl. Acad. Sci. U.S.A 105, 16731-16736, 2008) and P. copri were highly ranked (average ranks 31.7 and 37.2 respectively, 18th and 21st best ranks) but not in the top 15. The beneficial signature also included several known species such as E. eligens (16.6) and H. parainfluenzae (6.4) without clear roles in health, and additional species without cultivated representatives such as Roseburia CAG 182 (15.5), Oscillibacter sp 57_20 (13.6), Firmicutes bacterium CAG 170 (20.1). Oscillibacter sp PC13 (24.5), Clostridium sp CAG 167 (24.8), and Ruminococcaceae bacterium D5 (24.8). Species that were conversely consistent with indicators of poor overall health (FIGS. 28-1 and 28-2) included the already discussed set of Clostridia (C. spiroforme—149.7, C. bolteae CAG 59-149.9, C. bolteae—154.8, Clostridium CAG 58-157.5, C. symbiosum—157.4, C. innocuum—155.1). The two strongest microbial indicators of poor cardiometabolic and diet-related health were the mucolytic microbe R. gnavus (158.8) and F. plautii (169.1), again previously found to be associated with disease conditions (Hall et al., Genome Med. 9, 103, 2017; Azzouz et al., Ann. Rheum. Dis. 78, 947-956, 2019; Ni et al., Gastroenterology 152, S214, 2017; Valles-Colomer et al., Nat Microbiol 4, 623-632, 2019; Gupta et al., mSystems 4, 2019; Jiang et al., Brain Behav. Immun. 48, 186-194, 2015). Overall, this set of 30 species serves as a marker of overall good or poor general health and dietary patterns in non-diseased human hosts.

Discussion

PREDICT 1 represents the first diet-microbiome clinical intervention study to identify both individual components of the microbiome and an overall gut microbial signature associated with multiple measures of dietary intake and cardiometabolic health. These signatures reproduced across UK and US populations, across multiple previously-published study populations, and for multiple dietary, biometric, and blood markers of health and cardiometabolic risk, including individual food items, nutrients, dietary patterns, adiposity, BMI, circulating lipids, inflammatory markers, blood glucose, and interactions between baseline and postprandial response levels. Notably, microbiome signatures robustly grouped both microbiome and dietary components into health-associated and anti-associated clusters, the latter in agreement with dietary quality and diversity scores (such as the Plant-based Diet Index [PDI] and Healthy Food Diversity [HFD] index) known to be health-associated (Vadiveloo et al., Br. J. Nutr. 112, 1562-1574, 2014; Kim et al., J. Nutr. 148, 624-631, 2018) and often unlinked from macronutrient source (e.g. more vs. less healthy plant- and animal-based foods). The diversity of a healthy diet (measured by the HFD and PDI) was particularly predictable by the microbiome, surpassing other indices such as the Mediterranean diet index that has been independently linked with microbiome composition (Meslier et al., Gut, doi:10.1136/gutjnl-2019-320438, 2020). The segregation of favorable and unfavorable microbial clusters according to the heterogeneity of the food source (healthy or unhealthy animal or plant), quality (processed vs unprocessed), and dietary patterns highlights the importance of looking beyond nutrients and single foods in diet-microbiome research. The substantially greater detail and consistency in the results relative to prior diet-microbiome work (Zeevi et al., Cell 163, 1079-1094, 2015; Falony et al., Science 352, 560-564, 2016; Zhernakova et al., Science 352, 565-569, 2016; Thingholm et al., Cell Host Microbe 26, 252-264.e10, 2019; Fu et al., Circ. Res. 117, 817-824, 2015; McDonald et al., mSystems 3, 2018) may be due to the quality in the metagenomic profiling and the large sample size. However, given the limitations of FFQ dietary data (which can be highly scalable but noise-prone; Cade et al., Nutr. Res. Rev. 17, 5-22, 2004), future diet-microbiome studies would benefit further from more detailed weighed food record data complemented with nutritionist/dietitian support.

Several aspects of the gut microbiome associations and matched signatures across diet, obesity, and metabolic health measures are striking with respect to their potential novel epidemiology and microbial biochemistry. A surprising proportion of diet- or health-associated taxa in these results are represented solely by existing or newly generated metagenomic assemblies (Pasolli et al., Cell 176, 649-662.e20, 2019), in addition to very recently isolated organisms with limited cultured strains. This was true for Lawsonibacter asaccharolyticus, the taxon most strongly associated with individual food items (particularly coffee) and nutrient intake, for which only two recent publications with limited and conflicting microbial physiology and taxonomy exist (Sakamoto et al., Int. J. Syst. Evol. Microbiol. 68, 2074-2081, 2018; Hosny et al., New Microbes New Infect 14, 85-92, 2016). Both of the taxa most abundant in diets rich in healthy plant-based foods were represented only by previous metagenomic assemblies (Nielsen et al., Nat. Biotechnol. 32, 822-828, 2014; Firmicutes CAG 95 and Roseburia CAG 182), as was the strongest microbial association with adiposity (Clostridium CAG 58) and several of the most reproducible microbes associated with (un)healthy blood markers (C. bolteae CAG 59, Clostridium CAG 167). Other microbes found here to have dietary or cardiometabolic associations, such as Prevotella spp. or Blastocystis spp., have been characterized in greater biochemical detail, but their prevalence and population structure in the human microbiome have only recently begun to be appreciated (Tett et al., Cell Host Microbe, doi:10.1016/j.chom.2019.08.018, 2019; Beghini et al., ISME J. 11:2848-2863, 2017). The latter in particular may be only one of many examples of eukaryotic, fungal, or viral members of the gut microbiome not amenable to most current high-throughput experimental or analytical approaches, but with unexpected and potentially key positive roles in dietary metabolism or cardiometabolic health.

Likewise, these new, highly specific contributions of the gut microbiome to human dietary responses may help to explain some of the heterogeneity and apparent contradictions seen among previous population studies (Sze & Schloss, MBio 7, 2016; Zeevi et al., Cell 163, 1079-1094, 2015; McDonald et al., mSystems 3, 2018; Kurilshikov et al., Circ. Res. 124, 1808-1820, 2019). First, diet-microbiome-blood marker associations were overall strongest with respect to circulating lipid levels (triglycerides, lipoproteins, etc.) relative to glycemic indices (e.g. blood glucose, insulin sensitivity). This may have both biochemical and clinical implications. It is possible that gut microbial metabolism contributes relatively more to circulating lipid levels than to carbohydrate derivatives, either directly or via mediating processes such as gastrointestinal or systemic bile acid signaling (Kurilshikov et al., Circ. Res. 124, 1808-1820, 2019; Ko et al., Nat. Rev. Gastroenterol. Hepatol., doi:10.1038/s41575-019-0250-7, 2020). Alternatively, host metabolism may play a greater role in circulating glucose and insulin levels relative to microbial bioactivity. The lipoprotein features most closely associated with the microbiome (such as L-HDL-L) are also more strongly associated with cardiovascular risk compared with typically measured lipids (e.g. TC, HDL-C, LDL-C), suggesting a closer look may be warranted at their utility as clinical biomarkers or as targets for beneficial gut microbiome manipulation.

Finally, an important conclusion of these results with respect to overall microbiome epidemiology is the limitation and coarseness of phenotypic associations achievable by using simple diversity or microbiome summary statistics. Even when a variety of significant species-specific dietary and molecular associations in the gut were identified, their effect sizes were often limited, likely reflecting both strain-specific functionality not assessed in these profiles (Pasolli et al., Cell 176, 649-662.e20, 2019; Truong et al., Genome Res. 27, 626-638, 2017; Scholz et al., Nat. Methods 13, 435-438, 2016; Quince et al., Nat. Biotechnol. 35, 833-844, 2017) and ecological signals among multiple interacting microbes as captured by the richer machine learning models (Pasolli et al., PLoS Comput. Biol. 12, e1004977, 2016). Similarly, with respect to host physiology, many postprandial responses relative to individual-specific fasting values (e.g., triglyceride levels, lipoproteins, insulin concentrations) were moderately more associated with the gut microbiome than the pre-existing fasting values themselves. This may speak to the interaction of both host metabolism and microbial metabolism impacting digestive and metabolic pathways, shaping long- and short-term diet-host effects on health and disease (Rowland et al., Eur. J. Nutr. 57, 1-24, 2018). Overall, this is the first study to identify a shared diet-metabolic-health microbial signature, segregating favorable and unfavorable taxa with multiple measures of both dietary intake and cardiometabolic health. The hope is that these initial PREDICT 1 results, targeted clinical and microbial follow-up based on them, and future iterations of the PREDICT study will aid as a resource both in utilization of the gut microbiome as a biomarker for cardiometabolic risk and in strategies for reshaping the microbiome to improve personalized dietary health.

TABLE 1
List of foods and their assigned food groups and health classification.

| Foods | Food_Groups | Classifications |
|---|---|---|
| APPLES | Fruits | Healthy |
| AVOCADO | Vegetables | Healthy |
| BACON | Meat | Less healthful animal foods |
| BANANAS | Fruits | Healthy |
| BEANS | Legumes | Healthy |
| BEANSPROUTS | Vegetables | Healthy |
| BEEF | Meat | Less healthful animal foods |
| BEER | | |
| BEETROOT | Vegetables | Healthy |
| BISCUITS_REDUCED_FAT | Sweets_and_desserts | Less Healthy |
| BOILED_POTATOES | Potatoes | Less Healthy |
| BROCCOLI | Vegetables | Healthy |
| BROWN_BREAD | Whole_grain | Healthy |
| BROWN_RICE | Whole_grain | Healthy |
| BURGER | Meat | Less healthful animal foods |
| BUTTER | Animal fats | Less healthful animal foods |
| BUTTER_REDUCED_FAT | Animal fats | Less healthful animal foods |
| CABBAGE | Vegetables | Healthy |
| CARROTS | Vegetables | Healthy |
| CAULIFLOWER | Vegetables | Healthy |
| CEREAL_BARS | Sweets_and_desserts | Less Healthy |
| CEREAL_HIGH_FIBRE | Whole_grain | Healthy |
| CEREAL_SUGAR_TOPPED | Sweets_and_desserts | Less Healthy |
| CHEESE | Dairy | Less healthful animal foods |
| CHEESE_REDUCED_FAT | Dairy | More healthful animal foods |
| CHICKEN | Meat | More healthful animal foods |
| CHIPS_ROAST_POTATOES | Potatoes | Less Healthy |
| CHOCOLATE_BARS | Sweets_and_desserts | Less Healthy |
| CHOCOLATE_BISCUIT | Sweets_and_desserts | Less Healthy |
| CHOCOLATE_DARK | Sweets_and_desserts | Less Healthy |
| CHOCOLATE_MILK_WHITE | Sweets_and_desserts | Less Healthy |
| COCOA | Sugar_sweetened_beverages | Less Healthy |
| COFFEE_WHITENER | Sugar_sweetened_beverages | Less Healthy |
| COLESLAW | Vegetables | Healthy |
| CORNED_BEEF | Meat | Less healthful animal foods |
| CORNFLAKES_RICE_KRISPIES | Refined_grains | Less Healthy |
| COTTAGE_CHEESE | Dairy | More healthful animal foods |
| CRACKERS | Refined_grains | Less Healthy |
| CRISPBREAD | Refined_grains | Less Healthy |
| CRISPS | Potatoes | Less Healthy |
| DAIRY_DESSERT | Dairy | Less healthful animal foods |
| DECAFF_COFFEE | Tea_and_coffee | Healthy |
| DOUBLE_CREAM | Dairy | Less healthful animal foods |
| DRIED_FRUIT | Fruits | Healthy |
| EGGS | Eggs | More healthful animal foods |
| FISH_FINGERS | Fish or seafood | Less healthful animal foods |
| FIZZY_DRINKS | Sugar_sweetened_beverages | Less Healthy |
| FRENCH | Vegetable_oils | Healthy |
| FRIED_FISH | Fish or seafood | Less healthful animal foods |
| FRUIT_JUICE | Fruit_juices | Less Healthy |
| FRUIT_SQUASH | Sugar_sweetened_beverages | Less Healthy |
| FRUIT_TEA | Tea_and_coffee | Healthy |
| FULLFAT_YOGURT | Dairy | More healthful animal foods |
| GARLIC | Vegetables | Healthy |
| GRAPEFRUIT | Fruits | Healthy |
| GRAPES | Fruits | Healthy |
| GREEN_BEANS | Vegetables | Healthy |
| GREEN_SALAD | Vegetables | Healthy |
| GREEN_TEA | Tea_and_coffee | Healthy |
| HAM | Meat | Less healthful animal foods |
| HARD_MARGARINE | | |
| HOMEBAKED_BUNS | Sweets_and_desserts | Less Healthy |
| HOMEBAKED_CAKE | Sweets_and_desserts | Less Healthy |
| HOMEBAKED_FRUIT_PIES | | Less Healthy |
| HOMEBAKED_SPONGE | Sweets_and_desserts | Less Healthy |
| HORLICKS | Sugar_sweetened_beverages | Less Healthy |
| HOT_CHOCOLATE_LOW_FAT | Sugar_sweetened_beverages | Less Healthy |
| ICE_CREAM | Dairy | Less healthful animal foods |
| INSTANT_COFFEE | Tea_and_coffee | Healthy |
| JAM | Sweets_and_desserts | Less Healthy |
| KETCHUP | Vegetables | Healthy |
| LAMB | Meat | Less healthful animal foods |
| LASAGNE | Meat | Less healthful animal foods |
| LEEKS | Vegetables | Healthy |
| LENTILS | Legumes | Healthy |
| LIVER | Meat | Less healthful animal foods |
| LOWCAL_FIZZY_DRINKS | Sugar_sweetened_beverages | Less Healthy |
| LOWCAL_SALAD_CREAM | Miscellaneous animal-based | Less healthful animal foods |
| | foods | |
| LOWFAT_SPREAD | | |
| LOWFAT_YOGURT | Dairy | More healthful animal foods |
| MARMITE | Vegetables | Healthy |
| MARROW | Vegetables | Healthy |
| MEAT_SOUP | Meat | Less healthful animal foods |
| MELONS | Fruits | Healthy |
| MILK_PUDDINGS | Dairy | Less healthful animal foods |
| MUESLI | Refined_grains | Less Healthy |
| MUSHROOMS | Vegetables | Healthy |
| NAAN_POP_TORTILLAS | Refined_grains | Less Healthy |
| NUTS_SALTED | Nuts | Healthy |
| NUTS_UNSALTED | Nuts | Healthy |
| OILY_FISH | Fish or seafood | More healthful animal foods |
| ONIONS | Vegetables | Healthy |
| ORANGES | Fruits | Healthy |
| OTHER_DRESSING | Vegetable_oils | Healthy |
| OTHER_MARGARINE | | |
| PARSNIPS | Vegetables | Healthy |
| PEACHES | Fruits | Healthy |
| PEANUT_BUTTER | Nuts | Healthy |
| PEARS | Fruits | Healthy |
| PEAS | Vegetables | Healthy |
| PEPPERS | Vegetables | Healthy |
| PICKLES | Vegetables | Healthy |
| PIZZA | Miscellaneous animal-based | Less healthful animal foods |
| | foods | |
| PLAIN_BISCUIT | Sweets_and_desserts | Less Healthy |
| POLYUNSATURATED_MARGARINE | | |
| PORK | Meat | Less healthful animal foods |
| PORRIDGE | Whole_grain | Healthy |
| PORT | | |
| POTATO_SALAD | Potatoes | Less Healthy |
| QUICHE | Miscellaneous animal-based | Less healthful animal foods |
| | foods | |
| READYMADE_BUNS | Sweets_and_desserts | Less Healthy |
| READYMADE_CAKE | Sweets_and_desserts | Less Healthy |
| READYMADE_FRUIT_PIES | Sweets_and_desserts | Less Healthy |
| READYMADE_SPONGE | Sweets_and_desserts | Less Healthy |
| ROE | Fish or seafood | More healthful animal foods |
| SALAD_CREAM | Miscellaneous animal-based | Less healthful animal foods |
| | foods | |
| SAUCES | Vegetables | Healthy |
| SAUSAGES | Meat | Less healthful animal foods |
| SAVOURY_PIES | Miscellaneous animal-based | Less healthful animal foods |
| | foods | |
| SEEDS | Nuts | Healthy |
| SHELLFISH | Fish or seafood | More healthful animal foods |
| SINGLE_CREAM | Dairy | Less healthful animal foods |
| SMOOTHIES | Fruit_juices | Less Healthy |
| SPINACH | Vegetables | Healthy |
| SPIRITS | | |
| SPREAD_CHOLESTEROL_REDUCING | | |
| SPREAD_OLIVE_OIL | Vegetable_oils | Healthy |
| SPROUTS | Vegetables | Healthy |
| STRAWBERRIES | Fruits | Healthy |
| SUGAR | Sweets_and_desserts | Less Healthy |
| SWEETCORN | Vegetables | Healthy |
| SWEETS | Sweets_and_desserts | Less Healthy |
| TEA | Tea_and_coffee | Healthy |
| TINNED_FRUIT | Fruits | Healthy |
| TOFU | Legumes | Healthy |
| TOMATOES | Vegetables | Healthy |
| VEGETABLE_SOUP | Vegetables | Healthy |
| VERY_LOWFAT_SPREAD | | |
| WATERCRESS | Vegetables | Healthy |
| WHITE_BREAD | Refined_grains | Less Healthy |
| WHITE_FISH | Fish or seafood | More healthful animal foods |
| WHITE_PASTA | Refined_grains | Less Healthy |
| WHITE_RICE | Refined_grains | Less Healthy |
| WHOLEMEAL_BREAD | Whole_grain | Healthy |
| WHOLEMEAL_PASTA | Whole_grain | Healthy |
| WINE_RED | | |
| WINE_WHITE | | |

List of Nutrients.
Nutrients

| | |
|---|---|
| Alpha_carotene | Manganese |
| Beta_carotene | Monounsaturated_fatty_acids_MUFA_total |
| Calcium | Niacin |
| Carbohydrate_fructose | Nitrogen |
| Carbohydrate_galactose | Phosphorus |
| Carbohydrate_glucose | Polyunsaturated_fatty_acids_PUFA_total |
| Carbohydrate_lactose | Potassium |
| Carbohydrate_maltose | Protein |
| Carbohydrate_starch | Saturated_fatty_acids_SFA_total |
| Carbohydrate_sucrose | Selenium |
| Carbohydrate_sugars_total | Sodium |
| Carbohydrate_total | Total_folate |
| Carotene_total_carotene_equivalents | Vitamin_A_retinol |
| Chloride | Vitamin_A_retinol_equivalents |
| Cholesterol | Vitamin_B1_thiamin |
| Copper | Vitamin_B12_cobalamin |
| Englyst_Fibre_Non_Starch_ | Vitamin_B2_riboflavin |
| Polysaccharides_NSP | |
| Fat_total | Vitamin_B6_pyridoxine |
| Iodine | Vitamin_C_ascorbic_acid |
| Iron | Vitamin_D_ergocalciferol |
| Magnesium | Vitamin_E_alpha_tocopherol_equivalents |

List of Nutrients_% E
Nutrients (% E)

| | |
|---|---|
| Alpha_carotene_kcal | Manganese_kcal |
| Beta_carotene_kcal | Monounsaturated_fatty_acids_MUFA_total_kcal |
| Calcium_kcal | Niacin_kcal |
| Carbohydrate_fructose_kcal | Nitrogen_kcal |
| Carbohydrate_galactose_kcal | Phosphorus_kcal |
| Carbohydrate_glucose_kcal | Polyunsaturated_fatty_acids_PUFA_total_kcal |
| Carbohydrate_lactose_kcal | Potassium_kcal |
| Carbohydrate_maltose_kcal | Protein_kcal |
| Carbohydrate_starch_kcal | Saturated_fatty_acids_SFA_total_kcal |
| Carbohydrate_sucrose_kcal | Selenium_kcal |
| Carbohydrate_sugars_total_kcal | Sodium_kcal |
| Carbohydrate_total_kcal | Total_folate_kcal |
| Carotene_total_carotene_equivalents_kcal | Vitamin_A_retinol_equivalents_kcal |
| Chloride_kcal | Vitamin_A_retinol_kcal |
| Cholesterol_kcal | Vitamin_B1_thiamin_kcal |
| Copper_kcal | Vitamin_B12_cobalamin_kcal |
| Englyst_Fibre_Non_Starch_ | Vitamin_B2_riboflavin_kcal |
| Polysaccharides_NSP_kcal | |
| Fat_total_kcal | Vitamin_B6_pyridoxine_kcal |
| Iodine_kcal | Vitamin_C_ascorbic_acid_kcal |
| Iron_kcal | Vitamin_D_ergocalciferol_kcal |
| Magnesium_kcal | Vitamin_E_alpha_tocopherol_equivalents_kcal |

TABLE 2

| | | Energy | Carbohydrate | Sugars g | Fat g | Protein | Fiber |
|---|---|---|---|---|---|---|---|
| Meal | Description | kcal (kJ) | g (% E) | (% E) | (% E) | g (% E) | g |
| 1 | Metabolic | 890 (3725) | 85.5 (38.4%) | 54.5 | 52.7 | 16.1 | 2.3 |
| | Challenge | | | (24.5%) | (53.3%) | (7.2%) | |
| | Meal muffins + | | | | | | |
| | milkshake 1 | | | | | | |
| | Medium Fat & | 502 (2101) | 71.2(56.7%) | 40.9 | 22.2 | 9.6 | 2.2 |
| 2 | Carbohydrate | | | (32.6%) | (39.8%) | (7.6%) | |
| | muffins 1'2 | | | | | | |
| 3 | High Fat 1 | 500 (2092) | 40.5 (32.4%) | 20.3 | 34.8 | 9.0 | 1.1 |
| | muffins 1 | | | (16.2%) | (62.6%) | (7.2%) | |
| 4 | High | 504 (2109) | 95.4 (75.7%) | 54.2 | 9.0 | 9.4 | 1.7 |
| | Carbohydrate | | | (43.0%) | (16.1%) | (7.5%) | |
| | muffins 1 | | | | | | |
| 5 | OGTT drink 1 | 300 (1255) | 75.0 (100.0%) | 75.0 | 0.0 | 0.0 | 0 |
| | | | | (100.0%) | (0.0%) | (0.0%) | |
| 6 | High Fiber | 533 (2230) | 95.1 (71.4%) | 53.0 | 12.0 | 10.5 | 17 |
| | muffins and | | | (39.8%) | (20.3%) | (7.9%) | |
| | fiber bars 1 | | | | | | |
| 7 | High Fat 2 | 501 (2095) | 28.2 (22.5%) | 13.0 | 39.3 | 8.1 | 0.8 |
| | muffins 1 | | | (10.4%) | (70.6%) | (6.5%) | |
| 8 | High Protein | 502 (2100) | 70.8 (56.4%) | 50.3 | 5.7 | 40.8 | 2 |
| | muffins and | | | (40.1%) | (10.2%) | (32.5%) | |
| | protein shake 1 | | | | | | |

E: energy intake; OGTT: oral glucose tolerance test; 1Test meal consumed for breakfast; 2Test
meal consumed for lunch.

TABLE 3
Plant-based Diet Index, Healthy Food Diversity index, Food group classifications, animal groups,
Alternate Mediterranean score, and Healthy Eating Index (HEI) descriptions.
Plant-based Diet Index (PDI).

| | | |
|---|---|---|
| PDI Food | | |
| Groups | UK_FETA | US FFQ |

Healthy

| | | |
|---|---|---|
| Whole grain | BROWN BREAD, BROWN RICE, | oatmeal, rye/pumpernickel bread, |
| | CEREAL HIGH FIBER, PORRIDGE, | dark wholegrain bread, brown rice, |
| | WHOLEMEAL BREAD, | oat bran, bran |
| | WHOLEMEAL PASTA | |
| Fruits | BANANAS, DRIED FRUIT, | Raisins or grapes, prunes or dried |
| | GRAPEFRUIT, GRAPES, MELONS, | plums, prune juice, bananas, |
| | ORANGES, PEACHES, PEARS, | cantaloupe, fresh apples or pears, |
| | STRAWBERRIES, TINNED FRUIT, | oranges, grapefruit or grapefruit juice, |
| | APPLES | strawberries, blueberries, peaches, |
| | | apricots |
| Vegetables | AVOCADO, BEANSPROUTS, | avocado, tomatoes, tomato sauce, |
| | BEETROOT, BROCCOLI, | salsa, string beans, peas, broccoli, |
| | CABBAGE, CARROTS, | cauliflower, raw cabbage, brussel |
| | CAULIFLOWER, COLESLAW, | sprouts, raw carrots, cooked carrots, |
| | GARLIC, GREEN BEANS, GREEN | corn, mixed vegetables, yams or |
| | SALAD, LEEKS, MARROW, | sweet potatoes, orange winter |
| | MUSHROOMS, ONIONS, | squash, eggplant, kale, cooked |
| | PARSNIPS, PEAS, PEPPERS, | spinach, raw spinach, iceberg lettuce, |
| | SPINACH, SPROUTS, | romaine or leaf lettuce, celery, green |
| | SWEETCORN, TOMATOES, | or red peppers, onions as a garnish, |
| | VEGETABLE SOUP, | onions cooked, tomato ketchup |
| | WATERCRESS, MARMITE, | |
| | KETCHUP, PICKLES, SAUCES | |
| Nuts | NUTS SALTED, NUTS UNSALTED, | peanut butter, walnuts, peanuts, |
| | PEANUT BUTTER, SEEDS | other nuts |
| Legumes | TOFU, LENTILS, BEANS | beans or lentils, tofu or soybeans, |
| | | Soy milk |
| Vegetable | FRENCH, OTHER DRESSING, | olive oil, salad dressing |
| oils | SPREAD OLIVE OIL | |
| Tea and | DECAFF COFFEE, FRUIT TEA, | water, decaffeinated coffee, coffee, |
| coffee | GREEN TEA, INSTANT COFFEE | coffee drink, herbal tea, tea |

Less Healthy

| | | |
|---|---|---|
| Fruit juices | FRUIT JUICE, SMOOTHIES | apple juice or cider, orange juice |
| | | (calcium fortified), orange juice, |
| | | tomato juice or V-8 |
| Refined | MUESLI, NAAN POP TORTILLAS, | breakfast cereal, other cooked |
| grains | WHITE BREAD, WHITE PASTA, | cereal, white bread, crackers, english |
| | WHITE RICE, CRISPBREAD, | muffins/rolls, muffins or biscuits, |
| | CORNFLAKES RICE KRISPIES, | pancakes, white rice, tortillas, pasta |
| | CRACKERS | |
| Potatoes | BOILED POTATOES, CHIPS ROAST | french fries, boiled/mashed potatoes, |
| | POTATOES, POTATO SALAD, | potato/corn chips |
| | CRISPS | |
| Sugar | FIZZY DRINKS, FRUIT SQUASH, | low calorie beverage, low calorie |
| sweetened | LOWCAL FIZZY DRINKS, COCOA, | beverage with caffeine, coke, other |
| beverages | COFFEE WHITENER, HORLICKS, | carbonated beverage, fruit punch |
| | HOT CHOCOLATE LOW FAT | |
| Sweets and | BISCUITS REDUCED FAT, CEREAL | chocolate bar, dark chocolate bar, |
| desserts | BARS, CEREAL SUGAR TOPPED, | candy bar, candy without chocolate, |
| | CHOCOLATE BARS, CHOCOLATE | cookies, brownies, dougnuts, cake, |
| | BISCUIT, CHOCOLATE DARK, | low fat cake, pie, jam, regular |
| | CHOCOLATE MILK WHITE, | popcorn, popcorn, sweet roll, low fat |
| | HOMEBAKED CAKE, HOMEBAKED | sweet roll, breakfast bar, energy bar, |
| | SPONGE, JAM, PLAIN BISCUIT, | low carb bar, pretzels, splenda, other |
| | READYMADE BUNS, READYMADE | artificial sweetener |
| | CAKE, READYMADE FRUIT PIES, | |
| | READYMADE SPONGE, | |
| | HOMEBAKED BUNS, SUGAR, | |
| | SWEETS | |

Animal Food Groups

| | | |
|---|---|---|
| Animal fat | BUTTER, BUTTER REDUCED FAT | butter |
| Dairy | CHEESE REDUCED FAT, COTTAGE | skimmed milk, 1-2% milk, cottage |
| | CHEESE, LOWFAT YOGURT, | ricotta cheese, Whole milk, cream, |
| | CHEESE, DAIRY DESSERT, | non-dairy coffee whitener, frozen |
| | DOUBLE CREAM, FULLFAT | yogurt, ice-cream, plain yogurt, |
| | YOGURT, ICE CREAM, SINGLE | yogurt, cream cheese, other cheese |
| | CREAM, MILK PUDDINGS | |
| Egg | EGGS | eggs, omega eggs |
| Fish or | OILY FISH, ROE, SHELLFISH, | canned tuna, kids breaded fish |
| seafood | WHITE FISH, FISH FINGERS, FRIED | pieces, shrimp, dark meat fish, other |
| | FISH | fish |
| Meat | CHICKEN, BEEF, BURGER, | chicken/turkey sandwich, chicken or |
| | CORNED BEEF, HAM, LAMB, | turkey (with skin), chicken or turkey |
| | LASAGNA, LIVER, MEAT SOUP, | (without skin), chicken liver, beef or |
| | PORK, SAUSAGES, BACON | pork hot dogs, bologna, other |
| | | processed meats, extra lean |
| | | hamburgers, hamburgers, |
| | | beef/pork/lamb sandwich, pork as |
| | | main dish, beef as main dish, |
| | | chowder or creamy soup, beef liver, |
| | | bacon |
| Micellaneous | LOWCAL SALAD CREAM, SALAD | Pizza, diet mayonnaise, mayonnaise |
| animal based | CREAM, PIZZA, QUICHE, SAVOURY | |
| foods | PIES | |

Co-variate

| Margarine | HARD MARGARINE, LOWFAT | margarine | |
|---|---|---|---|
| | SPREAD, OTHER MARGARINE, | | |
| | POLYUNSATURATED MARGARINE, | | |
| | SPREAD CHOLESTEROL | | |
| | REDUCING, VERY LOWFAT | | |
| | SPREAD | | |
| Alcohol | BEER, PORT, SPIRITS, WINE RED, | beer, light beer, red wine, white wine, | |
| | WINE WHITE | liquor | |
| PDI Food Groups (18) | PDI | hPDI | uPDI |

Healthy

| | | | |
|---|---|---|---|
| Whole_grain | + | + | − |
| Fruits | + | + | − |
| Vegetables | + | + | − |
| Nuts | + | + | − |
| Legumes | + | + | − |
| Vegetable_oils | + | + | − |
| Tea_and_coffee | + | + | − |

Less Healthy

| | | | |
|---|---|---|---|
| Fruit_juices | + | − | + |
| Refined_grains | + | − | + |
| Potatoes | + | − | + |
| Sugar_sweetened_beverages | + | − | + |
| Sweets_and_desserts | + | − | + |

Animal Food Groups

| | | | |
|---|---|---|---|
| Animal_fat | − | − | − |
| Dairy | − | − | − |
| Egg | − | − | − |
| Fish_or seafood | − | − | − |
| Meat | − | − | − |
| Micellaneous_animal_based_foods | − | − | − |

Healthy Food Diversity Index (HFDI).

| ORIGINAL_HFDI | UK FETA | US FFQ |
|---|---|---|
| Vegetables, | APPLES, AVOCADO, BANANAS, | Raisins or grapes, prunes or dried |
| fruits, leaf | BEANS, BEANSPROUTS, | plums, prune juice, bananas, |
| salads, juices | BEETROOT, BROCCOLI, | cantaloupe, avocado, fresh apples |
| | CABBAGE, CARROTS, | or pears, apple juice or cider, |
| | CAULIFLOWER, COLESLAW, | oranges, orange juice (calcium |
| | DRIED FRUIT, FRUIT JUICE, | fortified), orange juice, grapefruit or |
| | GARLIC, GRAPEFRUIT, GRAPES, | grapefruit juice, strawberries, |
| | GREEN BEANS, GREEN SALAD, | blueberries, peaches, apricots, |
| | LEEKS, LENTILS, MARROW, | tomatoes, tomato juice or V-8, |
| | MELONS, MUSHROOMS, NUTS | tomato sauce, salsa, string beans, |
| | SALTED, NUTS UNSALTED, | beans or lentils, tofu or soybeans, |
| | ONIONS, ORANGES, PARSNIPS, | peas, broccoli, cauliflower, raw |
| | PEACHES, PEANUT BUTTER, | cabbage, Brussel sprouts, raw |
| | PEARS, PEAS, PEPPERS, | carrots, cooked carrots, corn, mixed |
| | SEEDS, SMOOTHIES, SPINACH, | vegetables, yams or sweet |
| | SPROUTS, STRAWBERRIES, | potatoes, orange winter squash, |
| | SWEETCORN, TINNED FRUIT, | eggplant, kale, cooked spinach, raw |
| | TOFU, TOMATOES, VEGETABLE | spinach, iceberg lettuce, romaine or |
| | SOUP, WATERCRESS, | leaf lettuce, celery, green or red |
| | | peppers, onions as a garnish, |
| | | onions cooked, peanut butter, |
| | | walnuts, peanuts, other nuts, |
| Wholemeal | BROWN BREAD, BROWN RICE, | oatmeal, rye/pumpernickel bread, |
| products, Paddy | CEREAL HIGH FIBRE, | dark wholegrain bread, brown rice, |
| | PORRIDGE, WHOLEMEAL | oat bran, bran, |
| | BREAD, WHOLEMEAL PASTA | |
| Potatoes | BOILED POTATOES, CHIPS | French fries, boiled/mashed |
| | ROAST POTATOES, POTATO | potatoes, |
| | SALAD | |
| White-meal | MUESLI, NAAN POP TORTILLAS, | breakfast cereal, other cooked |
| products, peeled | WHITE BREAD, WHITE PASTA, | cereal, white bread, crackers, |
| rice | WHITE RICE, CRISPBREAD, | English muffins/rolls, muffins or |
| | CORNFLAKES RICE KRISPIES | biscuits, pancakes, white rice, |
| | | tortillas, pasta |
| Snacks and | BISCUITS REDUCED FAT, | potato/corn chips, pizza, low calorie |
| sweets-sugar, | CEREAL BARS, CEREAL SUGAR | beverage, low calorie beverage with |
| cakes, sweets, | TOPPED, CHOCOLATE BARS, | caffeine, coke, other carbonated |
| snack, potato | CHOCOLATE BISCUIT, | beverage, fruit punch, chocolate |
| chips, fruit juice | CHOCOLATE DARK, | bar, dark chocolate bar, candy bar, |
| spritz etc | CHOCOLATE MILK WHITE, | candy without chocolate, cookies, |
| | CRACKERS, MARMITE, CRISPS, | brownies, dougnuts, cake, low fat |
| | FIZZY DRINKS, FRUIT SQUASH, | cake, pie, jam, regular popcorn, |
| | HOMEBAKED CAKE, | popcorn, sweet roll, low fat sweet |
| | HOMEBAKED SPONGE, JAM, | roll, breakfast bar, energy bar, low |
| | KETCHUP, LOWCAL FIZZY | carb bar, pretzels, tomato ketchup, |
| | DRINKS, PICKLES, PIZZA, PLAIN | splenda, other artificial sweetener |
| | BISCUIT, QUICHE, READYMADE | |
| | BUNS, READYMADE CAKE, | |
| | READYMADE FRUIT PIES, | |
| | READYMADE SPONGE, SAUCES, | |
| | SAVOURY PIES, SUGAR, | |
| | SWEETS, COCOA, COFFEE | |
| | WHITENER, HORLICKS, | |
| | HOMEBAKED BUNS | |
| Fish, low-fat | CHICKEN, OILY FISH, ROE, | canned tuna, chicken/turkey |
| meat, low-fat | SHELLFISH, WHITE FISH | sandwich, chicken or turkey (with |
| meat products | | skin), chicken or turkey (without |
| | | skin), kids breaded fish pieces, |
| | | shrimp, dark meat fish, other fish, |
| | | chicken liver |
| Low-fat milk, low-fat | CHEESE REDUCED FAT, | Skimmed milk, 1-2% milk, Soy milk, |
| dairy products | COTTAGE CHEESE, LOWFAT | cottage ricotta cheese |
| | YOGURT | |
| Milk, dairy | CHEESE, DAIRY DESSERT, | Whole milk, cream, non-dairy coffee |
| products | DOUBLE CREAM, FULLFAT | whitener, frozen yogurt, ice-cream, |
| | YOGURT, ICE CREAM, SINGLE | plain yogurt, yogurt, cream cheese, |
| | CREAM, MILK PUDDINGS | other cheese |
| Meat products, | BEEF, BURGER, CORNED BEEF, | eggs, omega eggs, beef or pork hot |
| sausages, eggs | EGGS, FISH FINGERS, FRIED | dogs, bologna, other processed |
| | FISH, HAM, LAMB, LASAGNA, | meats, extra lean hamburgers, |
| | LIVER, MEAT SOUP, PORK, | hamburgers, beef/pork/lamb |
| | SAUSAGES | sandwich, pork as main dish, beef |
| | | as main dish, chowder or creamy |
| | | soup, beef liver |
| Bacon | BACON | bacon |
| Oilseed rape, | NA | |
| walnut oil | | |
| Wheat germ oil, | NA | |
| soybean oil | | |
| Corn oil, | FRENCH, LOWCAL SALAD | diet mayonnaise, mayonnaise, |
| sunflower oil | CREAM, OTHER DRESSING, | |
| | SALAD CREAM | |
| Margarines, | BUTTER, BUTTER REDUCED | margarine, butter |
| butter | FAT, HARD MARGARINE, | |
| | LOWFAT SPREAD, OTHER | |
| | MARGARINE, | |
| | POLYUNSATURATED | |
| | MARGARINE, SPREAD | |
| | CHOLESTEROL REDUCING, | |
| | SPREAD OLIVE OIL, VERY | |
| | LOWFAT SPREAD, | |
| Lard, vegetable | | olive oil, salad dressing |
| fat | | |
| Not included: | BEER, DECAFF COFFEE, FRUIT | beer, light beer, red wine, white |
| | TEA, GREEN TEA, INSTANT | wine, liqueurs, water, decaffeinated |
| | COFFEE, PORT, SPIRITS, TEA, | coffee, coffee, coffee drink, herbal |
| | WINE RED, WINE WHITE | tea, tea |

Food Groups Classifications.

| | |
|---|---|
| Food Groups | UK FFQ |

Healthy

| | |
|---|---|
| Whole grain | BROWN BREAD, BROWN RICE, CEREAL HIGH FIBRE, PORRIDGE, |
| | WHOLEMEAL BREAD, WHOLEMEAL PASTA |
| Fruits | BANANAS, DRIED FRUIT, GRAPEFRUIT, GRAPES, MELONS, |
| | ORANGES, PEACHES, PEARS, STRAWBERRIES, TINNED FRUIT, |
| | APPLES |
| Vegetables | AVOCADO, BEANSPROUTS, BEETROOT, BROCCOLI, CABBAGE, |
| | CARROTS, CAULIFLOWER, COLESLAW, GARLIC, GREEN BEANS, |
| | GREEN SALAD, LEEKS, MARROW, MUSHROOMS, ONIONS, PARSNIPS, |
| | PEAS, PEPPERS, SPINACH, SPROUTS, SWEETCORN, TOMATOES, |
| | VEGETABLE SOUP, WATERCRESS, MARMITE, KETCHUP, PICKLES, |
| | SAUCES |
| Nuts | NUTS SALTED, NUTS UNSALTED, PEANUT BUTTER, SEEDS |
| Legumes | TOFU, LENTILS, BEANS |
| Vegetable oils | FRENCH, OTHER DRESSING, SPREAD OLIVE OIL |
| Tea and coffee | DECAFF COFFEE, FRUIT TEA, GREEN TEA, INSTANT COFFEE |

Less Healthy

| | |
|---|---|
| Fruit juices | FRUIT JUICE, SMOOTHIES |
| Refined grains | MUESLI, NAAN POP TORTILLAS, WHITE BREAD, WHITE PASTA, WHITE |
| | RICE, CRISPBREAD, CORNFLAKES RICE KRISPIES, CRACKERS |
| Potatoes | BOILED POTATOES, CHIPS ROAST POTATOES, POTATO SALAD, |
| | CRISPS |
| Sugar | FIZZY DRINKS, FRUIT SQUASH, LOWCAL FIZZY DRINKS, COCOA, |
| sweetened | COFFEE WHITENER, HORLICKS, HOT CHOCOLATE LOW FAT |
| beverages | |
| Sweets and | BISCUITS REDUCED FAT, CEREAL BARS, CEREAL SUGAR TOPPED, |
| desserts | CHOCOLATE BARS, CHOCOLATE BISCUIT, CHOCOLATE DARK, |
| | CHOCOLATE MILK WHITE, HOMEBAKED CAKE, HOMEBAKED |
| | SPONGE, JAM, PLAIN BISCUIT, READYMADE BUNS, READYMADE |
| | CAKE, READYMADE FRUIT PIES, READYMADE SPONGE, HOMEBAKED |
| | BUNS, SUGAR, SWEETS |

More healthful animal foods

| | |
|---|---|
| Dairy | CHEESE REDUCED FAT, COTTAGE CHEESE, LOWFAT YOGURT, |
| | FULLFAT YOGURT |
| Meat | CHICKEN |
| Eggs | EGGS |
| Fish or seafood | OILY FISH, ROE, SHELLFISH, WHITE FISH |
| Less healthful animal foods | |
| Animal fats | BUTTER, BUTTER REDUCED FAT |
| Meat | BEEF, BURGER, CORNED BEEF, HAM, LAMB, LASAGNA, LIVER, MEAT |
| | SOUP, PORK, SAUSAGES, BACON |
| Dairy | CHEESE, DAIRY DESSERT, DOUBLE CREAM, ICE CREAM, SINGLE |
| | CREAM, MILK PUDDINGS |
| Miscellaneous | LOWCAL SALAD CREAM, SALAD CREAM, PIZZA, QUICHE, SAVOURY |
| animal-based | PIES |
| foods | |
| Fish or seafood | FISH FINGERS, FRIED FISH |

Animal groups.

| | | |
|---|---|---|
| Variable | UK FFQ | USA FFQ |

More healthful animal foods

| | | |
|---|---|---|
| Dairy | CHEESE REDUCED FAT, | skimmed milk, 2% milk, |
| | COTTAGE CHEESE, | cottage cheese, full fat milk, |
| | LOWFAT YOGURT, | frozen yogurt, plain yogurt, |
| | FULLFAT YOGURT | yogurt |
| Meat | CHICKEN | chicken sandwich, chicken |
| | | with skin, chicken without |
| | | skin, chicken liver |
| Eggs | EGGS | eggs, eggs with omega |
| Fish or seafood | OILY FISH, ROE, | tuna, cooked shrimp, dark |
| | SHELLFISH, WHITE FISH | fish, other fish |

Less healthful animal foods

| | | |
|---|---|---|
| Animal fats | BUTTER, BUTTER | butter |
| | REDUCED FAT | |
| Meat | BEEF, BURGER, CORNED | hotdogs, chicken hot dog, |
| | BEEF, HAM, LAMB, | bologna, processed meat, |
| | LASAGNA, LIVER, MEAT | extra lean hamburger, |
| | SOUP, PORK, SAUSAGES, | hamburger, ham sandwich, |
| | BACON | pork, beef, creamy soup or |
| | | chowder, liver, bacon |
| Dairy | CHEESE, DAIRY | cream, coffee whitener, ice- |
| | DESSERT, DOUBLE | cream, cheese, other cheese, |
| | CREAM, ICE CREAM, | cream cheese cream |
| | SINGLE CREAM, MILK | |
| | PUDDINGS | |
| Miscellaneous animal-based | LOWCAL SALAD CREAM, | pizza, diet mayonnaise, |
| foods | SALAD CREAM, PIZZA, | mayonnaise |
| | QUICHE, SAVOURY PIES | |
| Fish or seafood | FISH FINGERS, FRIED | kids breaded fish fingers |
| | FISH | |

aMED

| AMED | UK_FETA | US FFQ |
|---|---|---|
| vegetables | AVOCADO, BEETROOT, | avocado, tomatoes, st.beans, broc, |
| | BEANSPROUTS, BROCCOLI, | caul, cabb, brusl, carrot.r, carrot.c, |
| | SPROUTS, CABBAGE, CARROTS, | corn, mix.veg, kale, spin.ckd, |
| | CAULIFLOWER, COLESLAW, | spin.raw, ice.let, rom.let, celery, |
| | GARLIC, GREEN SALAD, LEEKS, | peppers, onions, onions1, swt.pot, |
| | MARROW, MUSHROOMS, ONIONS, | yel.sqs, zuke |
| | PARSNIPS, SPINACH, PEPPERS, | |
| | SWEETCORN, WATERCRESS, | |
| | TOMATOES, VEGETABLE SOUP | |
| fruit | APPLES, BANANAS, DRIED FRUIT, | raisgrp, prun, ban, cant, apple, orang, |
| | GRAPEFRUIT, GRAPES, MELON, | gftrt, peaches, apricot, straw, blu, |
| | ORANGES, PEACHES, PEARS, | tom.j |
| | TINNED FRUIT, FRUIT JUICE | |
| wholegrain | BROWN RICE, BROWN BREAD, | oatmeal.bran, ckd.cer, rye.br, dk.br, |
| cereal | CEREAL HIGH FIBRE, | br.rice, oat.bran, bran, cold.cereal |
| | CRISPBREAD, MUESLI, PORRIDGE, | |
| | WHOLEMEAL BREAD, | |
| | WHOLEMEAL PASTA | |
| nuts | NUTS SALTED, NUTS UNSALTED, | p.bu, nuts, walnuts, oth.nuts |
| | PEANUT BUTTER | |
| meat | BACON, BEEF, BURGER, CORNED | Bacon, pork, beef02, sand.bf.ham, |
| | BEEF, HAM, LAMB, LASAGNA, | liver, chix.liver, hotdog, chix.dog, |
| | LIVER, MEAT SOUP, PORK, | bologna, proc.mts, xtrlean.hamburger, |
| | SAUSAGES, SAVOURY PIES | hamb |
| legumes | BEANS, LENTILS, GREEN BEANS, | beans, peas |
| | PEAS | |
| fish | FISH FINGERS, ROE, FRIED FISH, | Tuna, fr.fish.kids, shrimp.ckd, dk.fish, |
| | OILY FISH, SHELLFISH, WHITE | oth.fish |
| | FISH | |
| fatty acids | MUFA/SFA | MUFA/SFA |
| alcohol | alcohol | beer, liq, Spirits, r.wine, w.wine |

HEI.

| HEI | UK_FFQ | US_FFQ |
|---|---|---|
| Whole fruit | APPLES, BANANAS, DRIED FRUIT, | raisgrp, prun, ban, cant, apple, orang, |
| | GRAPEFRUIT, GRAPES, MELON, | gftrt, peaches, apricot, straw, blu, tom.j |
| | ORANGES, PEACHES, PEARS, | |
| | TINNED FRUIT | |
| Total fruit | APPLES, BANANAS, DRIED FRUIT, | prun.j, a.j, o.j.calc, o.j, oth.f.j, raisgrp, |
| | GRAPEFRUIT, GRAPES, MELON, | prun, ban, cant, apple, orang, gftrt, |
| | ORANGES, PEACHES, PEARS, | peaches, apricot, straw, blu, tom.j |
| | TINNED FRUIT, FRUIT JUICE, | |
| | SMOOTHIES | |
| Total | AVOCADO, BEETROOT, BROCCOLI, | avocado, tomatoes, broc, caul, cabb, |
| vegetables | SPROUTS, CABBAGE, CARROTS, | brusl, carrot.r, carrot.c, corn, mix.veg, |
| | CAULIFLOWER, COLESLAW, GARLIC, | kale, spin.ckd, spin.raw, ice.let, rom.let, |
| | GREEN SALAD, LEEKS, MARROW, | celery, peppers, onions, onions1, |
| | MUSHROOMS, ONIONS, SPINACH, | swt.pot, yel.sqs, zuke |
| | BROCCOLI, GREEN SALAD, | |
| | WATERCRESS | |
| Greens | BEANS, LENTILS, GREEN BEANS, | beans, peas, st.beans |
| and beans | PEAS, BEANSPROUTS | |
| Whole | BROWN RICE, BROWN BREAD, | oatmeal.bran, ckd.cer, rye.br, dk.br, |
| grains | CEREAL HIGH FIBRE, CRISPBREAD, | br.rice, oat.bran, bran, cold.cereal |
| | MUESLI, PORRIDGE, WHOLEMEAL | |
| | BREAD, WHOLEMEAL PASTA | |
| Dairy | SINGLE CREAM, DOUBLE CREAM, | milk, cream, cot.ch, yog.plain, yog, |
| | LOWFAT YOGURT, FULLFAT | cr.ch, ch.reg,skim.kids, milk2, ch.lofat, |
| | YOGURT, DAIRY DESSERT, CHEESE, | ch.nofat, bu, soymilk.fort, |
| | CHEESE REDUCED FAT, COTTAGE | ice.cr,margarine, cof.wht |
| | CHEESE, BUTTER BUTTER | |
| | REDUCED FAT, ICE CREAM, MILK | |
| | FREQUENCY, HARD MARGARINE, | |
| | POLYUNSATURATED MARGARINE, | |
| | SPREAD OLIVE OIL, SPREAD | |
| | CHOLESTEROL REDUCING, LOWFAT | |
| | SPREAD, VERY LOWFAT SPREAD, | |
| | COFFEE WHITENER, | |
| Total | BEANS, LENTILS, GREEN BEANS, | beans, peas, st.beans, chix.sk, chix.no, |
| protein | PEAS, BEANSPROUTS, EGGS, | chix.sand, eggs, chix.dog, bacon, pork, |
| foods | BACON, BEEF, BURGER, CHICKEN, | beef02, sand.bf.ham, liver, chix.liver, |
| | CORNED BEEF, HAM, LAMB, | hotdog, proc.mts,xtrlean.hamburg, |
| | LASAGNA, LIVER, MEAT SOUP, | hamb, tuna, fr.fish.kids, shrimp.ckd, |
| | PORK, SAUSAGES, SAVOURY PIES, | dk.fish, oth.fish, tofu, p.bu,nuts, walnuts, |
| | TOFU, SEEDS, NUTS SALTED, NUTS | oth.nuts |
| | UNSALTED, PEANUT BUTTER, FISH | |
| | FINGERS, ROE, FRIED FISH, OILY | |
| | FISH, SHELLFISH, WHITE FISH | |
| Seafood | TOFU, SEEDS, NUTS SALTED, NUTS | tuna, fr.fish.kids, shrimp.ckd, dk.fish, |
| and plant | UNSALTED, PEANUT BUTTER, FISH | oth.fish, tofu, p.bu,nuts, walnuts, |
| protein | FINGERS, ROE, FRIED FISH, OILY | oth.nuts |
| | FISH, SHELLFISH, WHITE FISH, | |
| | BEANS, LENTILS, GREEN BEANS, | |
| | PEAS, BEANSPROUTS | |
| Refined | WHITE BREAD, NAAN POP | wh.br, eng.muff, muff, pancak, wh.rice, |
| grains | TORTILLAS, CEREAL SUGAR | pasta, tortillas, brkfast.bars, pretzel, |
| | TOPPED, CORNFLAKES RICE | s.roll.lf, s.roll.c, cold.cereal |
| | KRISPIES, WHITE RICE, WHITE | |
| | PASTA | |
| Empty | ALCOHOL, PLAIN BISCUIT, BISCUITS | beer, liq, Spirits, r.wine, w.wine, milk, |
| calories | REDUCED FAT, CEREAL BARS, | cream, cot.ch, yog.plain, yog, cr.ch, |
| | CHOCOLATE BISCUIT, HOMEBAKED | ch.reg,skim.kids, milk2, ch.lofat, |
| | CAKE, READYMADE CAKE, | ch.nofat, bu, soymilk.fort, |
| | HOMEBAKED BUNS, READYMADE | ice.cr, margarine, cof.wht, wh.br, |
| | BUNS, HOMEBAKED FRUIT PIES, | eng.muff, muff, pancak, wh.rice, pasta, |
| | READYMADE FRUIT PIES, | tortillas, brkfast.bars, pretzel, s.roll.lf, |
| | HOMEBAKED SPONGE, READYMADE | s.roll.c, cold.cereal, chix.sk, chix.no, |
| | SPONGE, MILK PUDDINGS, ICE | chix.sand, eggs, chix.dog, bacon, pork, |
| | CREAM, CHOCOLATE MILK WHITE, | beef02, sand.bf.ham, liver, chix.liver, |
| | CHOCOLATE DARK, CHOCOLATE | hotdog, proc.mts,xtrlean.hamburg, |
| | BARS, SWEETS, SUGAR, CRISPS, | hamb, coke, oth.carb, punch, |
| | CHIPS ROAST POTATOES, PIZZA, | crax, pizza, cake.other, pie.comm,jam, |
| | QUICHE, JAM, KETCHUP, | mayo,mayo.d, |
| | CRACKERS, SALAD CREAM, | donut, choc, choc.dark, candy,coox.nofat, |
| | FRENCH, BACON, BEEF, BURGER, | coox.other, brownie, cake.lofat |
| | CHICKEN, CORNED BEEF, HAM, | |
| | LAMB, LASAGNA, LIVER, MEAT | |
| | SOUP, PORK, SAUSAGES, SAVOURY | |
| | PIES, SINGLE CREAM, DOUBLE | |
| | CREAM, LOWFAT YOGURT, FULLFAT | |
| | YOGURT, DAIRY DESSERT, CHEESE, | |
| | CHEESE REDUCED FAT, COTTAGE | |
| | CHEESE, BUTTER, BUTTER | |
| | REDUCED FAT, ICE CREAM, MILK | |
| | FREQUENCY, HARD MARGARINE, | |
| | POLYUNSATURATED MARGARINE, | |
| | SPREAD OLIVE OIL, SPREAD | |
| | CHOLESTEROL REDUCING, LOWFAT | |
| | SPREAD, VERY LOWFAT SPREAD, | |
| | COFFEE WHITENER | |
| Fatty acids | MUFA + PUFA/SFA | MUFA + PUFA/SFA |
| Sodium | Na | Na |

TABLE 4
P-values from the Mann-Whitney U test between presence/absence of Prevotella
copri, Blastocystis spp., and P. copri and Blastocystis spp. (Part 1). Effect size measured as the
ratio of the medians for P. copri and Blastocystis spp. presence/absence (Part 2).
(Part 1). Mann-VVhitneyU p-values preslabs

| | | | | | | P. copri & |
|---|---|---|---|---|---|---|
| | | | P. copri & | P. copri & | P. copri & | Blastocystis |
| | | Blastocystis | Blastocystis | Blastocystis | Blastocystis | (Y/P. copri I |
| Metadata | P. copri (Y/N) | (Y/N) | (Y/N) | (Y/P. copri) | (Y/Blastocystis) | Blastocystis) |
| HFD | 0.091726094 | 0.000337458 | 0.001848168 | 0.088051794 | 0.503878583 | 0.097679684 |
| visceral_fat | 0.005252767 | 2.27361E-07 | 8.92493E-06 | 0.019542182 | 0.32254405 | 0.020649254 |
| meal_jj_ho | 0.00223167 | 0.018192048 | 0.00699127 | 0.435043388 | 0.356376201 | 0.252778882 |
| spital_me | | | | | | |
| al_glucose_ | | | | | | |
| 120_iauc | | | | | | |
| cpep_0 | 0.002010901 | 4.7433E-05 | 0.001330307 | 0.246215757 | 0.513491699 | 0.212805516 |
| cpep_120 | 3.67802E-05 | 6.0799E-06 | 0.000971431 | 0.43339854 | 0.660244902 | 0.404138722 |
| cpep_60_rise | 0.004132966 | 7.34282E-05 | 0.000715316 | 0.17174951 | 0.473355081 | 0.152222313 |
| cpep_max | 0.00049577 | 0.000843029 | 0.004716621 | 0.496224788 | 0.525200889 | 0.371671313 |
| cpep_max_rise | 0.00190668 | 0.003416532 | 0.011063646 | 0.544885206 | 0.553694359 | 0.416426614 |
| trig_0 | 0.003359053 | 2.1823E-05 | 0.000106956 | 0.086897409 | 0.388884053 | 0.075465828 |
| trig_360 | 0.00834 | 0.000966481 | 0.010152018 | 0.407822838 | 0.737300089 | 0.422220534 |
| trig_360_rise | 0.153305702 | 0.060179226 | 0.440459983 | 0.93844867 | 0.711779682 | 0.768739154 |
| ins_0 | 0.006407223 | 4.34475E-05 | 0.010540768 | 0.439018736 | 0.998865922 | 0.581043215 |
| ins_30 | 0.412682943 | 0.015715245 | 0.220910448 | 0.565583207 | 0.864385937 | 0.76384535 |
| ins_30_rise | 0.667577631 | 0.038635367 | 0.347640015 | 0.587889022 | 0.820863241 | 0.809336546 |
| ins_max | 0.03470942 | 0.00081691 | 0.055599689 | 0.607423321 | 0.940704979 | 0.750943819 |
| ins_max_rise | 0.073259368 | 0.001838292 | 0.087701959 | 0.629724241 | 0.901961882 | 0.792523043 |
| Effect size preslabs. | | | | | | |
| Table 4 (Part 2). | | | | | | |
| HFD | 1.055315 | 1.097057 | 1.103467 | 1.044939 | 1.020238 | 1.046259 |
| visceral_fat | 0.874983 | 0.778522 | 0.767372 | 0.865512 | 0.960629 | 0.887536 |
| meal_jj_ho | 0.795871 | 0.843095 | 0.774927 | 0.935169 | 0.910803 | 0.916414 |
| spital_me | | | | | | |
| al_glucose_ | | | | | | |
| 120_iauc | | | | | | |
| cpep_0 | 0.908257 | 0.904545 | 0.87037 | 0.949495 | 0.944724 | 0.94 |
| cpep_120 | 0.877083 | 0.851464 | 0.83049 | 0.925178 | 0.957002 | 0.929594 |
| cpep_60_rise | 0.882038 | 0.854139 | 0.868027 | 0.969605 | 0.981538 | 0.969605 |
| cpep_max | 0.912207 | 0.916526 | 0.923469 | 0.995417 | 0.99908 | 0.994505 |
| cpep_max_rise | 0.9282 | 0.91841 | 0.941799 | 0.997758 | 1.013667 | 1.001125 |
| trig_0 | 0.967742 | 0.859375 | 0.87234 | 0.911111 | 0.993939 | 0.931818 |
| trig_360 | 0.878049 | 0.838415 | 0.820988 | 0.923611 | 0.967273 | 0.93007 |
| trig_360_rise | 0.875 | 0.815385 | 0.857143 | 0.964286 | 1.018868 | 0.981818 |
| ins_0 | 0.860909 | 0.833935 | 0.836431 | 0.95037 | 0.974026 | 0.955414 |
| ins_30 | 0.971208 | 0.896846 | 0.98571 | 1.006557 | 1.058772 | 1.035633 |
| ins_30_rise | 0.999599 | 0.908967 | 0.984835 | 0.989376 | 1.058439 | 1.017839 |
| ins_max | 0.926363 | 0.886731 | 0.912628 | 0.972941 | 1.011214 | 0.980629 |
| ins_max_rise | 0.938621 | 0.90409 | 0.933357 | 0.986591 | 1.004019 | 1.000279 |

TABLE 5
Ranks and average ranks for determining the two sets of positive and negative bacterial species according
to their correlations with a balanced set of personal, habitual diet, fasting, and postprandial metadata.
Table 5 (Part 1A). Spearman's correlation.

| Profile | quicki_score | amed_score | HFD | hei_score | HDL_size_0 | PUFA_pct_0 | HDL_size_360 |
|---|---|---|---|---|---|---|---|
| Positive/Negative | Positive | Positive | Positive | Positive | Positive | Positive | Positive |
| Paraprevotella_xylaniphila | 0.1008979 | 0.0600029 | 0.0022568 | 0.0058104 | 0.082487 | 0.0784278 | 0.0721128 |
| Paraprevotella_clara | 0.0987415 | 0.0407581 | 0.0035137 | −0.005448 | 0.070762 | 0.0752283 | 0.0598959 |
| Bacteroides_massiliensis | 0.0976011 | 0.0760443 | 0.0651124 | 0.0487547 | 0.0283254 | 0.1038991 | 0.010796 |
| Prevotella_copri | 0.0936779 | 0.0460914 | 0.0311296 | 0.0530606 | 0.0632046 | 0.1527947 | 0.0696674 |
| Rothia_mucilaginosa | 0.092378 | 0.0452159 | −0.018813 | 0.0518546 | 0.0890357 | 0.032277 | 0.0756649 |
| Haemophilus_parainfluenzae | 0.092202 | 0.0801567 | 0.1682534 | 0.0850939 | 0.1170721 | 0.1780617 | 0.1389612 |
| Firmicutes_bacterium_CAG_95 | 0.0875902 | 0.1602256 | 0.0252248 | 0.058799 | 0.1104126 | 0.1739469 | 0.1105213 |
| Firmicutes_bacterium_CAG_170 | 0.0855432 | 0.0678924 | 0.0474589 | 0.0492577 | 0.0967768 | 0.1591362 | 0.0929959 |
| Oscillibacter_sp_57_20 | 0.0818922 | 0.1291765 | 0.1289121 | 0.1440433 | 0.0510126 | 0.1811097 | 0.055281 |
| Bifidobacterium_animalis | 0.0814422 | 0.0869707 | 0.0953017 | 0.1625929 | 0.0831505 | 0.1106554 | 0.092912 |
| Sutterella_parvirubra | 0.0811679 | 0.0333066 | 0.0024322 | −3.69E−04 | −0.002962 | 0.0817303 | 0.0094145 |
| Clostridium_sp_CAG_167 | 0.0796534 | 0.1344553 | 0.0423839 | 0.127188 | 0.0800689 | 0.0851311 | 0.0709199 |
| Veillonella_dispar | 0.0745914 | 0.0498242 | 0.0915676 | 0.0576802 | 0.0476289 | 0.0906924 | 0.0491218 |
| Veillonella_infantium | 0.0701135 | 0.0436214 | 0.0765555 | 0.0888566 | 0.0517736 | 0.0804846 | 0.0608439 |
| Roseburia_sp_CAG_471 | 0.0657117 | 0.0870327 | 0.0364829 | 0.0490018 | 0.0594691 | 0.0937878 | 0.0722066 |
| Bacteroides_xylanisolvens | 0.0592196 | 0.0055548 | 0.0553754 | 0.021286 | 0.0175735 | 0.029645 | 0.0301535 |
| Veillonella_atypica | 0.0568184 | 0.0432855 | 0.0500656 | 0.0750177 | 0.0640909 | 0.072459 | 0.0625218 |
| Lactobacillus_rogosae | 0.055899 | 0.0731871 | 0.0376649 | −0.005037 | 0.0566438 | 0.0450159 | 0.0569764 |
| Roseburia_sp_CAG_309 | 0.0551092 | 0.027408 | −0.047601 | 0.0196 | 0.0626769 | 0.0485619 | 0.0737602 |
| Parabacteroides_goldsteinii | 0.0533229 | 0.0585964 | 0.0029002 | 0.0203696 | 0.0209185 | 0.0373069 | 0.0283556 |
| Bacteroides_sp_CAG_144 | 0.0521818 | 0.0178862 | −0.119061 | −0.037357 | 0.0468097 | 0.0258332 | 0.0366803 |
| Veillonella_sp_T11011_6 | 0.052079 | 0.0178074 | 0.0939981 | 0.0844662 | 0.0669139 | 0.0544433 | 0.0729158 |
| Bacteroides_finegoldii | 0.0518968 | −0.013606 | −0.012295 | 0.0340624 | 0.039123 | 0.0604151 | 0.0300303 |
| Slackia_isoflavoniconvertens | 0.0517058 | 0.0502789 | −0.00682 | 0.0338545 | 0.0563066 | 0.119152 | 0.0452785 |
| Roseburia_intestinalis | 0.0510537 | −0.00539 | 0.0159968 | 0.014429 | −0.010059 | 0.0336382 | 0.0090178 |
| Veillonella_parvula | 0.0495449 | 0.0337017 | 0.0696811 | 0.0405223 | 0.0428971 | 0.0823377 | 0.0507101 |
| Coprococcus_eutactus | 0.0493201 | 0.029225 | 0.0217796 | 0.0610756 | 0.0510846 | 0.1070335 | 0.0606219 |
| Holdemanella_biformis | 0.0478288 | 0.0321998 | −0.005094 | 0.018623 | 0.0382758 | 0.0806019 | 0.0238738 |
| Bacteroides_galacturonicus | 0.0456445 | 0.0576165 | 0.0308022 | 0.0084377 | 0.0073003 | 0.0363961 | 0.0049315 |
| Veillonella_rogosae | 0.0454441 | 0.0627839 | 0.1037374 | 0.0909477 | 0.0204195 | 0.0736019 | 0.0354318 |
| Bacteroides_intestinalis | 0.0452791 | 0.0194861 | 0.0227892 | −0.008545 | 0.0624061 | 0.036662 | 0.0567824 |
| Bacteroides_ovatus | 0.0406277 | 0.0596635 | 0.0448549 | 0.0517392 | −0.041837 | −0.019102 | −0.025128 |
| Firmicutes_bacterium_CAG_238 | 0.0402923 | 0.0607464 | 0.0429343 | 0.0218575 | 0.0371437 | 0.1099842 | 0.0263853 |
| Eubacterium_eligens | 0.0401487 | 0.1113062 | 0.0624273 | 0.100998 | 0.1312345 | 0.137495 | 0.1298428 |
| Streptococcus_australis | 0.0399914 | 0.0020312 | 0.0632512 | 5.65E−05 | 0.0047985 | 0.032085 | 0.0135396 |
| Desulfovibrio_piger | 0.0394799 | −0.020565 | 0.0082633 | −0.029149 | 0.0356602 | 0.0301369 | 0.0368223 |
| Oscillibacter_sp_PC13 | 0.0375716 | 0.0826832 | 0.0250491 | 0.0382229 | 0.1504939 | 0.1077377 | 0.1631763 |
| Flavonifractor_sp_An100 | 0.0346866 | 0.0066439 | −0.088668 | −0.011336 | 0.059317 | 0.0356821 | 0.0767549 |
| Agathobaculum_butyriciproducens | 0.0346561 | 0.1618834 | 0.0491266 | 0.1633205 | 0.0211392 | 0.0648056 | 0.0100515 |
| Coprococcus_catus | 0.0341136 | 0.0764626 | −0.024207 | 0.0450228 | 0.0531497 | 0.0622349 | 0.0470716 |
| Alistipes_shahii | 0.033651 | 0.0089914 | 0.0474433 | −0.020103 | 0.0082819 | 0.0158054 | 0.0088885 |
| Butyricimonas_synergistica | 0.0335482 | −0.028005 | −0.063652 | −0.007343 | 0.0707448 | 0.0241036 | 0.0414065 |
| Bacteroides_salyersiae | 0.0324435 | −0.022201 | 0.0061733 | −0.043744 | 0.0655336 | 0.0092356 | 0.0628737 |
| Ruminococcaceae_bacterium_D5 | 0.0316026 | 0.0579422 | 0.0831254 | −0.016824 | 0.0902596 | 0.0583282 | 0.0791039 |
| Ruminococcus_lactaris | 0.0314048 | 0.124049 | −0.027799 | 0.1128361 | 0.0610454 | 0.1141988 | 0.0728861 |
| Bacteroides_dorei | 0.0298447 | 0.0068724 | 0.0158306 | 0.049974 | 0.0059579 | −0.01145 | 0.0051836 |
| Roseburia_hominis | 0.02952 | 0.1243502 | 0.0097001 | 0.124358 | 0.0579736 | 0.0916822 | 0.0438033 |
| Lachnospira_pectinoschiza | 0.0289498 | 0.0358458 | 0.0083262 | −0.039687 | 0.0275612 | −0.003514 | 0.0242991 |
| Lactococcus_lactis | 0.0280034 | 0.0356267 | −0.016264 | −0.031924 | 0.0229923 | −0.0252 | 0.0260555 |
| Streptoccecus_parasanguinis | 0.0278828 | −0.025462 | −0.027056 | −0.03477 | 0.0679708 | 0.0432342 | 0.0616779 |
| Bacteroides_clarus | 0.0272304 | 0.0171445 | −0.024052 | 0.0211127 | 0.0399693 | 0.0331915 | 0.0365235 |
| Firmicutes_bacterium_CAG_110 | 0.0265821 | −0.016807 | −0.049097 | −0.084926 | 0.0638229 | 0.124232 | 0.059959 |
| Collinsella_stercoris | 0.0258798 | 0.0257042 | −0.067153 | 0.0027242 | −0.019574 | −1.43E−04 | −0.014434 |
| Roseburia_sp_CAG_182 | 0.0257688 | 0.1376297 | 0.1133022 | 0.1553229 | 0.0806325 | 0.1598725 | 0.0681123 |
| Haemophilus_sp_HMSC71H05 | 0.0250349 | 0.0380848 | 0.0788796 | 0.0578096 | 0.0642495 | 0.0743805 | 0.0639596 |
| Eubacterium_ramulus | 0.0248398 | 0.0367603 | 0.0097934 | 5.94E−04 | 0.0591117 | 0.0467169 | 0.0675634 |
| Turicimonas_muris | 0.0248165 | −9.94E−04 | 0.0040299 | 0.0047884 | 0.0284108 | 0.0107307 | 0.0104394 |
| Alistipes_indistinctus | 0.0241288 | 0.0049011 | −0.002431 | −0.05027 | 0.0212575 | −0.014557 | 0.0104868 |
| Methanobrevibacter_smithii | 0.0240176 | 1.49E−04 | −0.012127 | −0.054761 | 0.0322332 | 0.0563401 | 0.0058034 |
| Streptococcus_salivarius | 0.0225376 | −0.025788 | −0.022789 | −0.016304 | 0.0345519 | 0.0215465 | 0.0308781 |
| Faecalibacterium_prausnitzii | 0.021858 | 0.0891335 | 0.012352 | 0.0603414 | 0.0655734 | 0.097747 | 0.0507342 |
| Bacteroides_nordii | 0.0217146 | 0.069777 | 0.1058359 | 0.0642698 | 0.049332 | 0.0239538 | 0.0367402 |
| Parabacteroides_merdae | 0.0210218 | −0.064615 | −0.104293 | −0.100669 | 0.0571567 | −0.043236 | 0.0556087 |
| Actinomyces_odontolyticus | 0.0206795 | −0.013111 | 0.01671 | −0.007725 | 0.0498045 | −0.016457 | 0.0376312 |
| Eubacterium_hallii | 0.0205393 | 0.0733394 | 0.0206956 | 0.0676976 | 0.0399706 | 0.0368158 | 0.0476851 |
| Eubacterium_siraeum | 0.0195394 | −0.030688 | 0.0188672 | −0.069185 | 0.0337354 | 0.0127824 | 0.0371104 |
| Intestinimonas_butyriciproducens | 0.018438 | −0.019134 | 0.0256526 | −0.022089 | 0.1036209 | −0.023204 | 0.1166702 |
| Butyricimonas_virosa | 0.0183895 | −0.04224 | −0.060125 | −0.046429 | 0.0293919 | 0.0139468 | 0.012621 |
| Bacteroides_faecis | 0.0165344 | 0.0215236 | −0.009072 | −0.029146 | 0.0104825 | 0.0478588 | 0.0061021 |
| Actinomyces_sp_ICM47 | 0.0134231 | −0.02194 | −0.060214 | −0.013018 | 0.0158878 | −0.056642 | 0.010798 |
| Romboutsia_ilealis | 0.0127812 | 0.0823697 | 0.0956826 | 0.0391219 | 0.044951 | 0.1388015 | 0.0427064 |
| Eubacterium_sp_CAG_180 | 0.0117454 | −0.038649 | −0.059123 | −0.050939 | −0.032724 | 0.0351897 | −0.035012 |
| Gemella_sanquinis | 0.0113657 | −0.055406 | −0.059711 | −0.049182 | −0.071036 | −0.093984 | −0.08731 |
| Holdemania_filiformis | 0.0097869 | −0.058505 | −0.016972 | −0.101575 | −0.038487 | −0.032915 | −0.030157 |
| Bacteroides_vulqatus | 0.0095108 | 0.0017065 | −0.035617 | −0.018646 | 0.010692 | −0.073039 | 0.001284 |
| Streptococcus_sp_A12 | 0.0094969 | 0.0192981 | 0.0612216 | 0.0178644 | −0.019653 | 0.0056476 | −0.008412 |
| Barnesiella_intestinihominis | 0.0092959 | −0.033861 | −0.008046 | −0.038916 | 0.0154058 | 0.0048971 | 0.0120431 |
| Bacteroides_faecis_CAG_32 | 0.0085885 | 0.0594278 | −3.60E−04 | −0.013931 | −0.019404 | 0.0278971 | −0.029225 |
| Gemmiger_formicilis | 0.0067476 | −0.025491 | −5.07E−04 | −0.009919 | 0.0268744 | 0.0183608 | 0.0112357 |
| Roseburia_inulinivorans | 0.006399 | −0.024089 | −0.065825 | −0.073668 | −0.087237 | −0.021298 | −0.089932 |
| Anaerostipes_hadrus | 0.0058386 | 0.0951586 | 0.0559224 | 0.0951074 | 0.0451513 | 0.0211318 | 0.0483378 |
| Dialister_invisus | 0.0045137 | 0.0261694 | 0.0328763 | −0.025306 | 0.00468 | −0.018621 | 0.0144844 |
| Bifidobacterium_pseudocatenulatum | 0.0043829 | 0.0529275 | 0.0240955 | 0.0491911 | 0.0207823 | 0.0537215 | 0.0176152 |
| Dorea_formicigenerans | 0.0026752 | 0.0687506 | 0.05372 | 0.0348593 | −0.030762 | −0.010887 | −0.014717 |
| Firmicutes_bacterium_CAG_145 | 0.0023924 | −0.059023 | −0.126631 | −0.06502 | 0.0107782 | −0.097905 | 0.0261478 |
| Intestinibacter_bartlettii | 0.0022624 | −0.025646 | −0.008605 | −0.060715 | 0.022243 | 0.0084121 | 0.0114295 |
| Coprobacter_secundus | 0.0021785 | −0.015728 | −0.008581 | −0.025872 | 0.1181735 | 0.1056415 | 0.1012394 |
| Parabacteroides_distasonis | 0.0014827 | −0.005546 | −0.03601 | −0.016793 | 0.02794 | −0.1263 | 0.0091555 |
| Bacteroides_caccae | −0.002426 | −0.051507 | −0.026476 | −0.099292 | 0.0258435 | −0.017452 | 0.0119155 |
| [Collinsella]_massiliensis | −0.002519 | −0.065501 | −0.072745 | −0.057862 | −0.014554 | −0.026569 | −0.033241 |
| Olsenella_scatoligenes | −0.003626 | 0.0123205 | 0.0210905 | 0.0527618 | 0.004825 | 0.0430993 | 0.0043881 |
| Ruminococcus_bromii | −0.005803 | −0.03645 | −0.038632 | −0.051706 | 0.0126493 | 0.0120281 | 0.0101185 |
| Ruminococcus_callidus | −0.006802 | −0.036331 | 0.0293355 | −0.020154 | −0.006414 | 0.0591952 | 8.76E−04 |
| Fretibacterium_fastidiosum | −0.008364 | 0.0344852 | 0.0062544 | 0.0053658 | 0.0377775 | 0.0587911 | 0.0249659 |
| Dorea_longicatena | −0.009574 | −0.015241 | −0.04382 | −0.051592 | 0.0468123 | 0.0479813 | 0.0316172 |
| Eubacterium_sp_CAG_251 | −0.009875 | 0.0317994 | −0.028032 | 0.0031056 | 0.0055673 | 0.0423059 | −0.003815 |
| Streptococcus_mitis | −0.011553 | −0.04715 | −0.096649 | −0.116737 | 0.0407311 | −0.053324 | 0.0260987 |
| Bacteroides_cellulosilyticus | −0.01186 | 0.0278766 | −0.015365 | 0.04373 | 0.0498545 | −0.017082 | 0.0512939 |
| Clostridium_sp_CAG_253 | −0.011892 | 9.55E−04 | −0.001991 | 0.0561661 | 0.0255659 | 0.0330458 | 0.0208085 |
| Parasutterella_excrementihominis | −0.013269 | 0.0788959 | −0.009869 | 0.0345802 | 0.0031568 | 0.0367467 | −0.011368 |
| Bacteroides_thetaiotaomicron | −0.013302 | 0.0108222 | −7.85E−04 | 0.0373639 | −0.005103 | −0.047333 | 0.0020607 |
| Oscillibacter_sp_CAG_241 | −0.013952 | −0.013339 | −0.036191 | −0.084228 | 0.0316524 | 0.0587871 | 0.0040979 |
| Coprobacter_fastidiosus | −0.015331 | −0.016522 | −0.070333 | −0.056524 | −0.039858 | −0.078344 | −0.035031 |
| Streptococcus_thermophilus | −0.01691 | 0.0364457 | −0.018645 | 0.0248001 | 0.0706225 | 0.0085057 | 0.0851726 |
| Bacteroides_stercoris | −0.017208 | −0.012098 | −0.013227 | −0.037292 | 0.0193754 | −0.029578 | 0.0095745 |
| Lawsonibacter_asaccharolyticus | −0.017357 | −0.060166 | −0.168356 | −0.043701 | 0.0154725 | −0.082413 | 6.44E−04 |
| Bacteroides_eqqerthii | −0.017583 | 0.0498339 | 0.0459886 | 0.0171052 | 0.0079183 | 0.0817035 | −0.003284 |
| Alistipes_putredinis | −0.017787 | −0.051881 | −0.059061 | −0.09954 | 0.0201998 | −0.055133 | 0.0169562 |
| Victivallis_vadensis | −0.018066 | 0.0174813 | −0.004982 | −0.039017 | −0.02802 | 0.0657746 | −0.042556 |
| Collinsella_aerofaciens | −0.019929 | −0.02623 | −0.073549 | −0.048557 | −0.046113 | −0.002473 | −0.043884 |
| Eubacterium_sp_CAG_38 | −0.020089 | 0.0581003 | −0.002581 | 0.0566943 | 0.0291008 | 0.0575212 | 0.0431822 |
| Coprococcus_comes | −0.021178 | −0.071061 | −0.070263 | −0.062949 | 0.054804 | 0.0446007 | 0.0471512 |
| Odoribacter_splanchnicus | −0.021517 | −0.027488 | 0.0260371 | −0.078501 | 0.045471 | 0.053834 | 0.0424373 |
| Proteobacteria_bacterium_CAG_139 | −0.02181 | 0.0316122 | −0.01293 | 0.0156443 | 0.01082 | 0.0050314 | −0.005482 |
| Pseudoflavonifractor_capillosus | −0.023875 | −0.118016 | −0.101364 | −0.074848 | −0.012599 | −0.061231 | −0.012965 |
| Enorma_massiliensis | −0.024189 | −0.025719 | −0.137962 | −0.027839 | 0.0080053 | 0.0600716 | 3.17E−04 |
| Clostridium_disporicum | −0.024865 | −0.019365 | −0.024749 | −0.058286 | 0.0649288 | 0.0575964 | 0.0489548 |
| Ruminococcus_torques | −0.025877 | −0.05945 | −0.048827 | −0.094663 | 0.0266952 | −0.016012 | 0.022533 |
| Alistipes_onderdonkii | −0.027854 | 0.0103697 | −0.009208 | 0.0366529 | 0.0537466 | 0.0746172 | 0.0635165 |
| Turicibacter_sanguinis | −0.030998 | 0.0179818 | 0.0431482 | 0.00288 | 0.1106515 | 0.0830159 | 0.1048057 |
| Akkermansia_muciniphila | −0.031538 | 0.001566 | −0.051324 | −0.040092 | 0.0266353 | 0.0090844 | 0.0250258 |
| Flavonifractor_plautii | −0.038684 | −0.137189 | −0.099423 | −0.134203 | −0.072093 | −0.196707 | −0.072427 |
| Blautia_wexlerae | −0.039432 | 0.0485497 | 0.0432029 | 0.0247361 | −0.006839 | −0.009846 | −0.019301 |
| Bifidobacterium_adolescentis | −0.04172 | 0.0241443 | 0.0410906 | −0.010974 | −0.067153 | −0.02901 | −0.065037 |
| Bifidobacterium_longum | −0.041784 | −0.050024 | −0.116673 | −0.074897 | −0.024811 | −0.03616 | −0.030709 |
| Parabacteroides_johnsonii | −0.042486 | 0.0275618 | 0.0190568 | 0.0444287 | −0.003972 | −0.049817 | −0.015607 |
| Phascolarctobacterium_faecium | −0.047196 | 0.0093234 | 0.0147654 | −0.019581 | 0.0178153 | −0.017412 | 0.0118652 |
| Eubacterium_sp_OM08_24 | −0.047683 | −0.014995 | −0.014771 | −0.012495 | −0.015604 | −0.071427 | −0.025606 |
| Eisenbergiella_massiliensis | −0.052369 | −0.088633 | 0.0118499 | −0.055721 | −0.014692 | −0.098486 | −0.012284 |
| Clostridium_sp_CAG_242 | −0.053624 | −0.031898 | −0.013079 | 0.0117132 | 0.0492769 | 0.0542829 | 0.0603436 |
| Roseburia_faecis | −0.054506 | 0.0237273 | 0.0473492 | 0.0075482 | −0.02684 | 0.0352486 | −0.027987 |
| Bacteroides_uniformis | −0.055941 | −0.03693 | −0.015545 | −0.044245 | −0.064976 | −0.137773 | −0.079893 |
| Bifidobacterium_catenulatum | −0.056778 | −0.0548 | −0.060088 | −0.12255 | −0.038259 | −0.062717 | −0.033053 |
| Enterorhabdus_caecimuris | −0.057011 | −0.004762 | −0.036135 | 0.0191956 | 0.0647362 | 0.0168909 | 0.0584421 |
| Firmicutes_bacterium_CAG_83 | −0.057501 | 0.0040665 | 0.0029697 | 0.0010913 | −0.009577 | −0.041322 | 0.0109594 |
| Eubacterium_rectale | −0.058545 | −0.010014 | 0.0078943 | 0.0012331 | −0.070154 | −0.023092 | −0.070325 |
| Collinsella_intestinalis | −0.061063 | −0.075117 | −0.056688 | −0.086869 | −0.048842 | −0.08948 | −0.042583 |
| Blautia_hydrogenotrophica | −0.063664 | −0.08661 | −0.027964 | −0.066883 | −0.055512 | −0.077108 | −0.044265 |
| Ruminococcus_gnavus | −0.064674 | −0.097339 | −0.00603 | −0.081722 | −0.092702 | −0.156899 | −0.082778 |
| Blautia_obeum | −0.064787 | −0.024262 | −0.031808 | −0.023467 | −0.051088 | −0.017559 | −0.064944 |
| Dielma_fastidiosa | −0.06497 | −0.068704 | −0.001956 | −0.043241 | −0.008215 | −0.10753 | −0.018267 |
| Hungatella_hathewayi | −0.06568 | −0.087691 | 0.0226993 | −0.055643 | −0.010047 | −0.11699 | −0.022915 |
| Harryflintia_acetispora | −0.066444 | −0.075344 | −0.01772 | −0.096368 | 0.0026266 | −0.003312 | −0.012782 |
| Bilophila_wadsworthia | −0.067138 | −0.052476 | −0.068855 | −0.105457 | −0.027567 | −0.066971 | −0.025088 |
| Eggerthella_lenta | −0.067259 | −0.094184 | −0.039287 | −0.048405 | −0.041885 | −0.160741 | −0.043709 |
| Monoglobus_pectinilyticus | −0.067786 | 0.0364475 | 0.0409967 | 0.0292065 | −0.049037 | −0.005379 | −0.039515 |
| Bifidobacterium_bifidum | −0.068437 | −0.093434 | −0.031958 | −0.119512 | −0.023848 | −0.032761 | −0.029394 |
| Fusicatenibacter_saccharivorans | −0.069102 | 0.029359 | 0.0315068 | 0.0222682 | 0.0105468 | −0.047047 | 0.0081707 |
| Ruthenibacterium_lactatiformans | −0.070431 | −0.10345 | −0.071096 | −0.133843 | −0.032509 | −0.116218 | −0.046805 |
| Ruminococcus_bicirculans | −0.070739 | −0.005833 | −0.024359 | −0.033119 | 0.0082393 | −0.009373 | −0.002319 |
| Alistipes_finegoldii | −0.070911 | −0.041703 | −0.002758 | −0.065998 | −6.81E−04 | −0.050982 | −0.004477 |
| Eubacterium_sp_CAG_274 | −0.070941 | 0.0225675 | 0.0434762 | 0.0313895 | −0.016144 | −0.029525 | −0.015708 |
| Eubacterium_ventriosum | −0.071497 | −0.056923 | −0.022263 | −0.074825 | −0.086958 | −0.077432 | −0.09505 |
| Clostridium_spiroforme | −0.071647 | −0.070855 | −0.14004 | −0.094606 | −0.076539 | −0.128176 | −0.081013 |
| Clostridium_saccharolyticum | −0.073593 | −0.086479 | −0.094793 | −0.086727 | 0.0235884 | −0.082182 | 0.0151956 |
| Gordonibacter_pamelaeae | −0.07365 | −0.061856 | −0.011485 | −0.030745 | −0.02136 | −0.111951 | −0.025649 |
| Alistipes_inops | −0.075816 | 0.0252355 | −0.020954 | −0.018378 | 0.0232354 | 0.0485861 | 0.0125019 |
| Clostridium_lavalense | −0.082157 | −0.069282 | 0.0137232 | −0.031753 | −0.058082 | −0.130414 | −0.048715 |
| Clostridium_sp_CAG_58 | −0.082593 | −0.06197 | −0.056416 | −0.086051 | −0.022601 | −0.109872 | −0.025447 |
| Clostridium_bolteae_CAG_59 | −0.082682 | −0.096227 | −0.003154 | −0.069431 | −0.106942 | −0.150897 | −0.098182 |
| Adlercreutzia_equolifaciens | −0.082973 | 0.0092367 | −0.045691 | 0.0230181 | 0.0228653 | 0.0075089 | 0.0137571 |
| Escherichia_coli | −0.083619 | −0.109922 | −0.055991 | −0.090301 | −0.052932 | −0.095092 | −0.069596 |
| Ruminococcaceae_bacterium_D16 | −0.086218 | −0.047885 | −0.044549 | −0.099521 | 0.0214355 | −0.040654 | 0.023073 |
| Eisenbergiella_tayi | −0.087877 | −0.08905 | −0.064229 | −0.083681 | −0.022216 | −0.100708 | −0.034574 |
| Clostridium_citroniae | −0.09162 | −0.062763 | 0.0448075 | −0.042894 | −0.005502 | −0.149216 | 0.0086357 |
| Clostridium_bolteae | −0.093906 | −0.11707 | −0.04246 | −0.097 | −0.074955 | −0.205224 | −0.083621 |
| Asaccharobacter_celatus | −0.09402 | 0.0064875 | −0.049027 | 0.0132042 | 0.0523157 | 0.0112702 | 0.0505476 |
| Clostridium_innocuum | −0.094322 | −0.133374 | 0.0051836 | −0.118321 | −0.104554 | −0.180246 | −0.114069 |
| Anaerotruncus_colihominis | −0.094777 | −0.151726 | −0.065694 | −0.126757 | −0.081041 | −0.194457 | −0.085368 |
| Clostridium_asparagiforme | −0.108505 | −0.037726 | 0.0213946 | −0.014392 | −0.045232 | −0.08163 | −0.052398 |
| Firmicutes_bacterium_CAG_94 | −0.11262 | −0.140169 | −0.20902 | −0.126552 | −0.004161 | −0.079304 | −0.021525 |
| Pseudoflavonifractor_sp_An184 | −0.117704 | −0.103017 | −0.097949 | −0.141384 | −0.012427 | −0.069179 | −0.018962 |
| Clostridium_leptum | −0.122524 | −0.092373 | −0.105575 | −0.177189 | −0.015208 | −0.109057 | −0.015617 |
| Bacteroides_fragilis | −0.128982 | −0.041379 | −0.036113 | −0.028713 | −0.038194 | −0.048366 | −0.019771 |
| Clostridium_symbiosum | −0.144688 | −0.123482 | −0.025044 | −0.11135 | −0.087583 | −0.196164 | −0.092159 |
| Anaeromassilibacillus_sp_An250 | −0.148927 | −0.100187 | −0.117331 | −0.155697 | 0.0324247 | −0.058233 | 0.0109894 |

Table 5 (Part 1B): Spearman's correlations

| Profile | HDL_size_360 | ASCVD_10 yr_risk | visceral_fat |
|---|---|---|---|
| Positive/Negative | Positive | Negative | Negative |
| Paraprevotella_xylaniphila | 0.0721128 | −0.030306 | −0.092502 |
| Paraprevotella_clara | 0.0598959 | −0.024797 | −0.082246 |
| Bacteroides_massiliensis | 0.010796 | −0.011297 | −0.092452 |
| Prevotella_copri | 0.0696674 | −0.041895 | −0.112838 |
| Rothia_mucilaginosa | 0.0756649 | 0.0208184 | −0.158645 |
| Haemophilus_parainfluenzae | 0.1389612 | −0.078359 | −0.148303 |
| Firmicutes_bacterium_CAG_95 | 0.1105213 | −0.024449 | −0.150713 |
| Firmicutes_bacterium_CAG_170 | 0.0929959 | −0.110654 | −0.13699 |
| Oscillibacter_sp_5720 | 0.055281 | −0.085994 | −0.152019 |
| Bifidobacterium_animalis | 0.092912 | −0.012457 | −0.085896 |
| Sutterella_parvirubra | 0.0094145 | 0.0178711 | −0.033042 |
| Clostridium_sp_CAG_167 | 0.0709199 | −0.014776 | −0.124425 |
| Veillonella_dispar | 0.0491218 | −0.024305 | −0.09562 |
| Veillonella_infantium | 0.0608439 | −0.015789 | −0.107734 |
| Roseburia_sp_CAG_471 | 0.0722066 | −0.053176 | −0.048215 |
| Bacteroides_xylanisolvens | 0.0301535 | −0.027496 | −0.022134 |
| Veillonella_atypica | 0.0625218 | −0.05037 | −0.083424 |
| Lactobacillus_rogosae | 0.0569764 | −0.008089 | 0.0021415 |
| Roseburia_sp_CAG_309 | 0.0737602 | 0.0378596 | −0.074349 |
| Parabacteroides_goldsteinii | 0.0283556 | −0.027207 | −0.0576 |
| Bacteroides_sp_CAG_144 | 0.0366803 | −0.013959 | −0.008008 |
| Veillonella_sp_T11011_6 | 0.0729158 | −0.003648 | −0.097398 |
| Bacteroides_finegoldii | 0.0300303 | −4.23E−05 | −0.071703 |
| Slackia_isoflavoniconvertens | 0.0452785 | 0.0524628 | −0.069714 |
| Roseburia_intestinalis | 0.0090178 | −0.00134 | 0.0265612 |
| Veillonella_parvula | 0.0507101 | −0.04978 | −0.057781 |
| Coprocoecus_eutactus | 0.0606219 | 4.78E−04 | −0.083434 |
| Holdemanella_biformis | 0.0238738 | 0.017592 | −0.11156 |
| Bacteroides_galacturonicus | 0.0049315 | 0.0138762 | 0.030634 |
| Veillonella_rogosae | 0.0354318 | −0.069362 | −0.039487 |
| Bacteroides_intestinalis | 0.0567824 | −0.068696 | −0.047572 |
| Bacteroides_ovatus | −0.025128 | −0.006202 | 0.0457885 |
| Firmicutes_bacterium_CAG_238 | 0.0263853 | −0.053913 | −0.100161 |
| Eubacterium_eligens | 0.1298428 | −0.086388 | −0.095462 |
| Streptococcus_australis | 0.0135396 | −0.067204 | −0.010597 |
| Desulfovibrio_piger | 0.0368223 | 0.0409445 | −0.011395 |
| Oscillibacter_sp_PC13 | 0.1631763 | −0.027694 | −0.078856 |
| Flavonifractor_sp_An100 | 0.0767549 | −0.031112 | −0.053749 |
| Agathobaculum_butyriciproducens | 0.0100515 | 0.0537454 | 0.0424394 |
| Coproccocus_catus | 0.0470716 | 0.0518023 | −0.100739 |
| Alistipes_shahii | 0.0088885 | 0.0017851 | −0.05377 |
| Butyricimonas_synergistica | 0.0414065 | −0.009607 | −0.064359 |
| Bacteroides_salyersiae | 0.0628737 | 0.0404092 | 0.0036444 |
| Ruminococcaceae_bacterium_D5 | 0.0791039 | −0.044959 | −0.138359 |
| Ruminococcus_lactaris | 0.0728861 | −0.054695 | −0.06974 |
| Bacteroides_dorei | 0.0051836 | −0.044102 | 0.0075912 |
| Roseburia_hominis | 0.0438033 | −0.026098 | −0.028045 |
| Lachnospira_pectinoschiza | 0.0242991 | −7.26E−05 | 0.0359566 |
| Lactococcus_lactis | 0.0260555 | −0.038516 | −0.041305 |
| Streptococcus_parasanguinis | 0.0616779 | −0.020926 | −0.10747 |
| Bacteroides_clarus | 0.0365235 | −0.015292 | −0.020029 |
| Firmicutes_bacterium_CAG_110 | 0.059959 | −0.043419 | −0.120394 |
| Collinsella_stercoris | −0.014434 | 0.064591 | 0.0294562 |
| Roseburia_sp_CAG_182 | 0.0681123 | −0.098015 | −0.102745 |
| Haemophilus_sp_HMSC71H05 | 0.0639596 | 0.0089235 | −0.034984 |
| Eubacterium_ramulus | 0.0675634 | −0.028199 | 0.0012691 |
| Turicimonas_muris | 0.0104394 | −0.046206 | 0.02931 |
| Alistipes_indistinctus | 0.0104868 | −0.048741 | −0.009318 |
| Methanobrevibacter_smithii | 0.0058034 | 0.0135177 | −0.051829 |
| Streptococcus_salivarius | 0.0308781 | 0.0031857 | −0.071912 |
| Faecalibacterium_prausnitzii | 0.0507342 | −0.058587 | −0.0878 |
| Bacteroides_nordii | 0.0367402 | −0.034318 | −0.073092 |
| Parabacteroides_merdae | 0.0556087 | 0.0422602 | −0.036304 |
| Actinomyces_odontolyticus | 0.0376312 | −0.058214 | −0.03551 |
| Eubacterium_hallii | 0.0476851 | −0.028332 | 0.0178602 |
| Eubacterium_siraeum | 0.0371104 | −0.036799 | −0.078899 |
| Intestinimonas_butyriciproducens | 0.1166702 | −0.021647 | −0.055203 |
| Butyricimonasa_virosa | 0.012621 | 0.0033642 | −0.077931 |
| Bacteroides_faecis | 0.0061021 | −0.062577 | 0.0204147 |
| Actinomyces_sp_ICM47 | 0.010798 | −0.022237 | 0.0627508 |
| Romboutsia_ilealis | 0.0427064 | −0.051995 | −0.034322 |
| Eubacterium_sp_CAG_180 | −0.035012 | 0.0394454 | −0.018893 |
| Gemella_sanguinis | −0.08731 | 0.0296597 | 0.1068814 |
| Holdemania_filiformis | −0.030157 | 0.030341 | 0.0368681 |
| Bacteroides_vulgatus | 0.001284 | −0.023113 | 0.0776035 |
| Streptococcus_sp_A12 | −0.008412 | −0.018146 | 0.0332543 |
| Barnesiella_intestinihominis | 0.0120431 | −0.003102 | −0.005907 |
| Bacteroides_faecis_CAG_32 | −0.029225 | −0.004477 | 0.0283552 |
| Gemmiger_formicilis | 0.0112357 | 0.0075961 | −0.028954 |
| Roseburia_inulinivorans | −0.089932 | 0.0363865 | 0.1130501 |
| Anaerostipes_hadrus | 0.0483378 | 8.96E−04 | 0.067151 |
| Dialister_invisus | 0.0144844 | 0.0390319 | −0.026341 |
| Bifidobacterium_pseudocatenulatum | 0.0176152 | 0.0166191 | 0.0576346 |
| Dorea_formicigenerans | −0.014717 | −0.008249 | 0.045563 |
| Firmicutes_bacterium_CAG_145 | 0.0261478 | 0.0141045 | 0.057524 |
| Intestinibacter_bartlettii | 0.0114295 | 0.0275968 | −0.036622 |
| Coprobacter_secundus | 0.1012394 | −0.010266 | −0.021952 |
| Parabacteroides_distasonis | 0.0091555 | −0.035214 | 0.044787 |
| Bacteroides_caccae | 0.0119155 | −0.020669 | 0.0173502 |
| [Collinsella]_massiliensis | −0.033241 | 0.0570584 | 0.024428 |
| Olsenella_scatoligenes | 0.0043881 | −0.013389 | 0.0130877 |
| Ruminococcus_bromii | 0.0101185 | 0.0267426 | 0.0028055 |
| Ruminococcus_callidus | 8.76E−04 | −0.014411 | −0.051976 |
| Fretibacterium_fastidiosum | 0.0249659 | 0.0044675 | −0.055567 |
| Dorea_longicatena | 0.0316172 | 0.0277335 | −5.21E−04 |
| Eubacterium_sp_CAG_251 | −0.003815 | 0.1179033 | −0.030343 |
| Streptococcus_mitis | 0.0260987 | −0.00155 | 0.0100717 |
| Bacteroides_cellulosilyticus | 0.0512939 | 0.0039937 | −0.012872 |
| Clostridium_sp_CAG_253 | 0.0208085 | −0.002647 | −0.02408 |
| Parasutterella_excrementihominis | −0.011368 | −0.020085 | 0.0419844 |
| Bacteroides_thetaiotaomicron | 0.0020607 | −0.038505 | 0.0138817 |
| Oscillibacter_sp_CAG_241 | 0.0040979 | 0.0040114 | −0.071359 |
| Coprobacter_fastidiosus | −0.035031 | 0.010181 | 0.0229357 |
| Streptococcus_thermophilus | 0.0851726 | 0.0260141 | −0.042165 |
| Bacteroides_stercoris | 0.0095745 | 0.0200332 | −0.030931 |
| Lawsonibacter_asaccharolyticus | 6.44E−04 | 0.0657302 | 0.0066287 |
| Bacteroides_eggerthii | −0.003284 | 0.0630055 | −0.017251 |
| Alistipes_putredinis | 0.0169562 | 0.0337554 | −0.02304 |
| Victivallis_vadensis | −0.042556 | 0.0390547 | −0.051396 |
| Collinsella_aerofaciens | −0.043884 | 0.1526939 | 0.0615486 |
| Eubacterium_sp_CAG_38 | 0.0431822 | −0.022015 | 0.0347712 |
| Coprococcus_comes | 0.0471512 | 0.1034206 | −0.043717 |
| Odoribacter_splanchnicus | 0.0424373 | −0.046778 | −0.044415 |
| Proteobacteria_bacterium_CAG_139 | −0.005482 | −0.027521 | 0.0590119 |
| Pseudoflavonifractor_capillosus | −0.012965 | 0.0208161 | 0.0564652 |
| Enorma_massiliensis | 3.17E−04 | 0.0066599 | 0.0113687 |
| Clostridium_disporicum | 0.0489548 | −0.013689 | −0.053579 |
| Ruminococcus_torques | 0.022533 | 0.0158575 | 0.0034218 |
| Alistipes_onderdonkii | 0.0635165 | −0.054227 | −0.014462 |
| Turicibacter_sanguinis | 0.1048057 | 0.0146673 | −0.104074 |
| Akkermansia_muciniphila | 0.0250258 | 0.0648256 | −0.040453 |
| Flavonifractor_plautii | −0.072427 | 0.0864192 | 0.1668312 |
| Blautia_wexlerae | −0.019301 | −0.018601 | 0.054811 |
| Bifidobacterium_adolescentis | −0.065037 | 0.0279983 | −0.003509 |
| Bifidobacterium_longum | −0.030709 | 0.0979118 | 0.0674864 |
| Parabacteroides_johnsonii | −0.015607 | 0.0341116 | 0.0632366 |
| Phascolarctobacterium_faecium | 0.0118652 | −0.033475 | 0.0150257 |
| Eubacterium_sp_OM08_24 | −0.025606 | 0.059661 | 0.0065878 |
| Eisenbergiella_massiliensis | −0.012284 | −0.01005 | 0.0580386 |
| Clostridium_sp_CAG_242 | 0.0603436 | −0.02043 | −0.053676 |
| Roseburia_faecis | −0.027987 | 0.0830759 | 0.0452067 |
| Bacteroides_uniformis | −0.079893 | 0.0146537 | 0.0704485 |
| Bifidobacterium_catenulatum | −0.033053 | 0.0843948 | 0.0369933 |
| Enterorhabdus_caecimuris | 0.0584421 | −0.010825 | 9.31E−06 |
| Firmicutes_bacterium_CAG_83 | 0.0109594 | 0.0479539 | −0.00707 |
| Eubacterium_rectale | −0.070325 | 0.0077426 | 0.0515748 |
| Collinsella_intestinalis | −0.042583 | 0.0793574 | 0.065161 |
| Blautia_hydrogenotrophica | −0.044265 | 0.0785694 | 0.1012023 |
| Ruminococcus_gnavus | −0.082778 | 0.0284378 | 0.1548579 |
| Blautia_obeum | −0.064944 | 0.1109821 | 0.0246973 |
| Dielma_fastidiosa | −0.018267 | 0.0071655 | 0.0626463 |
| Hungatella_hathewayi | −0.022915 | 0.0638668 | −0.004247 |
| Harryflintia_acetispora | −0.012782 | −0.037275 | 0.064804 |
| Bilophila_wadsworthia | −0.025088 | 0.0503101 | 0.0493119 |
| Eggerthella_lenta | −0.043709 | 0.0471728 | 0.0897402 |
| Monoglobus_pectinilyticus | −0.039515 | 0.0306212 | 0.055641 |
| Bifidobacterium_bifidum | −0.029394 | 0.0275381 | 0.0217168 |
| Fusicatenibacter_saccharivorans | 0.0081707 | −0.028162 | 0.0573208 |
| Ruthenibacterium_lactatiformans | −0.046805 | −0.015426 | 0.0560687 |
| Ruminococcus_bicirculans | −0.002319 | 0.0934479 | 0.0652762 |
| Alistipes_finegoldii | −0.004477 | 0.0485964 | −0.005365 |
| Eubacterium_sp_CAG_274 | −0.015708 | 0.1217073 | 0.0948079 |
| Eubacterium_ventriosum | −0.09505 | 0.0281687 | 0.0569745 |
| Clostridium_spiroforme | −0.081013 | 0.044933 | 0.1634814 |
| Clostridium_saccharolyticum | 0.0151956 | −0.069931 | 0.064915 |
| Gordonibacter_pamelaeae | −0.025649 | 0.0160697 | 0.0763758 |
| Alistipes_inops | 0.0125019 | −0.030509 | 0.0042954 |
| Clostridium_lavalense | −0.048715 | 0.0117277 | 0.1018552 |
| Clostridium_sp_CAG_58 | −0.025447 | 0.038079 | 0.1170601 |
| Clostridium_bolteae_CAG_59 | −0.098182 | 0.073137 | 0.1079112 |
| Adlercreutzia_equolifaciens | 0.0137571 | −0.009827 | 0.0072902 |
| Escherichia_coli | −0.069596 | 0.0756429 | 0.044379 |
| Ruminococcaceae_bacterium_D16 | 0.023073 | 0.0059278 | −0.024027 |
| Eisenbergiella_tayi | −0.034574 | 0.0511229 | 0.0137691 |
| Clostridium_citroniae | 0.0086357 | −0.01419 | 0.1021057 |
| Clostridium_bolteae | −0.083621 | 0.0465657 | 0.1479338 |
| Asaccharobacter_celatus | 0.0505476 | 0.0080789 | −0.010281 |
| Clostridium_innocuum | −0.114069 | 0.0815986 | 0.1100507 |
| Anaerotruncus_colihominis | −0.085368 | 0.0038084 | 0.1189 |
| Clostridium_asparagiforme | −0.052398 | 0.0163417 | 0.1005458 |
| Firmicutes_bacterium_CAG_94 | −0.021525 | 0.0957409 | 0.0591975 |
| Pseudoflavonifractor_sp_An184 | −0.018962 | 0.0312262 | 0.0238006 |
| Clostridium_leptum | −0.015617 | 0.0837095 | 0.075378 |
| Bacteroides_fragilis | −0.019771 | 0.0082337 | 0.0982004 |
| Clostridium_symbiosum | −0.092159 | 0.0681861 | 0.0859997 |
| Anaeromassilibacillus_sp_An250 | 0.0109894 | 0.0656062 | 0.0402083 |

Table 5 (Part 1B): Spearman's correlations

| Profile | LiverFatProbability | uPDI | Total_TG_0 | VLDL_size_0 |
|---|---|---|---|---|
| Positive/Negative | Negative | Negative | Negative | Negative |
| Paraprevotella_xylaniphila | −0.047335 | −0.118085 | −0.072888 | −0.099676 |
| Paraprevotella_clara | −0.039972 | −0.109858 | −0.066752 | −0.093296 |
| Bacteroides_massiliensis | 0.0101143 | −0.073856 | −0.051671 | −0.049914 |
| Prevotella_copri | −0.035438 | −0.08003 | −0.127063 | −0.08046 |
| Rothia_mucilaginosa | −0.043918 | −0.106341 | −0.030329 | −0.065295 |
| Haemophilus_parainfluenzae | −0.032099 | −0.077406 | −0.153016 | −0.120413 |
| Firmicutes_bacterium_CAG_95 | −0.104629 | −0.153717 | −0.162654 | −0.159522 |
| Firmicutes_bacterium_CAG_170 | −0.107132 | −0.066913 | −0.154056 | −0.142703 |
| Oscillibacter_sp_5720 | −0.04818 | −0.116996 | −0.149627 | −0.120095 |
| Bifidobacterium_animalis | −0.009252 | −0.167053 | −0.065336 | −0.078999 |
| Sutterella_parvirubra | 0.0177683 | −0.082126 | −0.043432 | −0.013363 |
| Clostridium_sp_CAG_167 | −0.057293 | −0.076805 | −0.079709 | −0.072759 |
| Veillonella_dispar | −0.055159 | −0.083531 | −0.065895 | −0.049617 |
| Veillonella_infantium | −0.030568 | −0.060489 | −0.063127 | −0.059994 |
| Roseburia_sp_CAG_471 | −0.019003 | −0.087217 | −0.032331 | −0.047821 |
| Bacteroides_xylanisolvens | 0.0146477 | −0.021517 | −0.027225 | −0.027876 |
| Veillonella_atypica | −0.02362 | −0.054626 | −0.059517 | −0.045666 |
| Lactobacillus_rogosae | −0.077326 | −0.021451 | −0.043871 | −0.037462 |
| Roseburia_sp_CAG_309 | −0.034637 | −0.073075 | −0.020844 | −0.045088 |
| Parabacteroides_goldsteinii | −0.039912 | −0.006234 | 0.0016616 | −0.018607 |
| Bacteroides_sp_CAG_144 | −0.017392 | −0.005238 | −0.014876 | −0.03795 |
| Veillonella_sp_T11011_6 | −0.044687 | −0.041385 | −0.064475 | −0.069002 |
| Bacteroides_finegoldii | 0.028932 | −0.031647 | −0.023297 | −0.023248 |
| Slackia_isoflavoniconvertens | −0.02169 | −0.035699 | −0.055942 | −0.069671 |
| Roseburia_intestinalis | −0.031805 | −0.022706 | 0.0105699 | 0.0155929 |
| Veillonella_parvula | −0.014629 | 0.001184 | −0.081428 | −0.058872 |
| Coprocoecus_eutactus | −0.018832 | 0.0155636 | −0.072135 | −0.058154 |
| Holdemanella_biformis | 0.0074164 | −0.048848 | −0.062981 | −0.048909 |
| Bacteroides_galacturonicus | −0.071066 | −0.002556 | −0.00183 | 0.0091514 |
| Veillonella_rogosae | −0.008357 | −0.080422 | −0.046963 | −0.043604 |
| Bacteroides_intestinalis | 0.0252424 | 0.0108063 | −0.060872 | −0.074126 |
| Bacteroides_ovatus | 0.0653122 | −0.030776 | 0.0357033 | 0.0213147 |
| Firmicutes_bacterium_CAG_238 | −0.041193 | −0.051904 | −0.106578 | −0.100123 |
| Eubacterium_eligens | −0.067468 | −0.136281 | −0.076918 | −0.113639 |
| Streptococcus_australis | 0.0060736 | −0.05369 | 0.0174158 | 0.0016852 |
| Desulfovibrio_piger | −0.014811 | 0.0134328 | −0.032034 | −0.031763 |
| Oscillibacter_sp_PC13 | −0.056509 | −0.141532 | −0.112735 | −0.124977 |
| Flavonifractor_sp_An100 | −0.047197 | −0.012072 | −0.007347 | −0.054493 |
| Agathobaculum_butyriciproducens | −0.014195 | −0.119439 | −0.001906 | 0.0148452 |
| Coprococcus_catus | −0.075547 | −0.069853 | −0.061028 | −0.079802 |
| Alistipes_shahii | 0.0060406 | −0.012612 | −0.033613 | −0.041192 |
| Butyricimonas_synergistica | 0.0140329 | −0.017835 | −0.069731 | −0.117032 |
| Bacteroides_salyersiae | −0.023391 | 0.0066607 | −0.053372 | −0.072331 |
| Ruminococcaceae_bacterium_D5 | −0.080114 | 0.0084411 | −0.069595 | −0.089427 |
| Ruminococcus_lactaris | −0.061141 | −0.094988 | −0.092238 | −0.083766 |
| Bacteroides_dorei | 0.0452379 | −0.035715 | 0.0240777 | 0.0043614 |
| Roseburia_hominis | −0.003524 | −0.143393 | −0.052829 | −0.056587 |
| Lachnospira_pectinoschiza | −0.011549 | 0.0291291 | −0.002826 | 2.16E−04 |
| Lactococcus_lactis | 0.0072671 | −0.063242 | 0.0429886 | −0.002595 |
| Streptococcus_parasanguinis | −0.039643 | 0.0209289 | −0.040297 | −0.048973 |
| Bacteroides_clarus | 0.0357499 | −0.044097 | −0.051998 | −0.058298 |
| Firmicutes_bacterium_CAG_110 | −0.138539 | −0.009115 | −0.121512 | −0.115021 |
| Collinsella_stercoris | 0.0094014 | 0.0049337 | 0.0348652 | 0.0396658 |
| Roseburia_sp_CAG_182 | −0.075711 | −0.12954 | −0.129458 | −0.10814 |
| Haemophilus_sp_HMSC71H05 | 0.0119186 | −0.055373 | −0.064607 | −0.057924 |
| Eubacterium_ramulus | −0.035026 | −0.094042 | −0.042536 | −0.05047 |
| Turicimonas_muris | −0.025769 | −0.00181 | −0.003747 | −0.023646 |
| Alistipes_indistinctus | 0.0047674 | −0.014323 | 0.0185063 | 8.68E−04 |
| Methanobrevibacter_smithii | −0.082048 | 0.0172252 | −0.033724 | −0.040241 |
| Streptococcus_salivarius | 0.0016615 | 0.0172469 | 0.0033968 | −0.001207 |
| Faecalibacterium_prausnitzii | −0.032427 | −0.104372 | −0.086394 | −0.062252 |
| Bacteroides_nordii | 0.0416759 | −0.022677 | −0.039078 | −0.05385 |
| Parabacteroides_merdae | 0.0103838 | 0.0748368 | 0.0015805 | −0.049613 |
| Actinomyces_odontolyticus | 0.0116131 | −0.004034 | −0.003347 | −0.042268 |
| Eubacterium_hallii | −0.007558 | −0.103348 | −0.053592 | −0.07319 |
| Eubacterium_siraeum | −0.07857 | 0.0576132 | −0.049522 | −0.050094 |
| Intestinimonas_butyriciproducens | −6.95E−04 | 0.0052589 | −0.015223 | −0.0484 |
| Butyricimonasa_virosa | −0.04783 | 0.030829 | −0.053566 | −0.082262 |
| Bacteroides_faecis | −4.51E−04 | −0.064276 | −0.036378 | −0.03062 |
| Actinomyces_sp_ICM47 | −0.104815 | −0.032591 | 0.0602607 | 0.0583636 |
| Romboutsia_ilealis | −0.063496 | −0.015269 | −0.063186 | −0.055395 |
| Eubacterium_sp_CAG_180 | 0.0048812 | 0.034421 | −0.012871 | −0.012395 |
| Gemella_sanguinis | 0.0077289 | 0.0255135 | 0.0938277 | 0.0841489 |
| Holdemania_filiformis | 0.0202805 | 0.027696 | 0.0111477 | 0.0096767 |
| Bacteroides_vulgatus | 0.001137 | 0.0166292 | 0.0543934 | 0.0387902 |
| Streptococcus_sp_A12 | −0.018901 | −0.057561 | 0.0227361 | 0.014265 |
| Barnesiella_intestinihominis | 0.0160901 | 0.0244137 | −0.007778 | −0.017541 |
| Bacteroides_faecis_CAG_32 | 0.0184207 | −0.037727 | 0.009248 | −0.011996 |
| Gemmiger_formicilis | −0.049932 | 0.005174 | −0.045811 | −0.048993 |
| Roseburia_inulinivorans | −0.016935 | 0.0297042 | 0.0579643 | 0.0896153 |
| Anaerostipes_hadrus | −0.047864 | −0.135582 | −0.00323 | −0.018181 |
| Dialister_invisus | −0.036042 | 0.0164865 | 0.0240531 | 0.0118691 |
| Bifidobacterium_pseudocatenulatum | −0.001727 | −0.020785 | −0.03562 | −0.044088 |
| Dorea_formicigenerans | 0.0425148 | −0.050671 | 0.0446559 | 0.0402486 |
| Firmicutes_bacterium_CAG_145 | −0.040439 | 0.0256918 | 0.0987862 | 0.0529393 |
| Intestinibacter_bartlettii | −0.005074 | 0.0269639 | −0.033287 | −0.029919 |
| Coprobacter_secundus | −0.042569 | −0.012168 | −0.113949 | −0.155626 |
| Parabacteroides_distasonis | 0.0460826 | −0.009238 | 0.0674625 | 0.0271083 |
| Bacteroides_caccae | 0.0341976 | 0.0518427 | −2.03E−04 | −0.015664 |
| [Collinsella]_massiliensis | 0.0328266 | 0.0480453 | 0.0250345 | 0.0037229 |
| Olsenella_scatoligenes | −0.018979 | −0.020573 | −0.022542 | −0.019123 |
| Ruminococcus_bromii | −0.044831 | −0.006942 | −0.027216 | −0.029086 |
| Ruminococcus_callidus | −0.048257 | 0.0334486 | −0.00512 | 0.0163289 |
| Fretibacterium_fastidiosum | −0.058522 | −0.048383 | −0.086176 | −0.089822 |
| Dorea_longicatena | 0.0203304 | 0.0231524 | −0.00639 | −0.02077 |
| Eubacterium_sp_CAG_251 | −0.039046 | 0.0231955 | 0.0028231 | 0.0176996 |
| Streptococcus_mitis | −0.05753 | 0.0186263 | 0.0209465 | −7.94E−04 |
| Bacteroides_cellulosilyticus | −0.04664 | −0.04239 | −0.001268 | −0.052392 |
| Clostridium_sp_CAG_253 | −0.068015 | −0.009204 | −0.02963 | −0.047847 |
| Parasutterella_excrementihominis | −0.007444 | −0.055657 | −0.014797 | −0.017017 |
| Bacteroides_thetaiotaomicron | 0.0385762 | −0.003223 | 0.0409083 | 0.0228614 |
| Oscillibacter_sp_CAG_241 | −0.073005 | 0.0581801 | −0.080113 | −0.080314 |
| Coprobacter_fastidiosus | 0.0016304 | 0.0043542 | 0.0662611 | 0.0514423 |
| Streptococcus_thermophilus | 0.0060809 | −0.166879 | −4.81E−05 | −0.060462 |
| Bacteroides_stercoris | −0.016069 | 0.0118967 | 0.0274959 | 0.0110585 |
| Lawsonibacter_asaccharolyticus | 8.30E−04 | −0.062352 | 0.0614012 | 0.039503 |
| Bacteroides_eggerthii | −0.013285 | −0.009956 | −0.037385 | −0.012928 |
| Alistipes_putredinis | 0.0271548 | 0.0489851 | −0.013641 | −0.035138 |
| Victivallis_vadensis | −0.085771 | 0.0510195 | −0.074683 | −0.054655 |
| Collinsella_aerofaciens | 0.0018916 | 0.0450264 | 0.0111746 | 0.0049273 |
| Eubacterium_sp_CAG_38 | 0.0109603 | −0.063593 | 0.0093711 | −2.02E−04 |
| Coprococcus_comes | −0.036411 | 0.0299482 | −0.03934 | −0.049527 |
| Odoribacter_splanchnicus | 0.006957 | 0.0468952 | −0.068619 | −0.082589 |
| Proteobacteria_bacterium_CAG_139 | −0.007242 | −0.035426 | 3.84E−04 | −0.003895 |
| Pseudoflavonifractor_capillosus | 0.0265719 | 0.0922703 | 0.0479646 | 0.0500209 |
| Enorma_massiliensis | 0.0152606 | 0.0283944 | −0.054123 | −0.049907 |
| Clostridium_disporicum | −0.04424 | 0.0408635 | −0.112587 | −0.096551 |
| Ruminococcus_torques | −0.025928 | −0.001163 | −0.008411 | 2.69E−04 |
| Alistipes_onderdonkii | −0.036763 | −0.008198 | −0.085693 | −0.095927 |
| Turicibacter_sanguinis | −0.08253 | 0.0154632 | −0.113928 | −0.128187 |
| Akkermansia_muciniphila | −0.039009 | 0.0053188 | −0.01703 | −0.040804 |
| Flavonifractor_plautii | 0.0802682 | 0.1101024 | 0.1510286 | 0.1202539 |
| Blautia_wexlerae | −0.022301 | −0.023568 | 0.0163955 | 0.0447895 |
| Bifidobacterium_adolescentis | 0.0303383 | 0.0266175 | 0.0192257 | 0.0351333 |
| Bifidobacterium_longum | −0.020766 | 0.06606 | 0.0165738 | 0.0111946 |
| Parabacteroides_johnsonii | 0.0075519 | −0.004663 | 0.0509341 | 0.0162284 |
| Phascolarctobacterium_faecium | 0.0131703 | −0.019482 | −0.011917 | −0.013969 |
| Eubacterium_sp_OM08_24 | −0.011702 | 0.0230417 | 0.0664814 | 0.0482649 |
| Eisenbergiella_massiliensis | 0.0361614 | 0.0373117 | 0.0817945 | 0.0666876 |
| Clostridium_sp_CAG_242 | 0.0150071 | −0.010456 | −0.03396 | −0.048228 |
| Roseburia_faecis | −0.0246 | −0.031923 | −0.005413 | 0.02924 |
| Bacteroides_uniformis | 0.0230916 | 0.0187665 | 0.1171531 | 0.0868143 |
| Bifidobacterium_catenulatum | 0.035062 | 0.102221 | 0.0502064 | 0.0520068 |
| Enterorhabdus_caecimuris | −0.033521 | 0.0179457 | 0.0041678 | −0.023806 |
| Firmicutes_bacterium_CAG_83 | 0.0013578 | 0.0028313 | −0.003959 | −0.043973 |
| Eubacterium_rectale | 0.0217582 | −0.022552 | 0.0459054 | 0.0594763 |
| Collinsella_intestinalis | 0.0452711 | 0.1018303 | 0.060335 | 0.0822122 |
| Blautia_hydrogenotrophica | 0.0464728 | 0.0494624 | 0.0990899 | 0.1002876 |
| Ruminococcus_gnavus | 0.1041778 | 0.0844105 | 0.1470552 | 0.1381019 |
| Blautia_obeum | −0.027525 | 0.037505 | 0.0384083 | 0.0424194 |
| Dielma_fastidiosa | −0.037246 | 0.0642181 | 0.0760759 | 0.0495328 |
| Hungatella_hathewayi | 0.016025 | 0.0764112 | 0.0722947 | 0.0404845 |
| Harryflintia_acetispora | −0.018094 | 0.0717769 | 0.0205752 | 0.0263027 |
| Bilophila_wadsworthia | −0.009208 | 0.047491 | 0.023683 | 0.0086748 |
| Eggerthella_lenta | 0.0272697 | 0.111792 | 0.1062223 | 0.0926723 |
| Monoglobus_pectinilyticus | −0.032901 | −0.017789 | 0.0479925 | 0.0675756 |
| Bifidobacterium_bifidum | 0.0158029 | 0.1030354 | 0.033319 | 0.037754 |
| Fusicatenibacter_saccharivorans | −0.008209 | −0.038688 | 0.0596272 | 0.0406194 |
| Ruthenibacterium_lactatiformans | −0.021423 | 0.1304623 | 0.0997546 | 0.0591949 |
| Ruminococcus_bicirculans | −0.01434 | 0.0268329 | 0.0232486 | −0.003434 |
| Alistipes_finegoldii | −0.006322 | 0.0835273 | 0.0060641 | −0.022527 |
| Eubacterium_sp_CAG_274 | 0.0356975 | −0.026118 | 0.0152271 | −0.007002 |
| Eubacterium_ventriosum | 0.0202877 | 0.0275496 | 0.0816584 | 0.0780796 |
| Clostridium_spiroforme | 0.029696 | 0.1094929 | 0.1081196 | 0.0899547 |
| Clostridium_saccharolyticum | 0.0226403 | 0.0747183 | 0.0449978 | 0.0181202 |
| Gordonibacter_pamelaeae | −0.006167 | 0.0946307 | 0.07974 | 0.0627272 |
| Alistipes_inops | −0.01461 | 0.0043829 | −0.048916 | −0.044256 |
| Clostridium_lavalense | −0.002858 | 0.0685667 | 0.1174284 | 0.1038124 |
| Clostridium_sp_CAG_58 | 0.0616734 | 0.0377409 | 0.1219373 | 0.0842452 |
| Clostridium_bolteae_CAG_59 | 0.0566796 | 0.1035427 | 0.1376543 | 0.1253361 |
| Adlercreutzia_equolifaciens | −0.03499 | 0.0074422 | 0.0170912 | 0.0092864 |
| Escherichia_coli | 0.0215275 | 0.1346826 | 0.0869754 | 0.0867825 |
| Ruminococcaceae_bacterium_D16 | 8.31E−04 | 0.0376162 | 0.0565961 | 0.0210366 |
| Eisenbergiella_tayi | −0.018311 | 0.0818324 | 0.0834531 | 0.0329649 |
| Clostridium_citroniae | 0.0970807 | 0.0590528 | 0.1072136 | 0.0756267 |
| Clostridium_bolteae | 0.0765466 | 0.1123636 | 0.1949528 | 0.1549387 |
| Asaccharobacter_celatus | −0.057073 | 5.54E−04 | −0.007098 | −0.02069 |
| Clostridium_innocuum | 0.0674329 | 0.1501922 | 0.1388306 | 0.1231837 |
| Anaerotruncus_colihominis | 0.0832949 | 0.1328088 | 0.1742219 | 0.1492032 |
| Clostridium_asparagiforme | 0.0334723 | 0.0398848 | 0.0628373 | 0.0648395 |
| Firmicutes_bacterium_CAG_94 | −0.042677 | 0.1339602 | 0.0572215 | 0.025262 |
| Pseudoflavonifractor_sp_An184 | −0.034423 | 0.1133463 | 0.0346939 | 0.0263674 |
| Clostridium_leptum | −0.032007 | 0.1613224 | 0.0690155 | 0.0487026 |
| Bacteroides_fragilis | 0.0689512 | 0.0377413 | 0.0502686 | 0.0437603 |
| Clostridium_symbiosum | 0.043974 | 0.1619063 | 0.1615361 | 0.1358009 |
| Anaeromassilibacillus_sp_An250 | −0.023924 | 0.067891 | 0.0207633 | −0.002715 |

Table 5 (Part 1C): Spearman's correlations

| | | Meal_JJ_Hospi- | Meal_JJ_Hospi- |
|---|---|---|---|
| Profile | GlycA_0 | tal_meal_glucose_120_iauc | tal_meal_c-peptide_120_iauc |
| Positive/Negative | | | |
| Paraprevotella_xylaniphila | −0.086933 | −0.071254 | −0.080236 |
| Paraprevotella_clara | −0.089897 | −0.066741 | −0.066914 |
| Bacteroides_massiliensis | −0.114576 | −0.015901 | −0.072116 |
| Prevotella_copri | −0.140505 | −0.082477 | −0.069587 |
| Rothia_mucilaginosa | −0.049728 | −0.043752 | −0.091692 |
| Haemophilus_parainfluenzae | −0.170034 | −0.087716 | −0.15643 |
| Firmicutes_bacterium_CAG_95 | −0.167566 | −0.103633 | −0.133539 |
| Firmicutes_bacterium_CAG_170 | −0.167071 | −0.079309 | −0.090996 |
| Oscillibacter_sp_57_20 | −0.147928 | −0.034989 | −0.102806 |
| Bifidobacterium_animalis | −0.054494 | −0.051195 | −0.116422 |
| Sutterella_parvirubra | −0.025703 | −0.009435 | −0.023478 |
| Clostridium_sp_CAG_167 | −0.149194 | −0.064432 | −0.104643 |
| Veillonella_dispar | −0.115889 | −0.076653 | −0.166491 |
| Veillonella_infantium | −0.102008 | −0.065911 | −0.174745 |
| Roseburia_sp_CAG_471 | −0.067317 | −0.051611 | −0.060494 |
| Bacteroides_xylanisolvens | −0.063156 | −5.34E−04 | −0.037886 |
| Veillonella_atypica | −0.070093 | −0.021181 | −0.167937 |
| Lactobacillus_rogosae | −0.094088 | −0.030921 | −0.038951 |
| Roseburia_sp_CAG_309 | −0.087447 | −0.043901 | −0.098035 |
| Parabacteroides_goldsteinii | −0.043231 | −0.036805 | −0.052412 |
| Bacteroides_sp_CAG_144 | −0.006111 | −0.02081 | 0.0077327 |
| Veillonella_sp_T11011_6 | −0.079072 | −0.015592 | −0.128046 |
| Bacteroides_finegoldii | −0.0812 | −0.041295 | −0.007264 |
| Slackia_isoflavoniconvertens | −0.063776 | −0.038396 | −0.05858 |
| Roseburia_intestinalis | −0.047927 | −0.023791 | −0.017424 |
| Veillonella_parvula | −0.069688 | −0.074701 | −0.164268 |
| Coprococcus_eutactus | −0.118041 | −0.028689 | −0.070921 |
| Holdemanella_biformis | −0.08968 | −0.04699 | −0.069971 |
| Bacteroides_galacturonicus | −0.047475 | −0.038916 | −0.025058 |
| Veillonella_rogosae | −0.086747 | −0.038999 | −0.091881 |
| Bacteroides_intestinalis | −0.073762 | 0.0105848 | −0.044836 |
| Bacteroides_ovatus | 0.0223617 | −0.013678 | −0.035321 |
| Firmicutes_bacterium_CAG_238 | −0.064711 | −0.005139 | −0.06031 |
| Eubacterium_eligens | −0.152813 | −0.010302 | −0.026914 |
| Streptococcus_australis | 0.0238733 | 0.0306657 | −4.11E−04 |
| Desulfovibrio_piger | −0.045965 | −0.013907 | 0.0169344 |
| Oscillibacter_sp_PC13 | −0.130233 | −0.07369 | −0.078888 |
| Flavonifractor_sp_An100 | −0.011178 | −0.063097 | −0.110291 |
| Agathobaculum_butyriciproducens | −0.076296 | −0.057592 | −0.047558 |
| Coprocoecus_catus | −0.09057 | −0.04395 | −0.085806 |
| Alistipes_shahii | −0.03957 | −0.014801 | −0.041059 |
| Butyricimonas_synergistica | −0.028055 | 0.0051814 | −0.019979 |
| Bacteroides_salyersiae | −0.058731 | −0.073124 | −0.081623 |
| Ruminococcaceae_bacterium_D5 | −0.094866 | −5.33E−06 | −0.021369 |
| Ruminococcus_lactaris | −0.080063 | 0.0014464 | −0.054385 |
| Bacteroides_dorei | 0.0182885 | 0.010824 | 0.0084154 |
| Roseburia_hominis | −0.068508 | −0.071798 | −0.058119 |
| Lachnospira_pectinoschiza | −0.03805 | −0.013493 | 0.0373376 |
| Lactococcus_lactis | −0.027642 | 0.0105638 | −0.015395 |
| Streptococcus_parasanguinis | −0.057847 | 0.0091931 | −0.078574 |
| Bacteroides_clarus | −0.049393 | −0.051287 | −0.055358 |
| Firmicutes_bacterium_CAG_110 | −0.141447 | −0.069636 | −0.066537 |
| Collinsella_stercoris | 0.0160299 | −0.021805 | 0.0137512 |
| Roseburia_sp_CAG_182 | −0.158338 | −0.032236 | −0.088841 |
| Haemophilus_sp_HMSC71H05 | −0.095282 | −0.057339 | −0.100261 |
| Eubacterium_ramulus | −0.064953 | 0.0142874 | −0.030147 |
| Turicimonas_muris | −0.036987 | 0.0070131 | −0.055488 |
| Alistipes_indistinctus | −0.041308 | −0.070325 | −0.027055 |
| Methanobrevibacter_smithii | −0.083147 | −0.064873 | −0.030723 |
| Streptococcus_salivarius | −0.02992 | −0.010421 | −0.044821 |
| Faecalibacterium_prausnitzii | −0.117347 | −0.037334 | −0.123771 |
| Bacteroides_nordii | −0.093266 | 0.0316007 | 0.0064282 |
| Parabacteroides_merdae | −0.011089 | −0.016519 | 0.011991 |
| Actinomyces_odontolyticus | −0.041666 | −0.04054 | −0.091422 |
| Eubacterium_hallii | −0.090366 | −0.002057 | −0.061582 |
| Eubacterium_siraeum | −0.098469 | −0.056896 | −0.04975 |
| Intestinimonas_butyriciproducens | −0.048544 | −0.021979 | 0.0127415 |
| Butyricimonas_virosa | −0.0272 | 0.0285667 | 0.0087022 |
| Bacteroides_faecis | −0.028681 | 0.0053091 | 0.0101416 |
| Actinomyces_sp_ICM47 | 0.026482 | 0.0531545 | 0.0338132 |
| Romboutsia_ilealis | −0.137516 | −0.121208 | −0.103197 |
| Eubacterium_sp_CAG_180 | 0.0307549 | −0.030288 | −0.015274 |
| Gemella_sanguinis | 0.0947714 | 0.0917925 | 0.0295223 |
| Holdemania_filiformis | 0.0293698 | 0.0337035 | 0.0591484 |
| Bacteroides_vulgatus | 0.0240863 | 0.0691645 | 0.0535758 |
| Streptococcus_sp_A12 | 0.0280145 | 0.0285705 | 0.0496501 |
| Barnesiella_intestinihominis | −0.021585 | −0.002979 | 0.0104785 |
| Bacteroides_faecis_CAG_32 | 0.0155328 | 0.0255673 | 0.0155315 |
| Gemmiger_formicilis | −0.051416 | 0.0506661 | 0.0102215 |
| Roseburia_inulinivorans | 0.0786186 | −0.048195 | 0.0024353 |
| Anaerostipes_hadrus | −0.04869 | −0.030075 | 0.041231 |
| Dialister_invisus | 0.0211091 | −0.031226 | −0.020139 |
| Bifidobacterium_pseudocatenulatum | −0.032628 | −0.041074 | −0.032351 |
| Dorea_formicigenerans | 0.0571972 | −0.031037 | 0.0038407 |
| Firmicutes_bacterium_CAG_145 | 0.0354977 | −0.005614 | 0.0539236 |
| Intestinibacter_bartlettii | −0.042919 | −0.0437 | −0.114846 |
| Coprobacter_secundus | −0.06035 | 0.0300595 | −0.014877 |
| Parabacteroides_distasonis | 0.0655745 | 0.0443312 | 0.0567663 |
| Bacteroides_caccae | 0.0128504 | 7.12E−04 | 0.0442631 |
| [Collinsella]_massiliensis | −3.42E−04 | 0.0378777 | 0.0419651 |
| Olsenella_scatoligenes | −0.003925 | −0.0197 | −0.017384 |
| Ruminococcus_bromii | −0.010578 | −0.008389 | −9.71E−04 |
| Ruminococcus_callidus | −0.043032 | −0.02331 | −0.060435 |
| Fretibacterium_fastidiosum | −0.0758 | −0.024925 | −0.078856 |
| Dorea_longicatena | −0.08316 | −0.059771 | −0.026943 |
| Eubacterium_sp_CAG_251 | −0.038392 | −0.088055 | −0.054694 |
| Streptococcus_mitis | 0.0324974 | 0.0696096 | 0.0150035 |
| Bacteroides_cellulosilyticus | −0.039126 | 0.0021958 | 0.0311254 |
| Clostridium_sp_CAG_253 | −0.049477 | 0.0184603 | −0.045745 |
| Parasutterella_excrementihominis | −0.058061 | −0.037331 | −0.078345 |
| Bacteroides_thetaiotaomicron | 0.0272202 | 0.0357357 | 0.0168995 |
| Oscillibacter_sp_CAG_241 | −0.090353 | −0.056513 | −0.021935 |
| Coprobacter_fastidiosus | −8.12E−04 | −0.024519 | 0.039971 |
| Streptococcus_thermophilus | −0.004636 | 0.0185484 | −0.001556 |
| Bacteroides_stercoris | −0.020413 | −0.034823 | −0.022814 |
| Lawsonibacter_asaccharolyticus | −0.017152 | 0.002478 | −0.016317 |
| Bacteroides_eggerthii | −0.040889 | −0.06964 | −0.044029 |
| Alistipes_putredinis | 0.0228343 | 0.0080662 | 0.0210224 |
| Victivallis_vadensis | −0.025666 | −0.025079 | −0.059188 |
| Collinsella_aerofaciens | 0.0267451 | −0.028246 | 0.0306431 |
| Eubacterium_sp_CAG_38 | −0.00912 | −0.036533 | −0.072751 |
| Coprococcus_comes | −0.055348 | −0.027873 | 0.0065398 |
| Odoribacter_splanchnicus | −0.035408 | −0.001753 | 0.01413 |
| Proteobacteria_bacterium_CAG_139 | −0.015731 | 0.0167621 | −0.024732 |
| Pseudoflavonifractor_capillosus | 0.0484775 | −0.029142 | 0.007875 |
| Enorma_massiliensis | −0.038319 | −0.013461 | 0.0022521 |
| Clostridium_disporicum | −0.102789 | −0.059275 | −0.119528 |
| Ruminococcus_torques | −0.074981 | −0.004471 | −0.018873 |
| Alistipes_onderdonkii | −0.077928 | −0.013399 | −0.017462 |
| Turicibacter_sanguinis | −0.101523 | −0.014954 | −0.096447 |
| Akkermansia_muciniphila | −0.0323 | −0.036321 | 0.0227365 |
| Flavonifractor_plautii | 0.1537716 | 0.1020084 | 0.1355756 |
| Blautia_wexlerae | 0.0283155 | 0.0325512 | 0.0457522 |
| Bifidobacterium_adolescentis | −0.021073 | −0.088316 | −0.048055 |
| Bifidobacterium_longum | 0.0230829 | −0.016128 | −0.008382 |
| Parabacteroides_johnsonii | −0.010163 | 0.0200014 | 0.0353648 |
| Phascolarctobacterium_faecium | 0.0154349 | 0.0120618 | 0.0039326 |
| Eubacterium_sp_OM08_24 | −0.004505 | −0.008563 | −0.033628 |
| Eisenbergiella_massiliensis | 0.0490281 | 0.098919 | 0.1461804 |
| Clostridium_sp_CAG_242 | −0.016756 | −0.046456 | −0.049046 |
| Roseburia_faecis | 0.0037086 | −0.016635 | 0.012864 |
| Bacteroides_uniformis | 0.0555278 | 0.0486811 | 0.0080599 |
| Bifidobacterium_catenulatum | 0.0514963 | −0.02112 | −0.042528 |
| Enterorhabdus_caecimuris | −0.040602 | 0.0432937 | 0.0390146 |
| Firmicutes_bacterium_CAG_83 | 0.0297935 | 0.0030229 | 2.18E−04 |
| Eubacterium_rectale | 0.0848493 | 0.0089852 | −0.005467 |
| Collinsella_intestinalis | 0.0884776 | 0.0013261 | 0.0382442 |
| Blautia_hydrogenotrophica | 0.0688664 | 0.0270208 | 0.1159136 |
| Ruminococcus_gnavus | 0.1614136 | 0.0884851 | 0.1569871 |
| Blautia_obeum | 0.0208183 | 0.0522464 | 0.0240848 |
| Dielma_fastidiosa | 0.0818795 | 0.0578418 | 0.1295689 |
| Hungatella_hathewayi | 0.0444908 | 0.0506043 | 0.0351127 |
| Harryflintia_acetispora | 0.0154901 | −0.044983 | 0.0019713 |
| Bilophila_wadsworthia | 0.0279119 | 0.0093713 | 0.0534629 |
| Eggerthella_lenta | 0.1348898 | 0.1001074 | 0.1020255 |
| Monoglobus_pectinilyticus | 0.0311747 | 0.0024397 | −0.005115 |
| Bifidobacterium_bifidum | 0.0411526 | −0.023545 | −0.002843 |
| Fusicatenibacter_saccharivorans | −0.005745 | −0.008119 | −0.027894 |
| Ruthenibacterium_lactatiformans | 0.0730229 | 0.0106497 | 0.0421003 |
| Ruminococcus_bicirculans | 0.0429625 | −0.024064 | 0.0038682 |
| Alistipes_finegoldii | 0.0254674 | 0.0057007 | 0.0233008 |
| Eubacterium_sp_CAG_274 | 0.04118 | −0.005485 | 0.0185846 |
| Eubacterium_ventriosum | −0.014502 | −0.021231 | −0.058672 |
| Clostridium_spiroforme | 0.0876612 | 0.0408773 | 0.0775602 |
| Clostridium_saccharolyticum | 0.095208 | 0.0212946 | 0.064275 |
| Gordonibacter_pamelaeae | 0.0733212 | 0.0378865 | 0.0622312 |
| Alistipes_inops | −0.008755 | −0.03059 | 0.0224319 |
| Clostridium_lavalense | 0.0899339 | 0.0341244 | 0.1049723 |
| Clostridium_sp_CAG_58 | 0.0890606 | 0.0503458 | 0.1174403 |
| Clostridium_bolteae_CAG_59 | 0.1522146 | 0.0698157 | 0.0904404 |
| Adlercreutzia_equolifaciens | −0.040549 | 0.0323553 | 0.0413344 |
| Escherichia_coli | 0.1021338 | 0.0344255 | 0.0381649 |
| Ruminococcaceae_bacterium_D16 | 0.0211314 | −0.065448 | −0.030456 |
| Eisenbergiella_tayi | 0.0322273 | 0.028456 | 0.0606373 |
| Clostridium_citroniae | 0.1097563 | 0.0590924 | 0.1371222 |
| Clostridium_bolteae | 0.167541 | 0.073837 | 0.162996 |
| Asaccharobacter_celatus | −0.043063 | 0.0329906 | 0.0363072 |
| Clostridium_innocuum | 0.1384059 | 0.0697914 | 0.1000697 |
| Anaerotruncus_colihominis | 0.1291492 | 0.0474943 | 0.1540926 |
| Clostridium_asparagiforme | 0.0841046 | 0.1111808 | 0.1177005 |
| Firmicutes_bacterium_CAG_94 | 0.0501755 | −0.005726 | −0.01406 |
| Pseudoflavonifractor_sp_An184 | 0.0218252 | −0.049185 | −0.028348 |
| Clostridium_leptum | 0.05821 | −0.047527 | 0.0113558 |
| Bacteroides_fragilis | 0.1285656 | 0.0152987 | 0.0590632 |
| Clostridium_symbiosum | 0.1579175 | 0.0934586 | 0.1797507 |
| Anaeromassilibacillus_sp_An250 | 0.0241241 | −0.07611 | −0.026951 |

Table 5 (Part 1C): Spearman's correlations

| Profile | Meal_JJ_Hospital_meal_trig_360_iauc | GlycA_360 | VLDL_size_360 |
|---|---|---|---|
| Positive/Negative | Negative | Negative | Negative |
| Paraprevotella_xylaniphila | 0.0367052 | −0.074921 | −0.045707 |
| Paraprevotella_clara | 0.0241246 | −0.080957 | −0.038618 |
| Bacteroides_massiliensis | −0.009695 | −0.08963 | −0.039583 |
| Prevotella_copri | −0.029273 | −0.106952 | −0.063894 |
| Rothia_mucilaginosa | −0.064272 | −0.064885 | −0.093247 |
| Haemophilus_parainfluenzae | −0.080018 | −0.164744 | −0.133127 |
| Firmicutes_bacterium_CAG_95 | −0.114761 | −0.13961 | −0.1946 |
| Firmicutes_bacterium_CAG_170 | −0.026293 | −0.139838 | −0.088826 |
| Oscillibacter_sp_57_20 | −0.111679 | −0.133472 | −0.142893 |
| Bifidobacterium_animalis | −0.030158 | −0.03199 | −0.090056 |
| Sutterella_parvirubra | 0.0699018 | −0.009205 | 0.0261613 |
| Clostridium_sp_CAG_167 | −0.067484 | −0.129348 | −0.072992 |
| Veillonella_dispar | −0.075295 | −0.09358 | −0.06548 |
| Veillonella_infantium | −0.069708 | −0.100839 | −0.082198 |
| Roseburia_sp_CAG_471 | 0.0308403 | −0.08432 | −0.02578 |
| Bacteroides_xylanisolvens | −0.031592 | −0.061086 | −0.005429 |
| Veillonella_atypica | −0.076776 | −0.063739 | −0.080356 |
| Lactobacillus_rogosae | 0.0061446 | −0.071947 | −0.033117 |
| Roseburia_sp_CAG_309 | −0.041317 | −0.098626 | −0.076676 |
| Parabacteroides_goldsteinii | −0.020958 | −0.051205 | −0.009636 |
| Bacteroides_sp_CAG_144 | −0.042564 | −0.018924 | −0.012208 |
| Veillonella_sp_T11011_6 | −0.025021 | −0.08775 | −0.064489 |
| Bacteroides_finegoldii | 0.0535484 | −0.069082 | 0.0102812 |
| Slackia_isoflavoniconvertens | 0.0052906 | −0.038788 | −0.052683 |
| Roseburia_intestinalis | 0.0666048 | −0.057726 | 0.0201988 |
| Veillonella_parvula | −0.124305 | −0.069545 | −0.099129 |
| Coprococcus_eutactus | −0.068506 | −0.119009 | −0.069094 |
| Holdemanella_biformis | 0.0311195 | −0.074971 | −0.051827 |
| Bacteroides_galacturonicus | 0.051197 | −0.051912 | 0.0239311 |
| Veillonella_rogosae | −0.058924 | −0.068334 | −0.050761 |
| Bacteroides_intestinalis | 0.0048422 | −0.075938 | −0.024586 |
| Bacteroides_ovatus | −0.007436 | 0.0223488 | 0.0190406 |
| Firmicutes_bacterium_CAG_238 | −0.083334 | −0.037838 | −0.091877 |
| Eubacterium_eligens | −0.020407 | −0.136094 | −0.076053 |
| Streptococcus_australis | −0.003747 | 0.0130643 | −0.001605 |
| Desulfovibrio_piger | −0.001559 | −0.022263 | 0.0077738 |
| Oscillibacter_sp_PC13 | −0.054261 | −0.117191 | −0.105016 |
| Flavonifractor_sp_An100 | −0.084157 | −0.039657 | −0.05992 |
| Agathobaculum_butyriciproducens | 0.0307215 | −0.060566 | 0.0119894 |
| Coprocoecus_catus | −0.031314 | −0.079431 | −0.046724 |
| Alistipes_shahii | −0.030152 | −0.039545 | −0.076286 |
| Butyricimonas_synergistica | −0.041469 | −0.01388 | −0.0782 |
| Bacteroides_salyersiae | −0.081722 | −0.047563 | −0.064909 |
| Ruminococcaceae_bacterium_D5 | −0.082053 | −0.08442 | −0.116667 |
| Ruminococcus_lactaris | −0.013667 | −0.092879 | −0.077171 |
| Bacteroides_dorei | −0.035323 | −0.005518 | −0.010526 |
| Roseburia_hominis | −0.068143 | −0.071639 | −0.05234 |
| Lachnospira_pectinoschiza | −0.003212 | −0.035187 | 0.0080338 |
| Lactococcus_lactis | −0.032329 | −0.029686 | −0.014285 |
| Streptococcus_parasanguinis | −0.031188 | −0.062448 | −0.061177 |
| Bacteroides_clarus | −0.064793 | −0.06465 | −0.049668 |
| Firmicutes_bacterium_CAG_110 | −0.099099 | −0.100102 | −0.140911 |
| Collinsella_stercoris | 0.0452796 | 0.0307544 | 0.0491376 |
| Roseburia_sp_CAG_182 | −0.048807 | −0.142511 | −0.09037 |
| Haemophilus_sp_HMSC71H05 | −0.037404 | −0.100779 | −0.042073 |
| Eubacterium_ramulus | 0.0501281 | −0.064512 | −0.015906 |
| Turicimonas_muris | −0.091933 | −0.043308 | −0.057572 |
| Alistipes_indistinctus | 0.0095429 | −0.032929 | 0.0322876 |
| Methanobrevibacter_smithii | −0.033568 | −0.0495 | −0.021725 |
| Streptococcus_salivarius | −0.007108 | −0.028232 | −0.023467 |
| Faecalibacterium_prausnitzii | −0.067695 | −0.13145 | −0.06842 |
| Bacteroides_nordii | −0.007223 | −0.080701 | −0.027217 |
| Parabacteroides_merdae | 0.0349057 | −0.026509 | −0.05627 |
| Actinomyces_odontolyticus | −0.015929 | −0.035327 | −0.026579 |
| Eubacterium_hallii | −0.038648 | −0.075953 | −0.0291 |
| Eubacterium_siraeum | −0.040509 | −0.080591 | −0.062824 |
| Intestinimonas_butyriciproducens | −0.070517 | −0.081854 | −0.080002 |
| Butyricimonas_virosa | −0.007084 | −0.019962 | −0.067817 |
| Bacteroides_faecis | 0.0068678 | 0.016932 | −0.049271 |
| Actinomyces_sp_ICM47 | 0.0669282 | 0.0357105 | 0.0615622 |
| Romboutsia_ilealis | −0.050659 | −0.114173 | −0.071763 |
| Eubacterium_sp_CAG_180 | −0.009398 | 0.0427643 | 0.0076368 |
| Gemella_sanguinis | 0.0277373 | 0.0840453 | 0.070013 |
| Holdemania_filiformis | 0.0155863 | 0.0339077 | 0.0343608 |
| Bacteroides_vulgatus | 0.0683589 | 0.0178979 | 0.0600489 |
| Streptococcus_sp_A12 | −0.031841 | 0.0357728 | −7.47E−04 |
| Barnesiella_intestinihominis | −0.059405 | −0.03615 | −0.046387 |
| Bacteroides_faecis_CAG_32 | 0.0119311 | 0.0424992 | −0.03364 |
| Gemmiger_formicilis | −0.005116 | −0.037882 | −0.021683 |
| Roseburia_inulinivorans | 0.0455361 | 0.0705316 | 0.0839026 |
| Anaerostipes_hadrus | 0.0382609 | −0.040968 | 0.0235406 |
| Dialister_invisus | −0.123963 | 0.0240594 | −0.030702 |
| Bifidobacterium_pseudocatenulatum | 0.0172452 | −0.039699 | 0.0159214 |
| Dorea_formicigenerans | 0.0726064 | 0.0420404 | 0.0538901 |
| Firmicutes_bacterium_CAG_145 | 0.0461819 | 0.0103975 | 0.0349752 |
| Intestinibacter_bartlettii | −0.096863 | −0.03157 | −0.058315 |
| Coprobacter_secundus | −0.080462 | −0.057359 | −0.115224 |
| Parabacteroides_distasonis | 0.0434319 | 0.0359767 | 0.0547945 |
| Bacteroides_caccae | 0.0221511 | 0.0178342 | −0.001384 |
| [Collinsella]_massiliensis | −0.004579 | 0.0208656 | 0.0033983 |
| Olsenella_scatoligenes | 0.0158654 | 0.0377559 | −0.003804 |
| Ruminococcus_bromii | −0.059204 | −0.002773 | −0.040389 |
| Ruminococcus_callidus | −0.003663 | −0.010873 | −0.0263 |
| Fretibacterium_fastidiosum | −0.085445 | −0.071081 | −0.098163 |
| Dorea_longicatena | 0.0020641 | −0.06444 | −0.007113 |
| Eubacterium_sp_CAG_251 | −0.02333 | −0.042417 | 4.51E−04 |
| Streptococcus_mitis | −0.014229 | 0.0181445 | 0.0274397 |
| Bacteroides_cellulosilyticus | −0.03512 | −0.060575 | −0.057455 |
| Clostridium_sp_CAG_253 | −0.033795 | −0.047574 | −0.065921 |
| Parasutterella_excrementihominis | −0.083735 | −0.06604 | −0.041406 |
| Bacteroides_thetaiotaomicron | 0.030795 | −0.03272 | 0.0387553 |
| Oscillibacter_sp_CAG_241 | −8.15E−05 | −0.054048 | −0.041056 |
| Coprobacter_fastidiosus | 0.0681773 | 0.0010733 | 0.0539528 |
| Streptococcus_thermophilus | −0.042272 | −0.011392 | −0.056903 |
| Bacteroides_stercoris | 0.0057978 | 0.0033346 | −0.025825 |
| Lawsonibacter_asaccharolyticus | 0.0046579 | −0.062575 | 0.0236571 |
| Bacteroides_eggerthii | 0.0121319 | −0.050021 | 0.0018594 |
| Alistipes_putredinis | −0.040404 | 9.07E−04 | −0.037827 |
| Victivallis_vadensis | −0.061734 | 0.0074873 | −0.037881 |
| Collinsella_aerofaciens | 0.0326815 | 0.028144 | 0.0200454 |
| Eubacterium_sp_CAG_38 | 0.0043709 | −0.010262 | −0.008678 |
| Coprococcus_comes | 0.0302529 | −0.059614 | −0.002985 |
| Odoribacter_splanchnicus | −0.069794 | −0.048859 | −0.083757 |
| Proteobacteria_bacterium_CAG_139 | −0.092729 | −0.029834 | −0.041037 |
| Pseudoflavonifractor_capillosus | −0.03379 | 0.0125696 | 0.0158508 |
| Enorma_massiliensis | −0.032461 | −1.63E−04 | −0.023526 |
| Clostridium_disporicum | −0.121897 | −0.0763 | −0.113265 |
| Ruminococcus_torques | 0.0810557 | −0.039186 | 0.0536728 |
| Alistipes_onderdonkii | −0.005954 | −0.066661 | −0.074192 |
| Turicibacter_sanguinis | −0.16166 | −0.109741 | −0.163634 |
| Akkermansia_muciniphila | −0.069864 | −0.02992 | −0.064975 |
| Flavonifractor_plautii | 0.0754037 | 0.1084411 | 0.1381358 |
| Blautia_wexlerae | 0.0610585 | 0.0345754 | 0.0590841 |
| Bifidobacterium_adolescentis | −0.037425 | −0.010368 | 0.0068379 |
| Bifidobacterium_longum | −0.040747 | 0.0189439 | 0.0223041 |
| Parabacteroides_johnsonii | 0.0627814 | −0.008096 | 0.0079065 |
| Phascolarctobacterium_faecium | 0.0374975 | 0.0095266 | −0.03079 |
| Eubacterium_sp_OM08_24 | 0.0141451 | 0.0101123 | 0.0172921 |
| Eisenbergiella_massiliensis | −0.015082 | 0.0258854 | 0.0279678 |
| Clostridium_sp_CAG_242 | −0.044749 | −0.038486 | −0.096247 |
| Roseburia_faecis | −0.002434 | 0.0254573 | 0.0180427 |
| Bacteroides_uniformis | 0.055667 | 0.0420585 | 0.0858093 |
| Bifidobacterium_catenulatum | −0.034159 | 0.0479775 | 0.0167962 |
| Enterorhabdus_caecimuris | −0.026092 | −0.042296 | 0.0103076 |
| Firmicutes_bacterium_CAG_83 | −0.013565 | 0.0158811 | −0.059509 |
| Eubacterium_rectale | 0.0011488 | 0.066842 | 0.0520979 |
| Collinsella_intestinalis | 0.0589562 | 0.072168 | 0.083634 |
| Blautia_hydrogenotrophica | 0.0955704 | 0.0585118 | 0.087204 |
| Ruminococcus_gnavus | 0.1005774 | 0.1533818 | 0.1445508 |
| Blautia_obeum | 0.0505935 | 0.0353071 | 0.0623255 |
| Dielma_fastidiosa | 0.078738 | 0.0710061 | 0.0560768 |
| Hungatella_hathewayi | 0.0588472 | 0.0605132 | 0.0697622 |
| Harryflintia_acetispora | −0.031336 | −0.003374 | 0.0183292 |
| Bilophila_wadsworthia | −0.025051 | 0.0220127 | −0.014811 |
| Eggerthella_lenta | 0.0120437 | 0.1216403 | 0.0917042 |
| Monoglobus_pectinilyticus | 0.033108 | 0.0248928 | 0.0296633 |
| Bifidobacterium_bifidum | −0.023872 | 0.0329294 | 0.0435359 |
| Fusicatenibacter_saccharivorans | 0.0463316 | −0.006862 | 0.0735136 |
| Ruthenibacterium_lactatiformans | 0.015926 | 0.0528383 | 0.057308 |
| Ruminococcus_bicirculans | −0.053975 | 0.0311903 | −0.013777 |
| Alistipes_finegoldii | −0.089616 | −0.003298 | −0.053372 |
| Eubacterium_sp_CAG_274 | −0.008909 | 0.0371697 | 2.91E−04 |
| Eubacterium_ventriosum | 0.0167906 | 0.0076059 | 0.0955942 |
| Clostridium_spiroforme | 0.0458304 | 0.0668437 | 0.1210726 |
| Clostridium_saccharolyticum | −0.011155 | 0.0706687 | −0.00562 |
| Gordonibacter_pamelaeae | −0.073468 | 0.0451409 | 0.0293856 |
| Alistipes_inops | −0.012746 | −0.012193 | −0.048336 |
| Clostridium_lavalense | 0.0303449 | 0.0760153 | 0.071628 |
| Clostridium_sp_CAG_58 | 0.0521229 | 0.0726683 | 0.0932912 |
| Clostridium_bolteae_CAG_59 | 0.072894 | 0.1533151 | 0.1130611 |
| Adlercreutzia_equolifaciens | −0.028912 | −0.048718 | 0.0180517 |
| Escherichia_coli | 0.0045039 | 0.0737554 | 0.040412 |
| Ruminococcaceae_bacterium_D16 | −0.035566 | 6.79E−04 | −0.003799 |
| Eisenbergiella_tayi | 0.0064031 | 0.015539 | 0.0347513 |
| Clostridium_citroniae | 0.0388651 | 0.0804812 | 0.084774 |
| Clostridium_bolteae | 0.1144535 | 0.1532242 | 0.1701863 |
| Asaccharobacter_celatus | −0.049302 | −0.054365 | −0.018143 |
| Clostridium_innocuum | 0.0663685 | 0.1210644 | 0.1329868 |
| Anaerotruncus_colihominis | 0.0728038 | 0.1100322 | 0.1347263 |
| Clostridium_asparagiforme | 0.0144552 | 0.0903716 | 0.0727577 |
| Firmicutes_bacterium_CAG_94 | −0.07219 | 0.0285645 | 0.009476 |
| Pseudoflavonifractor_sp_An184 | −0.001755 | 0.0277326 | 0.0181648 |
| Clostridium_leptum | 0.0116638 | 0.0427034 | 0.0241528 |
| Bacteroides_fragilis | 0.0644633 | 0.0997092 | 0.0404366 |
| Clostridium_symbiosum | 0.0439888 | 0.1320768 | 0.1214708 |
| Anaeromassilibacillus_sp_An250 | −0.073755 | −0.01205 | −0.041868 |

Table 5 (Part 2A). Ranks.

| Profile | quicki_score | amed_score | HFD | hei_score |
|---|---|---|---|---|
| Category | Personal | Habitual | Habitual | Habitual |
| | | Diet | Diet | Diet |
| [Collinsella]_massiliensis | 90 | 149 | 159 | 133 |
| Actinomyces_odontolyticus | 64 | 98 | 56 | 80 |
| Actinomyces_sp_ICM47 | 70 | 109 | 149 | 86 |
| Adlercreutzia_equolifaciens | 161 | 78 | 135 | 49 |
| Agathobaculum_butyriciproducens | 39 | 1 | 22 | 1 |
| Akkermansia_muciniphila | 121 | 88 | 140 | 115 |
| Alistipes_fineqoldii | 151 | 130 | 84 | 138 |
| Alistipes_indistinctus | 58 | 84 | 82 | 125 |
| Alistipes_inops | 157 | 62 | 110 | 92 |
| Alistipes_onderdonkii | 119 | 76 | 94 | 40 |
| Alistipes_putredinis | 108 | 136 | 144 | 161 |
| Alistipes_shahii | 41 | 79 | 24 | 95 |
| Anaeromassilibacillus_sp_An250 | 176 | 166 | 170 | 175 |
| Anaerostipes_hadrus | 81 | 9 | 18 | 9 |
| Anaerotruncus_colihominis | 169 | 176 | 152 | 171 |
| Asaccharobacter_celatus | 167 | 82 | 138 | 62 |
| Bacteroides_caccae | 89 | 135 | 118 | 159 |
| Bacteroides_cellulosilyticus | 98 | 57 | 103 | 35 |
| Bacteroides_clarus | 51 | 73 | 113 | 53 |
| Bacteroides_dorei | 46 | 80 | 58 | 28 |
| Bacteroides_eggerthii | 107 | 35 | 26 | 59 |
| Bacteroides_faecis | 69 | 66 | 93 | 103 |
| Bacteroides_faecis_CAG_32 | 78 | 28 | 77 | 87 |
| Bacteroides_finegoldii | 23 | 100 | 98 | 43 |
| Bacteroides_fragilis | 174 | 129 | 127 | 102 |
| Bacteroides_galacturonicus | 29 | 32 | 41 | 64 |
| Bacteroides_intestinalis | 31 | 67 | 48 | 81 |
| Bacteroides_massiliensis | 3 | 18 | 14 | 32 |
| Bacteroides_nordii | 62 | 21 | 4 | 16 |
| Bacteroides_ovatus | 32 | 27 | 27 | 27 |
| Bacteroides_salyersiae | 43 | 110 | 69 | 119 |
| Bacteroides_sp_CAG_144 | 21 | 70 | 171 | 111 |
| Bacteroides_stercoris | 105 | 97 | 101 | 110 |
| Bacteroides_thetaiotaomicron | 101 | 75 | 79 | 39 |
| Bacteroides_uniformis | 132 | 126 | 104 | 120 |
| Bacteroides_vulgatus | 75 | 87 | 125 | 93 |
| Bacteroides_xylanisolvens | 16 | 83 | 19 | 52 |
| Barnesiella_intestinihominis | 77 | 123 | 90 | 112 |
| Bifidobacterium_adolescentis | 124 | 63 | 34 | 83 |
| Bifidobacterium_animalis | 10 | 12 | 7 | 2 |
| Bifidobacterium_bifidum | 147 | 162 | 124 | 168 |
| Bifidobacterium_catenulatum | 133 | 138 | 147 | 169 |
| Bifidobacterium_longum | 125 | 134 | 169 | 145 |
| Bifidobacterium_pseudocatenulatum | 83 | 33 | 47 | 30 |
| Bilophila_wadsworthia | 144 | 137 | 155 | 164 |
| Blautia_hydrogenotrophica | 138 | 157 | 121 | 139 |
| Blautia_obeum | 140 | 112 | 123 | 98 |
| Blautia_wexlerae | 123 | 37 | 30 | 48 |
| Butyricimonas_synergistica | 42 | 120 | 150 | 79 |
| Butyricimonas_virosa | 68 | 131 | 148 | 121 |
| Clostridium_asparagiforme | 170 | 127 | 51 | 88 |
| Clostridium_bolteae | 166 | 170 | 132 | 158 |
| Clostridium_bolteae_CAG_59 | 160 | 164 | 85 | 141 |
| Clostridium_citroniae | 165 | 147 | 28 | 116 |
| Clostridium_disporicum | 117 | 107 | 116 | 134 |
| Clostridium_innocuum | 168 | 173 | 70 | 167 |
| Clostridium_lavalense | 158 | 151 | 60 | 106 |
| Clostridium_leptum | 173 | 161 | 168 | 176 |
| Clostridium_saccharolyticum | 155 | 156 | 162 | 152 |
| Clostridium_sp_CAG_167 | 12 | 4 | 33 | 5 |
| Clostridium_sp_CAG_242 | 130 | 122 | 100 | 63 |
| Clostridium_sp_CAG_253 | 99 | 89 | 81 | 23 |
| Clostridium_sp_CAG_58 | 159 | 146 | 142 | 151 |
| Clostridium_spiroforme | 154 | 152 | 174 | 155 |
| Clostridium_symbiosum | 175 | 172 | 117 | 165 |
| Collinsella_aerofaciens | 110 | 118 | 160 | 123 |
| Collinsella_intestinalis | 137 | 154 | 143 | 153 |
| Collinsella_stercoris | 53 | 61 | 154 | 71 |
| Coprobacter_fastidiosus | 103 | 104 | 157 | 132 |
| Coprobacter_secundus | 87 | 103 | 91 | 100 |
| Coprococcus_catus | 40 | 17 | 114 | 33 |
| Coprococcus_comes | 112 | 153 | 156 | 136 |
| Coprococcus_eutactus | 27 | 56 | 50 | 17 |
| Desulfovibrio_piger | 36 | 108 | 66 | 104 |
| Dialister_invisus | 82 | 60 | 38 | 99 |
| Dielma_fastidiosa | 141 | 150 | 80 | 117 |
| Dorea_formicigenerans | 84 | 22 | 20 | 41 |
| Dorea_longicatena | 95 | 102 | 133 | 127 |
| Eggerthella_lenta | 145 | 163 | 131 | 122 |
| Eisenbergiella_massiliensis | 129 | 159 | 62 | 131 |
| Eisenbergiella_tayi | 164 | 160 | 151 | 148 |
| Enorma_massiliensis | 116 | 116 | 173 | 101 |
| Enterorhabdus_caecimuris | 134 | 92 | 128 | 56 |
| Escherichia_coli | 162 | 169 | 141 | 154 |
| Eubacterium_eligens | 34 | 8 | 16 | 8 |
| Eubacterium_hallii | 65 | 19 | 53 | 15 |
| Eubacterium_ramulus | 56 | 44 | 63 | 74 |
| Eubacterium_rectale | 136 | 96 | 67 | 72 |
| Eubacterium_siraeum | 66 | 121 | 55 | 140 |
| Eubacterium_sp_CAG_180 | 72 | 128 | 145 | 126 |
| Eubacterium_sp_CAG_251 | 96 | 53 | 122 | 69 |
| Eubacterium_sp_CAG_274 | 152 | 65 | 29 | 45 |
| Eubacterium_sp_CAG_38 | 111 | 30 | 83 | 22 |
| Eubacterium_sp_OM08_24 | 128 | 101 | 102 | 85 |
| Eubacterium_ventriosum | 153 | 140 | 111 | 143 |
| Faecalibacterium_prausnitzii | 61 | 10 | 61 | 18 |
| Firmicutes_bacterium_CAG_110 | 52 | 105 | 139 | 150 |
| Firmicutes_bacterium_CAG_145 | 85 | 142 | 172 | 137 |
| Firmicutes_bacterium_CAG_170 | 8 | 23 | 23 | 29 |
| Firmicutes_bacterium_CAG_238 | 33 | 25 | 32 | 51 |
| Firmicutes_bacterium_CAG_83 | 135 | 85 | 73 | 73 |
| Firmicutes_bacterium_CAG_94 | 171 | 175 | 176 | 170 |
| Firmicutes_bacterium_CAG_95 | 7 | 2 | 45 | 19 |
| Flavonifractor_plautii | 122 | 174 | 165 | 173 |
| Flavonifractor_sp_An100 | 38 | 81 | 161 | 84 |
| Fretibacterium_fastidiosum | 94 | 49 | 68 | 67 |
| Fusicatenibacter_saccharivorans | 148 | 55 | 39 | 50 |
| Gemella_sanguinis | 73 | 139 | 146 | 124 |
| Gemmiger_formicilis | 79 | 114 | 78 | 82 |
| Gordonibacter_pamelaeae | 156 | 145 | 96 | 105 |
| Haemophilus_parainfluenzae | 6 | 15 | 1 | 12 |
| Haemophilus_sp_HMSC71H05 | 55 | 43 | 11 | 20 |
| Harryflintia_acetispora | 143 | 155 | 107 | 157 |
| Holdemanella_biformis | 28 | 52 | 87 | 57 |
| Holdemania_filiformis | 74 | 141 | 106 | 163 |
| Hungatella_hathewayi | 142 | 158 | 49 | 130 |
| Intestinibacter_bartlettii | 86 | 115 | 92 | 135 |
| Intestinimonas_butyriciproducens | 67 | 106 | 44 | 97 |
| Lachnospira_pectinoschiza | 48 | 47 | 65 | 114 |
| Lactobacillus_rogosae | 18 | 20 | 36 | 77 |
| Lactococcus_lactis | 49 | 48 | 105 | 107 |
| Lawsonibacter_asaccharolyticus | 106 | 144 | 175 | 118 |
| Methanobrevibacter_smithii | 59 | 90 | 97 | 129 |
| Monoglobus_pectinilyticus | 146 | 45 | 35 | 46 |
| Odoribacter_splanchnicus | 113 | 119 | 43 | 146 |
| Olsenella_scatoligenes | 91 | 74 | 52 | 25 |
| Oscillibacter_sp_57_20 | 9 | 5 | 2 | 4 |
| Oscillibacter_sp_CAG_241 | 102 | 99 | 129 | 149 |
| Oscillibacter_sp_PC13 | 37 | 13 | 46 | 38 |
| Parabacteroides_distasonis | 88 | 94 | 126 | 90 |
| Parabacteroides_goldsteinii | 20 | 29 | 74 | 54 |
| Parabacteroides_johnsonii | 126 | 58 | 54 | 34 |
| Parabacteroides_merdae | 63 | 148 | 167 | 162 |
| Paraprevotella_clara | 2 | 42 | 72 | 78 |
| Paraprevotella_xylaniphila | 1 | 26 | 76 | 66 |
| Parasutterella_excrementihominis | 100 | 16 | 95 | 42 |
| Phascolarctobacterium_faecium | 127 | 77 | 59 | 94 |
| Prevotella_copri | 4 | 38 | 40 | 24 |
| Proteobacteria_bacterium_CAG_139 | 114 | 54 | 99 | 60 |
| Pseudoflavonifractor_capillosus | 115 | 171 | 166 | 144 |
| Pseudoflavonifractor_sp_An184 | 172 | 167 | 164 | 174 |
| Romboutsia_ilealis | 71 | 14 | 6 | 37 |
| Roseburia_faecis | 131 | 64 | 25 | 65 |
| Roseburia_hominis | 47 | 6 | 64 | 6 |
| Roseburia_intestinalis | 25 | 93 | 57 | 61 |
| Roseburia_inulinivorans | 80 | 111 | 153 | 142 |
| Roseburia_sp_CAG_182 | 54 | 3 | 3 | 3 |
| Roseburia_sp_CAG_309 | 19 | 59 | 136 | 55 |
| Roseburia_sp_CAG_471 | 15 | 11 | 37 | 31 |
| Rothia_mucilaginosa | 5 | 39 | 109 | 26 |
| Ruminococcaceae_bacterium_D16 | 163 | 133 | 134 | 160 |
| Ruminococcaceae_bacterium_D5 | 44 | 31 | 10 | 91 |
| Ruminococcus_bicirculans | 150 | 95 | 115 | 108 |
| Ruminococcus_bromii | 92 | 125 | 130 | 128 |
| Ruminococcus_callidus | 93 | 124 | 42 | 96 |
| Ruminococcus_gnavus | 139 | 165 | 88 | 147 |
| Ruminococcus_lactaris | 45 | 7 | 120 | 7 |
| Ruminococcus_torgues | 118 | 143 | 137 | 156 |
| Ruthenibacterium_lactatiformans | 149 | 168 | 158 | 172 |
| Slackia_isoflavoniconvertens | 24 | 34 | 89 | 44 |
| Streptococcus_australis | 35 | 86 | 15 | 75 |
| Streptococcus_mitis | 97 | 132 | 163 | 166 |
| Streptococcus_parasanguinis | 50 | 113 | 119 | 109 |
| Streptococcus_salivarius | 60 | 117 | 112 | 89 |
| Streptococcus_sp_A12 | 76 | 68 | 17 | 58 |
| Streptococcus_thermophilus | 104 | 46 | 108 | 47 |
| Sutterella_parvirubra | 11 | 51 | 75 | 76 |
| Turicibacter_sanguinis | 120 | 69 | 31 | 70 |
| Turicimonas_muris | 57 | 91 | 71 | 68 |
| Veillonella_atypica | 17 | 41 | 21 | 14 |
| Veillonella_dispar | 13 | 36 | 9 | 21 |
| Veillonella_infantium | 14 | 40 | 12 | 11 |
| Veillonella_parvula | 26 | 50 | 13 | 36 |
| Veillonella_rogosae | 30 | 24 | 5 | 10 |
| Veillonella_sp_T11011_6 | 22 | 71 | 8 | 13 |
| Victivallis_vadensis | 109 | 72 | 86 | 113 |

Table 5 (Part 2A). Ranks.

| Profile | HDL_size_0 | PUFA_pct_0 | HDL_size_360 | ASCVD_10 yr_risk |
|---|---|---|---|---|
| Category | Fasting | Fasting | Post | Personal |
| | | | Prandial | |
| [Collinsella]_massiliensis | 130 | 120 | 148 | 151 |
| Actinomyces_odontolyticus | 46 | 109 | 54 | 12 |
| Actinomyces_sp_ICM47 | 95 | 137 | 92 | 49 |
| Adlercreutzia_equolifaciens | 83 | 94 | 81 | 72 |
| Agathobaculum_butyriciproducens | 87 | 36 | 97 | 150 |
| Akkermansia_muciniphila | 77 | 91 | 70 | 156 |
| Alistipes_fineqoldii | 116 | 134 | 119 | 145 |
| Alistipes_indistinctus | 86 | 107 | 94 | 20 |
| Alistipes_inops | 81 | 51 | 84 | 35 |
| Alistipes_onderdonkii | 39 | 31 | 24 | 14 |
| Alistipes_putredinis | 91 | 136 | 78 | 130 |
| Alistipes_shahii | 104 | 84 | 102 | 87 |
| Anaeromassilibacillus_sp_An250 | 66 | 138 | 90 | 157 |
| Anaerostipes_hadrus | 53 | 81 | 44 | 86 |
| Anaerotruncus_colihominis | 170 | 173 | 170 | 90 |
| Asaccharobacter_celatus | 41 | 88 | 41 | 99 |
| Bacteroides_caccae | 78 | 112 | 86 | 53 |
| Bacteroides_cellulosilyticus | 45 | 110 | 38 | 91 |
| Bacteroides_clarus | 58 | 70 | 59 | 60 |
| Bacteroides_dorei | 109 | 106 | 107 | 24 |
| Bacteroides_eggerthii | 107 | 26 | 117 | 153 |
| Bacteroides_faecis | 103 | 54 | 105 | 10 |
| Bacteroides_faecis_CAG_32 | 135 | 76 | 143 | 77 |
| Bacteroides_finegoldii | 59 | 38 | 64 | 84 |
| Bacteroides_fragilis | 149 | 132 | 134 | 100 |
| Bacteroides_galacturonicus | 108 | 65 | 108 | 105 |
| Bacteroides_intestinalis | 29 | 64 | 35 | 8 |
| Bacteroides_massiliensis | 72 | 17 | 93 | 68 |
| Bacteroides_nordii | 47 | 79 | 57 | 32 |
| Bacteroides_ovatus | 153 | 115 | 138 | 76 |
| Bacteroides_salyersiae | 21 | 90 | 25 | 138 |
| Bacteroides_sp_CAG_144 | 51 | 77 | 58 | 64 |
| Bacteroides_stercoris | 92 | 123 | 98 | 115 |
| Bacteroides_thetaiotaomicron | 120 | 131 | 111 | 28 |
| Bacteroides_uniformis | 163 | 167 | 166 | 107 |
| Bacteroides_vulgatus | 101 | 144 | 112 | 48 |
| Bacteroides_xylanisolvens | 94 | 75 | 63 | 42 |
| Barnesiella_intestinihominis | 97 | 97 | 85 | 79 |
| Bifidobacterium_adolescentis | 164 | 121 | 162 | 123 |
| Bifidobacterium_animalis | 11 | 12 | 9 | 67 |
| Bifidobacterium_bifidum | 141 | 124 | 144 | 120 |
| Bifidobacterium_catenulatum | 150 | 140 | 147 | 167 |
| Bifidobacterium_longum | 142 | 126 | 146 | 171 |
| Bifidobacterium_pseudocatenulatum | 89 | 50 | 77 | 112 |
| Bilophila_wadsworthia | 144 | 141 | 137 | 146 |
| Blautia_hydrogenotrophica | 161 | 145 | 157 | 162 |
| Blautia_obeum | 159 | 113 | 161 | 173 |
| Blautia_wexlerae | 123 | 104 | 133 | 56 |
| Butyricimonas_synergistica | 16 | 78 | 53 | 73 |
| Butyricimonas_virosa | 69 | 85 | 83 | 89 |
| Clostridium_asparagiforme | 155 | 149 | 160 | 111 |
| Clostridium_bolteae | 168 | 176 | 169 | 142 |
| Clostridium_bolteae_CAG_59 | 176 | 169 | 175 | 160 |
| Clostridium_citroniae | 121 | 168 | 103 | 63 |
| Clostridium_disporicum | 22 | 44 | 43 | 65 |
| Clostridium_innocuum | 175 | 172 | 176 | 164 |
| Clostridium_lavalense | 162 | 166 | 159 | 103 |
| Clostridium_leptum | 132 | 159 | 129 | 166 |
| Clostridium_saccharolyticum | 80 | 150 | 79 | 6 |
| Clostridium_sp_CAG_167 | 14 | 22 | 19 | 61 |
| Clostridium_sp_CAG_242 | 48 | 48 | 30 | 54 |
| Clostridium_sp_CAG_253 | 79 | 71 | 76 | 80 |
| Clostridium_sp_CAG_58 | 140 | 160 | 139 | 134 |
| Clostridium_spiroforme | 169 | 165 | 167 | 141 |
| Clostridium_symbiosum | 173 | 174 | 173 | 159 |
| Collinsella_aerofaciens | 156 | 99 | 156 | 176 |
| Collinsella_intestinalis | 157 | 152 | 154 | 163 |
| Collinsella_stercoris | 136 | 98 | 126 | 155 |
| Coprobacter_fastidiosus | 152 | 147 | 151 | 102 |
| Coprobacter_secundus | 3 | 16 | 7 | 70 |
| Coprococcus_catus | 40 | 37 | 47 | 148 |
| Coprococcus_comes | 38 | 57 | 46 | 172 |
| Coprococcus_eutactus | 43 | 15 | 29 | 85 |
| Desulfovibrio_piger | 63 | 74 | 56 | 139 |
| Dialister_invisus | 113 | 114 | 80 | 135 |
| Dielma_fastidiosa | 124 | 158 | 131 | 96 |
| Dorea_formicigenerans | 146 | 105 | 127 | 74 |
| Dorea_longicatena | 50 | 53 | 61 | 122 |
| Eggerthella_lenta | 154 | 171 | 155 | 143 |
| Eisenbergiella_massiliensis | 131 | 156 | 123 | 71 |
| Eisenbergiella_tayi | 139 | 157 | 149 | 147 |
| Enorma_massiliensis | 106 | 39 | 115 | 95 |
| Enterorhabdus_caecimuris | 23 | 83 | 33 | 69 |
| Escherichia_coli | 160 | 154 | 163 | 161 |
| Eubacterium_eligens | 2 | 8 | 3 | 3 |
| Eubacterium_hallii | 57 | 62 | 45 | 37 |
| Eubacterium_ramulus | 33 | 55 | 22 | 38 |
| Eubacterium_rectale | 165 | 117 | 164 | 98 |
| Eubacterium_siraeum | 65 | 86 | 55 | 30 |
| Eubacterium_sp_CAG_180 | 148 | 68 | 150 | 137 |
| Eubacterium_sp_CAG_251 | 110 | 60 | 118 | 174 |
| Eubacterium_sp_CAG_274 | 134 | 122 | 130 | 175 |
| Eubacterium_sp_CAG_38 | 70 | 45 | 50 | 50 |
| Eubacterium_sp_OM08_24 | 133 | 143 | 140 | 152 |
| Eubacterium_ventriosum | 171 | 146 | 174 | 124 |
| Faecalibacterium_prausnitzii | 20 | 18 | 39 | 11 |
| Firmicutes_bacterium_CAG_110 | 26 | 9 | 31 | 25 |
| Firmicutes_bacterium_CAG_145 | 100 | 155 | 67 | 106 |
| Firmicutes_bacterium_CAG_170 | 8 | 5 | 8 | 1 |
| Firmicutes_bacterium_CAG_238 | 62 | 13 | 66 | 15 |
| Firmicutes_bacterium_CAG_83 | 125 | 128 | 91 | 144 |
| Firmicutes_bacterium_CAG_94 | 119 | 148 | 135 | 170 |
| Firmicutes_bacterium_CAG_95 | 6 | 3 | 5 | 46 |
| Flavonifractor_plautii | 167 | 175 | 165 | 168 |
| Flavonifractor_sp_An100 | 32 | 66 | 12 | 34 |
| Fretibacterium_fastidiosum | 61 | 41 | 71 | 93 |
| Fusicatenibacter_saccharivorans | 102 | 130 | 104 | 39 |
| Gemella_sanguinis | 166 | 153 | 171 | 126 |
| Gemmiger_formicilis | 75 | 82 | 89 | 97 |
| Gordonibacter_pamelaeae | 138 | 161 | 141 | 110 |
| Haemophilus_parainfluenzae | 4 | 2 | 2 | 5 |
| Haemophilus_sp_HMSC71H05 | 24 | 32 | 23 | 101 |
| Harryflintia_acetispora | 115 | 100 | 124 | 29 |
| Holdemanella_biformis | 60 | 27 | 73 | 113 |
| Holdemania_filiformis | 151 | 125 | 145 | 127 |
| Hungatella_hathewayi | 126 | 163 | 136 | 154 |
| Intestinibacter_bartlettii | 84 | 93 | 88 | 121 |
| Intestinimonas_butyriciproducens | 7 | 118 | 4 | 51 |
| Lachnospira_pectinoschiza | 74 | 101 | 72 | 83 |
| Lactobacillus_rogosae | 36 | 56 | 34 | 75 |
| Lactococcus_lactis | 82 | 119 | 69 | 27 |
| Lawsonibacter_asaccharolyticus | 96 | 151 | 114 | 158 |
| Methanobrevibacter_smithii | 67 | 46 | 106 | 104 |
| Monoglobus_pectinilyticus | 158 | 102 | 152 | 128 |
| Odoribacter_splanchnicus | 52 | 49 | 52 | 21 |
| Olsenella_scatoligenes | 111 | 59 | 109 | 66 |
| Oscillibacter_sp_57_20 | 44 | 1 | 37 | 4 |
| Oscillibacter_sp_CAG_241 | 68 | 42 | 110 | 92 |
| Oscillibacter_sp_PC13 | 1 | 14 | 1 | 40 |
| Parabacteroides_distasonis | 73 | 164 | 100 | 31 |
| Parabacteroides_goldsteinii | 88 | 61 | 65 | 43 |
| Parabacteroides_johnsonii | 118 | 133 | 128 | 131 |
| Parabacteroides_merdae | 35 | 129 | 36 | 140 |
| Paraprevotella_clara | 15 | 30 | 32 | 45 |
| Paraprevotella_xylaniphila | 12 | 29 | 18 | 36 |
| Parasutterella_excrementihominis | 114 | 63 | 122 | 55 |
| Phascolarctobacterium_faecium | 93 | 111 | 87 | 33 |
| Prevotella_copri | 27 | 6 | 20 | 26 |
| Proteobacteria_bacterium_CAG_139 | 99 | 96 | 120 | 41 |
| Pseudoflavonifractor_capillosus | 129 | 139 | 125 | 116 |
| Pseudoflavonifractor_sp_An184 | 128 | 142 | 132 | 129 |
| Romboutsia_ilealis | 54 | 7 | 51 | 17 |
| Roseburia_faecis | 143 | 67 | 142 | 165 |
| Roseburia_hominis | 34 | 20 | 49 | 44 |
| Roseburia_intestinalis | 127 | 69 | 101 | 82 |
| Roseburia_inulinivorans | 172 | 116 | 172 | 132 |
| Roseburia_sp_CAG_182 | 13 | 4 | 21 | 2 |
| Roseburia_sp_CAG_309 | 28 | 52 | 14 | 133 |
| Roseburia_sp_CAG_471 | 31 | 19 | 17 | 16 |
| Rothia_mucilaginosa | 10 | 72 | 13 | 117 |
| Ruminococcaceae_bacterium_D16 | 85 | 127 | 74 | 94 |
| Ruminococcaceae_bacterium_D5 | 9 | 43 | 11 | 23 |
| Ruminococcus_bicirculans | 105 | 103 | 116 | 169 |
| Ruminococcus_bromii | 98 | 87 | 96 | 119 |
| Ruminococcus_callidus | 122 | 40 | 113 | 62 |
| Ruminococcus_gnavus | 174 | 170 | 168 | 125 |
| Ruminococcus_lactaris | 30 | 11 | 16 | 13 |
| Ruminococcus_torgues | 76 | 108 | 75 | 109 |
| Ruthenibacterium_lactatiformans | 147 | 162 | 158 | 59 |
| Slackia_isoflavoniconvertens | 37 | 10 | 48 | 149 |
| Streptococcus_australis | 112 | 73 | 82 | 9 |
| Streptococcus_mitis | 56 | 135 | 68 | 81 |
| Streptococcus_parasanguinis | 18 | 58 | 27 | 52 |
| Streptococcus_salivarius | 64 | 80 | 62 | 88 |
| Streptococcus_sp_A12 | 137 | 95 | 121 | 57 |
| Streptococcus_thermophilus | 17 | 92 | 10 | 118 |
| Sutterella_parvirubra | 117 | 25 | 99 | 114 |
| Turicibacter_sanguinis | 5 | 23 | 6 | 108 |
| Turicimonas_muris | 71 | 89 | 95 | 22 |
| Veillonella_atypica | 25 | 34 | 26 | 18 |
| Veillonella_dispar | 49 | 21 | 42 | 47 |
| Veillonella_infantium | 42 | 28 | 28 | 58 |
| Veillonella_parvula | 55 | 24 | 40 | 19 |
| Veillonella_rogosae | 90 | 33 | 60 | 7 |
| Veillonella_sp_T11011_6 | 19 | 47 | 15 | 78 |
| Victivallis_vadensis | 145 | 35 | 153 | 136 |

Table 5 (Part 2B): Ranks

| Profile | visceral_fat | LiverFatProbability | uPDI | Total_TG_0 |
|---|---|---|---|---|
| Category | Personal | Personal | Habitual | Fasting |
| | | | Diet | |
| [Collinsella]_massiliensis | 112 | 152 | 140 | 123 |
| Actinomyces_odontolyticus | 59 | 126 | 82 | 88 |
| Actinomyces_sp_ICM47 | 147 | 3 | 51 | 145 |
| Adlercreutzia_equolifaciens | 98 | 52 | 97 | 111 |
| Agathobaculum_butyriciproducens | 126 | 86 | 9 | 91 |
| Akkermansia_muciniphila | 55 | 45 | 95 | 72 |
| Alistipes_finegoldii | 85 | 97 | 157 | 102 |
| Alistipes_indistinctus | 81 | 112 | 68 | 113 |
| Alistipes_inops | 95 | 84 | 91 | 47 |
| Alistipes_onderdonkii | 76 | 47 | 77 | 16 |
| Alistipes_putredinis | 70 | 147 | 141 | 76 |
| Alistipes_shahii | 42 | 114 | 69 | 61 |
| Anaeromassilibacillus_sp_An250 | 124 | 66 | 150 | 116 |
| Anaerostipes_hadrus | 153 | 28 | 7 | 89 |
| Anaerotruncus_colihominis | 172 | 174 | 171 | 175 |
| Asaccharobacter_celatus | 80 | 22 | 87 | 82 |
| Bacteroides_caccae | 106 | 154 | 144 | 94 |
| Bacteroides_cellulosilyticus | 77 | 32 | 44 | 93 |
| Bacteroides_clarus | 73 | 157 | 43 | 44 |
| Bacteroides_dorei | 99 | 163 | 48 | 122 |
| Bacteroides_eggerthii | 75 | 87 | 73 | 56 |
| Bacteroides_faecis | 108 | 104 | 29 | 57 |
| Bacteroides_faecis_CAG_32 | 115 | 137 | 47 | 103 |
| Bacteroides_finegoldii | 33 | 149 | 53 | 69 |
| Bacteroides_fragilis | 162 | 171 | 134 | 138 |
| Bacteroides_galacturonicus | 118 | 14 | 84 | 92 |
| Bacteroides_intestinalis | 50 | 145 | 99 | 36 |
| Bacteroides_massiliensis | 21 | 123 | 25 | 45 |
| Bacteroides_nordii | 31 | 160 | 58 | 55 |
| Bacteroides_ovatus | 131 | 169 | 54 | 128 |
| Bacteroides_salyersiae | 94 | 68 | 96 | 42 |
| Bacteroides_sp_CAG_144 | 82 | 79 | 80 | 74 |
| Bacteroides_stercoris | 63 | 81 | 100 | 124 |
| Bacteroides_thetaiotaomicron | 104 | 159 | 83 | 130 |
| Bacteroides_uniformis | 155 | 144 | 110 | 167 |
| Bacteroides_vulgatus | 158 | 107 | 105 | 140 |
| Bacteroides_xylanisolvens | 71 | 130 | 60 | 67 |
| Barnesiella_intestinihominis | 84 | 135 | 115 | 80 |
| Bifidobacterium_adolescentis | 87 | 151 | 118 | 114 |
| Bifidobacterium_animalis | 23 | 90 | 1 | 29 |
| Bifidobacterium_bifidum | 109 | 133 | 163 | 125 |
| Bifidobacterium_catenulatum | 123 | 155 | 162 | 137 |
| Bifidobacterium_longum | 154 | 72 | 149 | 110 |
| Bifidobacterium_pseudocatenulatum | 141 | 102 | 62 | 58 |
| Bilophila_wadsworthia | 132 | 91 | 139 | 120 |
| Blautia_hydrogenotrophica | 164 | 166 | 142 | 162 |
| Blautia_obeum | 113 | 62 | 131 | 129 |
| Blautia_wexlerae | 134 | 69 | 56 | 109 |
| Butyricimonas_synergistica | 37 | 129 | 65 | 24 |
| Butyricimonas_virosa | 29 | 29 | 127 | 41 |
| Clostridium_asparagiforme | 163 | 153 | 135 | 148 |
| Clostridium_bolteae | 173 | 172 | 168 | 176 |
| Clostridium_bolteae_CAG_59 | 168 | 167 | 164 | 170 |
| Clostridium_citroniae | 166 | 175 | 147 | 165 |
| Clostridium_disporicum | 45 | 35 | 136 | 11 |
| Clostridium_innocuum | 169 | 170 | 174 | 171 |
| Clostridium_lavalense | 165 | 101 | 151 | 168 |
| Clostridium_leptum | 156 | 59 | 175 | 152 |
| Clostridium_saccharolyticum | 150 | 143 | 153 | 133 |
| Clostridium_sp_CAG_167 | 7 | 21 | 24 | 19 |
| Clostridium_sp_CAG_242 | 44 | 131 | 72 | 59 |
| Clostridium_sp_CAG_253 | 68 | 15 | 75 | 66 |
| Clostridium_sp_CAG_58 | 171 | 168 | 133 | 169 |
| Clostridium_spiroforme | 175 | 150 | 165 | 166 |
| Clostridium_symbiosum | 159 | 162 | 176 | 174 |
| Collinsella_aerofaciens | 145 | 111 | 137 | 107 |
| Collinsella_intestinalis | 151 | 164 | 161 | 146 |
| Collinsella_stercoris | 117 | 122 | 92 | 127 |
| Coprobacter_fastidiosus | 110 | 109 | 90 | 149 |
| Coprobacter_secundus | 72 | 38 | 70 | 8 |
| Coprococcus_catus | 15 | 12 | 27 | 35 |
| Coprococcus_comes | 52 | 48 | 126 | 54 |
| Coprococcus_eutactus | 24 | 76 | 103 | 23 |
| Desulfovibrio_piger | 78 | 82 | 101 | 64 |
| Dialister_invisus | 67 | 49 | 104 | 121 |
| Dielma_fastidiosa | 146 | 46 | 148 | 154 |
| Dorea_formicigenerans | 130 | 161 | 40 | 132 |
| Dorea_longicatena | 88 | 140 | 113 | 83 |
| Eggerthella_lenta | 160 | 148 | 167 | 164 |
| Eisenbergiella_massiliensis | 142 | 158 | 130 | 157 |
| Eisenbergiella_tayi | 103 | 77 | 156 | 158 |
| Enorma_massiliensis | 101 | 132 | 123 | 39 |
| Enterorhabdus_caecimuris | 89 | 55 | 108 | 101 |
| Escherichia_coli | 127 | 141 | 173 | 159 |
| Eubacterium_eligens | 19 | 16 | 6 | 20 |
| Eubacterium_hallii | 107 | 94 | 15 | 40 |
| Eubacterium_ramulus | 90 | 51 | 17 | 52 |
| Eubacterium_rectale | 133 | 142 | 59 | 134 |
| Eubacterium_siraeum | 27 | 9 | 145 | 46 |
| Eubacterium_sp_CAG_180 | 74 | 113 | 129 | 77 |
| Eubacterium_sp_CAG_251 | 64 | 44 | 114 | 99 |
| Eubacterium_sp_CAG_274 | 161 | 156 | 55 | 108 |
| Eubacterium_sp_CAG_38 | 120 | 125 | 30 | 104 |
| Eubacterium_sp_OM08_24 | 96 | 88 | 112 | 150 |
| Eubacterium_ventriosum | 138 | 139 | 121 | 156 |
| Faecalibacterium_prausnitzii | 22 | 57 | 14 | 14 |
| Firmicutes_bacterium_CAG_110 | 8 | 1 | 76 | 7 |
| Firmicutes_bacterium_CAG_145 | 140 | 40 | 117 | 161 |
| Firmicutes_bacterium_CAG_170 | 6 | 2 | 28 | 2 |
| Firmicutes_bacterium_CAG_238 | 16 | 39 | 39 | 12 |
| Firmicutes_bacterium_CAG_83 | 83 | 108 | 89 | 86 |
| Firmicutes_bacterium_CAG_94 | 144 | 37 | 172 | 142 |
| Firmicutes_bacterium_CAG_95 | 3 | 4 | 3 | 1 |
| Flavonifractor_plautii | 176 | 173 | 166 | 173 |
| Flavonifractor_sp_An100 | 43 | 31 | 71 | 81 |
| Fretibacterium_fastidiosum | 40 | 19 | 42 | 15 |
| Fusicatenibacter_saccharivorans | 139 | 93 | 46 | 144 |
| Gemella_sanguinis | 167 | 121 | 116 | 160 |
| Gemmiger_formicilis | 65 | 25 | 93 | 49 |
| Gordonibacter_pamelaeae | 157 | 98 | 160 | 155 |
| Haemophilus_parainfluenzae | 4 | 58 | 23 | 3 |
| Haemophilus_sp_HMSC71H05 | 60 | 127 | 36 | 30 |
| Harryflintia_acetispora | 149 | 78 | 152 | 115 |
| Holdemanella_biformis | 10 | 119 | 41 | 34 |
| Holdemania_filiformis | 122 | 138 | 122 | 106 |
| Hungatella_hathewayi | 86 | 134 | 155 | 153 |
| Intestinibacter_bartlettii | 57 | 99 | 120 | 62 |
| Intestinimonas_butyriciproducens | 41 | 103 | 94 | 73 |
| Lachnospira_pectinoschiza | 121 | 89 | 124 | 90 |
| Lactobacillus_rogosae | 91 | 10 | 61 | 50 |
| Lactococcus_lactis | 54 | 118 | 31 | 131 |
| Lawsonibacter_asaccharolyticus | 97 | 105 | 32 | 147 |
| Methanobrevibacter_smithii | 47 | 7 | 106 | 60 |
| Monoglobus_pectinilyticus | 135 | 56 | 66 | 136 |
| Odoribacter_splanchnicus | 51 | 117 | 138 | 26 |
| Olsenella_scatoligenes | 102 | 74 | 63 | 70 |
| Oscillibacter_sp_57_20 | 2 | 27 | 11 | 4 |
| Oscillibacter_sp_CAG_241 | 34 | 13 | 146 | 18 |
| Oscillibacter_sp_PC13 | 28 | 23 | 5 | 10 |
| Parabacteroides_distasonis | 128 | 165 | 74 | 151 |
| Parabacteroides_goldsteinii | 39 | 42 | 79 | 98 |
| Parabacteroides_johnsonii | 148 | 120 | 81 | 139 |
| Parabacteroides_merdae | 58 | 124 | 154 | 97 |
| Paraprevotella_clara | 26 | 41 | 12 | 27 |
| Paraprevotella_xylaniphila | 20 | 30 | 10 | 22 |
| Parasutterella_excrementihominis | 125 | 95 | 35 | 75 |
| Phascolarctobacterium_faecium | 105 | 128 | 64 | 78 |
| Prevotella_copri | 9 | 50 | 22 | 6 |
| Proteobacteria_bacterium_CAG_139 | 143 | 96 | 50 | 96 |
| Pseudoflavonifractor_capillosus | 137 | 146 | 159 | 135 |
| Pseudoflavonifractor_sp_An184 | 111 | 54 | 169 | 126 |
| Romboutsia_ilealis | 61 | 17 | 67 | 32 |
| Roseburia_faecis | 129 | 65 | 52 | 84 |
| Roseburia_hominis | 66 | 100 | 4 | 43 |
| Roseburia_intestinalis | 114 | 60 | 57 | 105 |
| Roseburia_inulinivorans | 170 | 80 | 125 | 143 |
| Roseburia_sp_CAG_182 | 14 | 11 | 8 | 5 |
| Roseburia_sp_CAG_309 | 30 | 53 | 26 | 71 |
| Roseburia_sp_CAG_471 | 49 | 73 | 18 | 63 |
| Rothia_mucilaginosa | 1 | 36 | 13 | 65 |
| Ruminococcaceae_bacterium_D16 | 69 | 106 | 132 | 141 |
| Ruminococcaceae_bacterium_D5 | 5 | 8 | 98 | 25 |
| Ruminococcus_bicirculans | 152 | 85 | 119 | 119 |
| Ruminococcus_bromii | 92 | 33 | 78 | 68 |
| Ruminococcus_callidus | 46 | 26 | 128 | 85 |
| Ruminococcus_gnavus | 174 | 176 | 158 | 172 |
| Ruminococcus_lactaris | 35 | 18 | 16 | 13 |
| Ruminococcus_torques | 93 | 63 | 86 | 79 |
| Ruthenibacterium_lactatiformans | 136 | 71 | 170 | 163 |
| Slackia_isoflavoniconvertens | 36 | 70 | 49 | 38 |
| Streptococcus_australis | 79 | 115 | 38 | 112 |
| Streptococcus_mitis | 100 | 20 | 109 | 117 |
| Streptococcus_parasanguinis | 12 | 43 | 111 | 53 |
| Streptococcus_salivarius | 32 | 110 | 107 | 100 |
| Streptococcus_sp_A12 | 119 | 75 | 34 | 118 |
| Streptococcus_thermophilus | 53 | 116 | 2 | 95 |
| Sutterella_parvirubra | 62 | 136 | 20 | 51 |
| Turicibacter_sanguinis | 13 | 6 | 102 | 9 |
| Turicimonas_muris | 116 | 64 | 85 | 87 |
| Veillonella_atypica | 25 | 67 | 37 | 37 |
| Veillonella_dispar | 18 | 24 | 19 | 28 |
| Veillonella_infantium | 11 | 61 | 33 | 33 |
| Veillonella_parvula | 38 | 83 | 88 | 17 |
| Veillonella_rogosae | 56 | 92 | 21 | 48 |
| Veillonella_sp_T11011_6 | 17 | 34 | 45 | 31 |
| Victivallis_vadensis | 48 | 5 | 143 | 21 |

Table 5 (Part 2B): Ranks

| | | | Meal_JJ_Hospi- | Meal_JJ_Hospi- |
|---|---|---|---|---|
| Profile | VLDL_size_0 | GlycA_0 | tal_meal_glucose_120_iauc | tal_mealc_peptide_120_iauc |
| Category | Fasting | Fasting | Post | Post |
| | | | prandial | prandial |
| [Collinsella]_massiliensis | 107 | 109 | 151 | 147 |
| Actinomyces_odontolyticus | 66 | 69 | 45 | 21 |
| Actinomyces_sp_ICM47 | 151 | 128 | 162 | 136 |
| Adlercreutzia_equolifaciens | 112 | 73 | 144 | 146 |
| Agathobaculum_butyriciproducens | 118 | 38 | 26 | 54 |
| Akkermansia_muciniphila | 68 | 82 | 53 | 130 |
| Alistipes_finegoldii | 81 | 127 | 118 | 131 |
| Alistipes_indistinctus | 105 | 70 | 15 | 71 |
| Alistipes_inops | 62 | 102 | 60 | 129 |
| Alistipes_onderdonkii | 15 | 37 | 92 | 84 |
| Alistipes_putredinis | 72 | 122 | 120 | 128 |
| Alistipes_shahii | 67 | 74 | 87 | 60 |
| Anaeromassilibacillus_sp_An250 | 98 | 126 | 9 | 72 |
| Anaerostipes_hadrus | 86 | 60 | 62 | 145 |
| Anaerotruncus_colihominis | 175 | 169 | 156 | 173 |
| Asaccharobacter_celatus | 83 | 66 | 146 | 139 |
| Bacteroides_caccae | 89 | 111 | 109 | 149 |
| Bacteroides_cellulosilyticus | 45 | 75 | 112 | 135 |
| Bacteroides_clarus | 37 | 59 | 31 | 47 |
| Bacteroides_dorei | 108 | 116 | 127 | 112 |
| Bacteroides_eggerthii | 92 | 71 | 16 | 58 |
| Bacteroides_faecis | 74 | 84 | 117 | 114 |
| Bacteroides_faecis_CAG_32 | 94 | 114 | 136 | 124 |
| Bacteroides_finegoldii | 80 | 34 | 43 | 93 |
| Bacteroides_fragilis | 142 | 168 | 130 | 156 |
| Bacteroides_galacturonicus | 111 | 63 | 47 | 75 |
| Bacteroides_intestinalis | 26 | 41 | 125 | 56 |
| Bacteroides_massiliensis | 48 | 15 | 84 | 32 |
| Bacteroides_nordii | 44 | 23 | 143 | 107 |
| Bacteroides_ovatus | 125 | 121 | 89 | 63 |
| Bacteroides_salyersiae | 29 | 51 | 12 | 25 |
| Bacteroides_sp_CAG_144 | 70 | 103 | 79 | 109 |
| Bacteroides_stercoris | 114 | 92 | 55 | 78 |
| Bacteroides_thetaiotaomicron | 126 | 130 | 150 | 125 |
| Bacteroides_uniformis | 164 | 149 | 157 | 111 |
| Bacteroides_vulgatus | 135 | 125 | 165 | 153 |
| Bacteroides_xylanisolvens | 77 | 49 | 107 | 62 |
| Barnesiella_intestinihominis | 87 | 90 | 104 | 116 |
| Bifidobacterium_adolescentis | 133 | 91 | 3 | 53 |
| Bifidobacterium_animalis | 25 | 55 | 32 | 10 |
| Bifidobacterium_bifidum | 134 | 141 | 72 | 96 |
| Bifidobacterium_catenulatum | 149 | 148 | 78 | 59 |
| Bifidobacterium_longum | 115 | 123 | 83 | 92 |
| Bifidobacterium_pseudocatenulatum | 63 | 81 | 44 | 65 |
| Bilophila_wadsworthia | 110 | 131 | 123 | 152 |
| Blautia_hydrogenotrophica | 168 | 153 | 137 | 166 |
| Blautia_obeum | 141 | 117 | 161 | 132 |
| Blautia_wexlerae | 143 | 133 | 145 | 150 |
| Butyricimonas_synergistica | 8 | 85 | 116 | 82 |
| Butyricimonas_virosa | 21 | 87 | 139 | 113 |
| Clostridium_asparagiforme | 155 | 158 | 176 | 168 |
| Clostridium_bolteae | 176 | 176 | 169 | 175 |
| Clostridium_bolteae_CAG_59 | 172 | 172 | 168 | 162 |
| Clostridium_citroniae | 158 | 167 | 164 | 171 |
| Clostridium_disporicum | 14 | 16 | 25 | 9 |
| Clostridium_innocuum | 171 | 171 | 167 | 163 |
| Clostridium_lavalense | 169 | 163 | 148 | 165 |
| Clostridium_leptum | 145 | 151 | 35 | 117 |
| Clostridium_saccharolyticum | 123 | 165 | 135 | 160 |
| Clostridium_sp_CAG_167 | 28 | 6 | 22 | 13 |
| Clostridium_sp_CAG_242 | 57 | 94 | 37 | 52 |
| Clostridium_sp_CAG_253 | 58 | 58 | 132 | 55 |
| Clostridium_sp_CAG_58 | 162 | 162 | 158 | 167 |
| Clostridium_spiroforme | 166 | 160 | 153 | 161 |
| Clostridium_symbiosum | 173 | 174 | 172 | 176 |
| Collinsella_aerofaciens | 109 | 129 | 65 | 134 |
| Collinsella_intestinalis | 160 | 161 | 110 | 142 |
| Collinsella_stercoris | 137 | 115 | 75 | 121 |
| Coprobacter_fastidiosus | 148 | 108 | 69 | 144 |
| Coprobacter_secundus | 2 | 50 | 141 | 90 |
| Coprococcus_catus | 24 | 24 | 39 | 24 |
| Coprococcus_comes | 52 | 54 | 66 | 108 |
| Coprococcus_eutactus | 38 | 12 | 64 | 33 |
| Desulfovibrio_piger | 73 | 64 | 88 | 126 |
| Dialister_invisus | 116 | 118 | 57 | 81 |
| Dielma_fastidiosa | 146 | 157 | 163 | 169 |
| Dorea_formicigenerans | 138 | 150 | 58 | 104 |
| Dorea_longicatena | 82 | 32 | 24 | 73 |
| Eggerthella_lenta | 167 | 170 | 174 | 164 |
| Eisenbergiella_massiliensis | 156 | 146 | 173 | 172 |
| Eisenbergiella_tayi | 132 | 138 | 138 | 158 |
| Enorma_massiliensis | 49 | 77 | 91 | 102 |
| Enterorhabdus_caecimuris | 78 | 72 | 154 | 143 |
| Escherichia_coli | 163 | 166 | 149 | 141 |
| Eubacterium_eligens | 10 | 5 | 94 | 74 |
| Eubacterium_hallii | 27 | 25 | 105 | 38 |
| Eubacterium_ramulus | 46 | 46 | 129 | 68 |
| Eubacterium_rectale | 153 | 159 | 121 | 94 |
| Eubacterium_siraeum | 47 | 19 | 28 | 51 |
| Eubacterium_sp_CAG_180 | 93 | 136 | 61 | 89 |
| Eubacterium_sp_CAG_251 | 122 | 76 | 4 | 48 |
| Eubacterium_sp_CAG_274 | 95 | 142 | 101 | 127 |
| Eubacterium_sp_CAG_38 | 102 | 101 | 52 | 31 |
| Eubacterium_sp_OM08_24 | 144 | 106 | 96 | 64 |
| Eubacterium_ventriosum | 159 | 96 | 76 | 43 |
| Faecalibacterium_prausnitzii | 33 | 13 | 49 | 8 |
| Firmicutes_bacterium_CAG_110 | 9 | 8 | 17 | 37 |
| Firmicutes_bacterium_CAG_145 | 150 | 140 | 100 | 154 |
| Firmicutes_bacterium_CAG_170 | 3 | 3 | 7 | 22 |
| Firmicutes_bacterium_CAG_238 | 12 | 47 | 102 | 41 |
| Firmicutes_bacterium_CAG_83 | 64 | 135 | 115 | 100 |
| Firmicutes_bacterium_CAG_94 | 127 | 147 | 99 | 91 |
| Firmicutes_bacterium_CAG_95 | 1 | 2 | 2 | 6 |
| Flavonifractor_plautii | 170 | 173 | 175 | 170 |
| Flavonifractor_sp_An100 | 43 | 97 | 23 | 12 |
| Fretibacterium_fastidiosum | 17 | 39 | 68 | 28 |
| Fusicatenibacter_saccharivorans | 140 | 104 | 98 | 70 |
| Gemella_sanguinis | 161 | 164 | 171 | 133 |
| Gemmiger_formicilis | 53 | 56 | 160 | 115 |
| Gordonibacter_pamelaeae | 154 | 155 | 152 | 159 |
| Haemophilus_parainfluenzae | 6 | 1 | 5 | 5 |
| Haemophilus_sp_HMSC71H05 | 39 | 20 | 27 | 16 |
| Harryflintia_acetispora | 128 | 113 | 38 | 101 |
| Holdemanella_biformis | 55 | 28 | 36 | 34 |
| Holdemania_filiformis | 113 | 134 | 147 | 157 |
| Hungatella_hathewayi | 139 | 144 | 159 | 137 |
| Intestinibacter_bartlettii | 75 | 68 | 42 | 11 |
| Intestinimonas_butyriciproducens | 56 | 61 | 74 | 119 |
| Lachnospira_pectinoschiza | 103 | 78 | 90 | 140 |
| Lactobacillus_rogosae | 71 | 22 | 59 | 61 |
| Lactococcus_lactis | 99 | 86 | 124 | 88 |
| Lawsonibacter_asaccharolyticus | 136 | 93 | 114 | 87 |
| Methanobrevibacter_smithii | 69 | 33 | 21 | 66 |
| Monoglobus_pectinilyticus | 157 | 137 | 113 | 95 |
| Odoribacter_splanchnicus | 20 | 80 | 106 | 122 |
| Olsenella_scatoligenes | 84 | 107 | 80 | 86 |
| Oscillibacter_sp_57_20 | 7 | 7 | 54 | 15 |
| Oscillibacter_sp_CAG_241 | 23 | 26 | 29 | 79 |
| Oscillibacter_sp_PC13 | 5 | 11 | 11 | 27 |
| Parabacteroides_distasonis | 130 | 152 | 155 | 155 |
| Parabacteroides_goldsteinii | 85 | 65 | 51 | 50 |
| Parabacteroides_johnsonii | 120 | 100 | 134 | 138 |
| Parabacteroides_merdae | 51 | 98 | 82 | 118 |
| Paraprevotella_clara | 16 | 27 | 18 | 36 |
| Paraprevotella_xylaniphila | 13 | 30 | 14 | 26 |
| Parasutterella_excrementihominis | 88 | 52 | 50 | 30 |
| Phascolarctobacterium_faecium | 90 | 112 | 128 | 106 |
| Prevotella_copri | 22 | 9 | 6 | 35 |
| Proteobacteria_bacterium_CAG_139 | 96 | 95 | 131 | 76 |
| Pseudoflavonifractor_capillosus | 147 | 145 | 63 | 110 |
| Pseudoflavonifractor_sp_An184 | 129 | 120 | 33 | 69 |
| Romboutsia_ilealis | 41 | 10 | 1 | 14 |
| Roseburia_faecis | 131 | 110 | 81 | 120 |
| Roseburia_hominis | 40 | 44 | 13 | 45 |
| Roseburia_intestinalis | 119 | 62 | 71 | 85 |
| Roseburia_inulinivorans | 165 | 156 | 34 | 103 |
| Roseburia_sp_CAG_182 | 11 | 4 | 56 | 23 |
| Roseburia_sp_CAG_309 | 61 | 29 | 40 | 17 |
| Roseburia_sp_CAG_471 | 59 | 45 | 30 | 39 |
| Rothia_mucilaginosa | 32 | 57 | 41 | 20 |
| Ruminococcaceae_bacterium_D16 | 124 | 119 | 20 | 67 |
| Ruminococcaceae_bacterium_D5 | 18 | 21 | 108 | 80 |
| Ruminococcus_bicirculans | 97 | 143 | 70 | 105 |
| Ruminococcus_bromii | 76 | 99 | 97 | 98 |
| Ruminococcus_callidus | 121 | 67 | 73 | 40 |
| Ruminococcus_gnavus | 174 | 175 | 170 | 174 |
| Ruminococcus_lactaris | 19 | 35 | 111 | 49 |
| Ruminococcus_torques | 104 | 40 | 103 | 83 |
| Ruthenibacterium_lactatiformans | 152 | 154 | 126 | 148 |
| Slackia_isoflavoniconvertens | 30 | 48 | 48 | 44 |
| Streptococcus_australis | 106 | 124 | 142 | 99 |
| Streptococcus_mitis | 101 | 139 | 166 | 123 |
| Streptococcus_parasanguinis | 54 | 53 | 122 | 29 |
| Streptococcus_salivarius | 100 | 83 | 93 | 57 |
| Streptococcus_sp_A12 | 117 | 132 | 140 | 151 |
| Streptococcus_thermophilus | 34 | 105 | 133 | 97 |
| Sutterella_parvirubra | 91 | 88 | 95 | 77 |
| Turicibacter_sanguinis | 4 | 18 | 86 | 18 |
| Turicimonas_muris | 79 | 79 | 119 | 46 |
| Veillonella_atypica | 60 | 42 | 77 | 2 |
| Veillonella_dispar | 50 | 14 | 8 | 3 |
| Veillonella_infantium | 35 | 17 | 19 | 1 |
| Veillonella_parvula | 36 | 43 | 10 | 4 |
| Veillonella_rogosae | 65 | 31 | 46 | 19 |
| Veillonella_sp_T11011_6 | 31 | 36 | 85 | 7 |
| Victivallis_vadensis | 42 | 89 | 67 | 42 |

Table 5 (Part 2C): Ranks

| Profile | Meal_JJ_Hospital_meal_trig_360_iauc | GlycA_360 | VLDL_size_360 | Fasting |
|---|---|---|---|---|
| Category | Post | Post | Post | |
| | prandial | prandial | prandial | |
| [Collinsella]_massiliensis | 97 | 125 | 105 | 117.8 |
| Actinomyces_odontolyticus | 81 | 78 | 75 | 75.6 |
| Actinomyces_sp_ICM47 | 164 | 141 | 154 | 131.2 |
| Adlercreutzia_equolifaciens | 72 | 62 | 120 | 94.6 |
| Agathobaculum_butyriciproducens | 133 | 51 | 114 | 74 |
| Akkermansia_muciniphila | 26 | 84 | 35 | 78 |
| Alistipes_finegoldii | 11 | 104 | 48 | 112 |
| Alistipes_indistinctus | 116 | 80 | 136 | 96.2 |
| Alistipes_inops | 86 | 93 | 55 | 68.6 |
| Alistipes_onderdonkii | 95 | 40 | 27 | 27.6 |
| Alistipes_putredinis | 51 | 108 | 68 | 99.4 |
| Alistipes_shahii | 70 | 71 | 25 | 78 |
| Anaeromassilibacillus_sp_An250 | 22 | 94 | 60 | 108.8 |
| Anaerostipes_hadrus | 142 | 68 | 127 | 73.8 |
| Anaerotruncus_colihominis | 169 | 170 | 173 | 172.4 |
| Asaccharobacter_celatus | 42 | 55 | 84 | 72 |
| Bacteroides_caccae | 128 | 121 | 100 | 96.8 |
| Bacteroides_cellulosilyticus | 57 | 50 | 45 | 73.6 |
| Bacteroides_clarus | 33 | 43 | 53 | 53.6 |
| Bacteroides_dorei | 56 | 102 | 90 | 112.2 |
| Bacteroides_eggerthii | 120 | 59 | 104 | 70.4 |
| Bacteroides_faecis | 115 | 120 | 54 | 74.4 |
| Bacteroides_faecis_CAG_32 | 118 | 148 | 69 | 104.4 |
| Bacteroides_finegoldii | 155 | 38 | 112 | 56 |
| Bacteroides_fragilis | 161 | 168 | 142 | 145.8 |
| Bacteroides_galacturonicus | 153 | 57 | 129 | 87.8 |
| Bacteroides_intestinalis | 110 | 31 | 79 | 39.2 |
| Bacteroides_massiliensis | 88 | 20 | 65 | 39.4 |
| Bacteroides_nordii | 92 | 26 | 74 | 49.6 |
| Bacteroides_ovatus | 91 | 127 | 123 | 128.4 |
| Bacteroides_salyersiae | 17 | 64 | 36 | 46.6 |
| Bacteroides_sp_CAG_144 | 45 | 91 | 89 | 75 |
| Bacteroides_stercoris | 112 | 110 | 77 | 109 |
| Bacteroides_thetaiotaomicron | 134 | 81 | 140 | 127.4 |
| Bacteroides_uniformis | 156 | 147 | 164 | 162 |
| Bacteroides_vulgatus | 166 | 122 | 153 | 129 |
| Bacteroides_xylanisolvens | 65 | 49 | 95 | 72.4 |
| Barnesiella_intestinihominis | 36 | 77 | 57 | 90.2 |
| Bifidobacterium_adolescentis | 53 | 97 | 106 | 124.6 |
| Bifidobacterium_animalis | 69 | 82 | 16 | 26.4 |
| Bifidobacterium_bifidum | 77 | 137 | 143 | 133 |
| Bifidobacterium_catenulatum | 58 | 152 | 117 | 144.8 |
| Bifidobacterium_longum | 49 | 124 | 126 | 123.2 |
| Bifidobacterium_pseudocatenulatum | 127 | 69 | 116 | 68.2 |
| Bilophila_wadsworthia | 75 | 126 | 86 | 129.2 |
| Blautia_hydrogenotrophica | 174 | 154 | 165 | 157.8 |
| Blautia_obeum | 152 | 140 | 155 | 131.8 |
| Blautia_wexlerae | 159 | 139 | 152 | 122.4 |
| Butyricimonas_synergistica | 47 | 92 | 22 | 42.2 |
| Butyricimonas_virosa | 94 | 90 | 32 | 60.6 |
| Clostridium_asparagiforme | 122 | 167 | 159 | 153 |
| Clostridium_bolteae | 176 | 174 | 176 | 174.4 |
| Clostridium_bolteae_CAG_59 | 170 | 175 | 169 | 171.8 |
| Clostridium_citroniae | 143 | 165 | 163 | 155.8 |
| Clostridium_disporicum | 4 | 29 | 8 | 21.4 |
| Clostridium_innocuum | 162 | 171 | 172 | 172 |
| Clostridium_lavalense | 132 | 164 | 158 | 165.6 |
| Clostridium_leptum | 117 | 149 | 130 | 147.8 |
| Clostridium_saccharolyticum | 87 | 159 | 94 | 130.2 |
| Clostridium_sp_CAG_167 | 32 | 8 | 28 | 17.8 |
| Clostridium_sp_CAG_242 | 44 | 74 | 12 | 61.2 |
| Clostridium_sp_CAG_253 | 59 | 63 | 33 | 66.4 |
| Clostridium_sp_CAG_58 | 154 | 162 | 167 | 158.6 |
| Clostridium_spiroforme | 148 | 157 | 170 | 165.2 |
| Clostridium_symbiosum | 145 | 173 | 171 | 173.6 |
| Collinsella_aerofaciens | 137 | 133 | 124 | 120 |
| Collinsella_intestinalis | 158 | 161 | 161 | 155.2 |
| Collinsella_stercoris | 146 | 135 | 144 | 122.6 |
| Coprobacter_fastidiosus | 165 | 109 | 148 | 140.8 |
| Coprobacter_secundus | 18 | 54 | 7 | 15.8 |
| Coprococcus_catus | 67 | 28 | 56 | 32 |
| Coprococcus_comes | 131 | 52 | 98 | 51 |
| Coprococcus_eutactus | 29 | 9 | 30 | 26.2 |
| Desulfovibrio_piger | 103 | 89 | 108 | 67.6 |
| Dialister_invisus | 3 | 128 | 72 | 116.4 |
| Dielma_fastidiosa | 172 | 160 | 150 | 147.8 |
| Dorea_formicigenerans | 168 | 146 | 147 | 134.2 |
| Dorea_longicatena | 106 | 45 | 93 | 60 |
| Eggerthella_lenta | 119 | 172 | 166 | 165.2 |
| Eisenbergiella_massiliensis | 82 | 131 | 133 | 149.2 |
| Eisenbergiella_tayi | 114 | 118 | 138 | 144.8 |
| Enorma_massiliensis | 62 | 106 | 80 | 62 |
| Enterorhabdus_caecimuris | 74 | 67 | 113 | 71.4 |
| Escherichia_coli | 108 | 163 | 141 | 160.4 |
| Eubacterium_eligens | 80 | 5 | 26 | 9 |
| Eubacterium_hallii | 52 | 30 | 73 | 42.2 |
| Eubacterium_ramulus | 151 | 44 | 85 | 46.4 |
| Eubacterium_rectale | 105 | 156 | 145 | 145.6 |
| Eubacterium_siraeum | 50 | 27 | 39 | 52.6 |
| Eubacterium_sp_CAG_180 | 89 | 150 | 107 | 104.4 |
| Eubacterium_sp_CAG_251 | 78 | 66 | 103 | 93.4 |
| Eubacterium_sp_CAG_274 | 90 | 144 | 102 | 120.2 |
| Eubacterium_sp_CAG_38 | 107 | 98 | 92 | 84.4 |
| Eubacterium_sp_OM08_24 | 121 | 114 | 118 | 135.2 |
| Eubacterium_ventriosum | 126 | 112 | 168 | 145.6 |
| Faecalibacterium_prausnitzii | 31 | 7 | 31 | 19.6 |
| Firmicutes_bacterium_CAG_110 | 7 | 16 | 4 | 11.8 |
| Firmicutes_bacterium_CAG_145 | 149 | 115 | 139 | 141.2 |
| Firmicutes_bacterium_CAG_170 | 73 | 3 | 17 | 4.2 |
| Firmicutes_bacterium_CAG_238 | 15 | 76 | 14 | 29.2 |
| Firmicutes_bacterium_CAG_83 | 85 | 119 | 42 | 107.6 |
| Firmicutes_bacterium_CAG_94 | 24 | 134 | 111 | 136.6 |
| Firmicutes_bacterium_CAG_95 | 5 | 4 | 1 | 2.6 |
| Flavonifractor_plautii | 171 | 169 | 174 | 171.6 |
| Flavonifractor_sp_An100 | 13 | 70 | 41 | 63.8 |
| Fretibacterium_fastidiosum | 12 | 36 | 11 | 34.6 |
| Fusicatenibacter_saccharivorans | 150 | 101 | 160 | 124 |
| Gemella_sanguinis | 130 | 166 | 157 | 160.8 |
| Gemmiger_formicilis | 96 | 75 | 83 | 63 |
| Gordonibacter_pamelaeae | 23 | 151 | 134 | 152.6 |
| Haemophilus_parainfluenzae | 19 | 1 | 5 | 3.2 |
| Haemophilus_sp_HMSC71H05 | 54 | 15 | 59 | 29 |
| Harryflintia_acetispora | 66 | 103 | 122 | 114.2 |
| Holdemanella_biformis | 136 | 32 | 51 | 40.8 |
| Holdemania_filiformis | 123 | 138 | 137 | 125.8 |
| Hungatella_hathewayi | 157 | 155 | 156 | 145 |
| Intestinibacter_bartlettii | 8 | 83 | 43 | 76.4 |
| Intestinimonas_butyriciproducens | 25 | 24 | 21 | 63 |
| Lachnospira_pectinoschiza | 100 | 79 | 110 | 89.2 |
| Lactobacillus_rogosae | 113 | 34 | 70 | 47 |
| Lactococcus_lactis | 63 | 86 | 87 | 103.4 |
| Lawsonibacter_asaccharolyticus | 109 | 47 | 128 | 124.6 |
| Methanobrevibacter_smithii | 61 | 60 | 82 | 55 |
| Monoglobus_pectinilyticus | 138 | 129 | 135 | 138 |
| Odoribacter_splanchnicus | 27 | 61 | 18 | 45.4 |
| Olsenella_scatoligenes | 124 | 145 | 96 | 86.2 |
| Oscillibacter_sp_57_20 | 6 | 6 | 3 | 12.6 |
| Oscillibacter_sp_CAG_241 | 104 | 56 | 62 | 35.4 |
| Oscillibacter_sp_PC13 | 39 | 10 | 9 | 8.2 |
| Parabacteroides_distasonis | 144 | 143 | 149 | 134 |
| Parabacteroides_goldsteinii | 79 | 58 | 91 | 79.4 |
| Parabacteroides_johnsonii | 160 | 100 | 109 | 122 |
| Parabacteroides_merdae | 139 | 88 | 47 | 82 |
| Paraprevotella_clara | 129 | 25 | 66 | 23 |
| Paraprevotella_xylaniphila | 140 | 33 | 58 | 21.2 |
| Parasutterella_excrementihominis | 14 | 41 | 61 | 78.4 |
| Phascolarctobacterium_faecium | 141 | 113 | 71 | 96.8 |
| Prevotella_copri | 71 | 13 | 38 | 14 |
| Proteobacteria_bacterium_CAG_139 | 9 | 85 | 63 | 96.4 |
| Pseudoflavonifractor_capillosus | 60 | 116 | 115 | 139 |
| Pseudoflavonifractor_sp_An184 | 102 | 132 | 121 | 129 |
| Romboutsia_ilealis | 41 | 11 | 29 | 28.8 |
| Roseburia_faecis | 101 | 130 | 119 | 107 |
| Roseburia_hominis | 30 | 35 | 50 | 36.2 |
| Roseburia_intestinalis | 163 | 53 | 125 | 96.4 |
| Roseburia_inulinivorans | 147 | 158 | 162 | 150.4 |
| Roseburia_sp_CAG_182 | 43 | 2 | 15 | 7.4 |
| Roseburia_sp_CAG_309 | 48 | 17 | 24 | 48.2 |
| Roseburia_sp_CAG_471 | 135 | 23 | 78 | 43.4 |
| Rothia_mucilaginosa | 34 | 42 | 13 | 47.2 |
| Ruminococcaceae_bacterium_D16 | 55 | 107 | 97 | 119.2 |
| Ruminococcaceae_bacterium_D5 | 16 | 22 | 6 | 23.2 |
| Ruminococcus_bicirculans | 40 | 136 | 88 | 113.4 |
| Ruminococcus_bromii | 37 | 105 | 64 | 85.6 |
| Ruminococcus_callidus | 99 | 96 | 76 | 87 |
| Ruminococcus_gnavus | 175 | 176 | 175 | 173 |
| Ruminococcus_lactaris | 84 | 19 | 23 | 21.6 |
| Ruminococcus_torques | 173 | 72 | 146 | 81.4 |
| Ruthenibacterium_lactatiformans | 125 | 153 | 151 | 155.6 |
| Slackia_isoflavoniconvertens | 111 | 73 | 49 | 32.6 |
| Streptococcus_australis | 98 | 117 | 99 | 105.4 |
| Streptococcus_mitis | 83 | 123 | 132 | 109.6 |
| Streptococcus_parasanguinis | 68 | 48 | 40 | 47.2 |
| Streptococcus_salivarius | 93 | 87 | 81 | 85.4 |
| Streptococcus_sp_A12 | 64 | 142 | 101 | 119.8 |
| Streptococcus_thermophilus | 46 | 95 | 46 | 68.6 |
| Sutterella_parvirubra | 167 | 99 | 131 | 74.4 |
| Turicibacter_sanguinis | 1 | 12 | 2 | 11.8 |
| Turicimonas_muris | 10 | 65 | 44 | 81 |
| Veillonella_atypica | 20 | 46 | 20 | 39.6 |
| Veillonella_dispar | 21 | 18 | 34 | 32.4 |
| Veillonella_infantium | 28 | 14 | 19 | 31 |
| Veillonella_parvula | 2 | 37 | 10 | 35 |
| Veillonella_rogosae | 38 | 39 | 52 | 53.4 |
| Veillonella_sp_T11011_6 | 76 | 21 | 37 | 32.8 |
| Victivallis_vadensis | 35 | 111 | 67 | 66.4 |

Table 5 (Part 2C): Ranks

| | Habitual | | | Final |
|---|---|---|---|---|
| Profile | Diet | Personal | Postprandial | Rank |
| Category | | | | |
| [Collinsella]_massiliensis | 145.25 | 126.25 | 128.8333 | 129.533333 |
| Actinomyces_odontolyticus | 79 | 65.25 | 59 | 69.7125 |
| Actinomyces_sp_ICM47 | 98.75 | 67.25 | 141.5 | 109.675 |
| Adlercreutzia_equolifaciens | 89.75 | 95.75 | 104.1667 | 96.066667 |
| Agathobaculum_butyriciproducens | 8.25 | 100.25 | 79.16667 | 65.416667 |
| Akkermansia_muciniphila | 109.5 | 94.25 | 66.33333 | 87.020833 |
| Alistipes_finegoldii | 127.25 | 119.5 | 88.5 | 111.8125 |
| Alistipes_indistinctus | 89.75 | 67.75 | 85.33333 | 84.758333 |
| Alistipes_inops | 88.75 | 92.75 | 84.5 | 83.65 |
| Alistipes_onderdonkii | 71.75 | 64 | 60.33333 | 55.920833 |
| Alistipes_putredinis | 145.5 | 113.75 | 92.16667 | 112.704167 |
| Alistipes_shahii | 66.75 | 71 | 69.16667 | 71.229167 |
| Anaeromassilibacillus_sp_An250 | 165.25 | 130.75 | 57.83333 | 115.658333 |
| Anaerostipes_hadrus | 10.75 | 87 | 98 | 67.3875 |
| Anaerotruncus_colihominis | 167.5 | 151.25 | 168.5 | 164.9125 |
| Asaccharobacter_celatus | 92.25 | 92 | 84.5 | 85.1875 |
| Bacteroides_caccae | 139 | 100.5 | 115.5 | 112.95 |
| Bacteroides_cellulosilyticus | 59.75 | 74.5 | 72.83333 | 70.170833 |
| Bacteroides_clarus | 70.5 | 85.25 | 44.33333 | 63.420833 |
| Bacteroides_dorei | 53.5 | 83 | 99 | 86.925 |
| Bacteroides_eggerthii | 48.25 | 105.5 | 79 | 75.7875 |
| Bacteroides_faecis | 72.75 | 72.75 | 104.1667 | 81.016667 |
| Bacteroides_faecis_CAG_32 | 59.75 | 101.75 | 123 | 97.225 |
| Bacteroides_finegoldii | 73.5 | 72.25 | 84.16667 | 71.479167 |
| Bacteroides_fragilis | 123 | 151.75 | 148.5 | 142.2625 |
| Bacteroides_galacturonicus | 55.25 | 66.5 | 94.83333 | 76.095833 |
| Bacteroides_intestinalis | 73.75 | 58.5 | 72.66667 | 61.029167 |
| Bacteroides_massiliensis | 22.25 | 53.75 | 63.66667 | 44.766667 |
| Bacteroides_nordii | 24.75 | 71.25 | 83.16667 | 57.191667 |
| Bacteroides_ovatus | 33.75 | 102 | 105.1667 | 92.329167 |
| Bacteroides_salyersiae | 98.5 | 85.75 | 29.83333 | 65.170833 |
| Bacteroides_sp_CAG_144 | 108 | 61.5 | 78.5 | 80.75 |
| Bacteroides_stercoris | 102 | 91 | 88.33333 | 97.583333 |
| Bacteroides_thetaiotaomicron | 69 | 98 | 123.5 | 104.475 |
| Bacteroides_uniformis | 115 | 134.5 | 150.1667 | 140.416667 |
| Bacteroides_vulgatus | 102.5 | 97 | 145.1667 | 118.416667 |
| Bacteroides_xylanisolvens | 53.5 | 64.75 | 73.5 | 66.0375 |
| Barnesiella_intestinihominis | 110 | 93.75 | 79.16667 | 93.279167 |
| Bifidobacterium_adolescentis | 74.5 | 121.25 | 79 | 99.8375 |
| Bifidobacterium_animalis | 5.5 | 47.5 | 36.33333 | 28.933333 |
| Bifidobacterium_bifidum | 154.25 | 127.25 | 111.5 | 131.5 |
| Bifidobacterium_catenulatum | 154 | 144.5 | 101.8333 | 136.283333 |
| Bifidobacterium_longum | 149.25 | 130.5 | 103.3333 | 126.570833 |
| Bifidobacterium_pseudocatenulatum | 43 | 109.5 | 83 | 75.925 |
| Bilophila_wadsworthia | 148.75 | 128.25 | 116.5 | 130.675 |
| Blautia_hydrogenotrophica | 139.75 | 157.5 | 158.8333 | 153.470833 |
| Blautia_obeum | 116 | 122 | 150.1667 | 129.991667 |
| Blautia_wexlerae | 42.75 | 95.5 | 146.3333 | 101.745833 |
| Butyricimonas_synergistica | 103.5 | 70.25 | 68.66667 | 71.154167 |
| Butyricimonas_virosa | 131.75 | 53.75 | 91.83333 | 84.483333 |
| Clostridium_asparagiforme | 100.25 | 149.25 | 158.6667 | 140.291667 |
| Clostridium_bolteae | 157 | 163.25 | 173.1667 | 166.954167 |
| Clostridium_bolteae_CAG_59 | 138.5 | 163.75 | 169.8333 | 160.970833 |
| Clostridium_citroniae | 109.5 | 142.25 | 151.5 | 139.7625 |
| Clostridium_disporicum | 123.25 | 65.5 | 19.66667 | 57.454167 |
| Clostridium_innocuum | 146 | 167.75 | 168.5 | 163.5625 |
| Clostridium_lavalense | 117 | 131.75 | 154.3333 | 142.170833 |
| Clostridium_leptum | 170 | 138.5 | 112.8333 | 142.283333 |
| Clostridium_saccharolyticum | 155.75 | 113.5 | 119 | 129.6125 |
| Clostridium_sp_CAG_167 | 16.5 | 25.25 | 20.33333 | 19.970833 |
| Clostridium_sp_CAG_242 | 89.25 | 89.75 | 41.5 | 70.425 |
| Clostridium_sp_CAG_253 | 67 | 65.5 | 69.66667 | 67.141667 |
| Clostridium_sp_CAG_58 | 143 | 158 | 157.8333 | 154.358333 |
| Clostridium_spiroforme | 161.5 | 155 | 159.3333 | 160.258333 |
| Clostridium_symbiosum | 157.5 | 163.75 | 168.3333 | 165.795833 |
| Collinsella_aerofaciens | 134.5 | 135.5 | 124.8333 | 128.708333 |
| Collinsella_intestinalis | 152.75 | 153.75 | 147.6667 | 152.341667 |
| Collinsella_stercoris | 94.5 | 111.75 | 124.5 | 113.3375 |
| Coprobacter_fastidiosus | 120.75 | 106 | 131 | 124.6375 |
| Coprobacter_secundus | 91 | 66.75 | 52.83333 | 56.595833 |
| Coprococcus_catus | 47.75 | 53.75 | 43.5 | 44.25 |
| Coprococcus_comes | 142.75 | 96 | 83.5 | 93.3125 |
| Coprococcus_eutactus | 56.5 | 53 | 32.33333 | 42.008333 |
| Desulfovibrio_piger | 94.75 | 83.75 | 95 | 85.275 |
| Dialister_invisus | 75.25 | 83.25 | 70.16667 | 86.266667 |
| Dielma_fastidiosa | 123.75 | 107.25 | 157.5 | 134.075 |
| Dorea_formicigenerans | 30.75 | 112.25 | 125 | 100.55 |
| Dorea_longicatena | 118.75 | 111.25 | 67 | 89.25 |
| Eggerthella_lenta | 145.75 | 149 | 158.3333 | 154.570833 |
| Eisenbergiella_massiliensis | 120.5 | 125 | 135.6667 | 132.591667 |
| Eisenbergiella_tayi | 153.75 | 122.75 | 135.8333 | 139.283333 |
| Enorma_massiliensis | 128.25 | 111 | 92.66667 | 98.479167 |
| Enterorhabdus_caecimuris | 96 | 86.75 | 97.33333 | 87.870833 |
| Escherichia_coli | 159.25 | 147.75 | 144.1667 | 152.891667 |
| Eubacterium_eligens | 9.5 | 18 | 47 | 20.875 |
| Eubacterium_hallii | 25.5 | 75.75 | 57.16667 | 50.154167 |
| Eubacterium_ramulus | 49.5 | 58.75 | 83.16667 | 59.454167 |
| Eubacterium_rectale | 73.5 | 127.25 | 130.8333 | 119.295833 |
| Eubacterium_siraeum | 115.25 | 33 | 41.66667 | 60.629167 |
| Eubacterium_sp_CAG_180 | 132 | 99 | 107.6667 | 110.766667 |
| Eubacterium_sp_CAG_251 | 89.5 | 94.5 | 69.5 | 86.725 |
| Eubacterium_sp_CAG_274 | 48.5 | 161 | 115.6667 | 111.341667 |
| Eubacterium_sp_CAG_38 | 41.25 | 101.5 | 71.66667 | 74.704167 |
| Eubacterium_sp_OM08_24 | 100 | 116 | 108.8333 | 115.008333 |
| Eubacterium_ventriosum | 128.75 | 138.5 | 116.5 | 132.3375 |
| Faecalibacterium_prausnitzii | 25.75 | 37.75 | 27.5 | 27.65 |
| Firmicutes_bacterium_CAG_110 | 117.5 | 21.5 | 18.66667 | 42.366667 |
| Firmicutes_bacterium_CAG_145 | 142 | 92.75 | 120.6667 | 124.154167 |
| Firmicutes_bacterium_CAG_170 | 25.75 | 4.25 | 21.66667 | 13.966667 |
| Firmicutes_bacterium_CAG_238 | 36.75 | 25.75 | 52.33333 | 36.008333 |
| Firmicutes_bacterium_CAG_83 | 80 | 117.5 | 92 | 99.275 |
| Firmicutes_bacterium_CAG_94 | 173.25 | 130.5 | 99 | 134.8375 |
| Firmicutes_bacterium_CAG_95 | 17.25 | 15 | 3.833333 | 9.670833 |
| Flavonifractor_plautii | 169.5 | 159.75 | 170.6667 | 167.879167 |
| Flavonifractor_sp_An100 | 99.25 | 36.5 | 28.5 | 57.0125 |
| Fretibacterium_fastidiosum | 56.5 | 61.5 | 37.66667 | 47.566667 |
| Fusicatenibacter_saccharivorans | 47.5 | 104.75 | 113.8333 | 97.520833 |
| Gemella_sanguinis | 131.25 | 121.75 | 154.6667 | 142.116667 |
| Gemmiger_formicilis | 91.75 | 66.5 | 103 | 81.0625 |
| Gordonibacter_pamelaeae | 126.5 | 130.25 | 126.6667 | 134.004167 |
| Haemophilus_parainfluenzae | 12.75 | 18.25 | 6.166667 | 10.091667 |
| Haemophilus_sp_HMSC71H05 | 27.5 | 85.75 | 32.33333 | 43.645833 |
| Harryflintia_acetispora | 142.75 | 99.75 | 92.33333 | 112.258333 |
| Holdemanella_biformis | 59.25 | 67.5 | 60.33333 | 56.970833 |
| Holdemania_filiformis | 133 | 115.25 | 141.1667 | 128.804167 |
| Hungatella_hathewayi | 123 | 129 | 150 | 136.75 |
| Intestinibacter_bartlettii | 115.5 | 90.75 | 45.83333 | 82.120833 |
| Intestinimonas_butyriciproducens | 85.25 | 65.5 | 44.5 | 64.5625 |
| Lachnospira_pectinoschiza | 87.5 | 85.25 | 98.5 | 90.1125 |
| Lactobacillus_rogosae | 48.5 | 48.5 | 61.83333 | 51.458333 |
| Lactococcus_lactis | 72.75 | 62 | 86.16667 | 81.079167 |
| Lawsonibacter_asaccharolyticus | 117.25 | 116.5 | 99.83333 | 114.545833 |
| Methanobrevibacter_smithii | 105.5 | 54.25 | 66 | 70.1875 |
| Monoglobus_pectinilyticus | 48 | 116.25 | 127 | 107.3125 |
| Odoribacter_splanchnicus | 111.5 | 75.5 | 64.33333 | 74.183333 |
| Olsenella_scatoligenes | 53.5 | 83.25 | 106.6667 | 82.404167 |
| Oscillibacter_sp_57_20 | 5.5 | 10.5 | 20.16667 | 12.191667 |
| Oscillibacter_sp_CAG_241 | 130.75 | 60.25 | 73.33333 | 74.933333 |
| Oscillibacter_sp_PC13 | 25.5 | 32 | 16.16667 | 20.466667 |
| Parabacteroides_distasonis | 96 | 103 | 141 | 118.5 |
| Parabacteroides_goldsteinii | 59 | 36 | 65.66667 | 60.016667 |
| Parabacteroides_johnsonii | 56.75 | 131.25 | 128.1667 | 109.541667 |
| Parabacteroides_merdae | 157.75 | 96.25 | 85 | 105.25 |
| Paraprevotella_clara | 51 | 28.5 | 51 | 38.375 |
| Paraprevotella_xylaniphila | 44.5 | 21.75 | 48.16667 | 33.904167 |
| Parasutterella_excrementihominis | 47 | 93.75 | 53 | 68.0375 |
| Phascolarctobacterium_faecium | 73.5 | 98.25 | 107.6667 | 94.054167 |
| Prevotella_copri | 31 | 22.25 | 30.5 | 24.4375 |
| Proteobacteria_bacterium_CAG_139 | 65.75 | 98.5 | 80.66667 | 85.329167 |
| Pseudoflavonifractor_capillosus | 160 | 128.5 | 98.16667 | 131.416667 |
| Pseudoflavonifractor_sp_An184 | 168.5 | 116.5 | 98.16667 | 128.041667 |
| Romboutsia_ilealis | 31 | 41.5 | 24.5 | 31.45 |
| Roseburia_faecis | 51.5 | 122.5 | 115.5 | 99.125 |
| Roseburia_hominis | 20 | 64.25 | 37 | 39.3625 |
| Roseburia_intestinalis | 67 | 70.25 | 99.66667 | 83.329167 |
| Roseburia_inulinivorans | 132.75 | 115.5 | 129.3333 | 131.995833 |
| Roseburia_sp_CAG_182 | 4.25 | 20.25 | 26.66667 | 14.641667 |
| Roseburia_sp_CAG_309 | 69 | 58.75 | 26.66667 | 50.654167 |
| Roseburia_sp_CAG_471 | 24.25 | 38.25 | 53.66667 | 39.891667 |
| Rothia_mucilaginosa | 46.75 | 39.75 | 27.16667 | 40.216667 |
| Ruminococcaceae_bacterium_D16 | 139.75 | 108 | 70 | 109.2375 |
| Ruminococcaceae_bacterium_D5 | 57.5 | 20 | 40.5 | 35.3 |
| Ruminococcus_bicirculans | 109.25 | 139 | 92.5 | 113.5375 |
| Ruminococcus_bromii | 115.25 | 84 | 82.83333 | 91.920833 |
| Ruminococcus_callidus | 97.5 | 56.75 | 82.83333 | 81.020833 |
| Ruminococcus_gnavus | 139.5 | 153.5 | 173 | 159.75 |
| Ruminococcus_lactaris | 37.5 | 27.75 | 50.33333 | 34.295833 |
| Ruminococcus_torques | 130.5 | 95.75 | 108.6667 | 104.079167 |
| Ruthenibacterium_lactatiformans | 167 | 103.75 | 143.5 | 142.4625 |
| Slackia_isoflavoniconvertens | 54 | 69.75 | 62.16667 | 54.629167 |
| Streptococcus_australis | 53.5 | 59.5 | 106.1667 | 81.141667 |
| Streptococcus_mitis | 142.5 | 74.5 | 115.8333 | 110.608333 |
| Streptococcus_parasanguinis | 113 | 39.25 | 55.66667 | 63.779167 |
| Streptococcus_salivarius | 106.25 | 72.5 | 78.83333 | 85.745833 |
| Streptococcus_sp_A12 | 44.25 | 81.75 | 119.8333 | 91.408333 |
| Streptococcus_thermophilus | 50.75 | 97.75 | 71.16667 | 72.066667 |
| Sutterella_parvirubra | 55.5 | 80.75 | 111.3333 | 80.495833 |
| Turicibacter_sanguinis | 68 | 61.75 | 20.83333 | 40.595833 |
| Turicimonas_muris | 78.75 | 64.75 | 63.16667 | 71.916667 |
| Veillonella_atypica | 28.25 | 31.75 | 31.83333 | 32.858333 |
| Veillonella_dispar | 21.25 | 25.5 | 21 | 25.0375 |
| Veillonella_infantium | 24 | 36 | 18.16667 | 27.291667 |
| Veillonella_parvula | 46.75 | 41.5 | 17.16667 | 35.104167 |
| Veillonella_rogosae | 15 | 46.25 | 42.33333 | 39.245833 |
| Veillonella_sp_T11011_6 | 34.25 | 37.75 | 40.16667 | 36.241667 |
| Victivallis_vadensis | 103.5 | 74.5 | 79.16667 | 80.891667 |

### (X) CLOSING PARAGRAPHS

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect, in this context, is an alteration in the correlation between the presence, absence, or abundance of a microbe with a selected biological condition, or an alteration in a microbiome in a subject.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification shall be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, database entries, online resources, journal articles, and other written or otherwise memorialized text throughout this specification (referenced materials herein). Each of the referenced materials are individually incorporated herein by reference in their entirety for their referenced teaching, as of the filing date of this application.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

1. A method of using a group of microbes to determine a health condition in a human subject, wherein the group of microbes comprises:
at least two pro-health indicator microbes; or
at least two poor health indicator microbes; or
at least two pro-health indicator microbes and at least two poor health indicator microbes;
wherein at least one of the pro-health indicator microbes is selected from the group consisting of Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; and
wherein at least one of the poor health indicator microbes is selected from the group consisting of Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli;
wherein the method comprises:
obtaining a biological sample from the human subject; and
detecting the presence, absence, or abundance of the at least two pro-health indicator microbes and/or the at least two poor health indicator microbes in the biological sample.

2. (canceled)

3. The method of claim 1, further comprising:
identifying in the biological sample at least 10, at least 20, at least 30, at least 40, at least 50, at least 60, at least 70, at least 80, at least 90, at least 100, at least 125, at least 150, at least 175, at least 200, or more than 200 different microbes in the biological sample; and
determining the health condition of the human subject based on presence, absence, and/or absolute or relative abundance of the identified microbes in the biological sample.

4. The method of claim 1, comprising analyzing the biological sample to determine presence, absence, or abundance of:
at least three pro-health indicator microbes;
at least five pro-health indicator microbes;
at least ten pro-health indicator microbes; or
more than 10 listed pro-health indicator microbes.

5. The method of claim 1, comprising analyzing the biological sample to determine presence, absence, or abundance of:
at least three poor health indicator microbes;
at least five poor health indicator microbes;
at least ten poor health indicator microbes; or
more than 10 listed poor health indicator microbes.

6. The method of claim 1, wherein the group of microbes comprises Clostridium innocuum, C. symbiosum, C. spiroforme, C. leptum, and C. saccharolyticum.

7. The method of claim 1, wherein the group of microbes comprises P. copri and Blastocystis spp.

8. The method of claim 1, wherein the health condition comprises at least one of: overall good health, overall poor health, obesity, BMI, diabetes risk, cardiometabolic risk, cardiovascular disease risk, or postprandial response to food intake.

9. The method of claim 1, wherein the biological sample from the human subject is a microbiome sample from the human subject.

10. The method of claim 1, wherein the detecting comprises one or more of:
sequencing one or more nucleic acids of a pro-health or poor health microbe,
hybridizing a nucleic acid probe to a nucleic acid of a pro-health or poor health microbe,
detecting one or more proteins from a pro-health or poor health microbe, or
measuring activity of one or more proteins a pro-health or poor health microbe.

11. The method of claim 9, wherein the detecting comprises shotgun metagenomics.

12. The method of claim 1, wherein the biological sample comprises a stool sample.

13. A method of predicting a health condition in a subject, comprising:
determining presence, absence, or relative abundance of at least three pro-health indicator microbes in a microbiome of the subject;
determining presence, absence, or relative abundance of at least three poor health indicator microbes in a microbiome of the subject; and
predicting the health condition of the subject, based on the presence, absence, or relative abundance of the pro-health and/or poor health indicator microbes in the microbiome of the subject;
wherein at least one of the pro-health indicator microbes is selected from the group consisting of Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; and
wherein at least one of the poor health indicator microbes is selected from the group consisting of Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli.

14. The method of claim 13, wherein:
the health condition comprises at least one of obesity, increased cardiometabolic risk, diabetes risk, or overall poor health; and the health condition is predicted by the presence and/or abundance of more poor health indicator microbes than pro-health indicator microbes; and/or
the health condition comprises at least one of overall good health or absence of obesity, reduced cardiometabolic risk, or reduced diabetes risk; and the health condition is predicted by the presence and/or abundance of more pro-health indicator microbes than poor health indicator microbes.

15. A method, comprising:
obtaining a microbiome sample from a non-diseased the human subject;
isolating a nucleic acid fraction from the microbiome sample;
detecting, within the nucleic acid fraction, presence, absence, or relative abundance of at least one unique marker sequence indicative of:
a pro-health indicator microbe selected from the group consisting of Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; or
a poor health indicator microbes selected from the group consisting of Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, and Flavonifractor plautii; and at least one of
determining the human subject has overall good general health if the pro-health indicator microbes outnumber or are relatively more abundant than the poor-health indicator microbes; or
determining the human subject has overall poor general health if the poor health indicator microbes outnumber or are relatively more abundant than the pro-health indicator microbes.

16. The method of claim 15, further comprising providing to the human subject a dietary recommendation based on the presence, absence, or relative abundance of one or more poor health indicator microbes and/or one or more pro-health indicator microbes.

17. An assay, comprising:
subjecting nucleic acid extracted from a test sample of a human subject to a genotyping assay that detects at least one of (A) Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; or at least one of (B) Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus qnavus, Flavonifractor plautii, Clostridium leptum, Ruthenibacterium lactatiformans, and Escherichia coli, the test sample comprising microbiota from a gut of the subject;
determining a relative abundance of the at least one of the detected (A) microbe(s) that is below a predetermined abundance, or a relative abundance of at least one of the detected (B) microbe(s); and
selecting, when the relative abundance of the at least one detected (A) microbe is below the predetermined abundance or when the relative abundance of the at least one detected (B) microbe is above the predetermined abundance, a treatment regimen that comprises at least one of:
(i) modifying microbiota of the gut of the subject using at least one of a prebiotic, probiotic, or pharmaceutical, or
(ii) altering the diet of the human subject.

18-38. (canceled)
